SEQUENCE LISTING (119 sequences)

SEQ ID NO: 1 - DNA, 6447 bp, Bos taurus
  source 1..6447: mol_type = other DNA
ctctgcgggatttcccccctgctctttggctgcttgttgagaggtttgcttatgggttagctttgaaaagattcaggttttactttccaatgagttttacattttagcagaactaggagattacttgcctggttcgtttcatttctgtaacaatgtttgaaagttacttctttagtagtcagataaatttctgtggttctaaatagttcgacgaggtctgaaaaatactaacatgtattaatacttttttattatattacaattttttattatgtttagtagaagagacacctttgccatttaaactttaacttttctcagtccttcagtgaatctgcagacctattttctcaggagctcagcctgaccctacttcagtgataaaggaggaaaggctggctacagtaaatatcattcaagatgtccagcaaagggagcagcacagatggcagaacagacttagctaatggaagcctgtctagcagtccagaggagatgtctggtgctgaggaggggagggagacatcctcaggcattgaagtggaggcttcagacctgagcttgagcttgaccggggacgatggtggccccaaccgcaccagcacagagagtcgaggcacagatacagagagctcaggtgaagacaaggactctgacagcatggaggacaccggccattactcggtcaatgacgagagccaagtccatgaccgctcggaggaggaggaggaggaggagcatcctcgtcgtcgtgtgcagcgcaagcgggccaatcgtgaccaggactcatcagacgatgagcgggccctggaagactgggtgtcctcagagacatcagccctgccccgacctcgctggcaagcccttcctgcccttcgggagcgggagctgggctcaagtgcccgctttgtgtatgaggcttgtggggcaagagtctttgtgcagcgtttccgcctgcagcatgggcttgaaggccatactggctgtgtcaatactctgcactttaaccaacgcggcacctggctggccagtggcagcgatgacctgaaagtggtggtttgggactgggtgcggcggcagccagtattggactttgagagcggccacaagagcaatgtcttccaggccaagttccttcctaacagtggtgattccaccctggccatgtgtgcccgtgatggacaggttcgggtagcagagctgtctgctacacagtgctgcaagaacacaaagcgtgtggcccagcacaagggagcatcccacaagctagccctggaaccagattctccctgtacgttcctgtctgccggtgaagatgcagttgtcttcaccattgacctcagacaagaccgaccagcttcgaaactggtggtgacaaaagaaaaggagaagaaggtggggctgtatacaatctatgtgaatcctgccaacacccaccagtttgcagtgggtggacgagaccagtttgtaaggatttatgaccagaggaagattgatgagaatgaaaacaatggagtactcaagaaattctgccctcatcacctggtgaacagtgagtccaaagcaaacatcacctgtcttgtgtacagccacgacggcacagagctcctggccagttacaatgatgaagacatttacctcttcaactcctctcacagtgatggggcccagtatattaagagatataagggccacagaaataatgccacagtaaaaggcgtcaatttctatggccccaagagtgagtttgtggtgagcggtagtgactgtgggcacatcttcctctgggagaaatcatcctgccagatcattcagttcatggagggggacaagggaggcgtggtaaactgtcttgagccccaccctcacctgcctgtgctggcaaccagtggcctagaccatgatgtcaaaatctgggcgcccacagctgaaacttccactgaacttacagggttaaaggatgtgattaagaagaacaagcgggaacgggatgaagatagcttgcaccacactgacctgtttgatagccacatgctctggttcctcatgcaccacctgagacagaggcgccatcaccggcgctggcgagaacctggggttggggccacagacgccgactctgatgagtctcccagctcctcagacacatcggacgaggaggagggtcctgaccgggtgcagtgcatgccatcctgaggcctcatacccaggaggggtgggctggggctgccaacccaatcctgcctgggcagccctttcctgtgccaggccctaacattcagcagaaacgcactttggacttttttgctttagatttaaaaaaaaaaaaaaaagacatcccaggaggacaagccagaggggagtgaacctacagagagtgtctaggagtgggagttgagccctggaatggggctccatggagaggtgcataggactcagcagaaatggcttctccccaaaccctttttgggggggaagggagagcctattttattaactggtttggggtagggaatggggtttccttttctttggtctcccttgtttcttgggtggcgggtggggggataactggctgttcagtaccgaggggccagactgggggtggtagaaatgccacactctctttggtttaggtttttgactttattttcttttattttttaaaagtctatgacagttgcgttgtttttctccgcccgcctgccccccacccccgagcaaccccatcccaggatcctgtctttctgggaagtctttttaatacgagtccctaaccatcctacactcttcactttcctttccaccccattcattctctgaacttatcacagtgttttgcacttgattatgtatccttcactttcttctttatcccatcatcccctcagtaggtctggtgagggaggttggggggaggtgggaggaggggcagaagtggaagcataggaaggatgttacctcttctcttactttaaaaaaaaaagttgtttggtggcagcaatctccctgtccctatcactgttagaggcctaattttatatctataaatatattaaaaagcaactcaaaattggatgtatcccgttaacattattgtcaaagtttaaatacctatatattctctatgactgcaataaagggacttccaggagagagtgagtgaaagtaatggtatggaggtgtcacctggggtcagcctaccctctgtgcatggccattggagattggggcttaactctttaggtttcctggaggctcaggaatggaaggattctgcccttccatgctttcccaccatggtttcagggttgaggaaaactcctttcagctttttgccccagatatcctacctctccttaaatcttgtaagggttcttaggatggctcttctagccagagactggcttatctttaaaacttgactgaactatgacttcaggaagggtcctgccactgcagaccatttctcctagtaccgtgtgtgatagaagacacactcactctgacctttatctgcttgattctttaagctatccagcttcacaccagctcccttcctaatggaagagtatagggaaagacctcctgtgtttgactttctaccttgtccaccttcccatatcttgggattgaaggacaaggcattaatctaaggagtgattaaggtggctggggctcagacatctgttgtgtcagtggtcaataaataggaaagtcccagttgaattcttgcccttaaatgcttttgctgctatttgtttgttcccagctaggaattctgcatcctgggacagtcagattccactagaacaggccaggaaggaagggaagagtgaaaggatggaattcccagacactccttcctcctgtcccttttcctagcagctctcagaccagatgttagctgctacacttccttccagaggtagggaatgtgtggtggctgtgagtggtctgtgttcttattgctggtagggtgatggagtgggctgaaaccatgcactctggaatttgttgtgtattttctctcagtaaagcttttttttgtttttcctgactgctgctctatgtgtgatatgtgagccattcacttgtgatggtctatttttgctcctttcagtatggtgcctgaccttagttttacaattttaggaagtccagcttggtatatccatgtttccaacagttgggctccagctggagactgtgtaagtggaacaggcctcacagttcagtgatacttcgaagatgagggaagggagagatactacttagggatattttcctgacctctgttccaggctgcagttaaggatttgggggtaaattttgctctccctcccttcccagtcatccaaagtctccaaaatcttggtcactcacagaaattctaaaatcagttaggtacagtacaatcaatcacctttattccagggttagaacaaggccgtgcacactgcagacagaggagcagagaatggggcagtctcacagcagtatgggggtaggagggcaggttagtctttttagattattttgccttacagagaaatttactacactccgctgaaaatgaaccctgtctcctttctctcatctcatcctctcctctcttcaaaggaagcctcgctttgaagagcctctttattccccgcttctctctctctctgactccatctcacgtaacgtatcctggcttggaaggacaattcatgctcattttccacctcccaattctacaagtatctctctctccttgagggagaaagccagacagggctggggaaacttgggaaggtcctttaaggcaaaactgtggagtactcgaggtgtatgttttaaggcaggtgtccgcagcagtggccagaggggacgggctgggctgggctgggctggtagagggtgctctccagccagtctggcttcttccctctccaacagccagcccttccctcccactgaagttttgttcccaacatcccaaaagctccccacctcacttaaactctctaaatagtctcttgagattgcatgtgaaattaaagtttaaaaaagttttctttttcgactatatagcaatttgactctgccactcccttcactgtggcattctccttccacccccgcccgggccttgacaataagttaatgctagacaccacagaaaaagggtacacatggcagtgcgggtttaatatacatatatgtagaatatatatgtacacacagttagcacggtcagggtggggagggctcactcgggacaggcaggaggctggggtcactgtactctagtgatttggctaggaggcgttcacaagcccaaggtgccaggatattgagggagaggggttactaagccccctcctggactgaattggggatcagtgcaacggaaacactggagttagcttcttcccttctttctaccttgggggaggggcttctagaccacggagagagggaacgttgcctgcaataaccacgttgggactctgtaggtggaaacaggactggaattaaaacctaaggatttctattctaggaactcccaaggctagtatgtgcaacagtctgagtaggaataaaggagtagaaaagaggactcctctctgccttcatcccctcaggctcagtggtgggggtcggtctgttcccagcattgtctctccagaggtgggggatggggctggaatctagtgctgggttctcatggggtttgaacttgggggccgggaaaggatccagtgatctctctcttcaggtccctggctcacacccaccctaggcctatgaaaggggagcaaaaggggacctgctggtgcccaggccctctaacctgaggaacagtcggaaacaggactcctcagccagtccagcattggggtacaaacacaaactctggcttctctttgcccctgccaggtgggggttgggtggcttcttcatctttcctctcactgcttttcattagattcacccaactctcccatgccttactcttgccttgcctcctcaatgtaggaaaacacaggtcctttctgctcccacacttggaggtgggggaagagtggaggaaagggaagtaagagaatgtgtctatttctatttgcatagaaatagcccccaactccccctggagtggggaaactgagccccaccaccacccccagcccctgacccccatcccctcacaaggaggaggaactagatgagctagacatgtagacagacagccagagcgtgcgcacacgcacacacacgcgcacagtaaggggttaggaccaggttagtaagtgggtagatgggggagggctgcccttgcccctcccctgcaggatggggaaagagtagtctgatgaaggtccaaccttcctcctcttcctcccccttgctcaggctttcttcggtggaggagccaatttgatgatctcttcctcagag

SEQ ID NO: 2 - DNA, 2374 bp, Bos taurus
  source 1..2374: mol_type = other DNA
agaggcgcgcggcgaccgagcggcagcagggagctggcttatagctcaccacgggtggggagacaaggagcttcagtgggcagcgagtggctggatccagcaagccctcccttccccacgcctgcagcatagaggcctactctgtccttggaacagcctctgtcaggacgcagaatcagccctcctggccatgggaggggcagtgatcgacgagggcccgaccggcatcaaggccccggatgggggctggggctgggccatcctttggggctgtttcgtcatcacgggcttctcctacgccttccccaaggcggtcagcgtcttcttcaaggaactcatgcgagagtttgggataggctacagtgacacagcctggatctcctccatcctgctggccatgctgtatgggacaggcccactctgcagcgtgtgtgtgaatcgctttggctgccggccagtcatgcttgcgggcggcctcttggcgtccctgggcatggtgtctgcgtccttctgtggaagcatcatccagctctacctcaccacaggggtccttactggcttgggtttggcgctcaacttccagccctcactcatcatgctcaaccgctacttcaacaagcggcgccccatggccaacgggctggcggcagcaggcagcccggtgttcctgtgcgccctgtccccgctggggcaggtgctgcaggaccactatggatggcggggcggcttcctcatcctgggtggcctgctgctcaattgctgcgtgtgtgccgcgctcatgcggcccctggaggcgccccggcagagttcaggttcggggcctgcaccccagcggccgccccggcggctgttggacctgagcgtcttcagggaccgtggcttcgtcatctatgccctggccgcctccatcatggtgctggggctctttgtaccacctgtgtttgtggtgagctatgccaaggacctgggggtgcccgacactcaggcggccttcctgctcaccatcctgggcttcgtcgacatctttgctaggcccaccgctggcttcatcacggggctcaagaaggtgcggccctactctgtctacctcttcagctttgccatgttcttcaacggcttcgctgacctcacggggtccactgccagcaactacggtgggctggtggtcttctgcatcttcttcggcatctcctatggcatggtgggggccctgcagttcgaggtgctcatggccatcgtgggcacccagaagttctccagcgccattggcctcgtgctgctgctggaggccatagctgtgctcattgggccgccgtccggaggcaagctcctggatgcgacgcacgtctaccagtacgtgtttatcctggcgggggccgaggtgctggcctcctcccttgtgctggtgctgggtaacttcttctgcattaggaagaggcccgagacggccacggaggaggagcgccacaagcccccggaggatgtcagggtggactcccgggaggtggagcagttcctgaagacagagcctgagaagaacggggaggtggttcacaccccggaaacgagcgtctgagcccagggaggacggcaggggtgcagggaaccggacagagaccgtcaacgctcgtatttatttcacaaacaggaccagcttgggcggggccttcggctctgcgccggctgcccgggcagtggatcatcacccgatcagtgtttttcaggggaaggtggcggggtgggaacgtgtcattccgaggcggatctgtggtgaagccaagccgcgaggttatgagccgtctgcaccagggaacccacctgctgaccccagggagcccagtgcccaccgctgggctcaaggacccagagacccgacgctttggagacaacacgactttaatcagggggcggggccggggacagctacggagcgggcactgccacaacaggcctccccatgtgcccatgagtgccctgtcctaccgggtctcctgtgtgccccagcccaccccttttgagtgtcttagtggacaaagctccctccttcgcctctgaaaggtttgagataaacctgctcgctcttagggacagcggtctaaccgttgaccccaaatctctttctcaaaagacagcctgcttatttttacaaattggtttcagtcctcagcagccacgtcagccttctaagtactgctcgttctggaccctgtcctgctggacctcaaacggctcagcgtgttcacacagcaccctggtggtcggcagggaaggggccacactgcggggatgtggacacctaggctgggatgtacatgtggtttctgttcactggtgtgtgtttgaagaaaatgatctgcctgcttttgtttattca

SEQ ID NO: 3 - DNA, 1305 bp, Bos taurus
  source 1..1305: mol_type = other DNA
cgctgaggtccttggtcctctggctccgggcgtgggcgggatgacgcgcttcgctctgaccgtggtccggcatggagaaacaagacttaacaaggagaaaataattcaaggacaaggaatagatgagcctctttcagaaactggatttaaacaagcagctgctgctggtatatttcttaaagatgtgaagtttactcatgttttctccagtgaccttacaaggacaaagcagaccgtgcatgggattttggagaagagcaaattttgcaaagatatgacagtaaagtatgattcaagactccgagaaaggaaatatggggttgcagaaggcaggccgctgagcgagctgagggccatggccaaagcggcaggggaggagtgcccagcattcacaccgcctggaggagagaccttggaccagctgaaaaggcgtgggaaagacttttttgaatttctttgtcaactgatcctgaaagaagccggtcagaatgaacagttttcccaggaagccccgagcagctgtctggaaagttccctggcagagatatttcctttagggaagaactgtgcctccacgtttaactctgacagcggcaccccgggcctagcagccagtgtcttagtcgtgagtcatggcgcctacatacgaagcctgttggattactttctgactgacctcaagtgctcgttcccagcgactctgagcaggtccgagctcacgtcagtcagccctaacacagggatgactgtcttcatcctaaactttgagaaaggaggcaaagggagaccaactgcccagtgtgtgtgtgtgaacctccagggccacctggccggggtgaacaaaactccctaaatctgaatcatctgcatggaaatcagcacatgtgagcctctgagggagtgcctcttgactttacttccgtcctctgctaatgcagatttggaaacagttaaaaagctgtctgttacagcaggttataaaactcgagtggaatcagagccatgtaaaacactaatagaaaaccatcgaaaatggacttctttggtgcaagtacagttggaattttccagggtaattttaccaccctgctcagtgacctctctgtattgtaaatggatgagggaactcagtattgtgaattgaggcatgaataaccctgttactatggtttggggtttttttttggttttttttttcacctttttaatatcttttttttttttttaatgtttttcttttatgtctgaaaaatctaacttgttttcttggaaaaaaaaaaaaaaaaaaaaaaaaaaaaaaaaaaa

SEQ ID NO: 4 - DNA, 1326 bp, Salmo salar
  source 1..1326: mol_type = other DNA
gaaagttgctactttttcgagacctaggataatggaggagcgttcaagtcaggtgttgaacccggagcgactgaaggcgctggagatgtggctgcaggagactgacgtcaaactgacccaggtcaatggccagaggaaatatggaggtccacctgatgactggcttggcgccccccctgggccgggctgtgaggtgttcatcagccagatcccgcgggatgtctttgaggaccagctgattccgctgttccgtgccgtgggccctctctgggagttccgcctcatgatgaacttcagcggacagaaccgtggctttgcctacgccaagtacgacagccctgcctcggccgctgccgccatccgctcgttgcatggccgtgccctcgagtcaggggcacgcctcggtgtacggcgcagcacggagaaacgtcagctctgtcttggggagctgcccaccagcacaaggagggagcaactgctgcaggtgctgctggacttctctgagggggtagagggcgtgtccctgagagcagggcctggggaacaggggatgtctgcagtggtggtctatgcctcccaccatgcagcttccatggccaagaaggtgctgattgaagcctttaaaaaacgcttcgggctggccatcactttgaagtggcagtcctcttctaggcccaagcacgaagagcctcccagaccctccaaaacccctccttcctctcctcccaaacctcctcgctgctccctcctggacagcccccggcctcccctgcacctcgcccagcgtcagctccctgccttctcccgggctgtgagggcgccctctcccatggtgcacgctgctcctgaatcccccaggggggcgaccatggtgcctcctgtggatgcagcagccctgctccagggtgtgtgtgaggtgtacgggcaggggaagcccctctatgacctgcagtaccgccacatggggcctgacgggttcctgtgcttcagctaccgggtgtatgtgccggggctggccacacccttcactgggatggtgcagactctgcccggccccacccctggagccatacaggaagaggctcgcagagctacagcccagcaggtcctcagcgctctgtacagggcctgatggtgttgaagcacagatcccctactttgttttaattatgaaaatacttaaatgttttgcactcttttatatttagtaagtagatgcatgattttacttttttttttgaaccacttttgcatgtttctgcaccatttaattgtttctcattataataaaatgagatttgtcaaaaaaaaaaaaaaaaaaaaaaa

SEQ ID NO: 5 - DNA, 3400 bp, Sus scrofa
  source 1..3400: mol_type = other DNA
atggtgctacttgaagactctggatctgcagactttagaagatgttctgcccatttaagttccttcacttttgctgtagtcgctgttctcagtgcctgcttggtcactagttctcttggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcagatggatctgatttagagatgaggctggtgaatggaggaaaccggtgcttaggaagaatagaagtcaaatttcaagagcggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgcgatcatgctgaggatgctggagtgatttgcttaaatggagcagacctgaaactgagagtggtagatggactcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctgatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcagggaaccagagtcagacactatccccgtgcaattcatcatcctcggacccatcaagctctattatttcagaagaaagtggtgttgcctgcatagggagtggtcaacttcgcctggtcgatggaggtggtcgttgtgctgggagagtagaggtctatcctggggcatcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcagagttcatgtctctgagactgatcagtgaaaacagcagagagacctgtgcagggcgcctggaagttttttacaacggagcttggggcagcgttggcaggaatagcatgtctccagccacagtgggggtggtatgcaggcagctgggctgtgcagacagaggggacatcagccctgcatcttcagacaagacagtgtccaggcacatgtgggtggacaatgttcagtgtcctaaaggacctgacacactatggcagtgcccctcatctccatggaagaagagactggccagcccctcagaggagacatggatcacatgtgccaacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagagcccgcatttggccaggggactgggcccatatggctcaatgaagtgaagtgcaaggggaatgaaccctccttgtgggattgtcctgccagatcctggggccacagtgactgtggacacaaggaggatgctgctgtgacgtgctcagaaattgcaaagagccgagaatccctacatgccacaggtcgctcatcttttgttgcacttgcaatctttggggtcattctgttggcctgtctcatcgcattcctcatttggactcagaagcgaagacagaggcagcggctctcagttttctcaggaggagagaattctgtccatcaaattcaataccgggagatgaattcttgcctgaaagcagatgaaacggatatgctaaatccctcaggagaccactctgaagtacaatgaaaaggaaaatgggaattataacctggtgagttcagcctttaagataccttgatgaagacctggacta

SEQ ID NO: 6 - DNA, 3884 bp, Sus scrofa
  source 1..3884: mol_type = other DNA
tctgtatttttgtggaagtgaaatgatttttagaattcttagtggttctcttcttcaggagaacatttctagggaataatacaagaagatttaaatggcataaaaccttggaatggacaaactcagaatggtgctacatgaaaactctggatctgcagactttagaagatgttctgcccatttaagttccttcacttttgctgtagtcgctgttctcagtgcctgcttggtcactagttctcttggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcagatggatctgatttagagatgaggctggtgaatggaggaaaccggtgcttaggaagaatagaagtcaaatttcaaggacggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgtgatcatgctgaggatgctggagtgatttgcttaaatggagcagacctgaaactgagagtggtagatggagtcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctgatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcagggaaccagagtcagacactatccccgtgcaattcatcatcctcggacccatcaagctctattatttcagaagaaaatggtgttgcctgcatagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctaagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcggagttcatgtctctgagactgatcagtgaaaacagcagagagacctgtgcagggcgcctggaagttttttacaacggagcttggggcagcgttggcaagaatagcatgtctccagccacagtgggggtggtatgcaggcagctaggctgtgcagacagaggggacatcagccctgcatcttcagacaagacagtgtccaggcacatgtgggtggacaatgttcagtgtcctaaaggacctgacacactatggcagtgcccatcatctccatggaagaagagactggccagcccctcagaggagacatggatcacatgtgccaacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagaggccgcatttggccaggggactgggcccatatggctcaatgaagtgaagtgcaaggggaatgaaacctccttgtgggattgtcctgccagatcctggggccatagtgactgtggacacaaggaggatgctgctgtgacgtgttcagaaattgcaaagagccgagaatccctacatgccacaggtcgctcatcttttgttgcacttgcaatctttggggtcattctgttggcctgtctcatcgcattcctcatttggactcagaagcgaagacagaggcagcggctctcagaagaatgctacctgtctttggggaagaaacagggttatccgatggcatcctgttgcgtttgagttcgtgacatcatgagggaaaggcttggcagcgtttactcgttttctcaggaggagagaattctgtccatcaaattcaataccgggagatgaattcttgcctgaaagcagatgaaacggatatgctaaatccctcagaaaattccaatgaataagatggttttaatgatgtgggactaatttttgtgtctaaatctcttcctatttctggatgaaaaaaaggagaccactctgaagtacaatgaaaaggaaaatgggaattataacctggtgagttcagcctttaagataccttgatgaagacctggactattgaatgagcaagaatctgcctcttacactgaagattacaatacagtcctctgtctcctggtattccaaagactgctgctgaatttctaaaaaatagattggtgaatgtgactactcaaagttgtatgtaagactttcaagggcattaaataaaaaagaatattgctga

SEQ ID NO: 7 - DNA, 3390 bp, Bos taurus
  source 1..3390: mol_type = other DNA
atggtgctacttgaagactctggatctgctggttttaaaaggtgttctgtccacttcggtcccttcactcttgctgtggtctcagttctctatgcctgtttggtcaccagtgctcttgggggaacagacaaggaactgaggctggtagctggtcaaaccaagtgttctgggagagtggaggtgaaagtccaggaagagtggggaactgtgtgcaacaccggctgggacttggctgccgtctctgtcgtttgtaagcagctgggatgtccgtctgtcatcaaagccacaggatggaccaattccagtgcaggcactgggcgcatttggatggatcatgtgtcttgtcgaggcaatgaatcagctctctgggactgcaaacatgaggggtggggaaagcataactgtactcaccaacaggatgtaggagtcacctgctcagatggatctgatttggagatgaggctgatgaatggaggaaaccggtgctcaggaagaatagagatcaaattccagggccagtggggaacagtgtgtgacgataacttcaacttggatcacgcttctgtggtttgtaaacagcttggatgtggaagtgctgtcagtttctctggctcagctaattttggagaagggtctgggccaatctggtttgatgatctggtatgccatggaaatgagtcagctctctggaactgcagacacgaaggatggggaaagcacaactgtgatcacgctgaagatgccggagtgatttgcttagagggagctgacctgagcctgagactggtagatggagtcaccaagtgttcaggaagattagaagtgagattccaaggagaatgggggacagtgtgtgatgatggctgggacagtgatgatgctgctgtagcctgtcagcaattgggatgtccgactgccatcactgccgtcggtagagtcaatgccagtgagggaactggacacatttggcttgacagtgtttcctgccaaggacatgaatctgctgtctggcagtgcagacatcacgaatggggaaagcattactgcaatcataacgaagatgctggtgtgacctgttctgatggatcagatctcgaactgagactcaaaggtggaggtagccgctgtgctgggacagtggaggtggaaattcagaaactgatagggaaagtgtgtgacagaagctggggactgaaagaagctgacgtggtttgcaagcagctgggatgtggatctgcgctcagaacatcctatcaagtttattctaaaattcaggcaacgaacacatggctgtttttaaacaactgcaatggaaatgagacttctatctgggactgcaagaactggcaatggggtgggcttagttgtgaacactatcatgaagccaaagttacttgctcagcccacagggaacccagactagttggaggggacattccttgctccggtcgtgttgaagtgaaacatggcgacacttggggcaccatctgtgattctgacttctctctggaatcggccagcgtgctatgcagggaattagagtgcggcagcgttgtctccatcctggggggagctcactttggagaaggaaatggacagatctgggctgaagaattccagtgtgaaggcaacgagtcccatctttcactctgccccgttgcaccccgcctggatgggacgtgtagccacagcaaggacattggagtagtctgctcaagatacacagaagttcgcttggtgggtggcaataccccatgtgaaggaagagtggaggtcaagattcttgggacctggggacctctctgcaactctcactgggacatggaagatgctcatgttttgtgccagcagcttaaatgtggagttgctgcttctatcccaggaagagcaccttttgggaaaggaagtgggcagccctggagacacatgttccactgcactgggacagaacagcacatgggtgattgtcctgtaactgctctgggtgcatcgctgtgtcctgaagggcaagtggcctctgtaatctgctcaggaaatcggagtcagacactatatccatgcaattcatcatcttcagatccggaaagctccgttgttttggaagagaatggtgttccttgcatagggagtggacagctccgcctggtaaatggaggtgggcgttgtgctgggagaatagaggtctatcatgagggctcctggggcaccatctgtgatgacagctgggacctggatgatgcccacgtggtgtgcaggcagctgggctgcggtgtggccattaatgccacaggttctgctcattttggggaaggatcagggcccatctggctggatgaggtgaactgtaatggaaaagaacctcgcatttctcagtgccgttcccatggctgggggcggcagaactgcaggcataaggaggatgcaggagttatctgctcagagttcatgtctctcagactcatcagtgacagcagcagcgagacctgtgcagggcgcctggaagttttttacaacggagcttggggcagcgtgggcaagagcgacatgtctgcaaccacggtgggggtggtatgcaggcagctcggctgtactgacaaagggagtatcagaccagcaccttcagacaaggtggagaacaggtacatgtgggtggacaatgtccggtgtcctaagggacctgaaaccttatggcagtgcccatcatctccgtggaagaggagactggccagcccctcagaggagacctggatcacatgtgctgataaaataaggcttcaggaaggaaccactaattgttctggacgcgtggaggtctggcacggaggttcctggggcacagtgtgtgatgactcctgggaccttaacgatacccaggtggtgtgtcgccagctgggctgtggcttagctctggaggcaggaaaagaggcagcgttcggccagggaactgggcccatatggctcaatgaagtgaagtgcaagggcaatgagtcctccttgtgggattgccctgccagatcctggggacacagtgactgcgggcacaaggaggatgcctctgtgaagtgctcagaaattgcagagagcaaaggatcagtaaaggccgcaggtcactcatccactgttgcacttggaatccttggggtcattctgttggcctttctcatcgcaaccctcttgtggattcagaggcgaagacagagacagcggcttgcagtttcctcaagaggagagaactctgtccacgaaattcaataccgggagatgaattcttgcctaaaagcagatgatctggacctatataattcctcaggtctgtgggttcttagagggtccatagccctggggttcagactggtaactgcagctgaggctgagaggcattctacttga

SEQ ID NO: 8 - DNA, 4655 bp, Sus scrofa
  source 1..4655: mol_type = other DNA
gggctgcacccatggcatatgaaacttcttgggctaggggtctaattggagataaagctgccagccacagccacagcaaaaatccgagccacatctgccacctacaccatagctcatggcaatgctggatccttaacccactgagcaaggccagggatcaaacccacaacgtcatggtttctagtgggattcgtttctgctgtgccatgacaggaactcctaaggaagcctctttcagttttccaaaaccagtacaaaagctgttatatctaaaacaactgtttgactagtaatcaagtcatgacttttttgattctaacttgttcctggacactctttttaaccaagatttgggctttattctctccagtctttgttccataaagtgagctatttgcttccgaatggcagagctggatgggggacatcttagcatttgattggttggcttttaagggattacttctctggaaaggcactgtcccatattatcacattgctagcagtgtggatttagttgtgatcaaatttttaaattgaagatgaacatttaccaccaaagaggacataaacttcatgactcctgtgttatatagattcctatacatcatattggtttaatgtgtttgtgatgaagagctctgttttaaaacaaccttaaataccacatccagaaataggaactgactctggcaaagaggttattcggggtcccaatatgtttaataacgaagtcaatacaattttaaaaagaataaattccaactacacaaatggactttaataagattaacaaaactgaggaagactgatacgtcaataagtcaacaagtatatataacatacccagatgaaataaaacaatgacttcccatttttatcttgcatttcccagctacttaagagtaaacatgatcacttttcaaatcccacttaatattttatctgaaagaagaatttactgagtaaaatgcttagcttcaggatctcacatttgcaatccatcaggttacaatattagttaccactgtttcttccctcggtgtaagcattctatatgtgacttttacttacattctgccactttttatttcttcctaacttttatatgctcaagaatgaaactgtattggtgttacgtaacatctcaaatttgcaacatacatattggtgtatctcacttgaacctacatccctgaaaaataaacacaatgtatccaaaagaactacctcggagtgtatgaggaggaagagcgtagctgtgtagaaataaaagaatggaaaaacagttttgtggagatgtatctgtccttgttgacaacaatccccatgatttattaacagacacaggcagccaacttagtacacaacaaggtcctagcacggaagttggtaatcaagtgggaattaaggctttaaggtctcttacaaccaatgtgattcaaaaaaaatccaaagcaagcaaaccgtacaacttggagaaagtgctccaaacagagcttctttggcaacccaaattgaaccaaatgactcaggtggacgattaaaataaactgtgcgccgttttatttcaccaggtcaataaagcccataaattatttttggtccagaaaaccaggagttacaagcaccttccttttcatttatttaaaatattcaatatattcagtatcaatttttagtccctgctcttcattcacttattgactaagatcctgtgtgccttttggtgatcattccttccaagctagtaaaacagcaaactgaaatgcccctccatgcagagggtcgagaccgagaggttaagtcacccaaaggtggacgcaccttgctttttatttactgaagtatgcgagggactacgatggccatggctggactacgacgaggcgggaggcgaaggggcgcgcgggctgtgggctgtagctcaggggttggaacccgcaaaagagatgggggcagcagccaccctccactagctcggcccaaaggacccagaaaaagaccacccctccccccactgcacccttgacccctccccctctttccaggcctttcatccgcccgcccgcggggcagtccgctgcccccaggcctcgcactgcctgcctctttccacaacccagtggaggggcgagggtaggcccagggcgaccccgcgaagccctccgcccggcgagaaccccaacccccggcccctccggccgcgcccctccctttcatataacgcacctgcggccccgcccccggccaaacgctgaggcggctgcggggctggtgaggccgggcggcttggccctccaggcacagtcaccttcccttcccccgccattccgcccacccccctagctcagcaaccgagctgccgccgcgctcctcagccgccccgaaatctaaaggggtccgtctccggtaccacaaccaaatggctgagcctccctggccggctgcgcagcccgcccattggtcccttcccccccgcctccgccaccattcgttgttgcccggagaccctcggcggcgattggctaaggccgctgccactcgtccgcggagccgccgatcccgcctccacggtgatcaggttagtgtgcgccgcgggtgctgggggctcgagaaccgagcggagctggttgagccttcaaagtcctaaaacgcgcggccgtgggttcggggtttattgattgaattccgccggcgcgggagcctctgcagagagagagcgcgagagatggagatggacaaacggattcatttagagctgcggaacaggacgccctctgatgtgaaagagctcgtcctggacaattgtcggtcaaatgaaggcaagattgaaggcctcacagatgaatttgaagaactggagttcttgagtacaatcaatgtaggcctcacctcagtcgcaaacttaccaaagttaaacaaacttaagaagcttgaactaagtgataacagaatctcagggggcctggaagtattggcagaaaagtgtccgaacctcacacatctaaatttaagtggcaacaaaattaaagacctcagcacagtagagccactgaaaaagttagaaaacctcaagagcttagaccttttcaattgcgaggtgaccaacctgaatgactaccgagaaaacgtgttcaagctccttccacagctcacgtatctcgatggctacgaccgggacgacaaggaggcctctgactcagacgctgagggctacgtggagggcctggacgacgacgaggaggatgaggatgaggaagagtatgatgaagatgctcaggtagtggaagacgaggaggatgaggaggaggaggaggaaggtgaagaggaggatgtgagtggagaggaagaggaggatgaggaaggttataacgatggggaagtagacgatgaagaagatgaagaggagcctggtgaagaagaaaggggtcagaagcgaaaacgagaacctgaagacgagggagaagatgatgactaagtggaataacctattttgaaaaattcctattgtgatttgactgtttttacccttacccccccaaatcctgccccctgaaacttatttttttctgattgtaacgttgctgtgggaacgagaggggaaacgtgtactgggggttgtggggggagggagggtgggagggggtggaataaaatactatttttactgccactctttattttttttcccctactttttctttgtgtctggttttgtccctgtaaatgcgatagctgagtacccacaagtgagcgtttgttctgtgccctcagagaggatggttggagatctcttacctttcttggtatttctgagtctcttgagttggaaggccatggctggcctcagtttggtttctcaaagcccaggaggggctgagcaccagccgcatcttacctcttgttctggttcccatgatttctgaatttcttggacttgctagaagcagtcagcgccctggtttgtaaatagcaacctgaaggcgtattttggcaccggtcgggggacccttcccgtctcccatcccctttccctctttgcagatggtggtgggcttcgctctccgaagaagaccctgatgtcactcttgagttgatgagccagagagctgaggacagcaggcttctgagttaggagtaaaatggatttggtaattagaatcaaatgagaagaagcaaaccctcaaacttcaacctctgatattttttaaaaagctgtcctatatcttgttctgtaaaatagtagaacgctttgttttacaaaatgacaagagaattcttgtacgtgtcctcctgtgcttagaccatttttacaaacctaagcacaggagacgctgctgcatgtcagctgggtttcgtttttgtttttttcttttttttttcatacacctgagcacggaaaaactaacgcaaaattgtattttcttacctagtggaaacctgaaatgactgcaaattcctagtgaatgtacaggtttgctttcctgtccctcttctggattgcttttgaagtgacgtgcgatttccctagcccgtgtttcatctgtataaaagacatctgcaccagttttcctccccctcagaagagccaaactttgagttttatgtctgtttgtcattgataaatttcaataaatctttttatacaattt

SEQ ID NO: 9 - DNA, 1676 bp, Sus scrofa
  source 1..1676: mol_type = other DNA
caccgcggctgccgagccgagcggcgccgcgcggggccgagtcgagccgagcagccacccgcgccgcgtcgccggtttaagcgcagtgcggagccgcggccgggggcggcggtagtgagaccagcgctgcgctccagcccccttttccctccatggtttctctccgctcccgtgagtaacttggctccgggggctccgctctcctgcccgcacgccgccagccgccccggaccgcgccgccggcctctgcctcgagcagaccccttccgacggccctcgctgcgcaggctgggagcctctcccccctccgcccccgttgcggaaagttaagtttgaagaggggggaagagggaaacatggacatgaagaagagaatccacctggagctgaggaaccggaccccggcagctgttcgagaacttgtgctggataattgcaaatcaagtgatgggaaaattgagggcttaacagctgaatttgtgaacttagagttcctcagtttaataaatgtagggttgatttcagtttcaaatctccccaaactacctaaattgaaaaagcttgagctcagtgacaatagaatctatggaggtctggatatgttagcagaaaaacttccaaatctcacacatctaaacttaagtggaaataaactgaaagatatcagcaccctggaacctttgaaaaagttggagtgtctgaaaagcctggatctctttaactgtgaggttactaacctgaatgactatcgagagagtgtcttcaagctcctgccccagctgagctacctggatggctatgacagagacgatggtgaagcccctgactcagatgccgaggtggatggtgtggatgaagaagaggatgatgaagaaggagaagatgaagacaaggaggaggaagaggacggcgaggaagaagagtttgacgatgaagaggatgaagatgaagatgaagatgtagaaggggaggaggatgaagatgaagtcagtggggaggaagaagaatttggacatgatggagaagtcgatgaagatgaagacgatgaggatgaagatgaggatgaggatgaagaggaggaagaaaacgagaaaggtgaaaagaggaagagagaaacagatgatgaaggagaagatgattaagatcccaaataaccaacaaaaaaaagaactgttcagtattggttggactgctcatatggatttggtagctgtttaaaaaaaaaaagtagctgtgatacaaaccccaggacacccacccacccaaagagccaaagaatagttcctgtgacattccgccttcctccattttagtccctcttggaaatccaccaccaagcttggggacttaccccaacagaattgtaagcgatgttttaggttctcagtgtaagactcttgctgtagcgtggatagctgtgattggtgagtcaaccacctgtggctaccagttacaccaagattgtaacagcatttttactttctgtacaacaaagaagctttgtaaataaaatcttaacattttgggtctgtttttttttttttgggggttttttttttttttcatgcctttcttttcttcttaatgaaaattttttacattaggacattttatgtgacaactgccaaaaaagtatttttaagaatttaagtgaaataaacacgttcctctttggt

SEQ ID NO: 10 - DNA, 2061 bp, Bos taurus
  source 1..2061: mol_type = other DNA
agtgtgcgccgcgggtgctgggggctcgagaaccgagcggagctggttgagccttcaaagtcctaaaacgcgcggccgtgggttcggggtttattgattgaattccgccggcgcgggagcctctgcagagagagagcgcgagagatggacatggacaaacggattcatttagagctgcggaacaggacgccctctgatgtgaaagaactcgtcctggacaactgtcggtcgaatgaaggcaaaatcgaaggcctcacagatgaatttgaagaactggagttcttgagtacaatcaacgtcggcctcacctcagtcgcaaacttaccaaagttaaacaaacttaagaagcttgaactaagtgataacagaatctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgtgaggtgaccaacctaaacgactaccgagaaaacgtgttcaagctccttccgcagctcacgtatcttgatggctacgaccgggacgacaaggaggcccccgactcagatgccgagggctacgtggagggcctggacgatgacgaagaggacgaggatgaggaagagtatgatgaagatgctcaggtagtggaagacgaggaggacgaggaggaggaggaggaaggggaagaggaggacgtgagtggagaggaagaggaggatgaggaaggttataacgacggggaagtagacgatgaggaagatgaagaggagcttggtgaagaagaaaggggtcagaagcgaaaacgagaacctgaagatgagggagaagacgatgactaagtggaataacctattttgaaaaattcctattgtgatttgactgtttttacccgtacccccccaaatcccgccccctgaaacttatttttttctgattgtaacgttgctgtgggaacgagaggggaaaagtgtactgggggttgtggggggagggagggtgggagggggtggaataaaatactatttttactgccactctttatttttttcccctaatttttctttgtgtctggttctgtccctgtaaatgcaatagctgagtacacacacgtgaacgtttgttccttaccctcagagaggacaatttggagatctctgacatttcctgatacttcccaagtctcttgagttgattggaaggccggggctggcctcagtttggtttctcaaagcccaggagggctgagcaccagccgcgtcttacctcttgttccggatcccatgattttcacgtttcttgggcctgctagaggcagcaagcgccttggcttgtaaatagcaacctaaaggcgaatcttggccctggtctggggaccttccctgtcttccatcccctttctctcttcccagacggtggtgggctttgctctcctgagaagactcttatgtcactcttgagtgatgagccagagagctggaaacagcaggcttcagagtttaggagtaaaatggattcagtaattctaattaaaagagaagaagcaaaccctcaaacttcaacctctttcaaagatataaaaaaacaaaactctccctatcttgttgtgtaaaatattagaacactttgttttacaaaatgacaggagaactcttctgtgcgtcctgctcggcctagaccatttttacagacctaggcacgggacacactgctgcatgtcggctgggtttttttttcatacacccgagcacggaaaaacttaacgcaaaattgtattttcttacctagtggagatctgaagtgactgcaaattcctagtgaatgtacaggctcgctttcctgtccctcttctggattgctttccaagtgacgcgtgacttcctagcccatgtttcatctgtataaaagaacatctgcaccggttttcctccccctcagaagagccaaacttttgagttttatgtctgtttgtcattgataaatttcaataaatctttttatacaattgt

SEQ ID NO: 11 - DNA, 1058 bp, Bos taurus
  source 1..1058: mol_type = other DNA
tgagctgcgctccagcccccttttccctccatggtttctctccgctcccgcgagtaacttggctccgggggctccgctcgcctgcccgtacgccgcccgccactcaggaccgcgccgtcggcctctgcctcgagcagaccccttccgacggccctcgctgcgcaggctgggacgcctctcccccctccgcccccgccgcggaaagttaagtttgaagaggggggaagagagagaaacatggacatgaagaggaggatccacctggagctgaggaaccggaccccggcagctgttcgagaacttgttctggacaactgcaaatcaaatgatggaaaaattgagggcttaacagctgaatttgtgaacttagagttcctcagtttaataaatgtaggcttgatttcagtttcaaatcttcccaaactacctaaattgaaaaagcttgagctcagtgataatagaatctgtggaggtctggatatgttagcagaaaaacttccaaatcttacacatctaaacttaagtggaaataaactgaaagatatcagcaccctggaacctttgaaaaagttggaatgtctgaagagcctggatctgtttaactgtgaggttactaacctgaatgactaccgagagagtgtcttcaagctcctgccccagctgacctatctggatggctatgaccgagaggatcgtgaagccccagattcagatgccgaggtggatggtgtggatgaagaagaggatgatgaagaaggagaagatgaggataaggaggaggatgaagatggtgaggaagaagagtttgatgatgaagaggatgacgatgaagacgaagatgtagaaggggaggaggatgaagatgaagtcagtggggaggaagaagaatttggacatgatggagaagttgatgaagatgatgaagatgaggatgaagacgaagatgaagatgaagaagaggaagaaagcgggaaaggtgaaaagaggaagagagaaacagatgatgaaggagaagatgattaataagaccccaataacctgcaaaaaaaaaaaaaaaa

SEQ ID NO: 12 - DNA, 3653 bp, Sus scrofa
  source 1..3653: mol_type = other DNA
caggttctggtgggcagtgtcggtggctggcctggctcttcgtcccagggaggaggctctgagccggctggggttgagcgcccctgcggccgcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaatagtgcctggtccttcccgccacctggccccccgcacaagatgcccgcatgtgtccatcccagggcccacggcagggcccatgttcctgaagcccgaggcacctgcgtcctccctttagggacaaagcctgtggcccatgttatctccattctgccctggggcccaatccagtttctggtgaccagactgtccaggtgtctcccagccactgccccttgtgccaaccccaccctgggcctggcccagggcccttctcagggaagtccagctccaaggccagatgagcagaagcccttgatggatgatggatggccttgaagaactgccctctaccctctctcccctttttccataaagaccctgaacctgagaatcaacagggcatcagatctgtatatttttttcctaggagtaaatgtaaataaaggatttctagatgagc

SEQ ID NO: 13 - DNA, 3808 bp, Sus scrofa
  source 1..3808: mol_type = other DNA
tgggcagtgtcggtggctggcctggctcttcgtcccagggaggaggctctgagccggctggggttgagcgcccctgcggccgcagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaaggtaagcccctctccttggcattgccccagtggcctgagggccagctgctgccaggctgcaggagggactgctctgctgaagcctggggagaggagggccgggggtcaggtcagtccatggccttctcagcagcccctccccggctctgctccctccccacagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaatagtgcctggtccttcccgccacctggccccccgcacaagatgcccgcatgtgtccatcccagggcccacggcagggcccatgttcctgaagcccgaggcacctgcgtcctccctttagggacaaagcctgtggcccatgttatctccattctgccctggggcccaatccagtttctggtgaccagactgtccaggtgtctcccagccactgccccttgtgccaaccccaccctgggcctggcccagggcccttctcagggaagtccagctccaaggccagatgagcagaagcccttgatggatgatggatggccttgaagaactgccctctaccctctctcccctttttccataaagaccctgaacctgagaatcaacagggcatcagatctgtatatttttttcctaggagtaaatgtaaataaaggatttctagatgagc

SEQ ID NO: 14 - DNA, 3412 bp, Bos taurus
  source 1..3412: mol_type = other DNA
tgcccgttcctgagcctccccgagctccctccccattctcaccatggccaaaggattctacatttccaaggccctgggcatcctggccattctcctgggcgtggcggccgtggccaccatcatcgctctgtctgtggtgtacgcccaggagaagaacaagaatgccgagcgtggcaccgcggcccccacgagccccacaggcccgacaaccacctcggccaccaccttggaccagagcaagccgtggaaccgataccgcctccccacgaccctcttgcccgactcctatagggtgacgctgaggccctacctcactcccaacaataacggcctgtatatcttcacgggctcaagcactgtccgcttcacctgcaaggagcccacggacgtcatcatcatccacagcaagaagctcaactacacccagcacagtgggcacttggcggccctgaagggtgtgggggacacccaggcccccgaaatcgacaggaccgagctggtgttgctcacagagtacctggtggtgcacctcaagagctccctggaagcaggcaagacatacgagatggagaccacctttcagggggagctggccgatgacctggcgggcttctaccgcagcgagtacatggacggcaacgtcaaaaaggtcctggccaccacacagatgcagtcaacagatgcccggaagtccttcccctgctttgacgagcccgccatgaaggccacatttaacatcaccctcatccaccccaaggatctcacagccctgtccaacatgccacccaaaggtcccagtgtcccgtttgacggagattccaactggagcgtcactgagtttgaaaccacgccggtaatgtccacgtacctcctggcctacatcgtgagcgagttcacgagtgtggagtcagtggctcccaatgatgtccagatccggatctgggctcggccaaaagcaactgcagacaaccatggcctttacgccctgaatgtgacaggtcccatcctgaacttctttgccaatcattacaatacagcctacccactccccaaatctgaccagattgccctgcccgacttcaacgccggtgccatggagaactgggggctggtgacctaccgggagaacgcgctgctctatgaccctcagtcttcctccagcagcaacaaagagcgagtagtcactgtgattgctcatgagctggcccaccagtggtttgggaacctggtgaccctggcctggtggaatgacctgtggctgaacgagggctttgcctcctatgtggagtacctgggagctgactatgcagagcccacctggaatctgaaagacctcatggtgccgaatgacgtgtacagtgtgatggccgtggacgcgctggtcacctctcacccactgaccacccctgccaacgaggtcaacacccctgcccagatcagcgagatgttcgataccatctcctacagcaagggagcctcggtcatccggatgctctcgaatttcctgactgaggacctgttcaagaagggcctggcgtcctacttgcagacctttgcctatcagaacaccacctacctgaacctgtgggagcacctgcagatggctgtggaaaatcagttgtccatcaggctgccagacaccgtgagtgccatcatggaccgctggaccctacagatgggcttccctgtcatcaccgtggatactaacacagggaccatctcccagaagcacttcctccttgatcccaactccaccgtcacccgcccctcacagttcaactatctgtggattgttcccatctcatccatcagaaatggccagccgcaagaacactactggcttcggggcgaagagagaaaccagaatgaactcttcaaagccgcagcagacgactgggtcctgctgaacatcaacgtgacgggctattaccaggtgaactacgacgagaacaactggaagaagattcagaatcagctgatgtcaaggcgggagaacatccctgtcatcaaccgggcacaggtcatctacgacagcttcaacctggccagtgcccacatggtccctgtcaccctggcgctgaacaacaccctcttcctgaaaaatgagatggagtacatgccctggcaggccgccgtgagcagcctgaactacttcaagctcatgttcgaccgcacagaggtctatggccccatgcagaattacctcaagaaccaggttgaacccatcttcctatattttgaaaatctcaccaaaaactggactgagatcccggaaaacctgatggaccaatacagcgagattaacgccatcagcactgcctgctccaatgggttgcctaagtgtgaggagctggccaaaacacttttcaaccagtggatgaataaccctaacgttaacccgatcgaccccaacctgcggtccaccatctactgcaacgccatcgcccagggtggccaggaagagtgggactttgcttggaatcagttacaacaagcagagctggttaatgaagctgacaaactccgctcagcgctggcctgcaccaaccacgtctggctcctgaacaggtacctgagttacaccctgaaccccgacctcatccggaagcaggacgccacctccaccattaccagcattgccagcaatgtcatcgggcagtctctggcctgggacttcatccggagcaactggaagaaactctttgaggattatggtggtggttccttttccttctccaacctcatccagggtgtgacccgaagattctccactgagtttgagctccagcagctggaggagttcaaggagaacaacatggacgtcggcttcggctcaggaacacgggctctggagcaagccctggagaagactaaagccaacatcaactgggtgaaggagaacaaggaggtggtgctaaactggttcaaagaccacagctaaacagtctctggtccttacattcacctggtccctatgtgagacgcccacctgcctctgtcccagggacccatggcagggcccttgtttctgaagcctgaggcaccagcgtcctccccttggggacagagtctctggcccatgttcactccactcttcccaggggaccactccagcttctgatgcccagactgtccaagtgcctcccagctcctgcccctcatgccaatggcaccccaggcttggcatggctcctgtcacccaaggctctgggtctgatctcagggagtccagctccagggtcagatgagaagccctcagtggatgatggatggccttgggggggctgccctgtgccgtctcctatcctcccctaaagaccctggaactgaaaagtctacagagcaccagatctgtatatttttttctaagataaaatgtaaataaaagatttctagaaaaaaaaaaaaaaaa

SEQ ID NO: 15 - DNA, 3118 bp, Sus scrofa
  source 1..3118: mol_type = other DNA
ggggaggatcggaactccgagctgggccgaggaagccgcgagcttcggaggaggcggaggagaaggaagagcgaggggcggggagcgcgggacccggagcgcggcaggtcaacttgaaagttccaaatacttacctgtacctgatgctgctgataaaatcaggatggctttaaactcaggatcgcggccaggtgtcgggccttactatgaaaaccacggataccaacctgaaagtgtctaccccccgcagccccccggggcccacaggccctatggggcgtatccggctcagtaccacccgccctcggtcccccagtatgccccgagggtgcagacgcacgcttccacacccgccgtcgtcgtcagcaggcagcccaaaccccgatccaggacgatgtgctcttcaaagaccaagaaagcgctgtgcatcacctttgctctgggggccatcctggcaggagcagtgctggccactgtcctgctctggaagttcatggagaagaagaggtgctccacgcccgagatggagtgcggctcttctgggacctgcatcagcccctctcactggtgcgatggcatcctgcactgtcccggcggcgaggacgagaaccagtgtgttcgcctgtacggacccaacttcattcttcaggtgtactcggctcagaggaagtcctggtacccagtgtgccaggacgactggactgagaactacgggagggcggcgtgccaggacatgggctaccggaatagctttttttctagccaaggaatcgccgatgacagcggggccacgagctttatgaagctgaacaaaagtgccaacaatatggatctctataaaaagctgtaccacagtgacgtctgtacttcgaatacggtggtttctttacgctgtatagagtgcggagtctccgggaagatgagcaaccgccagagccggatcgtgggcgggtccagcgcagcgctgggcgactggccctggcaggtcagcctgcacgtccagggcatccacatctgcggaggctccatcatcacccccgactggattgtgacggccgcccactgcgtggaggaacctctgaacaatcccaagatctggacagcctttgcaggaattttgagacaatcgttcatgttttacggcagcggataccgagtggccaaagtgatttcccacccgaactatgaccccaagaccaaaaacaacgacattgctctgatgaagctgcagacgccaatgaccttcaacgacaaagtgaagccggtgtgtctgcccaacccaggcatgatgctggagccgacacagtcctgctggatttccgggtggggggccacctacgagaaagggaaaacctcggaggtgctgaacgccgctatggtgcgcctcatcgagccctggagctgcaacagcaagcaagtgtacaacaacctgatcacgcccgccatgatctgcgcagggtacctgcagggatccgtcgactcctgccagggtgacagcggaggtcctctggtcactctgaagagcagcatctggtggctgattggggacaccagctgggggtccggctgtgccaaggcttaccgaccgggagtgtacgcgaacgtgacattatttacagactggatttatcgacaaatgagggcaaacagctaatccctcgagccttggcccttcgcaccactccgcgagaaaacgaaggcactgatttgtcatccccgtgcgtaacctattttcaggaatgatccccaggtcctctggtgtttatgaaatagtgaacccgtctgatttggcgctctccagggtcctgtgctggctgcgcgttccttcccggcccctcatctgaatagtgaggctggccggcggtggccacccaaggccagtgcagagcagggggtggggtctggcccactggggtcaccctttggaaccccttcccggggggccactcttgcacgccctgggggctggtgacttctcagctgctggacggctggagatggaagagagaaagccaggcagacactgcgggggccaccctcgcgccccccgggacctctcttcccccgagagagtttaatcattctttttaagtgccttcttcgcagccccggctggtgcctggaaataaagggaccagctcttgccggcttatgactccatagccacttgcaaggggcacggaaacctggtgagggtgtagacggcgccctcagagcaggtgcccccccccgcccccatcctgagcactgctgggactgggggccctgtgtgttgggtggggctccacccccggagaccagccttacaccccgcctcctcccagtccctctccccagcctcccgggagactgtgggacaccccccccaacactgtggtcctggggggctggtgctgctgacccttttaagacctgttaatcacgtggaaattgaggtcccccggggaaatcagggatgctgattaaacgacactgtttcctactgtaaaatttctacacgttgctacctcagtgctcacggaatctgggccgtgccccctaaggcgttcctccccttggttctttgaaaccactgtggtcctttgccacgtgtgagcagtggccggcgtgagctgcttttacaaagcgactggctcctcattcaatgttctctacgggacgcactgaagcacggcactgtggcaaagaagatgggggcctcggggctccctgtggccccttctggcccctgaggttccccagagggaaggttgggcctgaattgcggagtgtgacgtgtcaacgcagagatcctcgcagacacactgagaacgaagcgagcagttctgcgctgggacctaagcttggatggaggcagtacgatcccctggcaggatgcatccgtgccacgcacgtcttgggccacagcatccccagggaggggccgtgcagacagacgtgtctgctcccagagccagattttaaaaaaagcgttgtcgcggggtgaaggcatcccttctttagggctccttttaatttatgtgacgtctgttctggtctctgttttgtatctttttttaactgtaaaattcagttgtgaaaaattgtgcaaataaattatacattttttttttccaaa

SEQ ID NO: 16 - DNA, 2413 bp, Bos taurus
  source 1..2413: mol_type = other DNA
gtcaacttgaacgttccaaatacctccctttacccgatgctgctgataacatcaggatggctttaaactcaggatcatcaccaggtgtcgggccttactatgagaaccatggataccagccggaaagcttgtaccctcagcagccgcccacggctccccctgctttcgaggtgtaccccgctcagtactacccgcccgtggtgccccagtacaccccaagggttcagacgcacgcctcgacacctgtcatctacaggcagcccaaacctccatccaggacagcgtgcacctcaaagactaagaaagccctgtgcatcaccttcaccctggtggtcctgctggcaggagcaatccttgccgccgtcctcctctggaagtacatggaggacgagtgttcagggatggagtgcagctcctccgggacctgtgttagcccctctctctggtgcgatggcatcctgcactgccccagtggcgaggacgagaaccgctgcgttcgcctctatggaccgaacttcatccttcaggtgtactcagcacagaggaagtcctggcaccccgtgtgccaggacgactggagtgagagctacgggagggcggcctgccaggacatgggctaccggaacagtttttattctagccaaggaatagcagatgacagcggagccacaagctttatgaagctgaacataagtgccaatgatatcgatctctataaaaaactctaccacagcgatgtttgttcttcaaaaacagtggtttctttacgctgtatagaatgcggggtctcggtgaagacgagccgccagagccggatcgtgggcggatcgaatgcttactcgggcgaatggccctggcaggttagcctgcacgtgcagggcatccacgtctgtgggggctccatcatcaccccggagtggatcgtgacagccgcgcactgcgtagaggaacctcttaacaatcccaagatctgggtggcctttgcaggaatcttgaaacaatcttacatgttctatggaagtggataccgagtagcaaaagtgatttcccacccaaattatgattccaagaccaagaacaatgacattgcgctaatgaagctgcagacgcctctgactttcaacgacaaagtgaagccagtgtgtctacccaatccaggcatgatgctggagccaactcagtcctgctggatttccgggtggggggccacctacgagaaagggaagacctcagatgacctgaacgcggccaaggtgcacctcattgagccccggaagtgcaacagcaagtacatgtatgacaacctcatcacgcccgccatgatctgtgcaggctacctgcggggcaccgtcgactcctgccagggtgacagtggaggtcctctggtcacactgaagagcagtgtctggtggctgattggagacacgagctgggggtcaggctgtgccaaggcgtaccgaccgggagtgtacgggaacgtgacagtgttcacagactggatctatcaacaaatgagggcaaacagctaatctgtatggcctgggtccttgacaacattgcacaagaaaaagaaggggctgattgttaatccccacgcattatgtattttttgaagtgattccaggtccttttgtttttatgcgatcatgatcctgtatgtcttggccctttccacagttctgtgcagaccccgtggctcccctacccccgggcccacccttccagacccctcatctgaccggggaggctggctggccatggacagcagagccagcgcagagcaaggggacggggtctgccccgcggggacctcccctctgagtcccactgcagggactcctcggattcacagggggctggcgacttctcagctgctggacggcttgagaaaggaaggcgaaaagcctggcagaacctgcaggggccagcctctggggccactgctggtgagaccacttagggtagccccagggcctgtctctccacgagggaatttgatgaacattcttgagtgccttcttagcagcccagagtgctggctggaaataaaggggccagctcttcatgggtgatgacttgatagacacttgaaaggggacccaaacctttttctttcggggggtgtgtagacagagccctgggaacatatgtgatcacccccagcattgctgggggcccccacccagggagacccgccttattctccagtctctctctggcctcccttgaagatggtgcaatgccccgggcccccactgaggtcttcaccccgagtagggtgcctgctgaccctcttaacgcctgctaatcattcacctggaaatcgagctcacccagggaaatcagggatactaattaaagaaacactgtttcctgtaaaaaaaaaaaaaaaaaaaaaaaaaaaaaaaaaaaa

SEQ ID NO: 17 - DNA, 1897 bp, Sus scrofa
  source 1..1897: mol_type = other DNA
gcgtcctttccgtccggcccgcgccggcggcggggaggcggcgcgcgcggcccgcagcccgcccatggaggctttcccctgggcgccccgctcgccccgccgcggccgcgtccccccgcccatggcgctcgtgcccagcgcccgctacgtgagcgcccagggcccggcgcacccgcaacccttcagctcgtggaacgactacctgggactcgccacgctcatcaccaaggcggtggacggcgagccgcgctttggctgcgcccgcggcgaggacggcggcggcggcggcggctccccaccctcctcttcctcctcgtcgtgctgctccccccacgcgggggccgggcctggggcgctggggcccgcgctggggccacccgactacgacgaggacgacgacgacgacagcgacgagccggggtcccggggccgctacctggggggcacgctggagctgcgcgcgctggagctgtgcgcggacccctcggaggccgggctgctggaggagcgcttcgccgagctgagcccgttcgcgggtcgcgccaccgctgtgctgctgggctgcgcacccgccactgccgccaccgccgaggcggcaccgcgcgaggagcgggccccggcgtgggcggccgagccccggctgcacgcagcctctggggcggccggcgcccggctgctcaagcccgagctgcaggtgtgtgtgttttgccggaacaacaaggaggcggtggcgctctacaccacccacatcttgaagggacccgacgggcgagtgctgtgccccgtgctgcgccgctacacgtgtcccctgtgcggcgccagcggcgacaacgcgcacaccatcaagtactgcccgctctccaaagtgccgccgccgcgcagcgtcagggacggcctgcccggcaagaagctgcgctgagggcccggactccggtctgctactgccacctgacgccaccagggtcgccgcctgcccaatgtctagtttggcctgcgcaccatctctctctctcgctgctgaggagcgtggagctcagctgttggttgaacttgagatgtactgattttttttttttttttttcaaaagaacccggcggtactgagtcctttcctgtcgaagagcgcttaagactagaagctaaaatcttgatttgtttatctctagtttgtgcacatccagacggtgaaggctgggtgttcgttccactaactgaaatgtggcaacttagaagtgtttatttactctatacgtcaacctattttagatgcgcatcagtatatgaaattgtctcaatctaatcttggatgtttaattttatgaatggaggcactttactaggcctagaatatttttttaaaagcctctaaactgaacttaactggcgattttatggaatgtcagcaaaatgacttttattgtttgaaacaagtaataatatttctgttgtccttaatcagttattctaattccaggtgaagcaaccctcacctgcctggtagcatcattaagtgaaggcttagtaaactttccagtgttagtttgggtgggtgttccccccgtggcttgtttctgtcctagctggaggtgtaaagatgtacaatctgtggcaggtagaatacagctccttatccttttatgtaccacatcttttattactgaacgagcaactagcgtttcccatctttcaaagtcgtgccaggttatataatattgtgtatacacttggaaatggtgctgtttaaaagaatttgtgtatttatacagtaacagtatatgaattcattaatcttgtctttcactctggtacttggccttttgtctgtatacctccacctcgccatttctagttcaaaaatatttgattattgaagatcaaatggagccgtatgcactct

SEQ ID NO: 18 - DNA, 946 bp, Sus scrofa
  source 1..946: mol_type = other DNA
atgcagctgccaccctttgacatgtggaaggactacttcaacctgagccaggtggtgttgggactgatccagaatcgtcgacaagggccagaggccccgggcaccggggagccaagacctgagcccccactggagcaggaccagggcccgggagagcggggggccagcggggggctggccaccctgtgcaacttttgcaaacacaatggggaatctcgccacgtgtactcctcgcaccagctgaagacaccggagggcgtggtggtgtgtcccatcctacgacactatgtgtgtcccctgtgcggggccaccggtgaccaggctcacacactcaagtactgcccgctcaacggcggccagcagtctctctatcgccgcagtgggcgcaattcagccggccgcaaggtcaagcgctgaggaccgcaaggtgcccacccctgcacacccagcctctgggctcagtcctaccgagtccctggactcctctccattcccggactcaccccgccttctggaagatctgacccccaggatgccacccctgctggatcctgacaccatggaggctgtggggagccaggccagccctgccccttggacctctcagcccggggggttctgtactgggctgcttgtgaattcttggtgcggcgggacccctgccccttggaccacacagccctatggcttcttctggttcctggatcttttagtccttctgggacactagccctgccccctttgacaactcaggatcccactgtgctctcccaggcccacccctcagaatctggggagcgtgaacaggcaggcctgcgccctccagcccaagaacttcaagatgggattctgactcaagccagccgtcaccttcttcagccaccggcgacccaccggctcaccatggacgccaccggctgggtccctccctgccctcagcggcccg

SEQ ID NO: 19 - DNA, 2395 bp, Bos taurus
  source 1..2395: mol_type = other DNA
agcagcgcgcgctcactgcccgcccgccccccgcgcgtccgcctccgctccgcctccgtcccgggcgcgggcgaggtggcccgggcgggggagggcagggggcagggccgcggcggagaagccgcggggcggggaggagcgggtcccgataaaaggcggcgaggcagccccaccccgaggcaggccggcgggcaggctcggctcgtcccttccgtccggcccgcgccggcggcggggaggcggcgcgcgcggcccgcaacccgcccatggaggctttcccctgggcgccccgctcgccccgccgcggccgcgcccccccgcccatggcgctcgtgcccagcgcccgctacgtgagcacccagggcccggcgcacccgcagcccttcagctcgtggaacgactatctgggactcgccacgctcatcaccaaggcggtggacggcgagccgcgcttcggctgcgcccgcggcggggacggcggcggggacggctccccgccttcttcttcctcctcgtcgtgctgctccccccacgtgggggccgggcctggggcgctggggcccgcgctggggccgcccgactacgacgaggacgacgacgacgacgacagcgacgatccggggtcccggagccgctacctggggggcgcgctggagctgcgcgcgctggagctgtgcgcggaccctgccgaggccgggctgctggaggagcgtttcgctgagctgagcccgttcgctggtcgcgccgctgccgtgcttctgggctgcgcacccgccgccgccgccgcggccaccgccgaagcagcaccgcgagaggagcgggccccggcgtgggcggccgagcccaagctgcacgccgcctccggggcggccgccgcccggctgctcaagcccgagctgcaggtgtgcgtgttttgccggaacaacaaggaggcggtggcgctctacaccacccacatcctgaagggacccgacgggcgggtgctgtgccccgtgctgcgccggtacacgtgtcccctgtgcggtgccagcggcgacaacgcgcacaccatcaagtactgcccgctttccaaagtgccgccgccgcctgcagcccgcccgccgccgcgcagcgcccgggacggcctgcccggcaagaagctgcgctaagggcccggaccccggtctgctgctgccacctgatgccactggggtagccgcccgcccactctcgtgtttggtctgcgcaccatctcttcctcgctgccggggagtgtggagctcgtcttggtttttccagaggaagccgacggtaccgagtattttcctaacgaagagcagttgagactagacgttaaaattttgattaatgtttctagtttgtgcacatccagatggtgaaggctgggtattccactaactgaaatgtggcaacttagaggcgctgtggtttatttatacgtcgacctattttagatgcgcatcagtatgaaattgtctcagtctaatcttggatgtttaattttatgaatggaggcactttactaggtctagaatatttttttaaaagcctctcaactgaacttaaaactggcgattttatggagtgtcagcaaaatgactattttattgtctgaaacaatatttctgttgtccttacccagttgtaattccaggtgaagccctgcgtggtagcatcattaagtgaagacttggtatgctttacagtgttagtttgggtgggtgttccctccttgtggcttgtttttgtcctagctggagatgtataaaatgtacaatttgtaggtagcaggtagaatacagctcatgtaccagatctttttattactgaacgagcaactactaccgtttttcccctttaaaaatagtgccaagttataatcatattgtgtatacacttgaaaatggtgctgtttaaaaaaattgtgtatttatacagtaacagtatatgaattcattaaccttgcctttaactctacttggctttttctttatgccccttcctattccagttcttcaaaaatatgtgatacttaagatcaaacgggtgcaataactcattcactctgaattgctccatttcagggtctctaaatagtggaaatctcattccagctgttgcctctcagactaaatgtaagatggaatcctttgagctctggaaggttaatgaaacaactggtgttcaggaaggttccactctggactgtgtcagctttaaaccatcacagaagtcctcaaaccagtataagtaccaattaaaggaactgactgggtgtagggggggtaacacaaggaacacagcctccatctattgtgttcccattctcattagaagacaacccttctggaatcccaccagttattttcatcggtgagattaaatctaatcttgg

SEQ ID NO: 20 - DNA, 2113 bp, Sus scrofa
  source 1..2113: mol_type = other DNA
tcacagactccccaggacttggattggtcagaaagaagcagaggaggagccactgtgcacattttttttttccccttcacacaccataaaaatcacagaggcaactaacactcacagcaaagcttcaggttgggaactgattctctctgcgaggcagctgatctgagcatgcgcacacagattcattcttctcccaatagcacagcagccgctaggaaaattattttgaaaagacctgaatgcattaagactaaagttaaagtggaagtgagaacaaaatatcaaacagcagactcgacagagaatgagcgtcttgaagcctaagatttcaaagtgatgctaatcagagccctacctgaaagagactaaaaactccatttcaagcttcggagcatgtgatatttattcacaacaggcattccaatttcagcctcataactttcagacagataaagacttggagaaaatcgctgaggctacctgacccaggagcttaaatcaggtcagaggggatctcaacccacctggcgcaggatgaactcaacccatcaccatggaatgcatacttctctccacttctggaaccgcagcacctacggactgcacagcaatgccagtgagccccttggaaaaggctactctgaaggaggatgctacgagcaactttttgtctctcctgaggtgtttgtgactctgggtgtcataagcctgttggagaacattctggtgattgtggccatagccaagaacaagaatctgcattcacccatgtactttttcatctgtagcctggctgtggctgatatgctggtgagcgtttccaatgggtcagaaaccattgtcatcaccctattaaacagcacggacacggacgcacagagtttcacagtgaatattgataatgtcattgactcagtgatctgtagctccttactcgcctcaatttgcagcctgctttcgattgcagtggacaggtattttactatcttttatgctctccagtaccataacattatgacagttaagcgggttggaatcatcatcagttgtatctgggcagtctgcacggtgtcgggtgttttgttcatcatttactcagatagcagtgctgttattatctgcctcataaccgtgttcttcaccatgctggctctcatggcttctctctatgtccacatgttcctcatggccagactccacattaagaggatcgccgtcctcccaggcactggcaccatccgccaaggtgccaacatgaagggggcaattaccctgaccatcttgattggggtctttgtggtctgctgggcccccttcttcctccacttaatattctatatctcctgcccccagaatccatactgtgtgtgcttcatgtctcactttaatttgtatctcatcctgatcatgtgtaattccatcatcgatcccctgatttatgcactccggagccaagaactgaggaaaaccttcaaagagatcatctgttgctatcccctgggtggcctctgtgatttgtctagcagatattaaatggggacagaggagacttataaatgcaagcataagagactttctccttacacagtctggacaatatgcttcaacaacagcattttcttgtaaggcatcagttgagacattctattgtataaatttaagttcgtgattctgctcagtctctgtgtatttttaaggtcttgctaccttttggctgtaaaatgtttatctatactacaggttataggcacaatggatttataaaaaagaaaaaagtccttatgaaaagttaattaatgtatcttgtcattcgaaaggatttgacacattgcttgttttagtaaaatggaaatcacagtttcattaaatatatcctaataaatggttgctaatattacactatacaacgctgaagtgtagaggtttgattctagcattgaggggagaaatactgaaacaagtgtttaatcattaaaaaataagctgaaatttcaactaatttaataaaacatgctcattctccctgtgcagaaggagaaatgaagcttctactgggagaaaaacagttactaaaaaaaagtggggggatattttgagtttgaaaactatgttt

SEQ ID NO: 21 - DNA, 1809 bp, Bos taurus
  source 1..1809: mol_type = other DNA
cagcctaagatttccaagtgatgctgaccagagccacacttgaaagagactgaaaacttcctttccagctccggagcatgggacatttattcacagcaggcatgccactctccgccgcctaactttcgtttggggcaagtcaagactggagaaaggtgctgaggctgccagatccaggaggttcagtcagtccagaggggacctgaatccaaaatgaactctacccagccccttgggatgcacacctctctccactcctggaaccgcagcgcccacggaatgcccaccaatgtcagtgagtccctggcaaaaggctactcggacggggggtgctatgagcagctctttgtctctcccgaggtgtttgtgactctgggggtcatcagcttgttggagaatattctggtgatcgtggccatagccaagaacaagaatctgcactcacccatgtactttttcatctgcagcctggctgtggctgacatgttggtgagcgtttccaacgggtcggaaaccattgtcatcaccctgctgaacagcacggacacggacgcgcagagcttcacggtggatattgacaatgtcattgactcggtgatctgtagctccttgcttgcctccatctgcagcttgctgtcgatcgcggtggacaggtacttcactatcttctatgcgctccagtaccataacatcatgacggtgaagcgggtggcgatcaccatcagcgccatctgggcagcctgcacggtgtcgggcgtcttgttcatcatttactcagacagcagtgctgttatcatctgcctcatcaccgtgttcttcaccatgctggctctcatggcgtctctctatgtccacatgttcctcatggccagactccacattaagaggatcgcggtcctgccaggtagcggcaccatccgccagggcgccaacatgaagggggcgattaccctgaccatactgatcggggtctttgttgtctgctgggcccccttcttcctgcacctgatattctacatctcttgtccccagaacccatactgtgtgtgtttcatgtctcactttaacctgtacctcatcctcatcatgtgcaattccatcattgaccctctgatttatgccctgcggagccaagaactgaggaaaaccttcaaagagatcatttgttgctctcctctaggtggcctctgtgatttgtctagcagatattaaatggggacaaacgcgatgctaaacacaagcttaagagactttctccttctcatatgtacaacctgaacagtctgtatcagccacagctttttcttctgtgtagggcatggagtgaaaatttctattgtatcagttgaagtttgtgatttttttctgatgtgaaacagtgcccagtcttggtgtatttttaatgtcatgctactttctggctgtaaaatgtgaatccacatcacaggttataggcactatgcatttataaaaaaagaagaaaaaaagtccttatgaggagtttaacagtgtttccttcttgttatttacaaggatgtgacactttgcttgcttttgtaacatggaaatcacagcttcattaagtatatcctcataagtggtttttttatgttatactttacaacactgaagtgtaaaaatttgattctagcatttaggggagaaatattgagaacatattgcttaatcataaaaaacaagctgaaatttcaggtaatttaataagactttctcattcattcttcctgtgcagaagttgaaatgaagcttgtattgggagaaaaacagttacttaaaaaaaaaaaaaaa

SEQ ID NO: 22 - DNA, 2387 bp, Bos taurus
  source 1..2387: mol_type = other DNA
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagaagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcgtggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 23 - DNA, 2426 bp, Bos taurus
  source 1..2426: mol_type = other DNA
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagatatcacagccttctcgccttgtgtctatgttttaataggagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcggggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 24 - DNA, 1878 bp, Sus scrofa
  source 1..1878: mol_type = other DNA
atgaaggaaaatgtgccatccacagtggttttcattttgctactttttctcaacatcagccttctgaatggacagtcacctccgggaaaacctgagatcttcaaatgtcgttctcccgaaaaggaaacattcacctgctggtggaagccgggggcggatggaggacttcctaccaactacacgctgacttaccacaaggaaggagaaacattcacccatgaatgtccagactacaaaactggcggccctaactcctgctacttcaacaagaagcacacctccatatggacaatatacatcatcacagtgaacgccacgaaccagatgggaagcagttcctcggatccacgttacgtggatgtgacttacatagttgaaccagaccctcctgtgaacctgactttggaattaaaaaagcccgaagaccaaaagccatatctgtggatcaaatggcttccacccaccctggttgacgtaagatctggttggctcacactccagtatgaaattcgattacaacctgagaaaacagctgagtgggagactcatttcgctgggcagcagactcagtttaagattctcagcttgtatccaggacagaaataccttgtccaggttcgctgcaagccggaccatggattctggagtgagtggagcccagagagctccatccagatacctaatgacttctcaatgaaagatacaaccatgtggatcttcgtggctattctttctgctgtcgtctgtttgattatgatctgggcagtggctttgaagggctatagcatggtggcctgcatccttccgccagttccagggccaaaaataaaaggatttgacacccatctgttggagaaaggcaagtccgaagagcttctgagcgccctgggatgccaagacttcccccccacttccgactgcgaggacctgctggtggagttcctggaggtggatgacagcgaggaccagcagctgatgccagcccactccaaggaacacccgagccaaggccggaagcccacacacctggatcccgacagcgattccggccggggcagctgtgacagcccttcccttttgtccgaaaagtgcgatgaaccgcgggccaatccccccaagtttcacactcccgagggcatcgagaagccaggggatcctgaaacaaaccttccccgtccccaggaccctcagagcacaagcgtggaaagcaagctgctctattttcacgcggatggatccaagtcttcgacttggcctttgccgcagcccccgatcctgcacgatccgggatcttcttaccacaaccttgccgacgtgggtcagctggtcctgggcaccgcaggggccaccgccgctttgctggaaaaaacggaccgacatgctttcaaccctccaaaaaccaccgagactggcggggaaggaagggcaaccgagcagaaggagtcggagagcttccactccaagactgaccaaggcacagtgtggctgctgccccaggacaaggggccctttgtctcccctaagcccatggagtacgtggagatacacaaggtcagcaaagatggagcactggcgttgctcccaaaacagcaggagaacggcgaccggccggagaaggctggcgcccctgaaaccagcaaggaatacgcccaggtgtcccgggtgatggataaccacatcctggtgttagtgcaggatccgcgagctcgaaacgtggctccgtttgaagaaccaaccaaggagaccccgccatcccggccgcagaatccagctgcgaaagacctggccggcttcaccacggccccgggccactgcagacacccgctgggtgggctggattacctcgatcccgcaggctttatgcactcctttcagtga

SEQ ID NO: 25 - DNA, 1244 bp, Sus scrofa
  source 1..1244: mol_type = other DNA
cgcacactcatcccgcttgaatcctggggcagggactcagaaaacttccagcccgggcagcgcgcgcttggtgcagaacgcaggagcaaccggtccgtccccctccaactctccggtgccgctgccgcctgctcccgccatccgaggaggcgcggtgccacccaccgctccgtcccctgcctgcgctcagtgcgcgacccgatccccgcagaatctccccatcctccctttgctttctgactgcccaaggcgctatttgttttctctctttctctttcttgctctgcgagaggggaggggggggaagaggaggaggaattctttccccgcctaacgtttcaagggacacaatccactccaagtctcttccctttccaagccgcttccgaagtgctcccggtgatcgcaacttttgatcccaaaccgagaggggagtctcccaacttcaaatcctcttttccttctccttcgctttcctcctctcactacctccaccgccaccgccacctccacctccggcacccacccaccgctgccgccgccaccagcagcgcctccttctctcctcctcctcctcccctcttctctttttggcagccgctggacgtccggtgttgatggtggcagcggcggcagcctaagcagcagcagccctcgccgcacgccagctctagcccaccctgccttattctctagtcctacctcccaatctcccgaaaggtgctgggcagattcggggcggctgaggcgaagcggcagcggtaggagcggcggcggcagcggtagcggcggcggcgggaggcaggatgagcgcacgcggtgaaggcgccgggcagccgtccacttcagcccagggacaacctgccgccccggtgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatggccacagcaagtcgttcagaagaagcctgctcaggaggagactgaagagacatcctcacaggagtctgcggaagaggattagggggcgccaacattcaatttctacctcagcagcagttgaatcttttgaagggagaagacactgcagtgaccacttttactctctatcgccatggtctttccactttca

SEQ ID NO: 26 - DNA, 1194 bp, Sus scrofa
  source 1..1194: mol_type = other DNA
cgcacactcatcccgcttgaatcctggggcagggactcagaaaacttccagcccgggcagcgcgcgcttggtgcagaacgcaggagcaaccggtccgtccccctccaactctccggtgccgctgccgcctgctcccgccatccgaggaggcgcggtgccacccaccgctccgtcccctgcctgcgctcagtgcgcgacccgatccccgcagaatctccccatcctccctttgctttctgactgcccaaggcgctatttgttttctctctttctctttcttgctctgcgagaggggaggggggggaagaggaggaggaattctttccccgcctaacgtttcaagggacacaatccactccaagtctcttccctttccaagccgcttccgaagtgctcccggtgatcgcaacttttgatcccaaaccgagaggggagtctcccaacttcaaatcctcttttccttctccttcgctttcctcctctcactacctccaccgccaccgccacctccacctccggcacccacccaccgctgccgccgccaccagcagcgcctccttctctcctcctcctcctcccctcttctctttttggcagccgctggacgtccggtgttgatggtggcagcggcggcagcctaagcagcagcagccctcgccgcacgccagctctagcccaccctgccttattctctagtcctacctcccaatctcccgaaaggtgctgggcagattcggggcggctgaggcgaagcggcagcggtaggagcggcggcggcagcggtagcggcggcggcgggaggcaggatgagcgcacgcggtgaaggcgccgggcagccgtccacttcagcccagggacaacctgccgccccggtgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatgggtgagtaataagataaaatttttgttcttttttttttttttctttggtaatgaaaactttgtctctctttttcttttttttaaaatgaaatgaattctattttatgaattacacgaatttcttttaacttccggtttga

SEQ ID NO: 27 - DNA, 1437 bp, Bos taurus
  source 1..1437: mol_type = other DNA
gcctctggcccgcggccaggcgccttggctcggcggtctgcctggcctccctcctcctcatacttttcctccagcgcagccccctcccctttatccgcccacgattagaggtgggcactccccccatctcgccgccccctccccaagcacacacacactcaacgcgctcgaattctggggtagaaactcagaaaacttccagcctgggcagcgcgcgcttggtgcaggacgcaggagtaatcagcccgtccccctccaactctccggtgccgctgccgcccgctcccgccacccgagaaggaacggtgccacccaccgctccgtcccctgcctgcgcccagtgcccgaccggatccccgcagaatctccccatcctcccttcgctttccgactgcccaaggcgttatttgttttctctctctttctctttcttgctctgcgagcgcggagcgggggggaagaggaggaggaattctttccccgcctaacatttcaagggacacaattcactccaagtctcttccctttccaagccgcttccgaagtgctcccggtgaccgcaacttctgatcccaaactgagaggggagcctcccaacttcagttcctcttttccttcttcgctttcctcctcctctcactacctccacctccaccgccacctccacctccggcacccacccaccgctgccgccgccaccagcagcgcctccttctctcctcctcctcctcccctcttctctctttttggcagccgctggacgtccggtgttgatggtggcagcggcggcagcctaagcagcagcagccctcgccgcacgccagctctcgcccaccccgcctcattctccagccctacctcgcaatctcccgaaaggtgccgggctgattccgggcggcagaggcgaagcggctgcagcggcggtaggagcggcggcggcagcggtagcggcggcgggaggcaggatgagcgcacgcggtgaaggcgccgggcagccgtccacttcagcccagggacaacctgccgccccggcgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatggccacaacaagttgttcagaagaagcctgctcaggaggagactgaagagacatcctcacaagagtctgcggaagaggattagggggcaccaacattcaatttctacctcagcagcagttgaatcttttgaagggagaagacactgcagtgaccacttactctctatcgccatggtctttccactttcatctggggtcgggggtgagggggtggggcgggggcgg

SEQ ID NO: 28 - DNA, 2710 bp, Sus scrofa
  source 1..2710: mol_type = other DNA
gattttgaaagggtcacgtaaacaaactacaactcccagcggtccccgcgcgcaacaggtcagggctaggctggggccgggttcgcggtgctagctgaggcggcggcggcggctacggctggaagagccgggccggggcccgcggcggaggccgtggcttgtattgggagggtggcggggagggactggggaggaagatggcgacgtcgggggcgaacgggcccggctcggccacggcctcagcttccaatccgcgcaagtttagtgagaagatcgcgctgcagaagcagcgtcaggccgaggagacggcggccttcgaggaggtgatgatggacatcggatccacccggttacaggcccaaaaactgcggctggcgtacacacggagctctcactacggcggctctctgcccaacgtgaaccagatcggctgtggcctggctgagttccagagccccctccactcgcctttggattcatctcggagcactcggcaccacgggctggtagaacgggtacagcgagatccccgaaggatggtgtccccgcttcgccgctacccccgccacatcgacagctctccctatagtcccgcctacttatcgccgcccccagagtccagttggcgcaggacaatgccctggggcaatttccctgcagagaaggggcagttgtttcgactaccatctgcgctaaacaggaccagctctgactccgcccttcacacaagcgtgatgaaccccagcccgcaggacacttatccaggccctccacctcccagcgtcctgcccagccgccgtggaggtttcctagacggcgaaatggactccaaagtacccgctattgaggagaacttgctagatgacaagcatttgctgaaaccctgggatgctaagaagctgtcctcctcctcctcccgacctcggtcctgtgaagtccctggaattaacatctttccatctcctgaccagcctgccactgtgcctgtcctcccacctgccatgaacacgggaggctccctacctgacctcaccaacctgcacttccccccaccgctgcccacccccctggacccagaggagactgcctaccccagcctgagcgggggcaacagtacctccaatttgacccacaccatgacccacctgggcatcagcgggggcctgggcctaggcccgggctacgaggcgccaggacttcattcacctctcagccacccatccttgcagtcctccctaagcaatcccaacctccaggcttccctgagcagcccgcagccccagctccagggctcccacagtcacccctcactgcctgcctcctccttagcccgccacacactgcccaccgcctccctgggtcacccctcgctcagcgccccggccctctcctcctcctcttcctcctcttccgcttcatctccggtgctgggtgcccctccttacccagcttctacccccggggcctccccccgccaccgccgtgtgcccctcagccccctgagtttgcccgcgggcccagccgacgccagaaggtcccaacagcaactgcccaaacagttttcgccaacaatgtcacccaccctgtcttccatcactcagggcgtccccctggacaccagtaagctgcccactgaccagcggctgcctccatacccatacagccccccgagtttggttctgcccacccagcctcccaccccaaagcctctacagcagccagggctgccatctcaagcctgctcagtgcagccctcgggtgggcagcccccgggcaggcagctgcactatggcatgctgtacccacccggctccagtgggcatgggcagcagtcttaccaccggccaatgggtgacttcagcctggggaacctggagccgttcagcatggacaacccatccaccagcctggcgctggatgcctctggcttttccgaagggcctggatttttagggggtgaggggccagtgagtggcccccaggagccccacaccctcaaccaccagaacttaacccactgttcccgccatggctcagggcccaacatcatcctcacgggagaatcctccccaggtttctctaaggagattgcggcagccttggctggagtgcctggcttcgaggtgtccacggctgggctggagctgggcctggggctggaggaggagctgcgcatggagccactgggcctggaggggctcaacatgctgagcgacccctgtgccctgctgcccgaccctgctgtggaggactcgttccgcagtgaccggctgcagtgagggcacctgatcgccatccctctcctcggccctgtcccccgccgtcactgtcccttttctcccttccccctggccagtagagactccactctctgcccccagatcctctttctaacgtgaacgagggatcctaagaatgagaagaagcaaggggtgtgtccaggtggcccctaaattctgcacaaagggtgggcctaggggagctcaagggagggcctgaagcacttgtaactttgaaccgtctgtctggaggtcagagcctgttggaaagcaggggggtagaggggagccccggaggcagggcttgtccggatgcctaggggtgggcagtgccagcccctcctcgccactcttccccttgcaatggaggaaagagccagagtggatattattttttattaaatatattatatgttaataaaaaaatcata

SEQ ID NO: 29 - DNA, 2609 bp, Bos taurus
  source 1..2609: mol_type = other DNA
ggaggccgtggcttgtattgggagggtggcggggagggacgggggaggaagatggcgacgtcgggagcgaatgggcccggttcggccacggcctcagcttccaatccgcgtaagtttagtgagaagatcgcgctgcagaagcagcgtcaggccgaggagacggcggccttcgaggaggtgatgatggacatcggctccacccggttacaggcccaaaaacttcggctggcatacacaaggagctcccactatggtggctctctgcccaatgtgaaccagattggctgtggcctagccgagttccagagccccctccactcccctctggattcatctcggagcactcgacatcacgggctggtggaacgggtgcagcgagatccccgaagaatggtgtccccgcttcgccgctacgcccgccacattgacagctctccctatagtcctgcctatttatctcctcccccggagtccagctggcggaggacaatgccctggggcagtttccctgcagagaagggacagttgtttcgactaccatctgcacttaacaggacaagctctgactctgcccttcacacaagcgtgatgaaccccagccctcaggacacgtatccaagccctgcagcccccagcgtcctgccgagccgccgtggaggttgtctggacggcgagacggactccaaagtccctgctattgaggagaacttgctagatgataagcacttgttgaaaccatgggatgctaagaagctgtcctcgtcctcctcccgacctcggtcctgtgaagtccctggaattaacatcttcccgtctcctgaccagcctgccactgtgcctgtcctcccacctgccatgaacacgggaggctccctacctgacctcaccaacctgcactttcccccaccgctacccacccccctggacccagaggagacagcctatcccagcctgagtgggggcagcagtacctccaatttgacccacaccatgacccacctgggcatcagcgggggcctggccctgggcccgggctatgatgcgccaggacttcattcacctctcagccacccatcctttcagtcctccctaagcaatcccaacctccaggcttccctgagcagtcctcagccccagctccagggctcccacagtcacccctcactgcctgcttcctccttggctcgccacgcactgcccaccacctccctgggccacccctcgctcagtgccccggccctctcctcctcctcttcctcctcttccgcttcctctccggtgctgggggcccccgcttacccagcttctgcccctggggcctccccccgccaccgccgtgtgcccctcagtcccctgagtttgcccgcgggcccagccgacgccagaaggtcccaacagcaactgcccaaacagttttcgccaacaatgtcacccaccttgtcttccatcactcagggcgtcgccctggataccagtaagctgcccaccgaccagcggctgcctccatacccatacagccccccaagtctggttctgcccacccagcaacccaccccaaagcctctgcagcagccagggctgccctctcaggcctgctcagtgcagccctcagggggccagcccccaggcaggcagctgcagtatgggacactgtacccgcccggacccagtgggcacgggcaacagtcttaccaccggtcgatgagtgacttcagcctggggaacctggagcagttcaacatggagaacccatcgaccagcctggcgctggatccccctggcttttccgaagggcctggcttcttagggggtgagggaccagtgagtggcccccaggaccctcacgccctcaaccaccagaacgtaacccactgttcccgccacggctcagggcctaacgtcatcctcacaggagacgcctccccaggcttctccaaggagattgcagcggccctggccggagtgccgggcttcgaggtgtcagcagctgggctggggctggggctggggctggaggaggagctgcgcatggagccactgggcctggaggggctcagcatgctgagtgacccctgcgccctgctgcctgatccggctgtggaggactcgttccgcagtgaccggctgcagtgaggccacctcagcgccatccctcttctcggccctgtcccttgtcactgtccctttcctcccttccccctggccagtagaggtgccactctctgcccccagatcctctttctgacatgaacaaaggatcccaagaatgagaaaaggcaaggggttttttccaggtggcccctgaattctgcacaagcggggggcacctgggggagctcaagggagggcctaaagcacttgtaactttgaaccgtctgtctggaggtcagagcctgttggaaagtggggggtagaggggagccccggaggcagggcttgtccggatgcctaggggtgggcagtgccagcccctcctcaccgctcttccccttgcagtggaggagagagccagagtggatattattttttattaaatatattatatgttaataaaaaaatcctattaaaaaaaaaaaaaaaaaaacaaaaaaaaaaaaaaaaaaaaaaaa

SEQ ID NO: 30 - DNA, 6582 bp, Bos taurus
  source 1..6582: mol_type = other DNA
gttgggttgccagccctccaggtgacttaaggttaaaaacattttttttaaggcttcttgtttttgtgttctcagtgggctatttacaccattttcaagcatccctcctcctttggaattttaattttctgtgaacgcttcccccatagggcattccctagtgccattttattcttgacccttttcaatacaaaatccttaataaagacaatggaaagtagggtgaaagcagggaagctgaaattttgtcatctagatactgagggtggcctctttgttaagaatctagcatccagaccttgaggtcacacgattagtattcccattggaatcgtagggactctctttcaaagagtcttctgtccagttttcaggccacgttaggcacgttcccgcccagaagcagggcctgaaaagaatgtcggtgttttccttcaggtataggtgcctgggggtcaagggagagggaactgcaaggggtggagtgggaggcgggtgatagacagggatagaaactgtgtctcctcctccctctgaatgcgaaatagccaatggggtggcgcggccgagccggcccggccgccagtgccatatagtatgcagcaaccggaggagtcacgttgggggaacggcagaaagcagctatccttttacactactttgctctagcagctatcttaatgattgcaagtgcggcggacatcaagcaggagaatgggatggaaagcgcctcggaggggcaggaggcgccccgagaagtggcggggggcgcggcggcggcggcggggctgagccccccggctccagcccctttccccctggagccgggggacgccgcggctgccgccgccagggtgagcggagaggaaggggcagtggctgcagtggcggcggcggccggggcggcggtggatcaggtacaactccactcggaacttctgggcaggcaccaccacgccgcggccgccgccgcgcagaccccactggccttctcgccagaccatgtcgcctgcgtgtgcgaggcgctgcagcaggggggcaacctggaccgcctggcccggttcctgtggtccctgccccagagcgacctgctacgtggcaacgagagcctgctgaaggcgcgagcgctggtggccttccaccagggcatctaccccgagctctacagcatcctcgagagccacagcttcgagtcggctaaccacccgctgctgcagcagctctggtacaaggcacgctacaccgaggccgagcgagcccgcggccggccgctgggcgcggtggacaagtaccggctacgcaggaaattccccctgccccgcaccatctgggacggcgaggagacggtgtattgtttcaaggagaagtcgcgcaacgcgctcaaggagctctacaagcagaatcgctacccttcgccggccgagaagcggcacctggccaagatcaccggcctctccctcacccaggtcagcaactggttcaagaaccgccggcagcgcgatcggaacccctccgagacccagtccaaaagtgagtcagatggcaatcctagcactgaagatgaatccagcaagggtcatgaagacttgtctcctcatcaactctccggttcatccgatggtgtcaccaacctcagcctttccagtcacctggagccagtatatatgcaacaaattggaaatgctaaaatatcgttaagctcctctggagttttattgaacggaagtttggtacctgcaagtacttcacctgtcttccttaatggtaattctttcattcagggacccaatggagttatccttaatggattaagtgtgggaaatacacagacagtatctttgaacccaccaaaaatggcatcaaacattatgagcaatggtatatccatgactgacatactggggtctacctcccaggacgtgaaggaattcaaagttctccagagctcttcagctaactcagcagccaccacctcctatagccccagtgctcccgtgtcgttcccagggctgatacccagtgccgaggtgaaaagagaaagtattcaaacagtggcttcccaggatggaggctctgtggtgacttttaccacaccagtgcaaattaaccagtatggcatcgtccagatccccaattctggagcaaacagccagttccttaatggaagcattggattctctccactgcagctgcctcctgtttcagtggcagcttcacaaggtaatatttcagtaaattcaagcacttcagatgggagcacatttacaagtgagtctgccacagtccaacaaggaaaggttttcttgagctctcttgctcccagtgcagtggtatacactgttcctaattcaggccagactataggatctgttaaacaggagggcgtggagaggagcctggtgttttctcagttgatgcctgtcaatcagaatgcacaagtaaatgcaaacctatcttctgaaagtatctccggaagtggcctccacccactgtcctcctcattagttaatgtatccccaactcacaacttctccctgacttccccaaccctactaaatcccactgagctaaaccctgacattgctgatagccagccaatgtctgcacctgtggcaagcaaatctactgtgacgtcagtcaacaacactaactatgcagctcttcagaactgctcccttatttctggtcaagatctactatcagtacccatgacccaggctgcccttggggaaatagttcccacaggtgaagaccaggtgggtcacccctccccaacagtacaccaggattttgtcagagaacatcatttagttatgcaaccagtagctaacataaaagaaaatttcctaacaaattctgagagcaaaacaacaagcaacttaatgatgctggactccaaatccaagtatgtcctagacagcatggtggagactgtctgtgaagacctggaaacagacaagaaagagcttgccaagctccagactgtccaattggatgaagatatgcaagacttgtaaacttaatttccttttttttccccctggcatcagcaggcaaatcttttcatgaagacttgaggacataaagcattttcccatttgaagggaaacattagttttgcaagtaaatgcattcaagagaccttggattgatctgcatgaagatcacagttaaatatgctgaagatcacagttaaatatgcaggagtagaactgcattactatagccttttggttctcttttcaatagatggagacagacatgagagatgaaatgtatcaagaagtcaaagtgtatcatttggttgacttaattctaaggtcaaatggaacttggtagttttttaaattaaaaatgtatacacttaaggtagaaaattaaatatcactacagaacattagaaacaatacagttctttttgtttacttttactccatgggcattggtaaggttaagaagcataagtggtacttcatacaattcgaaggatatatttttgaatatatgaattctgtttttcagcatttttgtgaaagtcttgctttgtctctatacagttccaaatgtgactgctaatagtttagttggctgagaaagttcacaactccactaagaaaactgaaacaagcttcagtatgagtgtaaatatattagtcctataagcaaggatttgggtaattacagtgaattttatttatgctgtatatgtttctcatacttcagacactatgaactgaaaaaaatgttttggtactgtttaatttatttttattgtttgaacaggaagtgaacagttatttccaacaaagttttactttttgtaggattttaaaagtaatgatttttatcagaagtctattaaggtcaatattaataacactgaaacaggaaatctaactcccattattggaattttggtactttttttcacattcttgttctttgtttagcaactctcttattcatttaaatatattttctatagaattctacgtgcctttttaccttgtggcctttttattcttattaaggtacagaagtgtcaacagaagcactgatgacttttttctactatccaacagggttactttcacagaattgctttaaatctgaagaacttaagtttcacttaatgttttatgatgcaaatggttgaacaactaattattgttgataacttttcattaaaagtaacaattacagactaccaacaaaaccaattctggaggctatgaaaacaaccattttaaagtaaggaaagagtttgtatcttctaatgagcagaattatgtcttgttgaaacttggttctattttgaatcggttgttgcattatgacgtatgactgttgtttgcactatgttatcgcatcttctttaaatcttttgggggtttctgtaagcagcttgtttgataatgctgtgagctgtggttatacagttgttatcaagactctgaagagaagatgtaataatacaaatacatttgtgtagtgagtaaagtctctggtctctgtaaagcaagaaaagtgaggaaattatctcttcagagatgaccgaaagttgtggaacatgaagatgtcatcacatcttgctttctttaagatcggaacaaagggtccactctagctctctgcatgctcgtcagtccagcatgttgagtaagggccatagaggatttcacttttgttgttattgcaaatggaacttgcaggcatacatgaagagccctttgaagaattcctcccccatcgtattttttctcctccatcattttgataacgaacttagatctcattttttaatggaattagtgaatgttcctaaactgctgaaattacatgttttttgttttgttttacaatgtcaggcattttagagttctcttccactccaccactacttatacagcttgtttgtttctttgggtgtgtgctgatcatgttactgagattaattacatggttctatagtgtagtttacataaaaatgatgttttccacttgattgctgcagatatcaagagtttgttgattttaaaatacccatccagtaagaaagatgggaaagtttaaaaagttatatatgttatacatacataactcgaaaagttttatattgtcttccaaaaatataataagcctctcttttattcacagttcttcctattttctaccttaagaaagaaaaaagatagcacaaaacttaagacttacctaggaaacctattcatactctaacattatgtaattatacccctacaattactccaggtagctacacaatataccaggtaagaatcaatgcacattataaagaacttactgttcttttctgttctttcagtaaaataagggccagttggtatgaagcccatatatatgtgaatgaaataagataaaccccatgatcttcctgcacagaattgcaaatattttttgcattgctaagggccagactcagaacccttggcagaatttctctcctacataaaccaatgcttaaagatgctgcaaatgaccctctaatgatcaccaaagaggaatcagaataggggtaaacaactctccttcaaaccccaacccctgctgctggctgcgggattttcaatgtgagcctctctatgcactcttatcagtcagaatatggcgtttagctcttagttacacccaataaaggacaatctattgtttaataaagagaacatgcgtttgtccctcagtcaagcaagagcacctgtgtcgtactgctttgtagctcacgtgtatccatagtaatgaaaagactgaatcgtacgcgtttcattgcctttcttgtgttatgaaaggcaagtagatgttatagccatagataaaattcatagttaaattgcacacagaccttcaatctccctgtatatgcccttgcgtcttgcatgttcaaagctagattatcgggtaggcgttgcagcctgctcgactacatgttagacaagtgtgcgttagtatataaccagaagttcttcattgtttaaaatgtttttgacagatcatctcatcataaaaatataaataattcaggaaggctgtggggaaaagaactacattttacagaagatgttttaaaagtcttcctagaacttagataatagagcgtaacagttattttgtatctaattgatacatttcatgttaatttcttattaccaccttacattttattaatcaaaattatatagcatgtgacttttggctttcagggttctcaaaaatttttattcttttgttgcatgtactgtatgtttagagacaaccagttatatactcaatatggttttactgtgccttacgcaaagagaaaatctataccgaatgtatatcgtggggtgggtaacggagtctctactgcactgtcaggtgatggcacacggagcgcgtcccagaacatttctgtgcttaattacccaaccaaagatctaaagtatgtatgttgtactgtaataggggtttatgcctggacaattaagtgttttatttgttttctgaaatgatgtgaacttatttttctaaacactatgctaaataaactttatatttatacataca

SEQ ID NO: 31 - DNA, 5924 bp, Sus scrofa
  source 1..5924: mol_type = other DNA
atgtcctcttcctcccccaccgggcagattgcaagtgcggcggatatcaagcaggagaatgggatggaaagctcctcggaagggcaggaggcaccccgagaagtggcggggggcgcgacggcggggctgagccccccggctccagccccctttcccctggagccgggggacgccgcggccgccgccgccagggtgagcggagagcaaggggcagtatcggcggcggcggctggagcagcggccgatcaggtacaactccactcggaacttctgggcaggcaccaccacgccgtggccgccgccgccgctgctgcgcagaccccactggccttctcgccagaccatgtagcctgcgtgtgcgaggcgctgcagcaggggggcaacctggaccgcctggcccggttcctgtggtccctgccccagagcgacctgctacgtggcaacgagagcctgctgaaggcgcgggcgctggtggccttccaccagggcatctaccctgagctctacagcatcctcgagagccacagcttcgaatcggccaaccacccgctgctgcagcagctctggtacaaggcacgctacaccgaggccgagcgagcccgcggccggccgctgggcgccgtggacaagtaccggttgcgcaggaaattccccctgccccgcaccatttgggacggcgaggagacggtgtattgtttcaaggagaagtcgcgcaacgcgctcaaggagctctacaagcagaatcgctacccttcgcccgccgagaagcggcacctggccaagatcactggcctctccctcacccaggtcagcaactggttcaagaaccgccggcagcgcgatcggaacccctctgagacccagtccaaaagtgagtcagatggcaatcctagcaccgaagatgaatccagcaagggtcatgaagatttgtctcctcatccactctctggttcatccgatggtgttaccaacctcagcctttccagtcacatggagccagtctatatgcaacaaattggaaatgctaaaatatctttaagctcttctggagttttgttgaacggaagtttggtacctgcaagtacttcacctgtcttccttaatggtaattctttcattcagggacccaatggagttatccttaatggattaagtgtgggaaatacacagacagtgtcattgaatccacccaaaatggcatcaaacattgtgagcaatggtatatccatgactgacatactggggtctacctcccaggatgtgaaggaattcaaagttctccagagttcttcggctaactcagcagccaccacctcctacagccccagtactcctatgtcatttccagggctgatacccagcactgaggtgaaaagagaaggtattcaaacagtggcttcccaggacggaggctctgtagtgacttttaccacaccagtgcaaattaaccagtatggcatcgtccagatccccaattccggagcaaacagccagttccttaatgggagcattggattctctccactgcagctgcctcctgtttcagtggcagcttcacaaggtaatatttcaataaattcaagcacttcagatgggagcacatttacaagtgagtccgccacagtccagcaaggaaaggttttcttgagctcgattgctcccagtgcagtggtatatactgttcctaattcaggccagactataggatctgttaaacaggagggcttggagaggagcctggtcttttctcagttgatgcctgtcagtcagaatgcacaagtgaatgcaaacctgtcttctgaaagcatctcggggagtggccttcacccactgtcttcctcattagttaatgtgtccccaactcacaatttttccctgactccccccaccctactaaatccaacagagctaaaccctgacattgctgatggccagccaatgtctgcacctgtggcgagcaaatctactgtgacatctgtcagcaacaccaactatgcaactcttcagaactgctcccttattactggtcaagatctattatcagtccccatgacccaggctgcccttggggaaatagttcccacagctgaagaccaggtgggtcacccctcccctacagtacaccaggattttggcagagaacatcatttggttatgcaatcagtagctagcataaaagagaatttcttaacaaattctgagagcaaagcaacaagcaacttaatgatgctggactccaaatccaagtatgtcctagagggcatggttgagactgtctgtgaagacctggaaacagacaaaaaagagcttgccaagctccagactgtccaattggatgaagatatgcaagacttgtgaacctaatttctttctcgcccctccccatcagcaggcaaatattttcatgaagacctaaggacaagcattttcccatttaaagggaaacactagttttgcaagtaaatgcattcgagaccctttggattgatctgcatgaagatcacagttaaatatgcagcagtagaactgcattactgcagccctttccacatatgttctcttttaaatagatggagacagaggagcaagataaaatacatcaagaagtaaaagtgtatcatttggttgacctaatataattctaaggtcaaatggaacttgacaaatagttttttaaattaaaaatgcatacacttaaggtagaagattaaatatcgctacagagcattagaaacaatgtaattctttttgtttacttttactccatgggcattggtaaggctaagaagtataaatggtactccacactattttaagggtatgtttttgaatatatacatttctgtttttcagctttttttgtaaaagtcctggttcgtctctacatttccaaatgtgattgttggtggttcagtgtggctgagcaaattcggaactccactaaaaactctgaaacttcagtatgagtgtaaatattttagtcttataagcaaggatttgagtaattacagtgaattttatttatgctgtatatgtttctcttatacttcagacactatgaactgaaaagaaaaatgttttcgtactgtttaatttatttttattgtttgaacaggaagtgaacagttgtttccaacaaagttttactttttgtagaattttaaaagtaatgatttttatcaggagtctattaaggtcaatattaataacactgaaacaggaaatctaactcccaatattggaattttggtactttttttttcacatacttgttctttatttagcaactctgattcgtttaaatatatcttctatagaattctatgtgcctttttaccttgtggcctttttattattcttactaaggtacagaagtgtcagcagaagcactgatgactcttttctactatccaacagggttaatttcaccacagaattgctttaaatctgaataacttaagtttcacttatattactgtagttaatgtttatgatgcaaatggttgaacaattattgttaatacttttcattacaatgaaagtaacagttttagactaccatgtaaaaccaattctagaggctatgaagacaaccattttaaagcaaagaaagaaaagtacattctatccagcagaattctgtcctcttgaaattatttttaaatattttgaatcagttattacattatgacatatgactgtttgcactatattatcatgtcttctttaaaagtaaatcttttggggtttctgtaagaagcttatttgataatgttgtgagctgtggttacagtttttatcaggactctgaccatagatgtaataatgcaaatacatttgtgtagtaaagtctctggtctctgaaaagcaggaaaagtgagaaaatggtcccttcagagatgaatgaaagttgaacatgcagatgtcacaacatcttgctttctttaagatgggaacaaagggtccactctaggtctttacatgcccatcagcccagcatgttgaacaagggctatagaggtaacttttgttgtcattacaaatggaacttgcaggcatagatgaagagtcctatgaagaattcctctcccataattttgatatgaacttagatccccccccttttttttaatggaactggtgaatgttcctgaactgctgaaactacgtggtttttgtttggttttgttttaaaacatcaggtttttagagttctcttacactccatcactccttatacagcttgtgtgtttctttgggtgtgtgctgatgatgttactgaggttatttacatggttctatagtgtaattgacatgaaaataatattatccactgattgctgcagatatcaagagtttgctaattatttcaaaatactacccatccaataagaaagatgggaatgtttggaaagttatatatgctatatataacaggaaaagttttatatgtcttccaaaatataagcttctcttattcatggttcttcctcttttcttccttaagaaaaaaaaaagatagcacaaaacttaagacttacctaggaaactattcatactccagtatgatgtaattatacccctacaattactccaggtagctacacaatataccaggtaagagtcaatgcacattataaagaacttattgttcttttctattctttcagtgaaatagggccagttggtatgtagcccacatatatgtgaatgaaataaaataaaccccatgatctccttgcacagaattgccttttcttgtttgtttgttttcctggaagtaccaagggccagactcagaacccttggagttcatgtagcagaatttctctcttacataaaccaatgcttgaagatgctgcaaatgatcctctaatgacccccaaggaggaatcagaataggggttaataactctccctacaaaccccaacccctgctgctggctgtggaattgttggtttgagcctacctatgcactctatcagccagaatttggcatttagcgcttagttacacccaggaaaggaccgtctatttaaggagaaggtgcatttgttccttaaacaagcaagaaaatctgtgttgtactgctttatagctcacgtgtattcatagtaatgtaaagactgaattgtacacaggtttcagtgcctttcttgtgttatgaaaggcaggtagatgttatagccatagataaaattcataggtgaattgcacactgaccttaaatctccctctatatgcccttgtgtcttgcatgttaaaagctggattattgggcagatgttgcagcctgccctactacatgttagacaattgtgcattagtacataaccagaagttcttcgctctttaaaatgtttttgacagatcatctcatcataaaaatataaataattcagaaaaactgtagggaaaagaattacgttttacgaaagatgtttcaaagtcttcctagaacttagatagagcctaatggttattttgtatctaactgatacatttcatgttaatttcttactaccaccatacattttattaagcaaaattatatagcatgtgacttggctttcagggttctcaaaacatttttactcttttgttgcatgtactgtatgtttagagacaaccagttatatactcagtatggttttactgtgccttacacaaagagaaaaactatacagaatgtatatcatggggtggggtaacagagtctttactgcactgttaggtgatggcacacggagcatgtcccagaacatttctgtgcttaaattacccaaccaaagagatctaaagtatgtatgttgtactgtaataggggtttatgcctggacatttaagtgttttatttgttttctgaaatgatgtgaacttatttttctaaacactatgctaaataaactttatatttatacat

SEQ ID NO: 32 - DNA, 1610 bp, Bos taurus
  source 1..1610: mol_type = other DNA
tatcgtctgcgctccgtggacgcgccctccgtccgccgaccgacagaactgcctatacccgcctgttctccttgcggcccggacacatagtatgaccattagatatttcgtctaccaaccattttccatggaaaaccaaagggaccaggccatgatagccactggcagctttgaagaacgggacatctttagagaagcttgaccttgaagacctcagcgtgggacctaacacagccggtctccggcagagttcctctgtctcgtcttgttgctgacagaaggtgcccccttctccagtttttgttcatctcctgggaggttgcaggaatcgtcattatggttgggttcaaggccacagatgtgccccctacagccactgtgaagttcctgggggcaggcacagctgcctgcattgctgacctcatcacctttcccctggatactgctaaagtccggctacagatccaaggagaaaggcaggggccaatgcaggctgcggccagtgcccagtaccgcggggtgctgggcaccatcctgaccatggtgcgcaccgagggcccccgcagcctctacagcgggctggtcgccggcctgcagcgccagatgagcttcgcctccgtccgcatcggcctctacgactccgtcaagcagttctacaccaagggctctgagcatgctggcatcgggagtcgcctcctggcaggcagcaccaccggtgccttggccgtggccgtggcccagccaacggatgtggtgaaggtccggttccaagcacaggcccgagctggagctggccggaggtaccagagcactgttgaggcctacaaaaccattgcccgagaggaggggtttaggggactctggaaagggacatctcccaatgtcgctcgcaatgccattgtcaactgtgctgagctggtgacctacgacctcatcaaggacactctcctgaaggcccacctaatgacagacgacctcccttgccactttacttctgccttcggggcgggcttctgcaccaccgtcatcgcctcccctgtcgacgtggtcaagacgagatacatgaactctgccctgggccagtacagcagcgctggccactgcgccctcaccatgctccagaaggagggaccccaagccttctacaaagggttcatgccctcctttctccgcttgggatcctggaacgtggtgatgttcgtcacctacgagcagctgaagagggccctcatggctgcccgcgcttcccgggaggctcccttctgagcagctgctgacctgatcagcgttggctctggctgcagcctggccctgcttccttttccttcctccccttctccccttccctctctccccatccctctcttctgctccctctcccaccacctcccttccccctccccacgttctcaccccttagctccctgtagcctctcacagtccaggtggacttgaccccagctgacactgtggagagcctggcatcagccaggatctcaagcccccagtcccctagaaagccctcagcttgactcttcttcctgcccccgagcccaactagcacgtcacccataaaacaagctcaaccttaaagaaaaaaaaaaaaaaaa

SEQ ID NO: 33 - DNA, 1366 bp, Sus scrofa
  source 1..1366: mol_type = other DNA
ggcacgaggaagcttgatcttgaaggcctcagtgtgagacctgacgaagccagattccggcagagttcctctatctcgtcttgctgctgagtggaggcgtctccttctgcggttcctctgtgtctcctgagagctagcaggaaatcagcattatggttggattcaaggccacagaggtccccccgactgccactgtgaagttcctgggggctggcacagctgcctgcatcgcagacctcatcacctttcccctggacacggctaaagtccggctgcagatccagggagaaaggcgggggccagtgcaggccgcggccagtgcccagtaccgcggggtgctgggcaccattctcaccatggtgcgcaacgagggcccccgcagcctctacaacgggctggtggccggcctgcagcgccagatgagcttcgcctccgtccgcatcggcctctacgactccgtcaagcatttctacaccaagggctcagagcatgctggcatcgggagccgcctcctggcaggcagcaccacgggggccttggctgtggccgtggcccagccaacagacgtggtaaaggtccggttccaagcgcaggcccgggccggcggaggccggcggtaccggagcactgtcgacgcctacaagaccatcgcccgagaggaggggctgcggggcctctggaaagggacctcacccaatgtcgctcgtaatgccattgtcaactgtgctgagctggtgacctatgacctcatcaaggacacgctcctgaaggccgacctcatgacagatgaccttccctgccacttcacgtccgccttcggggcgggcttctgcaccaccgtcatcgcctctcccgtggacgtggtcaagacgagatacatgaactctgccccgggccagtacagcagcgctggccactgtgccctcaccatgctccagaaggagggtccccgagccttctacaaagggttcacgccctcctttctccgattggggtcctggaacgtggtgatgtttgtcacctatgagcagctgaagagggccctcatggctgcccgcgcttcccgagaggctcccttttgagcctctcctgctgccgatccgactgcctcgggctttggctcctggccccgctgccctcctccctctatcacccgctgccccctttcccaccacgtcccttcccggtccccacattctcacccctcagttctccgttgcctctcatggtttgtccccatggagttgaccccaactgacactgtgggaagcctggcatcagccaggatctcaagtcctccgtcctttgaaaagtttagcccaactcctcatcctgccccacccagcccagttagcacatcacccat

SEQ ID NO: 34 - DNA, 1367 bp, Bos taurus
  source 1..1367: mol_type = other DNA
tcggcacgaggagcaatcagatccctgctgccgcctcctgggatggagccctgaggacccttaaagaggccccatgcttccgccaccctggctgcccctacagcctcctccatgcactgaagcccaaggctgggcagcagcctctctccttggacctccactgggccctggagggaccgggcagagcctttgggaccatggttggactgcaaccttcagagaggccccccacaacgtctgtgaagttcctggcggcgggcacagcggcctgctttgctgacctcctcacctttccactggacacagccaaggtccgcctgcagatccagggggagaaccaggcggccctggcggcccggagtgcccagtaccgcggagtgctgggcaccatcctgaccatggtgcgcaccgagggcccccgcagcctctacagcgggctggtcgccggcctgcagcgccagatgagcttcgcctccatccgcatcggcctctacgactccgtcaagcagttctacacccccaagggatcggaccactccagcatcattacccggattttggcgggctgtaccacgggggccatggcagtgacctgtgcccagcccacagacgtggtgaagatccgctttcaggccagcatgcacactgggctgggaggcaacagaaagtacagtgggacaatggatgcctacaggaccatcgccagagaggaaggggttcggggcctgtggaaaggaattctgcccaacatcacgaggaatgccattgtcaactgtggtgagatggtgacctatgacatcatcaaggaaaagctgctagactaccacctgctcaccgacaacttcccctgccacttcgtctctgcctttggagctggcttctgtgccaccctggtggcctccccggtggacgtggtgaagacgcggtatatgaactcgcccccaggccagtaccacagccccttcgactgtatgctgaagatggtgacccaggagggccccacagccttctacaagggatttacaccctcctttttgcgtttgggatcctggaatgtggtgatgttcgtcacctacgagcagatgaaacgggccttgatgaaagtccagatgctacgagactctccattttgactaagacaaggctactcagtatttaggacaaaaccagttaaggttggaataaagcagtgggtccgcgccacagggacacagacccacacatgtttgtttacagaactgtttacttgatgctgactcaagagacagaagaataggaaggattgtggtcttcagtgctttgtcttaagaacacctttgttttgcactgacaaggtggaaaataaattatattaatttgtgaaatcc

SEQ ID NO: 35 - DNA, 2245 bp, Sus scrofa
  source 1..2245: mol_type = other DNA
agggcactcgggtccctgctgccgcctcctggggtggagccctgaggagcctcaaggaggccctgcgcctcccccgccccggccggactgggcagggccagtcccctgctggtgctgcaggagccgggggaccgtcccagaagcccgggctccgtggctcggctcctgcagcctcccccgcacggaagcccaggccgggcagctctcctggaacctccctgctgggccctgggggacctggcagagcccctgggacatggtgggcctgaagcctcccgaggtgccgcccacgacggccgtgaagctcctgggggcgggcaccgcggcctgcttcgccgacctcctcaccttcccactggacacggccaaggtccgcctgcagatccagggggagaaccaggcggcccggagcgcccagtaccgcggggtgctgggcaccattctcaccatggtgcgcaacgagggcccccgcagcccctacaacgggctggtggccggcctgcagcgccagatgagcttcgcctccatccgcatcggcctctacgactccgtcaagcagctttacacgcccaagggatcggaccactccagcatcaccacccggattttggcgggctgcaccacgggggccatggcagtgacctgcgcccagcccacggacgtggtgaaggttcgattccaggccagcatacacgccgggcccaggagcaacaggaagtacagcgggacgatggatgcctacaggaccatcgccagggaggaaggggtccggggcctgtggaaaggaattctgcccaacatcacgaggaatgccatcgtgaactgtgccgagatggtgacctatgatgtcatcaaggagaaggtgctagactaccacctgctcacggacaacctgccctgccactttgtctctgccttcggagctggcttctgtgccaccgtggtggcctccccagtggatgtggtgaagacccggtacatgaactcgccccccggccagtaccagaaccctctggactgcatgctgaagatggtgacccaggagggccccacagccttctataaaggattcacaccttcctttttgcgcttgggaagctggaacgtggtgatgtttgtaagctacgagcagctgaaacgggccttgatgaaagtccagatgctgcgggaatctccgttttgagtaagacaaagccacctggcgtttcacgaaggcaaaaccagccaaggggtgacaggcgccgccgtgggtccgcgcacataaggccacaggcccacccgcatttacacaacagtctacttgccgccgactcggaaataggaggaggaaaactgtggtctccggtgcttcaccttcaggacacgttcgtgtggcactgacaagatgctcaataaattatattaatttgtgaaacccactaggatggatgcctaacatttaggcaaaagaagataaaccagaatggatccatatggataaaacagacgtttgcaaacttccgtcccctggaaaacgatcaggtgtcaacagataaggctgcaagcctgggacaggattcagactcgatacacttggaaatcagccgagtctagaggggcactgccaaagccacacaaggctttcaacggcagccttccgctacccacggatcaccagccacatttgccatgtgtctgcctggtagaacccaccaggattccgggattcgggaaaagaaaaagagcaaagccagctcctgtgacaaaggagccctgctgggccggctggggctgaggccgggatgctcgggagacccggagggtcaggagggcgggctcggcctcctctcccgtaaaatgggagcacaaagccctctctcctacctcccctggggtgtcacgaggatcaagcgagaagctgggcaaggaggcgctttataaaagctccatgaaatgtgtatataaggacgaggccttgggaatgttttcgtccgtcagtgaggcagtgccggggaagaagctttagaaagacacagatgtccagaggtcagaccaaaggaaccaggggctggaggggcagtggccggagccccgcgcccatgcggcctcccctccctgctggactgaggcaccttgaagggtgtggagtttggggacaaagaggtaaatgctgggcccagagcctcaagtaccaggactctgaatttggggaaaaggattatgactcaataaaggatgcgacaccttgggtgtgggaatgagaaga

SEQ ID NO: 36 - DNA, 7393 bp, Bos taurus
  source 1..7393: mol_type = other DNA
atgggggtccccaagaggaaggtctcgggcggccaggacggcgcggctgcccgggcgagcacagccaagcgggcccgcaccgaagagctcacgggcgtgcgcttcaaggctcagctgagggacccgcagggcgcggggccggccctggaggcatttgtttccgctgctaagaagctcccccgagaagatgtgtacgatgttgtggaggggtacatcaagatttctgtggagtgtgccgagattttccagctcctgagtggggagaagcgccctgaaagtgaaatgcttttagtatttcaagttttcgaggccatattattgcggacagccagtgacctttcccattttcacgtggtggggaccaacatcgtgaagaagctaatgaataaccacatgaagctcatctgtgagtccctgtacgcctcgggttacaggatggctcgagcctgcctgaacctgatggctgccatggtgacccagggtcccgaggccgccagggacgtctgcagccattttgatttgaataagaagaccttgtacaccctggtgaccaagagggatccaaagggagtacatgatgtccggctggcctacattcagttcgccctttcctttctgattgccggtgatgacagcacgatcacgcaagtgctggagttgaaagaatttattccttgcatttttagctcaggcatcaaggaagataggatctccaccatcagtatcctgttgtccacactgaaaacaaaggtagttcacaataaaaatatcacaaaaacccagaaggtgcgtttcttcactggccaagtgctgaaccacatagcatctctctacagctggaaggggattgtcgacgtgagcctggacagcgtcgagacttctgctaaagacgcagggaagaccatggtgcgggagcttgttcataacttcctgatggatctatgttgttcgctcaagcatgggattaatttttacgacgcatctctcggtaccttgggcagaggaggaaacctgactctcctgcacttcctgctgggtttgaaaaccgcagcggacgatgagctggtcgctgaactggtggtgaacattctgaaagtgtgcccggacctactgaacaagtacttcaaggaagtgacgttttcctttctcccaaggctgaagtccacctggtcaaataacatcagactgctaaacaagatctacgctgcccagccggacatctcccgggcgtttcagacccgagagttcatcccgctgcctcggctcctggccatggtgatggtgaccaccgcgcctctggtgtgcaacaagaccatgttcacgcaggcgctgaatttggacagcgtatccgtgaaacacacagctttatcccttgttgccgtcattttgaagagagccttaaagaccatcgagcactgcctgaataaagaggcctggcaggagtcaggcgtctacacggccgccatgatggaagacttcgtgcggctcttcagagaagctttgagcaagattttaccagacctgaatgccatcatctgggtctggcagtctctcaggaagcaagactcagaacacgatgacgagaaaggaaagaaaaaggccagcaagaccgctgctccctgcatggctcccgagagtgatgatgcagaaaccattcttctgaaagccgtgctgctccaggtcatctgcctctaccagaaggtggtcccgcacgtggtcatgcagtacaactttgacttcagcaagctcctgaaaggggtcatctcagaagagggcctgcgggaggaggtgcccccgcttctgcagcaccacattctcaaggtggccctggagctgcccgccagcaagtttctgtggttaaaggcccaggaaggcccagatgcagaaatcattggaggcaaacgatcggtgttttacttactgatgaaaatgtttgtgaccagtggccattcacagctcaagtcatcaaccaaacttctgattgtgaagatcctgcgggacacgggtgtgtttgagcacacctggccggagctggagctgtggctggagcacttggatgacatggcggaggagaacaaggaggctgtgattcagtttttggaacgcgtcctcctgacgttggtggcgaatccgtattcgtacacggacaaggcatctgacttcgtccaggaagccagcgccctgcaggcccccgggacgcggcaggacgctgatgacgccagcattcccatctcccacatcgacgatgtcctcgacatggtggatgtgctggtggaaggcagcgaaggcttggatgaggaggttggcttcgtcctgaatgaagacacgatcctcctcacgttcccgttcagcgccttggtccccgccgccctggaggccaggaataagttgctccttggaacagaaagtgaagcaggagagagcgttgtggcatatctgaccggggttctcacggacctgctccatacccagagagacccactggccctgtgtcttctgcttcagtcttacgaccagttggagcctccaagcctgccttgccgccaccggctgtccgggttcaacagatactacagcctctggatcccagagccagccctcgaggccctgccgctgcagacttcgggcagccctgccgcaccaggccccgcggccccgtccttctcctccctgttgcagacggcctacgccagtcaggaccaaggccagggcccaggcgggctcctactcctggacgacggggttcaggcgcagctgcgggcggccctcctgcgcctcccgatgcaccaggccctgcgcgccgccaaacacctgctgctgtacatcaggagcgccgtggagaacttcgcccagctgggccggagtgcggggccgcccctgctgcagctcctcctggatctcctccggcgcctggtcatccactgcctgcagctggacgcgcagaaccagcagaagtgcgaggccgcccaggccgagtccgacctctttgtggacatggagtccgtggcctcgctggagctggccagcgacaagacgctggaggaggtgctggtggctgttctcagacaccccacgctggagggctggttcctggccctggagcagcaggccctcccgccgcatgccctcagcccggtcctcgtgaagctcctggcagccagcctcagctcggggatccttccgctgctggtggccagtgctccagccctgcggagccttggtcagttgggcctgctggccaggtactcaaaggccatcacccagagtgtcctccgcgagctgcacagcctgagggccagctcggccacgtcaacgccaaaaaccctcctccctccgctcgaggccctgcgggggctgcacctctacatggaggacgccccactccgggaggtcaccctggccctgctgagcctgcccgaggcccgcctggcggcccagcgacccacccagcacccccggaaggagagaccgctgactgccctcgggaggaccctggtccagctgctgagtggccacgcccaggagcagccgcagagcggggagcccctctgggcctctgagtacgtgcggggcctggcagctctgctgcccgcgctggccgtggctgagctggacgccgtgctgcttggggctctgcaaagagagctggcgctggtccctgcggtcaggctggccctgcttgatgactgcctggcgcggcggacgccggcggccctggccacagcagccctgctcctgcagcacagctggtcgcaccggctgcgcttcgagcgctggtgccagcaggccggctccggactctgcctgcgggaggccccggacgacttcctccccatcgtccatgtgtacctgcagggcaggactcagggatgcttcacacgccccacagaagcttcctccgctgtcattcctgtcttgaggaaggctctgtggatgccgctgcaggccaggcttctcagcgccgacgggcccccagagtccgggctgccgcagcagatcctggcccagctggtcccgtttgccagagccaaggacctacgtgccctcatggaccgcttgcccagcttgctgcagactccagccagtcacacgagctgggtcgtggctgatgctgtctcagccgtcctggaagggtcagcagaagagctgcaagcctggaaaaagactctactgaaggcctgtgtgcagtggctaattgcatccttcagtggcggccagcaggaggacggtacccaggatcaggagaagcaaatgctgctgaggtttaacgaactgttgaattcacttaatgaagttgaccctggtgattggcagaaatttgtgaagaccggactcaagtttcggtatcaggaccgcgcgtttttggaagccctccgtgccgccacgcagctgttgtatctcccggaaagccccgtgtgctcgcagctcacccagctcccggtggtccacatgatgctcacccagcactccctgttcctgcccaccctgctgaggaccacagaggaggagcccgcagacacccccgtgagagaagcgctggtggacctgatggcggtggtggtgcggctgtgcccctctgtgtgtgcaagcagccacttcgcagtgctcctcggggcctatggtgcctcactcagcatcctggaccagaagatcttacttctcctccgggcctacgaacagaacaaccacagcctcgtcagcttcagggtactgctgtggggcccatcggccgtggagcatcagaagacgtgccggagcctgggcaagtcactatggcagcaaccgagcgttggggacatcctccgcctgctggatcgtgaccgcatgatgaagaccatcctccacttcccccagaaccggaggctgctgccccccgaggatgcacaggagccgatatttagagaccggagcacggtggatctcggggacctttatgacccctgtttcctccttcatctcttcagtgaactgaccaggccagaatttgtggtggattgtagaaaattcgtggattcaaatgctttgggcctgactgtcgcggccctcagcagctacgacccacagatgcgcgccgcagcctacgcggtgctggcggcctactactcacacctggagggggcgcgtttccgggagcagccccaggtactgtacctgttggacgtggtcaggaatgggatccgaaccaagaacatgagactcaccttcaccctgactctcttcattgccaaggcagccttgcagattttgaaaccagaggagcatatgtacatgaagatcaacaagttcctgctgtcccacgagtacctgaacatgagcaaagttccaggcttctaccagttcttctacagctcggacttcgagcagaagacggagcgggagtgggtgttggggctccttcgccagggaatgcgggacaagcactgcttcgagctgtgcgccaggcggggcgtcctccacatcgtcctgtccttcttcggcagcccgctgtgtgacgaggcggcccagaactggattctggagattctgcagaatgccgcccggaatgccaggtctgcctatgagatgctgcgggactacagcctcctgacgtgggtcctaaacatcctggaaagcaagtttctggagaccgagctgctttccaacctcatctccctgctgcacacactgtgggtgaccacgctgggcaacagagaagtggaggccggggcacagcctccctgcaagcctggctcccaggagcccccgaagctgctggccctgcacctggtggacgagttcctctacattctccttgtgctcacgaagcacctgaggcccaccttggcctccgcccagctgaccggcttcttcggggcactcgactcggtcctgaggtaccgggccaccgtgctgcaggccttcaaggacatgaaccgtctgaccatgaacgccacggtgctgtccacccgggatgtccttgtgctcctgcacaagtggagcctcatcgagagagacgtccggctccaggaggacctgagggccgccacggaaaagtgccaagtccgggagctgctgaagatgctcaaggagaagaacaagcccagcgcgccagcccgagccaaaggcatgcgggcccggaagcggagacctggcgaggcggaggaggcggccgaccccgagctgccggcgtccgccctggaggcgtgcagcggcctcctgaggtccatcctgacccactgggcacccgagttccccagcgccgacccggagcgcgaagacccgggccccgtgggtgccatcgcctccctggtagccggctgggtgctgcgctccgcagccaggagtacccttggccgggcggacgcggcaggcctcctgggctggctcaagagccacgtcctgcaacagccggcggtggtggctgagcttctccgcgatggcaccgtgacgagcagcctgttccgcctgtacagccggttctgcggtgctgtggagccggcgggatcagaggaccgcttggccggcctcttcaacgccgtcctgctgcgcctggtggcggcccggggcaccgcggggatccccctccagccggtgctggagaccgtccacctctcctccctggacgccgaggaggatgcggacacacgagccgccgccgcgttcctggtatctctgtacattaaggacatctggctgggggcccagcggccagacaccctcctgagccacgtccggatggtcctcgacactgcggaggacttgggagggggcaacgaggaggccatcgtccggctctgtagggacatcgcggccacggtccctgacatctgactcctgcggctggcacctccggctccaggtttcaggagtcgggttgagtcgggggtttccatggaaagcctgtgagacgcaggctgtgcgtgtcgacctgctgttctgacgggaaatgttggcttctctaggtgactgatacaaaagaagctcaagggaccgtgtatgcagagaggtcagggttacagtgggcctgggtggccctgggcccacgagccctggtcagggaagacccttcctctgaggtgcttgttgttcctcagagaaatacctcaaactgaggtaacgctaaagttgaaatgtctaaaagtatacgagacacagttgcatgaagagagatgtttcaagtttcctcttcataagaagtcacagcaaagctttgtgtaactttttgaataaataaacctttccagtattgtgttaaaggtattttactaaataaaaggatgttgtactaaattcttaaagtataaaactattttgtcttctgcttggaaaagactgctttatatttttaaaaattccatttgaaagcaaggactttgaattttataacttgttaataaatgtctatttttgttag

SEQ ID NO: 37 - DNA, 1655 bp, Bos taurus
  source 1..1655: mol_type = other DNA
cccgcggcggggaggaggcgctggcagcctgggccgggaggtggcagggggccgcggagacgtcggctaccgactcgtcctgccatgtgacgccgccctccgtcccgcgatcccccgtgcgccccgggcccgcgcccgcgctccgtcgcgcggcacatcatgctgctgccgggccacgcgcgcccaccgtccgccccgcaacccgcgcagcatcccggcctccgcaggcaggtagagcctccggggcagctcctgcgtctgttctactgcaccgtcctggtttgctccagagagatcgcggcgctcacggacttctccggttacctaaccaaactcctgcaaaaccacaccgcctatgcctgtgacggggaccatttgaatctgcagtgtcctcggcattccacgataagtgtccagtcggcattttatgggcaagattaccaaaagtgtagtccgcagcagcctgcctcccagagggaagacagcctaacctgtgtggtgcccaccacactgcagaaggtgctggacgagtgtcagaaccagcgggcctgccacctcctggtcaatagccgtgtttttggacctgacctttgtccaggaagcagtaaatacctcctggtctcctttaaatgccaacctaatgagttaaaaaacaaaactgtgtgtgaagaccacgagctgaaactgcactgccacgagtccaagtttctcaacatctactctgcgacctacggcaggaggacccaggagaggcatgtctgctcctcggaagccgagcggctccccccctttgattgtctgtcttactcggctctgcacgtgttatccaggagatgttacgggaagcagaggtgcaaagtcctcgtcaacaaccaccatttcggaagcccctgtctgccaggagtgaaaaaatatctctcggtcttctacgcgtgtgttcccaagaatatactcaccgcaattgacccatcagttgctaatctaaaaccttctgtgaagcagaaagatggcgactatggcgtagaccttgacccaagggaaccgagggttctgaggaaagatggtgtgattgtcagccactcgttggcagcatttgcttacattagaggctttcgatgcgatgcaaggaaggagacagcctccctggtggctcacatggtgaagcgtcggcctgcagtgcgggagacctgggatcgagccctgggtggggaagatcccctggagaaggaaatggcaacccactccggtactcttgcctggaaaattccatgggcggaggagcctggtgggctacggtccatggagtcgcaaagagccggacacaactgagaacttcactttcacacagaaggagacactgaaggggaggctgagtgtacgagggattctgtgactgttccacaggctctcgagagcctaatggaaggatgtcgggtcagcgtgagagacggctacatactctgtggctccttgggaccatcaaaaatgggaacaaaggacacgatgggaccacccaggatggtccccaagcagattacggggccctggacactctacattctagtgaagttcctcattcaacagctgctatttttttttttttttaattggaggataattaccttacaatattgtgttggtttctgccatacagc

SEQ ID NO: 38 - DNA, 2881 bp, Bos taurus
  source 1..2881: mol_type = other DNA
gcccccgcctcccgcgtcgctcgggaagaacggctgctcgagtctccgcttccctgggtgagccattgaggagctcccgccgctgctccagggtgagggcgagccgagccgcctctgcttttcgctctagctttgagttattttcagaagctaagcccagcacgcttcatgagtggacagaggatacccattttcacatgtagatcttcaacgaagttgagagttaacaactgactcatttttggtattaagaaatgactctgcaccatatttttatgtagaccaaaacatttaacctctcagctgcaaagagaatcacgttggattatttttcccttccagtggctaagttgaagacctgccatgacaatgaagaaagagaccgcgtttgctgtttccacctaaaaaaaaatgaccaacaagaaccaaagttttggtgtgggacaggactccatgtcctccatgacttgtctaatccacgtccttgaagcgtggtttggtgtggagcacttggaagactactggaattttgcaaactacctcttgtgggttttcacaccactcctccttttaatactcccttacttcaccatctttctgctctaccttacaataattttcttacacatttataagaggaagaatgtgctaaaagaagcctactcccataatctgtgggatggtgcgaggaagacggtggcgaccctgtgggacggacatgcggctgtctggcatggttatgaagttcatgggatggagaaaataccagaagaaggaccagcactgatcattttttatcacggagctattcccatagatttttactacttcatggctaaaatttttattcataaaggcagaacttgccgagtagtagcggatcactttgtctttaaaattccagggtttagcttattacttgatgtattttgtgctattcatggaccgagagaaaaatgtgttgaaatcctgcagagtggtcacttgctagctatctcaccaggtggtgttcgggaagcactgatgagtgacgagacctacaacattgtgtggggtaatcggaaaggcttcgctcaggttgcaatcgatgcaaaagtgcccattattcctatgtttacacaaaatattcgagaaggatttagatcacttggaggaacaaggctgtttaggtggctttatgaaaagttccgctatccatttgcccccatgtacggaggttttcctgtgaagttgcgtacgtacttaggtgatcccattccatacgacccaaagatcacagcagaagaactagccgaaaagacgaaggatgctgttcaagctctgattgacaagcaccaaagaatacccggaaacataatgagtgctttgctagaacgttttcgtaacaaacaaaagatcaaccagaagactctttaatacatttatattaatctgtttgtatctataggactgtattctgaattttgtaggttctataattagtatttttttttaaaaatcatgtcaagaagcatctttcattgaacttgtttgcttaaaattatgttgtcaattatgtttctgcaatcagttgtgtcaaatttaaacagtaactcactacatgcatcataattcagaaaatgatcatggttctttttataattttctaatcatgtatcataaacatagatccttcaatctatgatcttccctggtagctcatctggtaaagagtctgcctgtaatgcaggagacccaggtttgatccctgggtcgggaagatcccctggagaggggaatggctacccactccatgcctcataatttggaaaatgatcatggttctttttatagttgcctaatcatgtatcctacacatagatccttaaagtttagtttatcaataatagcagtggaggttaaacaaacattcctaaataatgcatctagtttctgcatctcttgcactgagctagttttgcaagatggcttaacctagggttttagtacagatgcttaagttagagaaaaacaagctctttcctctgcttctcattcctttcatgaccttcagcaaatcacatatttttgtgcctcaacaattcactttttaaaaacatgttaatcctttggtgaggaatattgttttaactctttatatttggtgtgattgatatccatagtatggaaattatgactgtataaactaaacaataccaagatcttttagaagtttgtgtatttattttgtccataggatgtttaaattattccttcagatacgatgtcaagttgccaagcaacaatgaaaaagatgtactgtttttcaaactgagtaaaacagaaattagggaaagtaaaatttctaattattgtggttattctttttagtattatatttttaatgagaatctgtagacaaaccatagaaggtatgctctgcgtcgtctgccctgtaatgtggcatttaatagtgtcaggttagaaatcacagtcatgccctgcatttcgtactttagaagctgactctaggctgctgacagccccgggagaaagctctgctcttctcagcattctggctcattcagcttgactctgcatccagacggatggtttattgtttcaataaaaatatacttgaatgataaatttatgtgaacatagggctctctgaaaaattaatgggtttcgatttgttgtctctttgaaatctgaatttaaaagtatttaaacagaatactgccaggaatcaaatgcagtatgtccctctccctttttaagatattaaattacttggatttttgtatacacttttgtataacaagtatgtatgtatgatatgtgtaatgtatgtacaattaagaaaaataaaaaaaagtaaagctgaaaaaaaaaaaaaaaa

SEQ ID NO: 39 - DNA, 3859 bp, Bos taurus
  source 1..3859: mol_type = other DNA
ccgccctcccgaaccacttccggcggcagctactcgcctgcgaccagcttgagccgtcccgggccggttcgtgccctgcagcaaggggtttttcctccgtccgcccctcggccccgggcagccgaaccgcgacggggtggccggcccgcgtcgtttgcagcgtggccgcctcctctgaggcgctgagatgtactgcaagaagtggaggcctgtggcggagatgtgtctcttcctcgaagacctgcgggagggctcgcgcatcctctgcctctgctcgcgggcgtttgtggaagatcggaaattgtacaatttgggactaaaaggctactacgttaaagatgatgacaacagtgcaggcgaacaagccacggaagaggaggaaggcggccactcccagggccctgcagagcctcatgacaacagatgcccagacccagatgagagtgaactggattccgaggctgaactcatgaggagtatgggattgccccttcagttcggtaggatgtccgcacataagaattttgaggaatctatgaatactagaaacaaagttaaaataatgaaaaagaaaaagaaaaaacatcaaaagaagtacttagatgagattaagagagaatcttggagacaatgtgaagaagatgacgtttcggcttcggacgatccttcttcatctgagaatgagaacactagaaagtacaaacttcaaagccaaaaaggcactgaggctgaaagttttcccgttgaaaacacagtaggtccaaagctggaaattacagagatgtgggaaaagtactggaatgaatacggtggaggactgctgtggcaaagctggcaagagaaacatgcagatcagaccctgtgtccagaaccatggaatgttccagacacaaaggaggagtgggagcagcactacagtcagatgtattggtactatctggaacagtttcagtactgggaagctcagggctggacttttgatgcctcacaaagccaggatacagaccctggtgggtctgaaatagaagttgacaacaagaaagatgaaaatcacatgaaagcagatgaacttttttctccatctttatctgttggcagcgaaagctctagttcaagtgataaagatcacagtgaaattctcaatggaattagtaacctaagtctgaattcagagaaagtagaacagagacaactagatccctccctaagttgtgatggacgtcagtgtttgagtgaagttcccagtagaagagaatgccctgcttctggccacagtgaaccgagtcatggaggaaacacggaaagcagcttgtctgggagcagaagcacagaccagccagctcaggattcacaggagtcctcagaagcaaacacagtcagagacggacgccaccgcagtgggactgatggggacgagagtggagacgatccgcctgaagagaagcgaagcaaactcaagaggagtcatgagctggacctcgatgaaaacccggactcagactttgacaacgatggttctcttctaggattcaagcacggctcagggcaaaaatatggaggaatttcaaacttcagtcaccggcgggtcaggtatttgcagaagaacatgaagcagaagtcaaagtccttggacatgagacgacaaatcaatatgaaaaacaagcacatcttcttcactgaagagtcagagaaaactggttttaagaaaagcagaactttgagtaaggtagaaaaatttctaaaatgggttaaggaaccagtggatgaagaagcatcgccagagtcggtttctcataacaacatgcaagacacttgcacaagcagtggctcagaggaaccagaagtgtctgttgagaagggggatgacccggtggaaatgagtgaggcagaacctggacagcatcctgctgtgtgtccagccggggaagtgggagcagagacgcatgacgggaccagcccagaagccgccgggacagatacagagggctgcgtgccccaggctgcttcggaccctcgccaggaagaggctgaagctgaaagtcagaaaaagaagaaaaagaaaaagaagaacaaaaaggagctgaatctgccccctgacatcgctgccgtgcctgagcttgtgaagtactgggctcagaggtacaggctcttctcccgtttcgatgatgggattaaattagacagagagggctggttttcagttactcccgagaagattgccgagcatatcgcgggcagggtcagccagtccttggagagtggcaccattgtggacgccttctgtggagttggaggaaataccattcagtttgccctaacagggaagagagtgattgccatcgatattgatcctgttaagatcgatcttgctcgcaataatgcagaaatatatggcgtagtggacaagatcgagttcatctgtggagatttcctgcagctggcttctagtctaaaggccgatgttgtgtttctcagcccgccttggggaggaccggactacgccactgccgagatcttcgacatcagcaccatgatgtctcctgatggctttgaaattttcagacttgcccagaagatcactaataatatcgttttctttcttccaagaaatgctgataccgaccagtagaacattcatgacaaaattcaccctaaatcactctgaagcatacggtccaggagcattaaatacattcgtgttgtgggacagccgtcagcaccatgtgcctcgggacatttctgttttctgacactgcagctctgtacccatcacagactgattccacacccgtccaccactgcctccagcgcccgccttctacccgctgtgtctgtaaatctgactgctcgtatatgcctcagtgagtggagccatacgatatttttccttctgtgtctggctattttcacttagtatactgtccttaaggttcatctgtgttgtagcgtgtgtcagaatcttcctttatttttaaggctgaatgacagtctgttgtatgcacacgcgttttgttcatcctgttacccgtctgcggacattcagggttgtttccaccttttgactgctgtggataatgccgctatgaacacgggtgtacacacatctggtcaagtccttgctttcggctttcttggttatatatccacaaatggaacagctggatcagatggtgattctatttttaggttttggactgttctccacagcagcagaatgattttacattcccaacagtagtcacaagagttccaacttgcccacagtctcagtaacatttgtttattttgtttggtaataactatcctagtgggtatggaacggtacctcactgtgcttttagcttgtgctttcctaagtgatgctgagcgtctttcatgtgcgtgtcttctgggtattaaccctttattggatatatgctttcccagttttttctctcatcccatgggttgcctcagaagtgttttttgttactcttgttctggccacaccgcacagcctgggggatcttcgttccccaaccagggactgaacctgagctccctgcagtgagggtgcagggtcctagggactggctcaccagggaagtccccctttactctctcagtaatgccttctaatgcacaaaatgttttcattttgatgaagttcagcttagctaatttttttgtttgttgcctgtgcttttgatgtcatctccagaaacgatggctaaattcagtgtcttgaagatgttctcctgttttcttcttggtacagttgtcactgagtagcttttttaaactactgatactcaagtctactggggaagattctctccagtttgttttctaggatgaggcccacacatggatttttaaa

SEQ ID NO: 40 - DNA, 2851 bp, Bos taurus
  source 1..2851: mol_type = other DNA
cgtcctgccccgccgggccgccccgtcgcggccccagctcggacccccggccaccgtggagcgccgccgccgccccgcgactcacacacggagctggaactatgggatgtataaagtcaaaaaggaaagacaatctgaatgacgatggagtagatttgaagacgcaaccagtacgtaatactgaccgaactatttatgtgagagatccaacgtccaataaacaccaaaggccagttgcagactctcagcttttaccaggacagaggtttcaaactaaagatccagaggagcaaggggacatcgtgatcgcattgtatccgtacgatggcatccacccagacgacttgtccttcaagaaaggggagaagatgaaagtcctggagcagcacggagagtggtggaaagccaagtccatttcgacaaagaaagaaggcttcattcccagcaactatgtggccaaagtcaacacgttggaaactgaagagtggtttttcaaggatataaccaggaaggatgcagaaaggcagcttctggccccagggaacagcgctggagctttccttatcagagaaagcgagacattaaaaggaagttactctctgtctgtcagagactatgaccccgtgcacggtgatgtgattaagcactacaaaatcagaagtctggacaatgggggttattacatctccccacgaatcacttttccttgtatcagcgacatgattaagcattatcaaaagcagtcagatggcttgtgccggagattggagaaggcgtgcattagccccaaaccacagaagccatgggacaaggatgcctgggagatcccccgagagtccatcaagctggtgaaaaggctgggcgccgggcagttcggggaagtctggatgggttactacaacaacagcaccaaggtggccgtaaaaacactgaagccaggcacgatgtctgtgcaggcgttcctggaggaggccaacctcatgaagacgctgcagcacgacaagctggtgcggctgtatgccgtggtcaccagagaggagcccatctacatcatcactgagtacatggccaagggcagtttgctggatttcctgaagagcgatgaggggggcaaggtgctgcttccgaagctgattgacttctctgctcagatcgccgagggaatggcgtacatcgagaggaaaaactacatccacagagacctacgagccgctaatgtgttagtctctgagtctctcatgtgcaaaatcgcagactttggtcttgctcgagtgattgaagataacgagtacacagccagagaaggtgctaagttccccattaagtggacagctccagaagcaatcaacttcggatgtttcaccatcaagtctgacgtgtggtcctttggaattctcctgtacgaaattgtcacctatgggaaaattccctacccagggagaactaacgcggacgtgatgactgccctgtcgcagggctacaggatgccccgcatggagaactgcccagatgagctctacgacatcatgaagatgtgctggaaggaaaaggcagaggagaggccaacatttgactacctacaaagcgtgctggacgacttctacacggccaccgagggccagtaccagcagcagccttagtgctggggactcaagccacgcacgggaccgccgcctccctccaaagggaaaaacaacaatccccggtcactagttatttcctgggctggcttccctggctatccttgcttccttcacacaaggctaaaactcaggaaggacttcttccatgggaaaggattctcctctcaagggcaatatgcccaccagcctgcacccagtgggcacactggctctgcgagtccaacacctggacatggatttctgagaagaaaacacctatcccaccaaagtgcacccacatcggcctcttgtttacaagggggcatgtgactcagaggttcggaggccatttcagtgagtcgcagacaggagcacaacagaaataaccagatgtctccattgcacttccttgtatgttctctatgcttgagcttatttgttaaaaaaaaaaaaaatctactaatccaacctgttagatttgcaggtgaagtcagatgcttccatgtccttcccagatttctgtgacctcccaccccatgaaatctggactggagatcacttccttccacggagatcactcagaaccccacctgggcagaggaatctctttccttgaaagtgtcatcagcaagcaggcagggccaccgtgttaagccatgcggattccccaaccacccaggtttgcccagctcccctggaagggagcttcaggaactgctctgccaatagaaggaatgttgttttaaagtccagcttttcagttccccaatttcctctctccagacttctttggaccttgtagtttggacgaacaagatgcttctaatctctgcggaaccttctagggccttacaaaacatcagagctttctttattgcttcaaatgattctgaacattattttattgatcaagtattcattttagttgtactctagaaagtaaaaatgccgtattcgggggctatttttgtgattcagcaatcttttctaaattgtgtgtggtctgtgtgagtctatttatatgaggctatgcgggaaactaaatgagtgaggaggcttttctgagtcactggagcaattagatagaggttttgtttgttaaatttttctactgtagcttgaatggaaaacgctatgtcatatcacatttcacctatgctgtttgtgtatatacctaatatttctatttttgatcttattttaatacacctgggcccgtaacatctcaa

SEQ ID NO: 41 - DNA, 5442 bp, Bos taurus
  source 1..5442: mol_type = other DNA
ccagcccggcgggagagccggcgcaggggcaggaggaggggagccgcacgccgggagggaagccggggcgcggcgaggaggtggcgggaggaggagacagcgcggaaaggtgccacataaaggagggcgctcctccgcgctggaggcatcatggccgctaagtcagacgggaggctgaagatgaagaagggcagcgacgtggcgttcaccccgctgcagaactcggaccactcgggctcggtgcagggactggcgcccggcctgccgtcggggtcgggggccgaggacgaggaggcggccgggggcgcctgctgcccggacggctcgcgctgctgctgctgctgctgctgcgccgggagcggcggctcgggcggcggctccgggggctcgggcggcccgggcggcggctccgggccgggctcggcggcgctgtgcctgcgcctgggcccggagcagcggcgctactcgctgtgggactgcctctggatcctggcggccgtggccgtgtacttcgcggacgtgggcactgacatctggctcgccgtggactactacttgcgcggccagcgctggtggttcgggctcacgctcttcttcgtggtgctgggctcgctctcggtgcaggtgttcagcttccgctggttcgtgcacgatttcagcaccgaggacagcgccgcggccgccgccggctgcgcgcagcccggggccgagggcaagaccgcggtgggcggcggggccgcggccgcggaaggcgaggctcgcccctccacgccgcagaggcaggcgtccagcgccggcaagggccacgtcgccgccaccaacagcggcagcaacagcagcggggccgcccgggccggcggcaaacccaggtctgcgtcctgttccttctgcatctggctcctgcagtcactcacccacatcttgcagcttgggcagttctggaggtatttccacacgatatacctaggtattcgaagtcggcagagtggggagaacgaccggtggaggttctactggaagatggtgtatgagtacgcggacgtgagcatgctgcacctactggccaccttcttggagagcgccccacagctggtgctgcagctctgcattatcgtacagacacacagcctgcaggccctgcaaggtttcacagcggcagcctccctggtatccctggcttgggccttggcctcctaccagaaggcccttcgggactctcgcgatgacaagaagcccatcagctacatggccgtggtcatccagttctgctggcacttcttcaccatcgcggccagggtcatcacctttgccctcttcgcctcggttttccagctctactttgggatcttcattgtccttcactggtgcatcatgaccttctggatcgtccactgtgagacggagttctgcatcaccaagtgggaggagatcgtgttcgacatggtggtggggattatctacatcttcagctggttcaatgtcaaggaaggcaggacacgctgcaggctcttcatttactatttcgtgatccttctggaaaacacagctttgagtgctctctggtacctctacaaggctccccagatcgcggacgcgtttgccatcccggctctgtgcgtggtcttcagcagcttcttaaccggcgtggtttttatgctgatgtattatgccttctttcaccccaacggaccccgatttgggcagtcaccaagctgtgcttgcgaggacccttctgctgccttcgctctgcctccagacgtggccacgagcaccttgcggtccatctccaacaaccgcagtgtcgccggcgaccgcgaccagaaattcgcagagcgagatgggtgtgtccctgtctttcaggtgaggcccacggccccatccaccccatcctctcgcccaccacggattgaagaatccgtcattaagattgacctgttcaggaataggtacccagcgtgggagagacacgttttggaccgaagcctccgaaaggccatcttagcctttgaatgttcgccgtctcctcctaggctgcagtacaaagacgatgctctcattcaggagcggctggagtatgagaccactttataaaacgagaggacttggaggggccacgacctccagacagaggggtgacagcagggctgtggcaataaggaaactctcccctagagcagggcgttgggctgtgagactcccagtttgaccctcatcggatacgtgtctgaccctcggacagcagagtgcccgctgctggcctcttttcaaagagcagccccaagaccctcctactctgtctgaagggaacagcccagctcagtgggactccttgttgccgccaacccctccagctcaatcttcagcacatccatccaagggtactgttgtgatgggaaatactgcctccaatcagtagggtgtttggatgagttaactgatctttgcataaaaatatactgaatcacacatacacacgagtacacacacactaacacacatgccaatcctctgtcaaaacagtttaggtgatttctcttgatgcaaagctgtgattcctgcaggaaaactaagagagaaaacatatccctcaagactccagaaaaacaaaaaacaaacaaataaaaaaaaatatagtgaagcttagaaagaattcataaaccatcctcatgtgaaaaccttacccaatggacagaaaacccaagtgtgtttacacatgtattagaattgatgaaatggttcattttcaacacacatcaggatttttttttcattttaaattttcagttgagaacatctcctattacagaaatcctatgcagcccatggatggcttctgacaagaccaaggagcttgataccttagtgatgtaagttcagtgataatcaggatgcagtattcagttgcaaaaacatgtaataaaaccacggagaggaagtgggagcaaaaggcagaatgagtgtctgatttcctggcctgtgcagctcacacgggaaggtcagggtggagcggcgggagagacagccagtgttcaggtgcaggaatggaggttctatgtgatggcgggcaggcaggatttcagaatacgccatgccacacccttctagtgccaaagtccatccaacacgagactgacgggaaagccctgaacggctgagcatgggtggcgtgagccccaccaaagcagaagactgctcaaacagtcaagtcctctttgtgtaaaggtgaccctctccccccacccccaaggtcgaagtgatgtgtcttttagaggctttgggacacatttttgtaagtatgagcccacaggtgcagtgaatatgctgtgagaaaacccttgtgaactgattgagggcttatcactagatccagctaagatctgtatttcagtcattttgtaaagtcccactcttacaacaagcttctgggttttaaatacctctgtccagcaagtaaacattccccactttctgttcccagtgtccccccccacccccagtcatggtgctttttgttgcattaaaagtgccggtcaaactttgatagtatttttttatagttggtgcagagtggaataactcatggattatttcaatatttttgtaataaaaaatatagggtatacatataggcatcatcactttttttataaacctgggatcgtttaaaatactttaagcatcataattacttgggatgtagaaactggtccacagatcccatctgcctgcctcagcagatcgagaacacatgtttcttgcaagatcatcctactttgccatgtcggtgcccccaggaacctggccaggggtgctatcagaatatcaggggacgagagaatcagcttaactagaaagggcttgtcaagactggccaatgtttcccaggaaaccaatgatggaagtgatgactcttccatctcttatgacaaacctgaccacagcgggcaatgtcttggatgtcctttcccagttttatatctacccaactgatgcactaaatattagtcaagtcactttttaaaaaaaaaaaagtcagtttaacttaattattcagttatctggactgtccaattcacttaaacagtcaagttctagagccactacctgtcatgactgtgtcgtacagaggcagccgtccctatgacgacacccacagcaagtgacagagggctcgaactaggactttttttggtactaagtccaaaggcagtgtttaaaaattagatgatttttatcagtagacatattttcctggtactccatgatggatcctccaagcagcagtccactaaaaaataatctttggtttggatgtgaaaacgttacctggtcaccaggaaaggcccagaaagatgctcttcctgccagcaataggcaaaaacattccactccccactctagaagggaaaaatgattatattggcaaattcttcactttggtgccagcccgtgggtcattagcttcataattaatgatcttttggaaaccaaattgctcagttgaactatccctttgattccatgagtaaatcgcagatgataatgtcagtcaacaaacacaacaggtcccatcactcttcgatcatgtaaatcctaagttatttctcaacttgatccttcgtttaacaggtgaagaatcaaaatgagcgtggtgtgactaaaaaaacgctgtactgtgctttgtcttcaaagtgagaatgcagtcaggagagtgcaagaagtttgatattttcagtgattttaccaaatactgtaaatactctgttatccaacatacttaaaaggtgagggtttaatcaaaaatacttgttaaggaaatgacatctccaccatgtcataaataacaagacttctcaagattaccgtcaagaacaacacgacaacaaatgttacttccaaggagaatcatggcttaatcttgcttccatgattcagaaagcactgtgttgccatcgagcatgtatttgtttcagatacactgataacagagcatttccagtattaggatttaaagagacattgtcaatgtacaaaggaacaaagtttgattttcatatttatactctagtataaaccctaagattttattttcagttgaccactaaattctccacaagagtagctcaagaatccaaaaggaacaggccttacaaattggaacctacaaatttgttaggatacaaatcggaaacactggattctttatctgtgagctccaacatcatgtgctgggtctgttgggtcacttgggggaggggtgcggtgaagggtctgtccttagtaattaattttgcataggaccatgagttttctaaaaggaaatattataaatcttcttcacttttaaatgaacatttctcaatgaaacttaagttgaggactgtatgtatcctaaccacactgagactttaagtatttgtgtggaagaaaaaaaaaaaaaaa

SEQ ID NO: 42 - DNA, 2090 bp, Bos taurus
  source 1..2090: mol_type = other DNA
atgcactcggcttccagtatgctgggagcggtgaagatggaagggcacgagccgtccgactggagcagctactacaccgagcccgagggctactcctcggtgagcaacatgaacgccggcctggggatgaacggcatgaacacgtacatgagcatgtcggcggccgccatgggcagcggctcgggcaacatgagcgccggctccatgaacatgtcgtcgtacgtgggcgcgggcatgagcccgtccctggccggcatgtcccccggggcgggcgccatggccggcatgagcggctcggccggggcggccggcgtggggggcatggggccgcacctgagcccgagcctgagcccgctaggggggcaggcggccggggccatgggcggcctggcgccctacgcgaacatgaactccatgagccccatgtacgggcaggcgggcctgagccgcgcgcgcgaccccaagacgtaccggcgcagctacacgcacgccaagccgccctactcatacatctcgctcatcaccatggccatccagcagagccccaacaagatgctgacgctgagcgagatctaccagtggatcatggacctcttccccttctaccggcagaaccagcagcgctggcagaactccatccgccactcgctgtccttcaacgactgcttcctcaaggtgccccgctcgcccgacaagcccggcaagggctccttctggaccctgcaccccgactccggcaacatgttcgagaacggctgctacctgcgccgccaaaagcgcttcaagtgcgagaagcagctggccttgaaggaggccgcgggcgccgcgggcggcggcaagaaggcggccgcctccgccggggcccaggcctcgcagggtccgctcggagaggcggccgccgggccggctcccgagactccggcgggcaccgagtcaccccactcaagcgcctccccgtgccaggagcacaagcgagggggccttggggagctgaaggggacgccggcctcggcgctgagccccccggagccggcgccctcgcccgggcagcagcagcagcaggccgcggcccacctgctgggccctccgcatcacccgggcctgccgcccgaggcccacctgaagccggagcaccactacgccttcaaccaccccttctccatcaacaacctcatgtcgtcagagcagcagcaccatcacagccatcaccaccaccagccccacaaaatggacctcaaggcctacgaacaggtgatgcactaccccggctacgggtcccccatgccgggaagcctggccatgggcccggtcacgaacaaagcgggcctggatacctcgcccctggccgcagacacctcctactaccagggggtgtactcccggcccattatgaactcctcgtaagaagacagcggcgacctggctagcccgggacccagatgggagagacaggaagtggggtggagactctgaggaagggctgctggaaaggtgcaagggagaaggacccttcacaccctgccctgcccccccacagaggtctcccctcaccctctgcagtccttccctccctcacgtgggccacactgatgtctaccatcctatataaggagggaaaaagaaaaagctaaacagtttttaaaaaagaagaaaagcctcagtttccactactgtgtagacgcctgcttctctaaaaacctgccaattcttgactttttgttgttgtggttcctcttcattgctgtcgttgcagggaagtcttactttattaaaaaaaagaaaaagaaaaaaacacaaacttttgtgagtgactcagtgtaaaactatgtagttttaacagaaaacaagagttgtactattgttttaaaaagagaaaaaaaaataataatgtaagggactgttgtaaatgcccaagaaaagaaaagaaaaaagcattcgcattcttgatacggtgaaatccaggtctcaagtctgactaatttatggtttctgcgtgctttatttatggcttataaatgtgtgttctgactgcaaggaccagagttccacaaatctatattaaagtgttattgccagttttttgctttgaa

SEQ ID NO: 43 - DNA, 5339 bp, Bos taurus
  source 1..5339: mol_type = other DNA
gaaggcctcgcactggcgaagccgcggtgagcactgctcgtgcgcgcgcgtgtacgtccgccgggagctccgctgctcggtgctcgtgcccgcctgcccgtcgccggcgctcgcgccggggagcgccgccgggtccttagagccggcaggcagctccctttgtgcggcgctcgcgccgcggtccctccggacggaatgtgggctctgtgggagtttttctcttcttcccaaattcttgaaacaatggactgtaaggggcgcggggattaaggggggcgagctgggaagcgacctgcactggggaatcatgacttctgctgcagagattaagaagccaccagtggcccccaaaccaaagtttgttctggcagctaataagccagctccacctcctgtcgcacctaaacctgagatcttggtttctagtgttccacagtcaacaaagaaaaccaaaccatccatagctccaaaaccaaaagttctgaaggcctcacctgtccaggacatcacacagtcaccatcaaggaaaatcgtcgtgaacctggaagagcatacacaggaattccctgaaagtattgacaactcgaactgcagaaacagagggcatccgagcagtgattatattttaccagtgtgttcctgccatcctggatgtaaccataagcttgagagcagaaagaatttgtatgtcaagcagcttgtcttagggcccctggaaatgcatgagaatttagaaaacaacaaaattggtgagtccccttcagctgtgaaaaatcagcataaatgtgattctgttggtgaaagggtcaagagccagagtaaagtagttttaaaggcaagcatcctcgaagagaagctcaaagatgttttaacacaaccaatgtcaccctttcttttcccccagaagcacagatccacagacagtccagaaatgagtgatggctgtaattcccacagacaattcagcattgaatttgcagatttgtcaccttccctgtccagctttgaaaacgtttctaatcatcacccttgccacctacaacttcctagcactgaatttcaaaattttgaaactaatcagaatgacagtgaaaaaagcaacacttgctatcattcatctgaacaagaagctctagaaaatggaaaaaggagtcctttcttatcttcagatggagatagtaagaaatcagaagccagagaccttggtcccttggaaatccacttagtgccatgtactcccaaatttccaactcccaagcccagaaaaacacatgttgcacgcctcttacgccagagacatatagatacttctagtgaaagtactgaagaaccagaggatgtagacagcttgcctcatgttttcgaagatagtttgaaaaacaataaaatcagtgtctttcatcagactgctttgtgtaaccaggaacaggtggacaaagtgaagccagaaaataaaggtgaattgaatgtggacctcactggtgacagacaaaactcggtctattcacagaaagccttgcgtcctgaaacattttcctctgaaaaaacggcacctcctttagaaacagactctaacttgagttctgacgacaagtcaggagatagttctggtatgtcacttgccgaggacaaaggaatcagttttataagatgcagtactctatctatgagcctgcccaagcagctcaaattaacctgcagcgaacatttgcctactgcctctaacccaggagtgtctgcccctcaaatgcaaaaggaatctatgataaaaggtgaaagttcctcaaaagttgtccccaaaaaacctcaaagacacagtttgcctgctgccggagtgcttaaaaaggctgcctctgaggagcttttggagaaaagttcttatccctcaaatgaagaaaaagatgcagagaagggtctagaaagagatcacgtgcagcatttatgtgccccaaacaatggtatgtcatcctcctttgatatgcctaaacgggcttcagagaagccagtgtggaaattacctcatcccattatccccttttcagggaacccagaatccttaaagtctgtcaccatatcctcaaacagtgagactcccacacccctgaccaagcctagagcaaaatcattatctgctatggatgtggaaaggtgcactaagccatgcaaagactctcacaagaagaactctttaaagaagttgctcaatatgaagctgtccatctgtttcatgaagagtgacttccagaaattttggtccaagcatagccatcttggagacacaacctcaggcagcctggcaggtggggagcaaagagggagtgagagtgattggcatggcctgttggtaggagaagagaagagaagtaaacccatcaaggcatattcggcagataactatagcctggagtcccacaagaagaggaagaagttgcggggccacaccaatgcagctaatggaccaagagccgagtctctggatgaccaaatgctgtccagggagtcgtccttgcaggcaccttgcaaatctgttaccagcgactgtgtaccagagtatgaaaatatacggcattatgaggaaataccagagtatgagaacttgccctttgttatggctatggggaaaactcccgagttggaacgacggaactccagcagcgtggaggacactgatgcaaatgtgtatgaggtacaggagccgtatgaagctccagacggccagctccaactcggacccagacaccagcattccagcagttctggagcatcccaggagggacaaagtgatctggaccttggtcttggtgaccttccttcagatgaagaggaagtcatcaacagttcagatgaagatgatgtcagctctgcttctagtaaaggggagcctgatctgctggacgataaacagggtgaagatactggaatgaaatgtaaagtccatcatattgccaaagagatcatgagctcagaaaaagtgtttgtggatgtgttaaaacttttgcatattgatttccgggatgcagtagcccatgcttccaggcaacttgggaaaccagtgattgaggaccggatcctaaaccagatcctgtactacttgcctcagctgtatgagctcaatcgggatctcctgaaggagctggaggaaagaatgtcaaactggacggaacaacaaagaattgctgatatctttgtaaagaagggaccatatctaaaaatgtattccacatacatcaaagaatttgataagaatatagctttgttggatgagcagtgcaagaaaaacccaggttttgctgctgttgttagagaatttgagatgagcccacgctgtgctaatctggccctcaagcactacctgctcaagccggttcagaggattccccagtacaggctgctgctaacagattatttgaaaaatctcttagaagactctggagattacagagacactcaagatgcccttgctgttgttatagaggtagccaaccacgccaatgacaccatgaagcaaggagacaacttccagaaacttatgcaaattcagtacagcttaaatggacaccatgaaatagttcagcctggacgggtttttctcaaagaaggaattctgatgaagctatctcggaaagtcatgcagccccgaatgtttttcctgtttaatgatgccctgttgtatacaacaccagtgcagtctgggatgtacaaactgaacaacatgctatctttggctggaatgaaggtcagaaaacccacccaagaagcatatcaaaatgaacttaagatcgaaagtgtagaacgttccttcatcctctcagccagttctgccccagaaagggatgaatggctcgaagccatttccaggtcaatagaagagtatgccaagaaaagaatcacattctgtcctagtcggagtcttgatgaggctgactcagaagataaagacgaggttagtcctcttggatctaaggctcccatctggattcctgataccagggccaccatgtgtatgatctgcaccagtgaatttactctcacctggagacgacatcactgccgggcctgcggaaagattgtatgccaagcttgttcatcaaataagtatggcttagattacctgaaaaatcaaccagcaagagtatgtgaacactgtttccaagaactgcagaaattagatcaccagcactctcctaagtttggatcccctggaaatcacaaatctccttccagcgccttatcatcagtcttacatagtattccatcagggaggaaacagaaaaaaatcccagctgctctgaaagaagtatcagcaaacacagaagattcctctatgagtggctacttgtacagatcaaagggcaataaaaaaccttggaaacacttgtggtttgtcataaaaaataaagtactgtatacatatgctgcaagtgaggatgtggcagctttggagagtcaacctttactaggattcacagttactcaagtcaaagatgagaattcagagtctagagtgtttcagttactgcacaaaaacatgctattttatgtattcaaagcagatgacgctcactcagctcagaagtggatagaagcatttcaagaaggcacaatactgtagttgcgttggtttcgtctcctgtggttccaaaagggtggaatttcatcagaataaatgcagtacaaacattttatataaatgaacaatgccaagatgaatccaactaaaatcttcatcagagagattattttattaggtataaggtaatttttaaaaaatgtttgtttttcatgtatgttatttattaaaattttaatgtttacttaatggtcagagttatttctgagactcacactgtattctgaagtaccttttttttagaaaccagaacacttaccttaataccgtattaactgtttgtgacacggtccctcctgttttctcagtgtgttttacaatgttactttgtaacattctcaccagtggtatgaatttctatacagtctttgggttggaaattatgattaaacagctgatgagaaaaatgtatgtctttgaatccatacatttagatgaattttttttatcttttaatggaaaaacatatggccaatttctacctcagtaactgaaacaaaattccagtaaattgtctaaagtatttctactgttacgtacctcttgttccatgagataatgttatatatcatcaaggaattactgcaaagggatagaatctgaattttcacttaactcaattgtgagtaggaaaaagaatgtttccattttcagggttacacaaataattaactctatggcctttacattaagtctaatctttaaaaaa

SEQ ID NO: 44 - DNA, 7737 bp, Bos taurus
  source 1..7737: mol_type = other DNA
atgctgtccctggtagggaagctgccctgtagtccgtccccagtttcagtatatgtacggctgaagtgggctctggcattgtcaggagccggcccttggctgtccccaaggcctggcctcacttaccctctgacccctgcccagtgccaggatggtggagactggcctgaagggctggttgctctgggccctgctcctgcactcggcccagagtgaggtctacacacccatacaccagcctggctactgtgccttctacgatgagtgcgggaagaacccggagctgtccggaagcctggcttcgctgtccaatgtgtcctgcctggataactcacctgctcgccacatcacaggtgaccacctggccctcctacaaagcatctgtccccgtctctacactggcgccagcaccacctacgcctgctgctcctccaagcagctggtggccctggacatgagcctgcggatcaccaaggccctcctcactcgctgtcccgcctgctctgacaacttcgtgagcctgcactgccacaacacctgcagccccaaccaaagcctcttcatcaatgtgacccgagtggtcacacagggggacagccagtcccaggccgtggtggcctacgaggccttctaccagcgcagctttgccgagcagacctacaactcctgcagccgtgtgcgcatccccgcagctgccacactggccgtgggctccatgtgtggcgtctatggctctgccctctgcaacgcccagcgctggctcaatttccagggggacacgagcaatggcctggcacccctggacatcaccttccacctgtgggagcctagccaggccgaggggagcacgatacagcctctaaatgatgaggtcgtgccctgcaaccagtcccagggggacggtgcagcggcctgttcctgccaggactgtgccgcctcctgccccgtgattgcccagccccgggccctggaccccaccttccgcctgggccggatggaagggagcctggtcctcattatcatcctctgttctctctttgtcttgctcacggccttcctcctgcggtcccgcctggccgagtggtgcaggggcaagcgcaagacgcccaagcccaaggcaagcatcaacctggcccacaggcttagcctctccacccacaccctcctcagccgctgcttccagtgctggggcacttgggtggcctcatggccactgaccatcctggcggtatctgtgattgtggtggtggccctggcagggggcctggcttttatagaactgaccacggaccccgtggagttgtggtcagcccccaacagcctagcccgacgtgagaaggcgttccacgacaagtatttcggccccttcttccgaaccagccaggtgttcatgacagcacctcaccggcccagctataggtatgactccctgctgctggggcccaagaacttcagcgggatcttgtcctcggatctgctgctggaggtgctggaattgcaggagaggctgcggcatctgcaggtgtggtcgccagaggagcagcggaacgtctccctgcgggacacctgctatgcccccctcaacccacataacgccagtctgtccgactgctgcgtcaacagcctcctgcagtacttccagaacaaccgcactcagctgctgctcacagccaaccagacgctgtcagggcagaccgcccaggtggactggagggaccacttcctctactgcgccaatgcccccctcacctacaaagatggcacggccctggccctgagctgcatggctgactatggggcacccatcttccccttcctcgccgtggggggctacaaaggaaaggactattctgaggcagaggccctgatcatgaccttctccctcaacaattaccctcctggggaccccaagctggcccaggctaagctctgggaggcagccttcttggaggagatgcgagcctttcagcgtcggacggctggtgtgttccaggtcacattcatggcggagcgttccctggaggatgagatcaacagcaccacggccgaggacctgcccgtctttgctgttagctaccttgtcatcttcctgtacatctccctggccctgggcagctactccagctggcgtcgggtaccggtggactccaaggccacgctgggcctgggcggggtggccgtggtgctgggagcagtcgtggcctctatgggcttcttctcctacctgggtgtcccttcctcactggtgatccttcaagtggtgcccttcctggtgcttgccgtaggggccgacaacatcttcatcttcgttcttgagtaccagaggctgcctcggaggcctggggaagaacgggaggcccacatcggccgagccctgggcagagtggcccccagcatgcttctctgcagcctctctgaggccatctgcttcttcttaggagccctgacccccatgccagctgtacggacctttgccctgacctccggctttgcagtgcttcttgacttcctgctacagatgtcagcctttgtggccctgctctccctcgacagcaggaggcaagaggcctcccggatggacatctgctgttgcaagacagcccagaagctgcccccgccgagccaggatgaggggctcctgctccgattcttccgcaagttctacgtcccattcctgttgcactggctcacccgcgtggtcgtggtgctgctcttcctggccctgtttgcagcgagcctctatttcatgtgctacattaatgtggggctggaccagcagctggctctgcccaaggactcatacctgatagactatttcctctttatgaaccgctactttgaggtgggggccccggtctattttgttaccactgggggctacaacttctccagtgaggaaggaatgaatgccatctgctccagtgcaggctgcaacaacttctccttaacccagaagatccagtacgccaccgacttccctgatatgtcttacctggccatccctgcctcctcctgggtggacgacttcattgactggctgacttcatcttcctgctgccgcctttatatctctggtcccaataaagatgagttctgcccctcgactgtcaactccctggcctgcttgaagacctgtgtgagccccacagcgggctctgcccggccatcagtggagcagttccacaaatatcttccctggtttctgagtgacgagcccaacatcaaatgtcccaaaggtggcctggcagcatacagcacctcagtgaatatgagctcagatggccagattttagcctcgcggttcatggcctacaacaagccgctgaagaactcgcgggatttcacggaggctctgcgggcaactcgggcactggcagccaacatcaccgctgacctgcggaaggtgccaggcactgacccagattttgaggtcttcccttactcggtcaccaacgtgttctatgagcagtacctgaccatagtccccgaggggctctttatgctcaccatctgcctggtgcccacctttgtcgtctgctgcttcctgctgggcatggacgtccgctccggcctcctcaacctcttctctatcatcatgatcctggtggacactgtgggcttcatgatgctctgggacatcagctacaatgctgtgtccctcatcaacctggtcacggcggtgggcatctctgtggagtttgtgtcccatatcacccgctcctttgccatcagcaccaagcccacccggctggagagggctaaggaggccaccatctccatgggcagtgcggtgtttgcaggagtagccatgaccaacctgcctggcatccttgtcctgggcctggccaaggcgcagctcatccagatctttttcttccgtctcaacctcctcatcactgtgctgggcctgctgcatggcctggtcttcctgcccgtcatcctcagctacctggggcctgatgtcaacccagctctggtgcagcagcagaagcagcaggaggaggcagctgcggccaaggagacctcttgctcaaagcaccccgctcagatgagcactgattatggtgtctacgtcaactgcagctttgaacatcctgccaagagtgtaggaggcctcgagagctctccatcagagaacaggcagaagttctgatggggccagagcccctgtccaagctcatcagccatgacctgaggggccatgaaggctcctcctgggggtcctttgacttatccttcccccaggccctgagagaggctgtctccacaggaccgtccaccatcggtcatcctctagccaccagatctcacccataaggacactgattgtgatagaggtgccacgtgatggcttgggtgtgacctggtccaaatccatctcttgaatttgacatgtgtcctcctcatgcaagttttaaaactttagttccatatatgtgaacaacatatttgttcacatttaaaattttccttaattgataccatatgtgacctatccttttgcaatgaggtttttttttttttttatgcagcatacattttttcgatttattcgtgacgatacatggagatctagtccaatcattttaactgtgacacagctttccattgtgtgaatgaacaggatgtttttttatgcattcccctgctagtgggcaatcacattgtcctgaattattttttctgctataagtccacagtcacttatgtggaatttcagaacccccagaactctgaaaacctagtgtgtttgtttttttcttttgtaagtttgggacaaactcatttcatagcaaagactgatcagacacgatgtatctatttatactctttataaataggtttccctggtggctcagatggtaaagaatttgcctgcaatgcaggagattcaggttctatccttgggatgggaagatcccctggagagggaaatggcaacccactccagtattcctgcctggagaattccatggacagaggagcctggcgggctatagtccacggggttgcaaagagtcagacacaactgagtgactaacacttctctttttttttttttctcccataaatagcatacttacactcttcagccaacttgatgtgaatagtcatatatttcagcacattaacaagtttgatttcatgttgcttccccagatcctgatgaggtctaatatctaatgtactgtaagcatcaaattacctttctaaaaaaacgttctaaattctgaaattcagcttgctatgggatttcaggtatggaaccatggacctgtgtgatgagcaggtgtttacacgtctcctgtctatgtgtggggagctaccatcgctgtagcactgaatggatacagactagtgtcttccagggcgtctgccctattcacacctccaccagcaggggataagggtgcctctttccctgcaccaataccaacacaacgctccacttgtttctgggagggaggggagtggggttatagattgttatccctttctaaaaaatacttttttaaaagaaaactttttttattttagaatagttttacagaaaaattacaaagataatacagagagttcctgtatgccccccgcccgccccccaaaatttacttgagtaacacattagtgtgataacacttgtcagtgtaccatgaatgaaccagtactgataattactactaacccaaatccatattttgtttagatttccacaattttacccagggtccttttcctgagccaggatcctgtccaggacactccacaacactgttgttatgtctacttaggctccttttgtttgtgacagtgtttcaggctctccttttctttgtttttggtgacccggacagttgaagattggtcaggtattttaaagaatgtccttcaaatggggtttgtctagtgtttctcccgattagacaaaggtgatgggtttttcggagggagatcacgaggcagagagccggtctcagcctattgtgtcaagggtgcctgcagtcaccgtaacttgtcgctgtcacactggccctgaccacctggctgaggttgtgttggccaggtgtctcctctgtacacttatcttgcacctgtttgcgtggtgcactctttgcagcgaagttgctatgcacagcctacacttaagggtgagtggttatgctctacttcctggagggtggagtgtttacataaattatttggaactcttctgaacaggaaaattgttctccatttattatttattcagtatgctatctttatcagtatggaaccaaggatgctcattttttactttgggttttaatatagtgactatttgctccacatttggccattgggaactcttccagttggctcctaggtccctttgatgtattcctttcactgtggggttccccccactttttgagcacttttgtacttcctggcattataagatagtataggctcaacttgtatatttctatcccagtcctagagtcagccatttctcccaagagttccagtccttcatctggagataagattagaaatcaggatctgggtgttcagtgtgttttgctgctggggctttgttgcttctgatcctctcagctgagagagcaagggcatacatatgtgaatactgacctgtatatgtacacatttctagaagtatttctatgaaaagccatctgtatctacattaagctaaacaggattttatgctgatacctccagttataacccataatcacatgaatcattctagactcctttcttgcttctctgtagcctctcatgccactaatgagaagccgggctccacaaccctcaccatttacttagttgttgattacctttatacatatgtaggagtctgagaaatgataacctacactcctgtggaaaacaactttagaatttagtgtttacatgcaattccttttgcctttagtcttacagacatcattcctttccaaagttacttcggttagcatctttcagatggggtcagtatccctttcagagaagctgcttcatacattgaaaaaatatttatttatttatttttggctgagctaggtctttgtttctgccttcaggccttctctagttgcggagatcaggggctactctttagttgtggtgcgtgggcttctcattgcagtggcttctgttgttgcagagcacaggctctaggtgcatgggttcagtagtagtggggcatgggtttagttgttctgcagcatgtggaatattcccagaccagggattgaactcgtttctcttgcattggcaggattcttaacccctggaccaccagggaagcccttatacatttttaatatagttaagattcttgcatcacattctttattccattctgattttacaaaatcatgtgtgtaaaagtttgttgtgggtattcagcagaacacgatccctggttgaccctctggctagacccaggcaatgggagactcctcacgccttcctctgtccttgtaatgtatgctctgctgctctgcgtaccattctcacagcgagagttgttttcaacaacatcttcctctcaacaccttgagagaggaaggtgtttttgagaccatctggacagtgtatgtgattgaacccagttaaggtgtctgtataaacatttgcgattctggcaaagtggacgtgaaaatacactggtcttgtggctgcccaggacaagtaactttccttgcttattaaacctcctacctatca

SEQ ID NO: 45 - DNA, 1600 bp, Bos taurus
  source 1..1600: mol_type = other DNA
catcaccttgcatttcacccacgactacgttagtaacgggaaacacaccaacagaaaggtctcagtttcctcttgggactcgcaagcaaagggttctcgctgtcccgcctagtttctggatatccagtctggaacacagcccacaaaccacgtgacaacagcgtggctcacgtaacagttgttgcccgcgctacaacttttccgccagtttccaacatggccactaggcgccctcaatgccgcagccccgggccctcttgcgcttcccggcccacttctgctcctcagcgagacctcccgagaagcccgggggcaataggggcggtgcctgggtccagtcggctccagcgcagtccagcccatctccaccgcgggggcgaacaagagcggaccggaagtttcccgggcgcgctagcggccgaggccgttactgcagagcaccggaagtggccataggccggcgtccgggccgcgcccgcagtgagtgagctttcctccggccggcgacgccatggagcccggggcggccgagctgtacgatcaggccctgctgggcatcttgcagcacgtgggcaacgttcaggactttctgcgtgtgcttttcggcttcctctaccgcaaaacggacttctaccgcctgctgcgccacccgtcggaccgcatgggctttcccccgggggcagcgcaggcgctggtgctgcaggtattcaaaacctttgaccacatggcccgtcaggatgatgaaaagaggagaaaagagctggaggagaaaatcaggaggaaggaagaagaggaggccacggcggtggcagctgccacagcggagcaggagccagttccagccccggttcaggaggtggaggtagactccaccacagactcaggtgggccccaggagcccccaggcccacggggcaccctgcaggaagtggctgtcagctctggggaggcagaggcaccgggagcagatgctggcgctgctgaagcccccagggaaccgctagctcttcccaagagacaggagcagttccagagaaaccccgacagctacaacggtgctgtgcgtgagaactacacctggtcccaggactacaccgacctagagctcaaggtgccggtgcccaagcacgtggtgaagggcaagcaggtctcggtgaccctcagcagcagctccatccgggtggctgtgctggagggaaatggggagcgtgtcctcatggaagggaagttcacccacaagatcaacaccgagagctccctctggagcctcgaacccgggaagtgcgtcttggtgagcctgaacaaggtgggtgagtactggtggagcgccatcctggagggcgaggagcagatcgacatcgacaagatcaacaaggagcgctccatggccacggtggacgaggaggagcacgccgtcctggacaggctcaccttcgactaccaccagaagctgcagggcaagccgcagagccacgagctgacacggcaggagcagtttggccactgagctccatcagctgaggtccaaggaaaatggggtgcctgcctgcgaaatcaccctgtctccggtgttggtttgcaataaatctgtatttaagca

SEQ ID NO: 46 - DNA, 3426 bp, Bos taurus
  source 1..3426: mol_type = other DNA
gggctctgaggcgcgggactctgaggcgcgatggcggcgcgctgcttgggccgcggcgtcgggcggctactgggcggcctccgcggggttggggtgcgggcgctggcggccccccggctctggagcagcgcctccgcctccgtagcgccacccagctcctaccaagagcgaatcgcgcttgcagctcgcgagcccgccgccttctgggggccactggcgcgggacgctctcgtgtgggacaccccttaccacacggtgagcgactgcgacttccgcagcggcaggatcagctggttcctgggaggccagttaaacgtgtctgtcaactgcttggatcagcacgttcagaagtccccagagagcatcgctttgatctgggagcgagatgagcctggaactgaagtgaagatcacctacagggagctactggagaccacgtgccgcctggccaacacactcaagaggtatggagtccgccgaggggaccgagtggccatctacatgcctgtctccccactggctgtggctgccatgctggcctgtgccaggatcggagccatccacaatgtcatctttgctggcttcagtgtggggtccttggctgggaggatcaacgatgcccaatgcaaggtggttattaccttcaaccaaggactccggggagggcgcgtggtgcagctgaagaagatcgtggacgaagccataaaggtctgcccatccgtccagcatgtcctggtggctcacaggacagacaacaaggtccacatggggcatctggatgtttcccttgagcaggagatggccaaggaggagccggtatgtgccccggagagcatgggcagtgaggatatactcttcctcctgtacacctcggggagcaccgggaagcccaaggggctcgtccacacccaggcgggttacctgctctatgctgccctgacgcaccggcttgtgtttgactaccggccaggtgacgtcttcggctgcgtggctgatatcggctggatcacgggacacagctatgtggtgtacgggcccctctgcaatggagcgaccagtgtcctttttgagagcactccggtttaccctgatgcgggtcggtactgggagacggtgcagaggctgaagatcaatcagttctatggcgccccgacagcctaccggctgctgctgaagttcgaggactcctgggtgaagaagtatgaccgctcttctctgcggaccctgggctcagtgggagagccgatcaaccacgaggcctgggaatggctgcacagggtggtgggggacagccggtgcacgctggtagacacctggtggcagacagaaacggggggcatctgcatctctccacggccctcagaggagggggcagagatcctgccctgcatggcgatgaggcccctgtttggcattgttcccgttctcatggatgagaagggaaatgtcctggagggcggagacgtgtctggggctctgtgcctgtcccaggcctggccgggcatggcccggaccatctacggagaccaccagcgattcctggacgcctacttcgagacctaccctggctactacttcacaggagacggggcttacagaacagaggagggctactacgagatcactgggcgcattgacgacgttatcaacatcagcggccaccggctggggaccgcggagatcgaggacgccatggtgagcacggccgccgctggtctgggggctgagtctgagtcagtgcctcacctctgcgtgttttgtcctgcccaggctgaccacccctcggtgcccgagaccgctgtcattggctacccgcatgacatcaagggagaagctgccttcgccttcgttgtgttgaaagatgatgtgggtgatgtggacgtggtggtgaaggagctccggtcagtggtggccgacaagatcgccaagtacgcagtgcccgatcaggtcctggtagtgaagcgtcttccaaaaactcggtctggaaaagtcatgcggaggctcctgaggaagattgtcatgggccgagctcaggacctgggggacaccaccaccctggaggaccctggtgtcatcacggagatcctgagtgcctaccaggagtacaaagacaagcggggaggtgccaagtgatggctctgctgagaccaggcccctaatgcgtgctcctagagcacagctcccgagtcggcttgttcctagacaccccagcagaacccaggctgccctcctcgcgaagcctgaagtccccgcctcccctcctggggacccacgacaggtcctcgcccagggcgagcttcctcccgcttgtcccgaatctggtttcctggtgaaaagtccatccccctgtccccttgacagcacaggaaggtggagcccctgtgtctgctcaggtggactgggagcaaacagcagtcatgtttcctccagatgtttctagaaacaaaaggtgaagattttatttttatacttttatattttagaagaaacatggtgcccacacacgtgcagtgtatgctgtacttgtacacactcttcttaacagttatgaaaagctccttttttgcctccgctcgtggggaagtgtggcgggctggagggtgcactttgggttactgtttggcttgttggaaatttaagttggaaatgtttgtgcctctgtcttaacacagctatcttgaaacctctcaagcccacagcatctatttttggtgtgtttccacttctcttggtttgtgtgtgatgaaacctagctgctagaagcacctgtggggatgagttctggtcgtgtgtgtgtgtgcaggctgtggtctgggatgattaccctcccctggcctgggaccctcttagctgccggttggtgtgctccactcttggggctcttcagggaagctggggactcactaatgcaactgaaactgggtccctgcaaaccgccctacctccctgctctgagccagtctttccctttgccaatgtgccttgagtcctctggacttaacaaaatgatgtggaccgctaggctgcaaagggtctatttatagccaagtacatgagggcccacttgtggatcaaggggaggatggcctgctgagctgggtcccggagaacagggagggctgcccccacccggccctgtgccccacagtgggcggtgccctgcaggtgtacaggtgttcacagggcattgggttgcttaacacctgctgtggccatgctgggtggccaggtgaggatgggtggcctcggctatagcacatggctctaacaaaaccacacacctgcatctgtggtgtttcactggacttggttcattatagtcagggcactacttaggaaatgacttcgctataaactgggacagtgaccagggcctcaaaatgatctgcagggccgttcaaataaagctttattttgttcattctc

SEQ ID NO: 47 - DNA, 4289 bp, Bos taurus
  source 1..4289: mol_type = other DNA
ccttgcagcttccaccctgaaagcccatgggacagcaacaggtgaaagttacacaggaactgaaaaacattcaagttgaacagatgacaaaacttcaagccaaacatcaagcagaatgtgatttgcttgaagatatgaggacattcagtcagaagaaggctgctattgaaagagagtatgcacagggtatccagaagttggctagtcagtatctgaagagagattggcccggaataaaagctgatgatcggaatgattacaggagcatgtatcctgtttggaaatcttttctcgagggaacaatgcaggtagcccagtctcggatcaatatatgtgaaaactataaaaacttcatttctgagcctgcaaggacagtgagaagcctaaaagaacagcaactaaaacggtgtgtggaccagttgacaaagatccaaactgaactgcaagagacagtaaaagatttagctaaaggcaagaagaagtactttgagactgaacagatggctcatgcagtacgagagaaagctgacatcgagtcaaaatctaaacttagtctttttcaatcaagaatcagtttacagaaggcaagcgtaaagttaaaagcccggcgatctgagtgtaattccaaagctacccacgcaaggaatgattatcttcttacattagcggcagcaaatgcacaccaggatcgctactatcaaacagatttagttaatatcatgaaggctcttgatggaaatgtgtatgaccatcttaaggattatttaattgccttcagccggactgagctagagacatgccaagctgtgcagaacacattccagtttttattagaaacctccagcaaggtggtacgagactataatcttcagctgtttttacaagagaatgctgtgtttcacaaaccccagcccttccagttccagccttgtgacagtgatactagccgacagctagaatcagaaacagggaccacagaagagcacagtttaaacaaagaggctcggaaatgggccacacgtgtggcacgggagcacaaaactattgttcaccaacaacgggttctagatgatctggaatgccatggagtttcagtatcagaacaaaaccgagcagagttggaacagaaaatagatgaagctaaagaaaatattcgtaaagcagagataattaagttgaaagctgaagcccgactggacctactaaagcagattggcgtttctgtcgacacatggctaaagagtgcaatgaaccaagtgatggaagaactggaaaatgagcgatgggcccgccttcctgcagtgactaataatggaaccttacactcgcttaatgcagatactgaaagagaagaaggagaagagtttgaggacaacatggatgtttttgatgacagcagttccagcccttctggtaccttaagaaattacccactcacctgcaaagttgtttattcctataaggcttctcaaccagatgagttgaccattgaggaacatgaggtgttagaagtgattgaagatggagatatggaagactgggtaaaggctcgaaataaagtcggccaagtgggttatgtgccagaaaagtacctacagtttcccacctcgaacagtctgctgagcatgctgcagtccttggctgccttggacagtcggtcacacacatccagcaattccacggaggccgaactcgtttcaggcagcctcaacggagatgccagtgtctgtttcgtgaaagcactttatgactacgagggccagacagatgatgaattatcttttcctgagggagcaatcatccgaatcttgaacaaagaaaaccaggatgacgatggcttctgggaaggagaattcaatgggcggattggagttttcccatcggtgctagtggaagaactttcaacctcagaaaatggtgacactccatggatgcgagagattcaggtctctccttcccccaaaccgcccggctccctgcctccactgccgctgtacgaccagcctcccagcagcccttaccccagcccagataagaggggctcccagtacttctcccgttctccttcagcaaacgaaaacagcttccatgccgaatcgccaggattctcgcaggcatccaggcatactcctgagacctcatatgggaaactgcgacctgtccgggcagctccccctcctcctacgcagaaccaccgaaggcccgcagagaagatcgaggatgtggaaatcacactggtgtgatgatgggtttgcctgtccgttactgctacaatcaaggccaggcttggagtttggccagtcttgttttttaggcacctttgcttgcatgatgatgactcgaacagagcaaaaacaaggaggactatatgtgaccgggtggcctggttgacacctcccacgtttttaatatttcgtgcctttttttgttgtcattttctatgtcatctctcctagcacagcacaaatcctagtgggccctagaagcagagaagggggacagtcctcttgcctaccagggacccatttttgtatgagactcaagaccaggcctcgttcagggcacagtcacagagatgctgatcatatgcactcgtacagtatattactgtggccacgaggacgtggcaaagatgctcatctttcttcaagtggctttggcttatctgattgagtttaatcagatcatgttggctacataaggaaagcagcaggagggatttcaggagaggctggctcctccccgcagttggtccccaaactgagaaactgaaaccttattggaaaaaagtagactgccctgagtttagcaccgattgcaataatgcacatctcttccacagctaacagatttaaaacaattaacagtgtcctttgtattaatatttatgccattcatttagtattagcagagctaacatgaaggggctgaactagtagccgaatctcatccatcacatagactaatagaaagggggctgcggctaaagcagaaatggagcttccagatctgcaatgagccgacttagtgttctttttatatttgggtatttttcatcttaatttttgcagcatatactatcatgaccagtatccttagaatttgaatgtcttgataaaactggggacacatacttgccttgtcaagcttctctactggagaggcacctagcattattttattcccaagctagtccactggcccaggcagccaagttgtggctggcccagaccagcagccacccaccacatgaaatgcagcttcccaggtcccactgacctgacatttccttgggaggaagaatctgtggctcaaggagtaaaccagcttcctatgaagaaggacaaaaggattgcacggtgcaaattgacaaggaaaggacaagttctccatgtcacagggagagccaggaggagccttccctgcagactaacaaacccacagcagtggctcccagaggcctctggtggccctccacctccaatggaagatagggagtgcattacttaggaactaagggatgtttgccagttgctttttttttccttttttttaaaaaatcaaaattctcagaatttaatccaaaagttgtcttttgctttgaaaatgccatccctcctaagaatctctaaaaacttgaaatgctcgccaaatgtccccatgggatttttgaccagaaataaaactgaagaagttttcagatagtcatttgatcactttaatgacagtatccattgatgaatcttctctgatttgaggatttttattttttttttgcatttcttaaaaccttttgatctatttgaggtctttttgtgtttatcaaacttattcttaagtttacaaaagaaattaaaagggtgggttttttttttttaggattttaaggtttgtggacaagttaaaaatcttcaaagcactttaaaaatcttcaaagtgctgaaattgggcagttcttgccacccagtcttgtattctctctttagctagcaaaattgtccttgaatctgggttttccacattctcagagagttatttgaaatcttttgttatattttaaatattttttggaagagctgctaattttattttaaaaaaacgaagttgtaaaaatatgcctcctttttaataagaacccgtccctccaggacatgtaaccctgcgccacagtgcacagggctgggctcagaggaggcacctctgtgcagaagtgctttctttacagtggctgtaaaaactttgtatttattatgtataaaatgttgaataataaaaaaaaatggaattac

SEQ ID NO: 48 - DNA, 5748 bp, Bos taurus
  source 1..5748: mol_type = other DNA
gagcgcatgcgcactggccgaagggcacctccctgcccggatctcgcgaagttacgttagattggccgccggtgacgggtggccgggccgggggcgggagggcttctgaggtaggaagggaggtccgttcggccgggcgcgccgccccagtgctctgtgggatactggaagtctcgagcgtctcctcctggttcccagccctcctctggccgcactcatagaaacagtcacacaccccgtgcctcccctctcttccctctccgcctcccccttccccccctcgcgataagaagacccggcggcaggagaggggatgaagatggcggacgcgaagcagaagcggaacgagcagctgaagcgctggatcggctccgagacggacctcgagcctcctgtggtgaagcgccagaagaccaaggtgaagttcgatgatggcgccgtcttcctggctgcttgctccagcggcgacacggacgaggttctcaagctgctgcaccgcggcgccgacatcaattacgccaatgtggacggactcactgccctgcaccaggcttgcattgatgacaatgttgatatggtgaagtttctggtagaaaatggagcaaatattaatcaacctgataatgaaggctggatacctctgcatgcagctgcttcctgtggatatcttgatattgcagagtttttgattggtcagggagcacatgtaggagctgtcaacagtgaaggagacacacctttagatatcgctgaggaggaggcaatggaagagctactgcagaatgaagttaatcggcaaggggttgatatagaagcagctcgaaaagaagaggaacgaataatgcttagagatgccagacagtggttaaatagtggtcatataaatgatgtccggcatgcaaaatctggaggtacagcgcttcacgttgcagccgccaagggctatacagaagttttaaaacttttaatacaggcaggctatgatgttaatattaaagattatgatggctggacacctcttcatgctgcagctcattggggtaaagaagaagcatgtcggattttagtggacaatctgtgtgatatggagatggtcaacaaagtgggccagacagcctttgatgtagcagatgaggacattttaggatatttagaagagttgcaaaagaagcaaaatctgctccatagtgaaaaacgggaaaagaaatctccactaattgaatcaacagctaatatggataataatcagtcacagaagacctttaaaaataaagagacattgattattgagcctgagaaaaatgcatcccgtattgaatctctggaacaagaaaaggttgatgatgaagaagaaggaaagaaagatgaatctagttgctctagtgaagaagatgaggaagatgactcagaatcagaagctgaaacagataagacaaaacccatggcttctgtaactaatgccaatacttctagtacacaagcagctcctgtagctgttacaacacctactgtgtcatctggtcagccaacacctacatcacctattaaaaagtttccagcttcagctacaaaaatttctcccaaagaagaagagagaaaagatgagtcccctgcatcttggaggttaggacttagaaagacgggcagttacggtgcacttgcagaaatcacagcatctaaagaagctcagaaggaaaaagacactgcaggtgtcatgcgctcagcttccagtcccagactttcctcctctttggataataaagaaaaggagaaagatagtaaaggaactaagcttgcatatgttacgcctacaataccaaggcggcttgccagtacatctgacattgaagagaaagaaaacagagattcttcaagtttgcgaacaagtagttcatacacaagaagaaaatgggaagatgatcttaaaaagaatagctcaattaatgaaggatcaacttatcacaaaagttgctcctttggtagaagacaagatgatttgattagttctagtgttccaagcaccacatcaacaccaacagttacctctgcagctgggcttcagaaaagcctgctttccagcacaagcactactacaaagattacaacgggttcttcctcagcaggcacacaaagcagtacctcaaatcgtttgtgggctgaggatagtactgaaaaggaaaaggacagtgttcctacggcagtgaccattcctgttgctccaactgttgttaatgctgcagcttctaccacaaccctgactacaactactgctggcactgtctcctccactgcagaggtcagggagagacgcagatcatacctcactcctgttagagatgaagagtctgaatcccaaagaaaagcaagatctagacaagcaagacagtctagaagatcaacacagggggtaacattgactgatcttcaggaagctgaaaaaacaatagggaggagtcgttccacccgaaccagagaacaagaaaatgaagaaaaagaggaaaaagagaaacaggataaagaaaagcaggaagaaaagaaggaatcagaaacatctagagaagatgaatataaacaaaagtactccagaacatatgatgaggcttatcaacgctacagaccagtatcaacttcaagttcaaccactccctcctctttactttctacaatgagcagttcactttatgcttcaagtcaattaaacaggccaaatagtcttgtaggtataacttctgcttactccagaggattaactaaggaaaatgaaagagagggagaaaagaaagaagaagaaaaagagggggaagataaatcacaacctaaatcaatcagagaacgaaggcgaccaagagagaaaagaagatctacgggagtttcgttttggacacaagatagtgatgaaaatgaacaagaacaacaatctgatacagaggagggatccaataagaaagaagttcagacggattccatttctagatatgaaatcagttctacatcagctagtgatcgatacgattccttgctgggtcgctctggatcatacagttactcagaagaaagaaaaccttacagtagcaggctagaaaaggatgattcaactgactttaaaaagctttatgaacaaattctagctgaaaatgaaaagctgaaagcgcagctccatgatacaaatatggaactgacagatcttaaattacaattggaaaaagccacccagagacaagaaagattcgctgataggtcactattagaaatggaaaaaagggaacgaagagctctagaaagaagaatatctgaaatggaagaagagctgaaaatgttaccagacctgaaagcagacaaccagaggctaaaagatgaaaatggggccttgatcagagttataagcaaactttccaaataaaaaaaagcagcaagtaatggaattgcacatactagtaacccagtggaccataattgaccgtcactggaagcctgggaagaatccttggagactgtcatttttggatgtcctgccaaatgccctcttatctaggagttttgtttaattttctgttttttttttttttttttttctcttgtatcaagaccattgtttcatgttaatgcagctgctgagaagaattttttttatgactgagaaaacttgtttacagctccagcatataaggaaagtgttcaaggccagatatgcctcagatatttaaccagtaagccttagttgtacataaatacttttgtgtcaacaaaaactcagctctcacagaagacagttattcagcattttttgatgtgccatgccacagtttgcagtttttcaatatttaattttattttattttgttttgttattgctttacatctaagtttgggtttgtatcttttccttctaactcttcatgtggcagactttctgtcgtctcacagctttctcatttacagaaaagaacacttgctcctctgaaatcattgtcatatattaggctaatgttgttgtccccacctggaactgaattgcttggtggaacatttgctttcactgtttgtgcaatatgcatttattccttttatgaatgctttaaagtcatttgagattagattcttttaagtcctattttctgccttcattggtcacttttttgtttttgtttgttttttattattgtagtgtaagatgttaggttctgtaatcttcacattcattttagcaggtactgagtgatgctgtatatataaataagtgtattgtttgatttttagaccaccacatggcatgcttgactattatttcaaatgtctgttaatgcaaagtaggctactccataatagtgttaagaacaaaacttactaacaaagtgatataaagacttaaattaacacattatgtggagccctatctttacaaaagttttctactgtaaaatgcttttacttttatttgcagttttcatttgatagtattcaaccataattaaagttgcataagataattgcttcacatttcacatacctatatttatctgagtgctgtctaaaactgttgtgctagccaaagtaatgctatgaaatcatttgcagacttaacccgtgagtttgtgttaaatgcactgttattgccatgtgaagaggcattgactttgataccaccatcatgttcagaccattttatacatttcagtggccttttttaaaaaaaagaaaaagaaaagcaaaaccaagtaaatagtgaagatggcatttatttggacaaatagcttttatattttcattaaaccatgcaaaaagtactatatctttctggcacataactgtctccttaaccactgaacagttcagccatttgaataaattgtacattgtaaagcttatagtagctaattgtattattgattgtattgtatactattcaatgtgaatttgtaccccttcattttaaaaggtcattgtgttctactgcctattttagattacgttggggttgggaaagggtgttttggtaagcaggattttaagtcctctcttgattggttttatgaagatataaggttatgctcttactaccaagctcaggattcttgatttttaaaggtacaaggatagttgtgggaactttatttttggttacatttgaactgtatgaatggtgggtctgaatatagagaatttccatggtcttacgtggacgtaaaacatacagataattgatcagtttgagtgactgcaacatattctggttgcacaacagttaggcatgctaaggattatatttgtgaagtttggatgcctgtgaagaatgattaaatacgtgtttctaatgttttagtgttttgtatttaggttatttattctttgtatctaaaactgaacagctactgtgctatattgattttattggtagtattgagcagaccttgtttctgcatgaaactagttgcaaaacccactattttggaaataaaaatgtattttgacatatacaaataaaatgaataaaactattttaaatgtgtgaacttttactgaagacgtttaaaactttgttctgatatttaaattctgcatgggtatcttcactaactgctttacttggattccattggtataatttttgaaattgtaatattgtgataacttgcacaacgttgtacatgcacatctgtaaatttaagcttaattgccatatttatgcaatctgtgtatcaatttgaacaaatgtttatatattttacataaagctatctgtgtgcagaa

SEQ ID NO: 49 - DNA, 3762 bp, Bos taurus
  source 1..3762: mol_type = other DNA
gctccgggggctctatatagggcgcgcgctcgcgggccgctgagttccaccagtccgcgagctgccgtcggctccgcgcggggggggcgggccgggcaccccggggcgcggaggtgcgctctagcttaattcttctttccccccaactccgcactcccgggcaccctcaaacccagtccacgctgctgcccccgctccctcttttctcggcccccagcaaactttcggtcccttcctcgcccctcgcccgttactcgtcgtggctcgggcccggccgcgccgccccgagggctcctccggatctcccagtttccagctccgaccattagaggatccagaaacatgtcgaccacacttctgtccgccttctacgatatcgacttcttgtgcaagacggagaaatccctggccaacctcaatctgaacaacatgctggacaagaaggcggtggggacacccgtggccaccgcccccagctcgggcttcacgccgggcttcctccgacggcactcggccagcaacctgcacgcgctcgcccaccccgcgcctagccccggcagctgctcgcccaagttcccgggcgcggctaacggtagcgcggcggccggcggctcggcctcctacggcaccctcaaggagccgtcggggggcggcggcaccgccctgctgaacaaggagaacaaattccgggaccgctcgttcagcgagaacggggagcgcagccagcacctcctgcacctgcagcaacagcagaaggggggcggcggctcccagatcaattcgacgcgctacaaaactgagctgtgccggcccttcgaggagagtggcacctgcaagtacggcgagaagtgccagttcgcacacggcttccacgagctgcgcagcctgacgcggcaccccaagtacaagaccgagctgtgccgcaccttccacaccatcggcttctgcccctacgggccgcgctgccacttcatccacaacgctgatgagcggcgacccgcgccgtccgggggcgcctccggggacttgcgcaccttcagcgcccgcgatgcgctgcacctaggcttcccgcgggaaccgcggcccaagctgcaccatagcctcagcttctcgggcttcccgtcgggccaccaccagccccctgggggcctagagtcgcccctgcttctcgacagccccacgtcgcgcacgccgccgccaccctcctgctcctcggcctcgtcttgctcgtcgtcgcttcgtcctgctcctcggcctcggcagcctccacgccctccggcgcccctacgtgctgcgcggccgcggcggcggccgcgctgcctgtacggcaccgggggcgccgaggacttgctcgcacccggcgcaccctgcgccacctgctcgtcggcctcgtgcgccaacaacgccttcgccttcggcccggagctgagcagcctcatcacgccgctggccatccagacccacaacttcgccgccgtggctgctgccgcctactatcgcagccaacagcagcagcagggcctggtgccccccgcgcagcccccggcgccgcccagcgcgcccctccctgccagcgccgccgcgccgccctcgccgcccttcagcttccagttgccacgccgcctgtccgagtcgccggtgttcgacgcgccccctagccccccagactcgctgtcggaccgcgacagctacttgagcggctccctgagctcgggcagcctcagcggctctgagtcccccggccttgacccaggtcgccgcctacccatcttcagccgcctctccatctccgacgactgaggcaagagggcgccagtgaggaggaggaagggaaggccgttctgggggtgttggagggtgcccctgcttaggggcagggggccacgggagtgtggggggtgacatcggccacaagcagactgcaggctgtgccgagcacttggactcgaacttgagtgccgggaggggcccccacccctcccctttcggttccctcttcgttttttgtttttttattattattattatttttattattattacgaagtttcattcttgttgagcgggaaaaaagccaacgaactttttgtattgatgaacaaaagactcacaacagagcagttgtgatgcgaatagaacaaaaaccaaacaaacaacaaacttttttaggtctccgatgagtttgggattttaggaaaaatcaacccagcaccagcagctatcaaccaccattccatatcttcacttgaaaagcattagttacagtccagatgtcaggactcttatcttgagagaggttccttcctaattgtttgtcccagaccagtgtaaacaaaccagccaaccaccgccttgctaataaacctataagcttttagaatccaatattctgccaagaatatgccttgatagtagacctcagctccataggtgtttgttttttaacaaagttatatatgtctggaaaaaaaaaaaccttgcattccaaagtttcatactggttactggtttcattgccaccacttagtggatgtttaatttagaaccattttgtctgctctttctggaagccttgggcagagcttagttcataattgatggggaataactgctgaatttttagctgttttgagttgattcgcaccactgcaccacaactcaatatgaaaaaaaactatttaacttatttattatcttgtgaaaagtatacattgaaaattttgttcatactgtatttatcaagtatgatgaaaagcaatagatatatattcttttattatgttaagttatgattgccattattaatcggcaaaatgtggagtgtatgttcttttcacagtaatatatgccttttgtaacttcacatggttattttattgtaaatgagtaaaaaaatcttaatttaagagattgtatgtaatatttatttcattaatttctttccttgtttacgtaaattttgaaagattgcatgatttctttacagaaatctatcttgatgctgtggaagtagtttgaggaatatcttacgagtttttcttagaatgtataatggttgtagcccatccaactttaaaagaaaaaatgaccacattgcaatcaggctcacatgtggtatgcttttctcattccaactttataaattagcaaaagatgtttttgtttgcttgttccaccagttctactgtgacatactctgcatataaagacatgtagcaataatggggcgagggtgatctcacagtgcctttggaagggccagaacttgccttaaattttcctcaaccaaataagtattttgtctattagtgcttgagagaatctgaatgtaggatgggttcaactgcacaaaaggagaagatttttaccactttttttttatatagatataaagtgaagaggccacctcttagtgctaaaatggtatgtagtacatgaatatgccttgtttaattacagaaaattccaaaacttgtactattttttttcccccgtgtggaaaggcaggaatgcttttctggacagcagctgaatgagcagtagctttagttcgtttgtacgtaggtacagtcggagcactatgtatgtgtgtattgtggactactttgacagaagtaggtattttgaatgtaacaaggttaagtcaacttgagttgtaatatatttggggaatcagttcactacaaattgtgactgtaaacattgtactgtaaatgttttgtagttttcccccaataaaacttttgggaaaaaaaaaaggta

SEQ ID NO: 50 - DNA, 3888 bp, Bos taurus
  source 1..3888: mol_type = other DNA
gtggcagcagcagcagcagtatcttcaaaatggctgataatttggatgaatttattgaagagcgaaaagtcagattggccagagacaaagcagaattggaaagtgatccaccgtacatggaaatgaagggaaaagcatcagagaagctttctgaaaacagtaaaatattaatttccatggctaaggaaaacataccacctaacagtcaacagacaaggcgttctttaggaattgattatggattaagtttaccacttggggaagagtatgaacagaagaaacataaactgaaagaagaattgcgacaagattacagacgctatcttactcagggtattacacaagcaaaaagaaagaaaaattttctatccacgagtgaaacagatccatctactctgggagtttcccttcctattggtgagaggttatctgctaaggaaaggttgaaacttgaacgtaacaaagaatacaatcaatttctcagggataaggaagaatccactgaaaagttcagacaggtagaaaagagtactgagcagaagaatcaaagaagtaaaaaacctatcagtcaggttaaacctgatttaccttcacaaatacaatcatctttcgaaaattcagagggtcctagaaaagatgtcttaactccttcagaggcatatgaagaacttctaaaccaaagacgactagaagaggacaggtaccgaaaattagatgatgaaattgaattaaggaacaatagaagagtcattaaaaagactgatgaagaagtgggcgtttccagtaaaaaacatcaaaggtttgacagcacgactgacattccagatagaagatttcacaggtttaatgaggatcttgcttttgataaacagtcttacagaccaggctatgatccagaagtaagtgaagacatggatgagaggtttagatatgaaagtgattttgatagaagacttttgagagtttatacaaaaaacaggatgtacgggaacagacgaggcaatgagcctcctatggagtgtgatggtgatgtcacagaacagtcaaacatacgaatttcatctgctggaaatcaaagtgctcgagacaatgaaccatccaaacctacttatcgagagacatgcagtccttttgcgggaatgctctttgggggtgaagatcgagaacttattcagagaaggaaagagaaatatagacaagaactattggaacaaatggctgaacaacagaggaacaagagacgagaaaaagatttggatctcagggttgcagcttctggagcacaagaccctgagaaatcgcctgatagactaaagcagtttagtgtggcaccaagacactttgaagagatgataccacctgagagacccagaatagctttccagacacctctccctcctctgtccgccccatctgtcccacccatcccatcagtttactccatcccatctcagagtgaagatgtgcacagtggactcagcagcactcttggtgacatggtgcctcccagagttgcacctctgcctccacctcccatgctgccccctttggctgcgaactaccgaactccttacgatgacgcgtactatttctatggggccaggaatactttggatcctagtcttgcttattatggttcagggatgatgggagtacagcctgctcctgctccttatgttacagctcctgtcacccaccagccagtacagcctattgtgagtacagttggacagaatgagctgaaaattacaagtaatcgagcaataaattcaggattgttttttgaagataaaccaaagccttcaaaacagtcatttcagtcttaccaggaagctttgcagcagcagattcaagaaagacaagaaagaagaaagaaagaacgtgaagaaaaagaagtatatgaagccaaattagaagctgaaatgagaagttacaacccctgggggaaaggtggaggtggtgctcctctgagagatgacaaaggaaatctgataactgatttgaataggatgcacagacaaaatatagatgcctaccataacccagatgcaagagcatatgaagataaaagggctgttgtatctctagaccaaaatttagccacttcaaatgcggagaaccaagaagatgctgcaaataaaaactcaggtcatatgcaatcacagagctctccttttgctcggggaaatatttttagtgagcctccaactgaacttcaaattaaacagcaggaattatacaagaattttcttcgttttcagattgaggaaaagaagcaaagagaagaagcagagcgagagagattaagaattgctgaagaaaaagaagaaaaacggcttgcagaacagagagctcgaatccagcaagagtatgaagaggagcaggaaaagaaaagggagaaagaggaggagcaaagactgaaaaacgaagagcttattcggttagctgaagaaagacgaaaagaagcagaaagaaagaagaaagaagaagaagaaaaacataatctacaacttcagcactactatgaaagagaaaacatgattggggaagatacaaagcacttgagacagccttctcctgtggttcctgctcttcagaataagattgcaagcagactccaaagacctccttcagttgacagcattataacttcctttactcaggacagttccatgtccatggcacaatctcctccggtacctgccaggaaaaaccagcttcgtgcagaagaggagaaaaaaaatgtaattatggaattatcagaaatgagaaaacagcttcgtagtgaagagcgacgtctgcaggggcggttactacacattgatagtgacgacgacattcatataaggaaaagagaaagttccatggatgtatttgatatggccagacatcgattacaagctcctgtcagaagaccgtcacctaagagcttagactctgcttcttctcagaatattcatgactttaaggagctgaaagatagagattcagaaacgcgagctgatctgaaatctatgtacccagatgatccaagagataatgacaccttggagatagaacaacaagccttgctaagagagcagcagaagagactgaataaactaaaaatgcaggaagttgacttggatgctgtcccaagcactaaagtacgggaccacagaatgcccagagaggacactgatgatttcttgaaaaactcattattagaatctgatagtgcttttattggtgcgcatggcgagacgtacccagccattgaggatgggggcttcctgctgctatctccgctgccctccgccagagagcgcagaaggaacaaactgaaaggattggactttgataacagtcaccctgatgtgccaccagatggtgtctctttaaaatctgtatccagtataaatattgatcagcttagaatgagaaatgaagaacgaatgcgaagactgaatgaacttcagaataaacctattactacagatgatgagagttcactggtggaccctgatgacatcatgaggcaccggggtgacgacggatcaaactctgtagccactgagccctggctccgcccaggcacctcagaaacactgaagcgattcatggtggaacagctgaggcaggagcagcagcagggtcctggcaaaccaggcgctttcacctggcagggactgtctactgcacacggttaaaatcagtctgtactggacccagtagtgcctttgaaggtgaaaggcatgtgaaacctgaacctgaataatatgtgctacttttggcctgtacatggcagttactcatattccatctgtatagaaggtatattttttccatgatgatgtatatgatgtatttatattatgtacataaataaaaagccatggctatttatat

SEQ ID NO: 51 - DNA, 2343 bp, Bos taurus
  source 1..2343: mol_type = other DNA
acttcttgcttccttcccataaaaactcagaggcataaaaggcccagagaatgagcatcccagagaaactgaccagggatatgggagtgatcgccatggatcagaagtctttctgggaaggtttagtggtcttgctgcttcttcaggaaggatgtgcctacaaactggtttgctactttaccaactggtcacaagacaggcaggagccagggaagttcacccttgagaacactgatcccttcctatgttctcacctcatttactcattcgccagcatcagtaacaacaaggtcatcatcaaggacaagaatgaggcgaagctttaccagaccatcaacagtctcaaaaccaagtcagtaggagaggagtggggagcacccagggaatcccaaactgaaaattctgttgtctattggagggtatctgtttggttccaaaggaagttagcagaagcttttcagcaggactttgtaaaatccaccaaagaaaggcttctcttgactgcaggagtttctgcagggaggcagatgattgacaacagctatcagatcaaggaattggcaaaagacctggatttcatcaacctcctgtcttttgacttccatgggtcttgggaaaagccccttgtcactggccacaacagccccctgagaaagggacagctggacagagggacaagctcctactacaatgtggaatatgctgtgggatactggataaacaaggggatgcctgcagagaaggtggtcatgggcatccccacgtatggacgttcctttacactggcctctgcagaaactgccgtgggggcccctgcctctggtcctggagccgctggccccatcaccaagtcttcaggcttcctcgcttattatgagatctgtcagttcctacaaggagccaagatcacaagacttcaggatcagcaagttccctatgcggtcaaggggaaccagtgggtgggctatgatgatgtggagagcgtggagaccaaggttcagtttctaaagaatctaaacctgggaggcgccatgatctggtctattgacatggatgacttcactggaaaactctgcagccagggcccctaccctcttgttcaagctgtcaagaggagccttggctctctagaaaggtgagggtgctgctgaccaaccaggtgtgctgacatcatctcagtggaggagcaggacactggaaatcactgcagacagacaactggagaggactggccatattgtcctggagaagggcccagagcagtgcagaatctcctaaactatatgattgggcagcagcctttcaactctgaacatctatgtcttagtaacttgactgaggccttatgttactgattggatttgaaatactttctttttctttccaggcttaattcatagctaagtaggaaaggtgaccttgctgcgtactcctgctacaggaattcatgtgtgggattcttggaggttagatgaggccctcaggatgtctcttctccaagtctccccttgactttctctcagacctcagactgggtctggctttgttctcctgcagcgctgactgcttcccttgaggcacaataaagaaaaagcatgttgttccagccagcgtggcctcctctgtgcaatcttggaccttgtggtataaggctgtcctctcccaccatttatgggtacaagtgtaggcctgcgtatcatatgtctaaatatttaagagccaagggttaaaatatgatttatactcttaacgaacatgattttgcaatgacccaaaagaccaggtttgaagttagaattcccagactcctgggaattctgtgccagaatgcagcagcttgtgcccttggttcccagcccactggtagcccctttcccttcacatctaaagccctgactcagagagcaaggacctcacatacacactggcagatgttggaaaggggaagttgttaggggaaacaatgactgaaactgcccaccctgtcaggcactacagtaactatttgcatgagttattttacaacaggaggtcctggtaaggaacatagaactaacaagccatcaccaactggaagaattcaggagaactcaaaaggagactccatgtgtcctaccacctcccagaatcctcactgaaatccatctcggctgagcaatgcatgtgccaccaagaagcaccctgattcagagtgagtggctggaaacaacccagaaactaaccccatcgccataaaatccgagactgagggccatgtggtagagcagccctgctgggttccccttaccttcctgctctctgcctgggtgcctttttccaataaaatcgcatgctttgtcagaa

SEQ ID NO: 52 - DNA, 1053 bp, Bos taurus
  source 1..1053: mol_type = other DNA
tagaaagagcgataagtcccccagcacagtgcccacactttgggaggggaggcaggagagtatggagggagtgaaggacaagcagcctgcagggagctttcctcctgccgctcggtatctcggtttggacctgaggcagctggcaggtaacagcctgtgcaccatgtcctggatggatgctcctcctccagcatccatccaggagtgcagaaaggagcccaccacggaatacagaggtttctgtctgggctccacagtgcagtctgaaaccaagggaatctggatgtggtatgtgccccacccctccaaggagaaccacaccctggtccttctggacactgagggtctgggtgatatggaaaagggtgactccaaaaatgactcatggatctttgccctggctgtgctcctgagcagtacctgtatctacaacagcatgggcaccatcaaccaccaggccctggagcagctgcactatgtgactgaattaacagagctgatcagaatcaaatcatctcccatctctgatgatgtagaggactcagtcgagtttgtgagattctttccagactttgtctggactgtgcgggatttcatgttggagctggagtttgatggaaaccccatcactgaagatgagtacctggagaatgccttgaagctgattccaggcatcagagcaggggcaaggtggtagaaagctcagtaggaagtgggatcaagagctctgatattcagcacttgatgtcactcttaatttggggttggaatgaggtcatgctaatggtgcctggtgaatgactggtgggtggattcagtcattgtttttactggaagaacttcagagaccagagcataagttcgctgaggaaatttagaatctagtattaaacgaacaattttagaaaaatttatatcatgaagaaaaagtctcacatattgtattaaatattaagtgcctcttttttcctgtttgagaaaaataacccatcaaattgaaattggtagatgtagaatcataaggtaaataaaaggattagtttgtgaaatatt

SEQ ID NO: 53 - DNA, 393 bp, Bos taurus
  source 1..393: mol_type = other DNA
atgaggctggtgtacctttttcttggccccatgaccctcctgctcctggctggctgtggctgtgtcatcagcacctccagtgagaacctgcgcacatttgtgggctgtgcagtgagggagtttacttttctggccaagaagccgggctgcaggggccttcggatcaccacggatgcctgctggggccgctgtgaaacctgggagaagcccattttggaacccccctacatcgaagcccatcatcgggtctgtacctacaatgagaccaaacaggtgacggtcaagctgcccaactgtgcccctggagtcgaccccttctacagctaccctgtggcggtccgctgtgactgcggggcctgctctaccgccaccacggagtgtgagaccatctga

SEQ ID NO: 54 - DNA, 5228 bp, Bos taurus
  source 1..5228: mol_type = genomic DNA
ggaggggaggtacctgggactgggagtgatgtcagctcccagctcggtgcctgcccggattcctgacatggtgtagggccgagagggtggggaaggcgggagccgggcgagagaggactcggggcagccagcctgcggcggggccggagtgccggcgctttgaactccgggcgaaggtgaagcacaacttttgcaactcgtgctggcggctctcctccccccagtataaaaagaacttcatggatcagactgctaacaagtacatggaagatttgaagagctgaagaaaaaaaaactcactgttaaacttgttttttaaaatatctaggttggaatttgcccttgacaaataacaagatgatgagtgatgcaagtgacatgttggctgcagcgctggagcagatggatgggattatagcaggttctaaggccctggaatattccaatgggattttcgattgccagtcccccacctccccattcatgggaagcttgcgagccctgcaccttgtggaagaccttcgtgggttgctggagatgatggaagctgatgagaaggaaggcttgaggtgccagatcccggactctacagcggaaacgctcatcgagtggctccagagtcaaatgacgaatggacacctccctgggaatggagatgtgtatcaagaaagactggcccgtttagaaaatgataaagaatccctcgttcttcaggtgagtgtgttgacagaccaggtagaggctcagggagagaagattcgcgatttggagttttgccttgaagagcacagagagaagttgaatgccacagaagaaatgctgcagcaggagcttctgagtaggacatccttagaaactcagaagttggacctgatggctgaaatatctaacttaaagttaaaactgacagctgtagagaaggacagactggattatgaagatagattcagagacacggaggggctaatgcaggagatcaatgatttgaggttaagagtcagtgaaatggaaaatgaaagacttcagtatgagaaaaagctgaaatctactaagtctttaatggccaaactttctagcatgaaaatcaaggtgggtcagatgcagtatgaaaagcagcggatggaacaaaagtgggaatcactgaaggaggagctgacatctctgaaagaacaactggaagagaaggaatctgaagtgagaagactacaagaaaaattggtttgccagatgaaaggagaagggattgaaattcttgatcgagatgaaaattttaaaaagaagctcaaagacaaaaatattgaagtacaaaaaatgaaaaaggctgtggagtctttgatggcagcaaatgaagaaaaggatcggaaaatagaagatcttcgacagtgcctgaacaggtataagaaaatgcaagaaacagtggttttggctccaagtaaaaaaggccaagatggcgactttgaagacctgcttccatccagctccttttccaccctgctggatgctcagggctttagtgatctggagaagaacctgtcgccgacgccagtaaggggatctcccagtcatgacccatttaacacaagtgctccagaagagttccacaccagcatcttgcaagtttcaatcccttcattattgccagcatctaaaggcttggaaacttctgaaaaagcaaaattgccttctaagccagagactccatttgaggagagtgatgaaattggttctgctgctgaaactcaactgtgtgatagtcttgcaacttcaagtttgcagaagtctagcagcctgggcaatctgaagaaagagtcatcagacggggaaaaggagtctattccaaagccttcagaagataaagctccggtagagagcagtccctttgggaccctccctcccaaagctccaggacatgatgcctctggggatgacaatccctttggcactcggaaagccagatcttcctttggtcgtggtttttttaaaatcaaaagtaacaagaggacagcaagtgcgccaaacttggatcgtaaacgaagtgccagtgcacccaccttagctgaaacggagaaggagacaccggagcacctagatctggctggtgtatcttctcggccgaaaaattcacagggaagtagtcccttccagatgtctccaccatctccagattccaaaaagaaatcccggggtattatgagactctttggaaaacttaggagaagtcagtccactacattcaacccagatgacatgtctgagtctgaattcaaaagaggagggacaagggcaactgcggggccccgattgggttggtctcgtgatttgggacagtctaacagtgacttggatatgccatttgccaagtggaccaaggatcaggtttgcagttggctggtggaacagggtttggggtcctacctgaattctggcaagcactggattgcatctggccagacacttttgcaggcttctcaacaagacctagagaaggagcttggaatcaagcattcccttcatcgaaagaaactccaactggcactgcaagccctgggatctgaagaagaaaccaatcatggaaagctggatttcaactgggtcactagatggttggatgatattggcctcccccagtacaagacccagtttgatgaaggacgggtagatggtcgaatgcttcattacatgactgttgatgacttactgtcgttgaaggtcgtgagtgtgctccaccatctcagtatcaaaagggccatccaggtcctaaggatcaataactttgagccaaactgtctacggaggcgaccctctgacgagaacagcatcaccccatcagaagttcagcagtggaccaatcaccgagtgatggagtggctgcgctccgtggacctggcggaatatgcccccaaccttagaggcagtggtgtccatggtgggctcatggttctagaacctcgttttaacgtagaaacaatggctcagttactgaacatcccaccaagtaagaccctgttgcgaagacatctggccactcacttcaaccttctgatcggtgcagaagcccagcaccagaagcgggatgccatggagttaccagattatgtacttctaaccgcaaccgccaaagtgaagccaaagaagcttaccttcagcaattttgggaacctgagaaagaagaaacaggaagatggggaggaatatgtttgtccgatggagttgggacaggcatcaggagggacatctaagaaaggatttaagcctggtttggatgtacgcctgtatgatgaggatgacctggaccgattagagcagatggaagactcagaagggacagtgagacagataggtgcattctctgagggcatcaacaacctaactcacatgttaaaggaagatgacatgtttaaagattttgctgcccgttcccccagtgccagcatcaccgatgaagactcgaacgtctgaccatagcacctggatgagagcagggagcccttcgtctttgttccactctattcctcggacactcacagtatctacagcaagccacaggctgtgtgttgtttatcgttcccgcttctcatgtggaagtggaggtggaaagcagggattaggtgctaattctaaagttgccatgacaattgagacactggtgaatgagagtataattgtttctcctctatttaatgtaaaaaaaaatctgtgatatattatatttaaagtgttgcatttaatatgagtattttaccatagtctttccatccacattcacagcatggaggatttgggaagcagtgaccagtatgtgaatgattttttgtcacgttgatgtgatagcactgcggccctcgaagcaggatgttacatgcttttgaaatcaaattgtggaatttcttaggcattcagtgtgtccactatatgccagccacacctccacttgcttctttttgtcctatttttatatatttttctaaatatatgtatatacttagtacatagaaaatagaacttttattttgtaacataaggaagatggtaaaaagatcacgttaaaaaaacacagtgagcaaaatttttgtataccagctgtttcttgaagaggtccaaatgatatttctgtcccccaaactgaactctaatgatgttgatcattcagactaaattaacatatgtgaaataccccttctttgatgacacttcacaaaattattgaatagctttttaaaaatttcagccttagtcttggatcctcttaactcataaggaagaatctgagggacattttaagtgctattaacctgagtgtattcaagaaactccccccaaatttgtagaaataattcctgaagaaaccaaaacaaaaccctttacagtattaagcttcttatcttcctctttgctaaacatatcattgcatcaaaatactaacagtctgcctaatgttattaattgcacatttctcagactattagcaaatggattttttttaaagctgattacttgtctgaacattttgtctcagacttcagtaactcaaagactgcaaaattagtttcataatggctatagtgtgatgataaaatgcagttcatgtttttctgtagcacagggcccaaatattctttatcaagaaaattgaaagctgcaataaaaactgttattgtgtttattattttctagaggtatcagaaaaatatccaattttttgttcactactatctcattacttatgatagtcagtggtggtaaagacaggttttcaaaaattagcgtagggtgggcagttcaatatatgtctgaagacttgcctaataaactatatttgaatgtcagtggcatgacctttcaagttagcaagtttccaagtttcttatcaaggaaccctgtgatactaaattactatttattcataattaaaatgactcaatgctaataatatttttggtccctatcatgtactatttggtaactgtattggactctttgtgtatttgtgttttattttaatgtgaacatatgtcaccatttgaaaaaccatttgttattcaatcctggttaaaaatactaggagactgttacagatgccaaatattccttattcaggacagtgtaacatcactgatgatatgtgataggttaaatttttttgatgatatatatcctttttacaagatatttacactgctggtattaccatatgaaaagaaataaagtcagttgataactgcctcata

SEQ ID NO: 55 - DNA, 1609 bp, Bos taurus
  source 1..1609: mol_type = other DNA
ctctcagaaatttgtttgaggggggtctataacctgtatcagggtttccccagtggctcagcagtaaagaactacttgcaaagcaggagaggtaagagacatgggttcaatccctgagtcaggaagatcccctggaggagggcacagcaactcactccagtactgttgcctggagaatcctatggacagaggagcctggaaggctacagtccatagggttgaaaagagtcagacactactgaagccactaagcatgcatagcctgtatcaacagatgaaaatttaaggcctttacttcagaaactttctcatgaaaggaaacacaggcagctgagcttgggggatgaggaatcatggcaaaggggtatcggaaaagcaaaccaaaatgacttgcctacaagtttgaagaagttagttgtagaaagaagttagttttatacatatgtttctttctttaaagaattaaatagtattttttaataaaaactaaaacttgaaaaaaggaaacttgtctcagaagaggttaccaaatatttgaagttaggtgaatataactagaagacagatgattttttgtgaaagtatctctgacttaataaaccagcaggactccatatgagtacaacacacccagttggcaaacaggaagacctaacatgtttgtttagctgtgtgtatatatgcttaattgtagagttcgaaaactgggaccacattcacagatgtgacttcactgcagaatttttactctgaaaaaaaacagttctcgtgctcaatatctagaagtagtgttcagcgtgtgcagggaaatggggcagaaaccatcacaacagctggctccaaaggacaaagaggttctgaacgtctgtgaggtggtcagtggagctatcatccacgcagctcagaaactgaaggcctatcttggatttgaagacacactgagcaatctgtgcccagattcaaacactctcaatgagatcttcttaatccactttgtcactttctgccaagagaaaggagctgacaagtggctcaccaccaccaagatgaccaagcaccaagccttcctgtttggggcagattgggtttggaccttctggggatctgataagcaaatacagcttcagctggccgtgcagacactgcagatggcttctcttcctcctgtggaatctgacccctccaacccagattccaaggcagagaagtctttcagcaagaaaagtaggtttgacaagctggaagaattctgtaacctgataggagaggactgtcttggcctgtgtatcatctttggtgtgccaggaaaccctaaagacatccgaggagtcgtcctggacagtgtcaagagagagacgacgaagggtcatctgccaggagggacagctgtggcacgattcatcctggaaacggaagattgtatctccatcagggagctgcttggaaactgtctgagtaagaaagatgggctgagagaggtgggcaaggtttatattagcatcctctgaactggccgtgtgggatgagactgacctgtaagataaaacaaaaatatgttcttctaataagtttatcctctcatgggatttttttctcccctcgctccacc

SEQ ID NO: 56 - DNA, 2111 bp, Bos taurus
  source 1..2111: mol_type = other DNA
tggctggcttagtgtggatcctggcagttttttcttgggtcagggtcagaccacgccgtcccaggaggaggaggggagttgtgcagtacaagcagctcttgacagaaggatgctggagctgagcctatctcggctgggactcgggccgctggcagcctccccgtggctgctgccgctcctggctggggtctcctggatcctggcccgcgtcctggcctggacctacaccttctacaacaactctcgccgcctccgatgtttcctgcagcccccaaaaccgaactggttcttgggtcacatgaacctggtgccctccacggagcagggcctgatctacttcactcagatggcggccaactacccccgtggatatttgatctggtttggccccattatcccaatggtcatcttctgccaccctgacatgctccggagcatcaccaacgcctcagctgccattgcacccaaagacatgcagttctatggcactctaaaaccctggcttggggatgggctcctgctgagtgccggtgacaagtggagcagccaccgtcgcatgctgacacccgccttccacttcaacatcttgaagccctatatcaagattttcaccaagagtgcagagatcatgcacgccaagtgggagcacctgatcacagagggccacacccatctggacatgtttgaacacatcagcctcctgaccctggacaatctgcagaaatgtgtcttcagttttgacagcaattgccaagagaagcccagtgaatatatcgccgccatcttggagcttagtgcgcttgtgtcaaaaagaaaccaacaactcttcctgtatatggacttcctgtactatctcacctcagacgggcagcgcttccgcaatgcctgccgcctagtgcacgacttcacagatgccgtcattcaggagcggcgccgcactctccccaaggagaatattgatgacttcctcaaggccaaggcgaagaccaagactttggacttcattgatgtgcttctgctgaccaaggatgaggatgggaagggattgtcagatgaagatatccgcgctgaagctgacaccttcatgtttgagggccatgacaccacagccagtggtctctcctggatcctgtacaaccttgcaaagcacccagaataccaggagcgctgccggcaagaagtgcaagatcttttgagggaccgtgagtctaaagagattgaatgggacgacctggcccagttgcccttcctgaccatgtgcatcaaggagagtctgcggttgcaccccccagtcaccagcatctcccgccgctgtacccaggacattgtgctcccagatggccgggtcatccccaaaggtgttgtctgcctcattgatattttcgggacccaccacaacccatctgtgtggcaagaccctgaggtctatgaccccttccgcttcgacccagaaaacatcaaagggaggtcacctctggcttttattcccttctcagcaggtcccaggaactgcattgggcagacattcgccatgactgagatgaaagtgatcttggcgctcaccttgctgcgcttccgcgttctgccggacaaggaaccgtgccggaagccagagctgatcctgcgcaccgagggaggactctggctgcgggtggagccgctgagcgcaggccagcagtgacccagcaccttctctcctgagatcctctctgaccctaaacacctcacgtgcagattggaaatcaaacttcgtggtcacctctgccctagtctcgtctagaagacagcaaggagcacatgggatctgctaggttccaggcaggtgggggttgctgtaaacctggtggcctttagcggggcggctcagaagggaacttggatcctaagtgtaagactactcattaggaaagtccaactgcttgatgcagagagggtcagatgaccttggatttgctccctgacatggtaatataaaggtcaccgtcccagtctgccacttactagttgtttggcttaagaaaattgtttacctctcagttcctttattacataattgtagccactttactgggctgttggggagattgaataaacaagcatatgcaaaatga

SEQ ID NO: 57 - DNA, 3174 bp, Bos taurus
  source 1..3174: mol_type = other DNA
cccgccttccggctccggcggcgacgctcgcttcttgccgccgccgcgctgccggtgtcagcctccctcgggagactccatttgctctcagattggtctggtcgccgcgaagtcaggcagagagcgatgtccccggcccgtggactggtgtcccccgggccaagcgccctctccctcgctggctggcttcggtggtgacatcctggttgctggcgaccacggaggaatccgggctccctctccatgagcaaagacgtcccgcgtttctgcggggaaaggccctcgctaacacggctgctctctgacctcttaatttcaggaggttttcggaggcttggagtgcctatacagcccccgacaggaactgcttctgttggcccatcgaggatcatctttcaccaggattatcgtaacatcttcactgagccttccaccccatagtctctaccatgagtcctgatttctgccaatgaagttttgtttttttttttttttttacgctttctttaatcatgtgacaccacttctggtcagccttttcccttttcagttgccaaacacattaggtggaagtttgtgtcgtgttaaagtacggagtttcttgcttgtccactctcagccttgtttgcactgctcacttctgttcagcttgccttctcctgcttgcctatgatttccattatcactctcttgcgtaacctgtagcagtccttgcccggggaggcagcattccatacagtgcactgcctgacaaggacagcagtcagggggtgagttttcccattataatttcttattaagtttgcaagtcctgtcttaaaaactttgttccctagcacatttctctaaattcgtccaccagtgacttagttcctttaacatagagcctcacccataggatggcaaaaacaattaagtacacaataggactgttttagagaccttgctaatacagtatgggttatcatcaccattgagattacagttcatgaaaagatcttaaacttaggagaagcaagatagaagataggattaattggagctagtctttgttgttttgagtttgtaaattttaaaagagcaaagtgaagagagtcacataggcttggcctggaaattggccaacacactgtccatctaggtcctagtggggcaggaaagggatcttaaaacaccattttttaaatctcattgtgtccaaatctaacttattaaaggtcttgttgtagtaatcctctaaaatattaggtacaatgttggggaaacgaaagcgtgtggtgttgacaattaaggacaagcttgacattattaagaaattggaggagggcatctctttcaaaaagctttccgtggtatatggaattggtgaatccacagttcgtgatattaaaaagaacaaagaaaggataataaactatgccaacagttcagatcctacaagtggggtatccaaacgtaaatctatgaagtcgtccacatatgaagaacttgacagagttatgatagagtggtttaaccaacagaaaacagatggaattccagtgtcgggaacgatttgtgcaaaacaagccaagttcttttttgatgctctggggatggaaggtgattttaatgcatcctctggctggctgactcgattcaagcagcgccatggtattccaaaggctgctggtaaaggaacaaaattaaaaggagacgaaactgctgccagtgagttttgtggtaactttcaggaattcgttgagagagagaatctacaaccagaacaaatttatggtgctgatcaaactggattgttctggaaatgtctaccatcaaggacattagctcttgaaactgagcaaagttcttctggttatagatcaagcagagagagaatcattattatgtgttgtgcaaatgccactggtttacacaaacttaatctttgtgttgtgggaaaagcaaaaaaacctcgtgcattcaaaggagctgatctttcaaaccttcctgtcacttatttcagtcaaaaaagtgcatggatagaacattctgttttcagacagtggtttgaaaaatactttgtgccacaggtacagaagcatttgaaatccaaggggcttctagaaaaagcagtgctccttttggactttcctccagcacatccaaatgaagaattgttgagttcagatgatggcagaataattgtgaaatatttaccaccaaatgtcacaagtctcattcaacctatgagccaaggagttctagccacagtaaaaagatactaccgagcaggacttctccagaaatacatggatgaaggaactgaccccagaatgttctggaagaacttgacagtgttagatgcaatttatgaagtgtcaagagcttggaacatgataaagtcaagtaccataaccaaagcatggaaaaaacttttccctggcactgaagagaattcaggcatgaacattgatgaaggagccattttagcagctaacttagcaacagttttacagaacacagaagactgtgaacatgttgaccttgagaatattgatcagtggtttgactctcggagtaatgattcaagctgtcaggtcctaactgacagtgaaggtgtggaggaccaggccaggcctgccgagaccaagccttccagtaagactagaaaaacagaactgaatccagagaagcatatcagccataaagctgcacttgaatggaccgaaaatttactagattatctagaacaacaagatgacatgcttctgtctgataaactggtattgcgaaggcttcgaaccataataagaagaaaacagaagatccagaataacaaaagtcattaataaggctcttaagagtttcagtgtatttgcatctgtgtgactttatctgcagtggaacttaatttcttatttgaattgctgtggatttcaaagccaaatgcattttataaatgactttagaattagatgccattttggattacgtaaattactgcacttgaatgttgattctaggaagttgacatgtaccttgtttgtctactgttcctaatctgtattgtactaatttagatcataaggtacctgaggccagggcgcctatgtctgttttgtacctgaatgtcattgtctaatagaggaccacgcacactataggcaatcaataaatctttcttaaattgaa

SEQ ID NO: 58 - DNA, 1354 bp, Bos taurus
  source 1..1354: mol_type = other DNA
agagcaccggggggcagggccaggcccgtggggctggattcactggtatcgattagagctgcccgggtggccgaggacgtgctcggtcgcagctgccagaccatgctgagtggagtgtgcgcccgactcgcgtcagcgctgcgagggacacgcgcgccgccgtccgcggtcgccctgaggtgtctgcacgcgtccagctcgcggccttccgcagacaagagcgataggacggagaaaccgccccgcgccttcgaccaggccttgctggagttcctggtgtgtccgctctccaagaagccgctcagatatgaagcatcgacaaatgaattgattaatgaagagttaggaatagcctatccaattattgatgggattcctaatatgataccacaggcagctaggatgacacatcaaaataagaagcaagaagaagtggagcagcattagatcatacttaaaaacaacacacacaactaaatattctaaataccacctaccttttaaaaagtcagagtggcgggtattaagtggagaagaatgtttctgtctcttcctatgctgactgtcctgattccactggtatctttagcaggactgttttactcagcctctgtggaagacgacttccctcagggctgcactagcaccaccagtctgtgcttttacagtctgctcttgccgattaccatacctgtttatgtgttcttccacctttggacttggatgggaattaaactcttcaggcataattaacgcagccagagccaagatggtatattctaaagatatgcttgagtgcctgtctctgaaggctcgtctacatgggaatactggaaacccgcttaatttgcaggagagatgctttgcctagctttctgtcaggggtctagggtctatgagaggcttaagctgcagaggcgtctttattgtactttggttctgcccttgttttttgaaggttctcatttatacctggaatatcaaaaggaacattacaaactgtctttttggaaattaacagccccaaccatcacttgatggcttatcccattcataattaaaaattactattttgaaattttatgtttattttagattaaaggctttggtaatgtcacatgttaacgatgactaaaataattccgaaggagctatgttggagtagttgtagagaaatgcatttgaactaatgtgatataaaactatagtaaaatggaaatttcatggacttgccaaaattctgagtttttaaaatcaccatcatttctttggcatcaaatgcttacattaattttaataaaataataaataaaaagatattgacacttgccataaagcaaaaaaaaaaaaaaaa

SEQ ID NO: 59 - DNA, 1386 bp, Bos taurus
  source 1..1386: mol_type = other DNA
cctctctgaggtggctttggaaggagacaccaggtgacacgtgtttgaaaagggtttaaaagttggatgcagttggagtggacggtgtttatctagtaggagcagtgaactggaactgctgtggacttcagtgagcgtttgagcagaacagccagcaatggataactcaatggtctgtttggccaatgcaacagtttgcaatggcacgtcctgtctggtaccagagagcaacttcaacgccattctaagtacaatcctgagcgccgtcctgactatcctgttggctctggtgatgttctccatgggatgcaatgtggaactcaagaaattccttgggcacataaagcggccatggggcatctgtgttggcttcctctgtcagttcgggatcatgcccctcacaggattcatcctctccgtggcctttgacatcatgcccatccaagctgtagtggtcctcatcatgggatgctgtccaggagggactgcctccaacatcctggcctattgggtggatggcgacatggacctgagcgtcagcatgaccacctgctccacgctgctcgccctagggatgatgccgctgtgcctgttaatctacaccaaaatgtgggttgattcggggatgatcgtgattccctacgataacataggtatatctctggtggctcttgttgttcccgtctcccttgggatgtatgttaaccacaagtggccccaaaaagccaagatcatccttaagattggatccatcacaggcgccctgctcatagtgcttatagccgtggttggaggcgtgctgtaccggagcgcctggatcatacagcccagactctggatcataggaaccatatttcccatagctggctactccttagggttttttctggctaggatagccggtcagtcctggcacaggtgccgaaccgttgctttggaaacagggatgcagaacacacagctgtgctccaccatcgtccagctctccttcacccccgaggagctcaacctcatattcaccttcccgctcatctacagcatcttccagatcatcgcagcggctctgttcctggcagtgtacgtggtatacaagaaatattataggaaaaacaatgtggaatctccggagggcagagacaacgaaccagtacctaagtcatcgttctaacaggtcaatgaaggatttcaaccagacgaaaagcaaacaccagtggaccaaaaagcatcctaaagaatatgctttagctctaacaaatacttaattctttgtttggataggatatttggcagaaaaaaagttacagtgaaaatttaaatttgaattgaattcgtgtattggttttggtaccaagcgactggcgatccaaatatttaatgtaacaaata

SEQ ID NO: 60 - DNA, 7511 bp, Bos taurus
  source 1..7511: mol_type = other DNA
atggcggacggggccccccggccccagctatatcgcagcgtgtcgttcaagctcctggagcgctggagcggcgggcccgggcagagggaagaggccacggacgctccgggactgcggcgccgctcctcgtgccgaccggtcgcggccgtcccggcccagccctcccggcgcgtgtccaagctggcgtcggggcccccggccgcccccgcgcagccgcgcccgctccgcagcctctcgccgtctgtgcgccagctctcccggcgcttcgacgcacagcgtctggacgacaactccaccgggccccgagacgggggcgtctccgcggcggcagcagaagaagcggcagagggagccgcgcgcggagcctggcccagcgtcatggagatgcgcaagctcttcgggggccctggctccaggcggcccagtgccgactcagaggcctcagggacgcccagccctgacggggccgcgtgggagcccgcgacccgggaaccccggcagcccccgacgccacctcctcggacgtgcttccccttggcgggcctgcgttcggcgcggccgcttcccggcccggggatcgaggggaggaggcggcggcagcaacaggagcgagcgcagcgtccggcggatggtttacattcttggcatagcttctcccaaccgcaggcctgggcccgggcctcctcctccttctcctcctccatcgtttcctcctatcctgtcagccgcagccgggctgccagctccagcgaggaggaagaggagggcccgccgccgccgctgcccggggcgcagagtccggcctaccacggcggccactcctcaggcagtgacgaggaccaagacggtgagggtggccactgccggggagggaggacgggacccaggcctggaagctcctctttggataaggactgtaggccggacagttatgggttaaatccgagcagcatggactcggcagggggatctgggagccccgatcccccaactcctccaagagctcctagcgaggaaggaccccaagggtggggtgctggctctcccccctgtgtaccaggcccccaggaaggacttcggcccttgtcagacactgtggggggaacgttccgcgtggccaaggtgagctttcccgcgtacctggccagccctgcaggctcccgcggtagcagccgttattccagcacagagactctcaaggatgaggagctgtgggctagccggggttctgggaactggggtgtgtatcgctcccccagctttggagccggggaagggctcgtcaggccccaggtgcgaagccgcaccaagggacctgtgggcaccacgagggtatcgagggatggaggattggatctggacaagagccggcaacggaagtccctgtcgaatccagatattgcctcggagaccctgacgctactcagttttctgcgttcagacctttcagagctgaggatccgaaagcctggtgggcgacctggagaccttggaagtggcccggtagatggcagagattcaccatccgcagatagctcggtggggcaacttgggcccatgcctgccccagccccagcatcatctggcacccgccccacactgaaggacttgacggccaccctccgaagggcaaagtcctttacctgctctgagaagcccatggcccgccgcctgtcccgcactggtgtcctgaagcccagctcctctgaactcctgctggcaggcccaggtgccgaggaggatccactgcccctcgtcgtccaggatcagtatgttcaagaggcccgtcaggtttttgagaagatccagcggatgggtgcccagcaggatgatggcaatgatgtcccccctgggagtcctgactgggcaggagacgtgaccggagggcagcggtctcaagaagaactctccggccctgagtcgagtctgacagatgagggcattggagcggaccctgagccttctgtttcagcattttgcagcctgggtcccacaggtgtttggcgacccctctcctcatcctcagcccagaccaaccaccatggccctggggctgaagagagtctgggagggtgggccctggtgtcccctgatacccctcccacacctggtgccctccgccgcagacgcaaaatcccaccttcaggccctggtgggactgaactgagcaatggggaggcaggtgaggcctaccggtctctgagtgacccaattccacagcgccaccgggccgccacctctgaggagccctctgggttctctgtggatagcaacctcctgggttcactgagccccaagacagggcttccgacagccccagctatggatgagggcttgaccagtggccacagtgactggtctgtaggcagtgaagagagcaagggataccccgaggttattcagagcattgttcagggcccgggtgccttagggtgtgtggtggatgacagggttgctggcaaagcccccaagaagaaatctctgagtgaccccagccgccgtggggaactggctgggcctggattcgagggtcctggaggggagcccatcagagaggtcgagcccatgttgcctccatccagcagcgagccaatcctggctgagcggcgggcagaaccagaagagcccagtcctgccaggggccgggcacagtctgagagggccctacctgaggccccacctgcctcttccactgcccaccatgatttccaccttgacccgaaacttgctaacattctgtccccacggctcattcgccgtggttccaagaagcgcccagcccggagcagtcatcaggagctctcaagagaggagggcaatcaggaccaggctggcagcctgtctctggcccgaccctcctccaaacacgttcgccatgctagtgtgcctgccaccttcacacccatcgtggtgcctgagacaccgacttccattggcccccctgtggctgtgccagagcccatgggcttccctgccaaagcccaccccacgatgcaggcaccatctcttgaggacgtcactaagcagtacatgctgaccctccactccagtggggtccctgccccagtgcccgtggatatgccctgcctgactccgactgcaccaccatcttcggaggtcaagccccctgaggcagcccgggctccagatgagcctgccccaaccagcaagtgctgcagcaagccacaagtggatatgcggaagcacgtgaccatgaccctgctggacacagagcagtcgtatgtggagtcactgcgcactctgatgcagggctatatgcagccactgaagcagccggagaactccttgctctgtgacccgtcactggtggatgagattttcgaccagatcccggagctcttggagcaccatgagcagttcctggagcaggtgcggcactgtgtgcagaactggcacgcgcagcagaaggtgggagacctgcttgtccagtcgttctccaaggacatcctggtcaacatctactctgcctacatcgataactttctcaatgcgaaggatgccgtgcgtgtggctaaggaggcgaggcctgcctttctcaagttcctggagcaaagcatgcgtgagaacaaggagaagcaggcactgtccgacctcatgatcaagcctgtgcagcggatcccacgctacgagcttctggtgaaggacctcctgaagcatacgcctgaggaccacccggactaccctctcctgctggacgcgcagcggaacatcaagcaggtggccgagcgcatcaacaagggtgtgcgaagtgctgaggaggcagagcggcacgcccgcgtgctgcaggagattgaggcccacatcgagggcatggaggatctccaggcccctctgcggcggttcctgaggcaggagatggtcattgaagtgaaggcagtcggtggcaagaaggaccggtccctcttcctgttcacggacctcatcgtctgcaccaccctgaagcggaagtcgggctccctgcggcgcagctccatgagcctgtacacggcagccagcgtcatcgacacggccagcaagtacaagctgctgtggaagctgccgctggacgacgcggacatcatcaaaggggcatctcaagctgccaatcgagagaacatccagaaggcgatcagccgcttggacgaggacctgaccaccctgggccagatgagcaagctctcggagagcctcggtttcccccaccagagcctggatgatgcgctgcgggacctctcagctgccatgcaccgggacttgtcggagaagcaggccctgtgctacgcactctccttccctcctaccaagctggagctgtgcaccacgaggcccgagggcaccgactccttcatttttgacttcccccaccctgatgcccgcctcggcttcgagcaggcctttgatgaggccaagaggaaactggcctccagcaaaagctgtctagaccctgaattcctgaaggccatccccatcatgaaaacccgcagcggcatgcagttctcgtgcgcagccccgaccctgagcagctgccccgagcccacccccgaggtgtgggtgtgcaacagcgacggctacgtgggccaggtgtgtctgctgagcctgcgcgctgagcccgatgtggaggcctgcatcgcagtctgctccgctcgcatcctctgcatcggggcggtgccaggtctgcagcgctgctgccaccgggaacagactgcgtcgctgaggaaccccccagagacggcacccgagcctaccgggccagacctggacgtcgaggcggcggacgaggaagcagcgacgctggccgatccagggccccagccatgcctgcacatctccatagcgggctcaggcctggagatgacgccaggccccgccgagggtgatccccgcccagagctagtgccctttgacagcgactcagatgatgagtcttcgcccagcccctcggggaccctgcagagccaggccagccggtccaccatctcctctagctttggcaatgaggagaccccgagctccaaggaggccacagcagagaccaccagttcagaggaggaacaggagcctggcttcctgccactgtctggctccttcgggcctggcggtccctgcggcaccagtcccatggatgggagagcccttcgtcggtccagtcgtggctccttcacccgcggcagcctggaggacctgctgagcgttgaccctgaggcctaccagagttccgtgtggctgggcaccgaggatggctgtgtccacgtgtaccagtcctctgacagcatccgcgatcgcaggaacagcttgaagctccagcactcggcctctgttacctgcatcctgtatctgaataaccaggtgtttgtgtctttggccaatggagagctcgtagtctaccaaagagaagcaggccatttctgggacccccataacttcaaatcggtgaccctgggtgctcaggggagccccatcaccaagatggtgtctgtgggtgggcggctgtggtgtggctgccagaaccgagtccttgtcctgagccctgacacgctgcagctagagcacacgttctccgtggggcaggattccagtcgcagcgtggcttgcatggtggactccagcctgggcgtgtgggtgacattgaagggtagtgcccatgtgtgtctctaccatccagacacgtttgagcagctggcggaggtagatgtcacacctcctgtgcacaggatgctggcaggctcagacgccatcatccggcagcacaaggctgcctgcctgaggatcacagcgctgctggtgtgtgaagagctgctgtgggtgggcaccagtgccggcgtggtactcaccatgcccacctcacccagtaccatcagctgtccacgggcgcctctcagccctgctggcctcggccagggacacaccggccatgtccgcttcttggccgcagtccaacttccagacggcttcaacctgctctgcccaactccaccacctcccccagacgcaggcccggagaagctgccctcattggagcatcgggactcccctcggcaccgaggccctgcctcggccaggcctaaaatgctggtgatcagtggaggcgacggctacgaggacttccgactcagcagtgggggcggcggcagcagtgagaccgtgggccgagacgacagcacaaaccacctcctcctatggagggtgtgaccctgtccacggtggcccaggactcgcccacccacccgccctccgcctgcttgcctcttccaagcccactggcagaccctcacacgagtgtctgactggggcacacctgtgcctgcacagactgcctcccatccttgcggaagcattgccaatggagcacctgctggccagcggggtctaggcctttcccagccctctggctgctccggtgcacccagtcatgatgggagggtggagagggggcacatgggctgcccaggacctctgtccctggagcttctaccaaagacacggccgggcctggaccccacagggctggggagggccatgtgcaatatttggagggttttcctggggacagtgggagggctgggggatggaatcctttcccgtgtacagtatttatgtttctttttagatgtgtaccttcccaagcacttatttatgcaatgacctggggcagggagtgattggagaaaataacagaagagggaaaaaaaaccctgttcctgccctgggagtcaccctgggaccccaaccagccttcctggagggaccggtcccccctcattatgtacgcacacacacactgcatgtctggggcactgaaacactgccccttaacataaactttccacggccttgtcctgactgggttgccctccagtatccagagcaaaggaccaagtcccctcagtcatccagaacaaggcgatgaccggcggggtgggcagtgactcccatccggggggtttccccaggctcggcgtttccttgcttgttggcagtgcctttagcggtttccgaatgtgggaaccctgtcggggcctcgtccagggccctctcctggcctgagcagggaaggggccctcgctgtccgcacgttgccccccagcccctgtgtctcatgcactggcacacatggtcaccgcagagggtgggcctaggggcccctcactctgaccactgactccctctctccacatcctcctcttgcagcggcagcccttccaaggacgcccaaagctgaggggccagacatggcctccctggctgggaaggccactctggagactagcttcccctgcccccacccccaaaccgcagaagccagtctgaccccagccccacgcttcttcagctgcagctccaaaggtctggtgtcagatggggtttgtttggttctgtttttgtttggggtgggtgggtcattgcggtcttagattatgtttttcttgctaccaaacagtcatgtattaactttccttggatgatgaagtttaaagggtcaataaatagaaacaccaaacaactggtcccca

SEQ ID NO: 61 - DNA, 2567 bp, Bos taurus
  source 1..2567: mol_type = other DNA
gccgtgcgtcttccaggccggggcggcgattcattcaaaaggcgcgcagcttgcgcggccgcccgggcgctcaccgacacgggcccgggcaccgcgtagacccaggagctgggctgctggagggggcggccagccccgtccgggaacacacgctgggaacacagtggccgcctcttccgtctccacccgctggggcctgaggatgaagctgagcccgccaccctggtccctgtcctgtctcctcgtgctgctgccctggcctctggccactgccacaccgacagcccctcggcagtgcccacctgggcaggagcccaacctggaaccagggtggggcacattatgtaggtcctgccccccaggcaccttctcggcttcctggggctctcgcccgtgccagccacactcacgctgcagccctcgagggaggctagaggcccaggtgggcacggcaacacaagacgcactatgtggagactgccagcctgggtggttctcccctgcagaggcccccggtgttccctgctggccatgctcccggacccctctgggtggccgcagctgtttcaaacgggggcgacgggcccgacgtggcgtggaggtggccgcaggggccggtggtgcagcggagacgcggcagcctgggaacagcacgcgggcaggcagccccgaggagacggctgcccagtacgcagtgatcgccatcgtgcccatcttctgtctcatggggctgctgggcatcctggtgtgcaacctgctcaagcggaagggctaccactgcacggcccagaaggaggtcgggcctggccccggaggcggaggcagcgggacgaaccctgcctgccggcctgaggacgccagtgaggacaccattggggtcttggtgcgcctgatcacagagaagaaagagaatgccgcggcgctggaggagctgctgaaggagtaccacagccgacagctggtgcagaccagccaccggcctctgcccaggctgccgccgggcccgcccagcatgccgcacgtttgcccgcatcgccagcacctccacaccgtgcagggcctggcctcgctctcgggcccctgctgctcccgttgtagccagaagaagtggcccgaggtgctgctgtcccctgaggctgcagctgccaccactcctgcccccagactcctgcccaacccagccaggacccccaaggctggggccaaggccggacgccagggcgagatcaccatcttgtctgtgggcaggttccgcgtggcccgaattcccgagcagcgcgtgagttcagtggcctccgaggtgaagaccatcacggaggctgggccctcagggggtgagctccccaactccccacgacctggcctccccagtgaacagcgggcacttctaggaagtggtggaagccatgctaagtggccaaagcccccagcagagaacaaggctgaggagaaccgctatgtggtccggctaagtgagagcaacctggtcatctgaggagctgtcccgtctgaggaccctgcagccctgccctgggcggttccgagggcttcctggaggagggggagctgcagctgggcccatgaggatccagaaacgccagcagacaaaaccgccaaggcctggaggtgggagtgtccaccccggtgaggagacaggaggccggccatcgggtgctgtgtgcaggagcaggtggaggccccctcccgtccccgccacccatttccctcccgcaggtccttgagcctgtgccctgccctgacctctgctccactgggcggctaggccttgtggtggggaagggccctctgcctggcacccctggccccacccttgcccctgtaggggggacttctccctccttttctgacaggtcctataaaggacctgggctgggagctgagttttgggttctcgtccaggtcaagcccctggccattactgggtcccagttctttcgactgtgaagtgggggtagcggcaccgcggcgcacagggccttcccgcctcgcaggctctgggctgggtcgtgggggtggagagctcgactccacactgcccctcccactagtagctttatctgaccccgtttttgctgcttcaggccctccgccttcaagtccccccagggctgcctgaatgtggctctgcatacataaggggcctaatgcatgtgcctgcctctgcccctgctgtttttattgaaactgaagaaacagacttgacctgggcaggactgtggagcagagccccagccacccccacgatctcaggcgcttcggagggaagtggcgtgtgcgacggggctgggaaggggcctctggcctcggtggtgtgtgtccctgcggtgttggtgtttgtcccctggcctgggcagtgcaggcgactgctccacctccgtttccacactggctcagcagtctcgttatttatgtggggctgtgcaggcacgggcccacggctgtccccgtttctcttatttattgaaacttggctgttgtccatccttgtgtctccatactcatccattgttgaataataaaaatgtgggaaagtagtgtcaggagga

SEQ ID NO: 62 - DNA, 7158 bp, Bos taurus
  source 1..7158: mol_type = other DNA
gcggagcggtggcggcggttcgggcggcggctctccgcgctctgaggcgcggggcgcggcgggtgcagccagggctcatgtaatcaaagaagtttctttgttgtgtgtatcttttcagaacacaacaggaattgaaaatgaatcagaacgcagctgagcctgtggtagccacggagaccctggccggggtacctgaacatgtgtttcgaggacttccggaggaagtgaggcttttcccatctgctgttgacaaaactcggattggtgtctgggccactaaaccaattttaaaaggcaaaaagtttgggccatttgttggtgacaagaaaaaaagatctcaggttaagaataatgtatacatgtgggaggtctattacccaaatttgggatggatgtgcattgatgccaccgatccagagaaggggaactggctgcgctatgtgaattgggcttgctcaggagaagagcaaaatttatttccactggaaatcaacagggccatttactataaaactttaaagccaatcgcgccgggcgaggagctcctggtctggtacaatggggaagacaaccccgagatagcagctgcgattgaggaagagcgagccagcgcccggagcaaacggagctccccgaaaagcaggaaaggccccaaagacgaagacgagaatccttccgcttccaccaccgagcaaccagccatccctcaggaggcggtgagccaggatgtgcctccggatctcgccctccctccccctgcccacgagccccagacaggaccagactcggagctggaagcagcgctctgtgaggcgaacgaggtggtggaggaggaggaggaagaggaagaggaggagctggaagaagagggggacgaagcagctgatgtgccaaatgaaagttccctgagagagctggagatccggtgtgacgagaagcctgaggatttgttagaagaaccaaaaacggtttcaaaggaaactctggcagactctgcggaggcagcccctgtcgtgaaaactcccagagccagagaagaggccaacggggacgtgtttgaaacatttctgttcccatgtcagcactgcgagaggaagttcacgaccaagcaggggctggagcggcacatgcacatccacatgtccacggtcaaccacgccttcaagtgcaagtactgcgggaaggccttcggcacacagatcaaccggcggcgccacgagcggcgccatgaggccgggctgaagcgaaagctcgaggatccggccggcggcaaggcgcccagcgacggtgctcctccccaggacgacgcacagccccccggtcttgggcaggacggtctcaccttgagctcagagaaggcgtcccaggactcagtgactccttccgttgtagaagagaacggcgaagcaaaagagcttcacccgtgcaagtactgtaagaaggttttcggcactcacacgaatatgagacgacatcagcgcagagttcacgagcggcacctgattcccaagggggtgcggcggaaagggggcctcctggaagagccacagcccccagcagagcaggcccctcccaccccgagtgtctatgtccccagcacagagccagaggaggaaggggaggcggacgacgtgtacatcatggatatttccagcaatatctctgagaatttaaattactacattgatggtaaaattcagaccagcagcagcaccagtaactgtgacgttattgagatggaatccaattcggcagacttgtacggaataaactgtctgctcactccagtcacagtggaaataactcaaaacataaagaccacgcaggtccctataactgacgatctccctaaagagccttccagcagcacaaacagtgagtccaagaaacggaggactgctagtccgcccgtgttacccaaaattaaagcggagactgagtctgatcctgccacaccttcctgttccttgagtctgcctctcagcatatcaaccacagaggcggtgtctttccacaaagagaaaagcgtgtatttgtcatcaaagctcaaacagcttcttcagacccaggataaattaactcctcctgtggggatttcagccgctgagatacccaaattagggcccctctgcgtgtctgctcctgcgtcgatgctacccgtgacctcaagcaggtttaagaggcgaaccagctctccccccagctctccccagcacagccccgccctgcgagactttggaaaaccaggagatgggaaagccgcgtggactgaggcagttctaagctccaaaaaacccaaattagagagtcacagcaactcaccagcatggagtttgtctgggagagatgaaagagaaactgggagcccaccaggctttgatgaatataaggtgtctaaagagtgggcagctagctctactttcagcaatgtgtgcaaccagcagccactggatttgtccagtggtatgaaacagaaggctgaggggccaggcaaggctccggtccagtgggaagctgtgttagatctcagtgtgcataaaaagccctgtggtgactctgaaggcaaggaattcaaagaaaaccatgttctgcagccatcctgcagtgctataaagaaaaggaagcccaccacctgcatgctgcagaaggtccttctcaacgaatacaacggcgtcgacctccctgtagaaaacactccagatgtgtccggaagtgccagtccctgtaaacctctagatcctcagccagactctgaccgtggtctcaactccagcttgtctgccgctgtcgttgagtctcttcctgacgtttcctcgtcgtcacctgccctgcaaccatcttccctctcttgcgggcggctgccgcctctcctgacccccaccaccccctcctcccctccaccctgccctcccgtgttaactgtggccaccccgccccctccactgctccctactgtccctcttccagccccctcttccggggcatcccctcacccgtgtccctctccactctcgaatgccaccgcccagtccccacttccaattctctcccccacagtgtctccctcgccgtcccccattccttctgtggagcccgttatgtccgctgcttcaccggggcctccaacactctcctcatcttcctcctcctcttcctcctcttctttctcatcttcctcctcttcctcttccccttcgccacctcctctctcagcagtgtcatccgtggtttcctctggggataatctggaaacctctctccccatgatatcttttaagcaggaggaactagagagtgaagacctgaaagccagggaggaagccccatccaccactgagcaggagatcgtccaggagacattcaacagaaactttgtctgcaacgtctgtgaatcaccttttctttccattaaggatctaaccaaacatttatccattcacgctgaggaatggcccttcaaatgtgaattttgtgtgcagctcttcaaggtgaaaactgatttgtcagaacatcgctttatgctccacggcgttgggaatatctttgtgtgttcagtgtgtaaaaaggagtttgcattcttgtgcaatctgcagcagcaccagcgagatctccacccggataaggtgtgcactcaccatgagtttgaaagtggcaccctgaggccccagaactttacggatcccagcaaggcccatgtcgagcacatgcagagtttgccggaagaccctctagaagcgtctaaagaagaagagttgaacgattcttccgaagagctttacacaaccatcaaaataatggcttccggaataaagacaaaggatccagatgttcgattgggtctcaaccaacattacccaagctttaaaccacccccatttcagtaccatcaccgcagccccctgggcattggtgtgacggccacaaacttcactacgcacaacatcccacagacgtttaccaccgccatccgctgcaccaagtgcgggaaaggcgtggacaacatgcctgaactgcacaaacacatcctggcatgtgcctctgcgagtgacaagaagaggtatacccccaagaaaaatccggtcccgctgaagcagactgtgcaacccaaaaatggcgtagtggttctggacaactctgggaaaaacgccttcagacgaatgggccagcccaaaagactgaactttagtgtggagctcagcaaaatgtcctcaaataagctcaagttaaacgcattgaagaaaaaaaaccagctcgtccagaaagcaatcctacagaaaaacaagtccgcaaagcagaaggctgacctaaaaaccactcccgagtcgtcctcgcacgtctgcccatactgccacagagagttcacgtacattgggagtctgaacaagcatgccgctttcagctgtcccaaaaaacctgtttctccttccaaaaaaaaactttcccactcttccaagaaaggggggcacccgtcacctgccggaagtgacagaaacaacagcagcagccaccgcagacggacagcagacgccgagatcaaaatgcagagcatgcaggcacctctgggcaagaccagagctcgaagctcaggcccctcgcaggtcccgccacccccctcgtccttcaggtccaagcagaacgttaaatttgccgcttcggtcaaatccaaaaaaccaagctcctctttaaggaactcgagccccatacgcatggccaaaatgactcacagcgagaccaagaagcccaaagctgcagccaagaaccatgcagctcccctctcaggcaagatgtcgaggagcctgcatgtgcgggcgcagaagagcagagccgtcttacagagcaagtcggccttggccagtaagaaacggacagacaggttcagtgtaaaatccagagaacggagtggggggccgatcacccgaagcctgcagctggcggcttctgccgacccgagtgagaacaggaaggaggacgggagtggcaagcaggagctgaaggacctcagctacagtctccgcctggcatcccgatgtcctccgccagctgccccgtacatcaccagacagtgtaggaatgtcaaaggccacagctgcagcccagctccagggatccctcttcaaagagtaggtgcttcgcctgctccctgacagcacctgaagtgacctggagtcagtgaagccaaagggactggccttctgccctgcaggaagtaccgacctattccagatccatgagcctgcgagagaggagtgcgtgtgtatatgtgtgtgagcgtatgcgtgtgcctgtggtcttcagacgagggtcctgaaccccagggaggctggaaagtagctgactcggcaccgaggatgacacttgggtgctgtcccgggccgtgacctgcagctcagccttcctgttgggagggtcccctcctactatgcaatttttcaagagctccttgatcctgctttttgcttctccggttgtcttttgccacggcggggatttgggtctggccccgggggtcagctccagccctgtccaagcaaggccttcaggaggaggccaagttccctttctggccagctccgctcccgccccctcctccacggcccccctcatctccccccacccctgccctccctcacccctaatgtctctgggaatctgtagcctgtagacacttcggagcctcgctgatcatcccccttggcccttgcacttggaagacttttctctctgcctgagcgagcaatctgagctgctggctcttgagttgtttccagatttgccattctcatccgtcaggccctgggcgtccgggaaaaagtcctggtctccatttatagatcagactcctcctgtgggagaggcagaggacaggaagggatctggtcagggcagggggtgagctcctcgtgtacttctttgcctcacttggtgggaccatcaacacctttagtgtctgagcttctcaaattggctgaaaagggaaacaaacaaacagaaactgggaaatgaaaaataagttggacgattaaaaagcctgggggttggggggctggggaggggggaagagatttccggagaccgtccatcgcctgccagggaacaatggagctggcttcgtctctgatggagcagtccatgtctcggagaaacgggtgagctgagctaggagtttggacccagttcagtgagggtcttgggttttgtgcctttggggcagaccccagacaaggatgtctgagaccactttggcgctgtcttctcagccccatttttgatggtgatgcgccagacccaaagtggaaagaggggaccctttagagtatgtaggaatggtcatgcaaaaaaaaaaaaagggacttttttcagggctattatggttgatcttgcaactctgtaaatatgtatgtagacacttttaaaagcacgtatttatgtccctgactgtaaatgccccatttttaaagttttataacttgtgttatttaatgaatccgtcgactggctgcagcatggggtctgataacggtccaggaggaaagttttgtgagagcctcacgtgggcagaaaagtgctgatcgttggcttgttcccatcctttttcccacccacccagctacagaccgtgatttgttttaaattctcacaaactggaatcctttagcccgctcctaaaattaaccctcatgagcatgaaaatctgtggccgttttttatcctaacaatacaattttgtcccctccatgttgacactcgtgaactgtaccccagcctctcagttcttgagggtttgtttttcctttcagcagtgaggaaggcccacaaacccaaagcttggaaagttgccttcagccagtggaaaattctggaaaatgtggtgggagggctggggggcactcatttttttttgcatgtgcaaacgtgctggtcatcctccgaccctcagactatcaggagaactttcacgaatgttccaatcatgaaaatgggggtgggtgggttggtctttttgttttttaaaaagcggtgtttcctggtcagtgatggtcttcgagcgtgggtttggattgcagaagaggacatgtgaagaaaattaacaatgaaagttaatttatctgccatgtgaagaaaattaacaataaaagtgtgaggcgagattgtgaggaa

SEQ ID NO: 63 - DNA, 5661 bp, Bos taurus
  source 1..5661: mol_type = other DNA
ttcccgggggccgccccacccggcccaggggcgggcgctcgacgcgggctgggtggggatgggggtggggtgggactccttttcctcgccgtggcggcggaggcgcacggcgtggctggggaggcgcggcgtcgtcggtcggtgtcggagtcagagtcggagtcggagtctgagcagccggatctgaggaggaggaggaggaggaggaaggccgaggcgtctgaggaggccgaggccgggcacccagccgcgtccgcggcgccgcgagaaatggaggcccccggcccgcgccccgtgccggccctcggcggtcccggcccactgggggagttcctgccgccgcccgagtgcccggtcttcgagcccagctgggaggagttcgccgatcccttcgccttcatccacaagatacggcccatagccgagcagaccgggatctgcaaggtgcggccgccgccggattggcagccaccatttgcctgtgatgttgacaaacttcattttaccccgcgtatccagaggctgaatgaactggaggctcaaacacgtgtaaaattaaatttcctggaccagattgcaaagtactgggaattacagggaagtactctgaaaattccacacgtggagaggaagatcttggacttgtttcagcttaataagttagttgcagaggaaggtggctttgcagttgtttgcaaggatagaaaatggaccaaaattgccaccaagatgggctttgctcctggcaaagctgtgggctctcatataagaggacactacgagcggattctgaacccctacaacttatttctgtctggagacagcttgaggtgtctgcagaagccaaacctgagcacagacacaaaggacaaggagtacaaaccccatggtattccccagaggcagtctgtgcagcccgcggaaacctgccccccagcccgacgagcaaaacgcatgagagcggaggccatgaatattaaaatagaacctgaagagacaacagaagccagaactcataacctgagacgtcgaatgggatgtccagctccaaagtgtgagaatgacaaagacgtgaaaagcagcgtcaagcaagaacctgttgaaaagaaagagtgtgttacagaaagtgagaaggaaaaacccaagagtcgagctaaaaaaactaccaatgctgttgacctgtacgtctgcctcttgtgtggcagtggcagcgacgaggaccggcttctcctgtgcgatgggtgtgatgacagctaccacaccttctgcctgatcccgccgctccatgacgttcccaagggagactggaggtgtcctaagtgtttggctcaggaatgcagtaagccacaagaagcattcggctttgagcaggcggccagggattatactctccgtacttttggtgaaatggcagatgcattcaaatctgattacttcaacatgccagtccatatggttcccacggaactggtagagaaggaattttggcgactagtgagcactattgaagaggatgtcaccgtggaatatggagccgatattgcctcgaaggagttcggcagtggcttccctgtccgagatgggaaagtcaagctctcacctgaggaggaggagtatcttgatagtggctggaatctgaacaacatgccagtgatggagcagtctgttcttgctcacatcactgccgacatatgcggcatgaagcttccttggctgtacgtgggaatgtgcttttcgtccttctgttggcacattgaagaccactggagctattcaattaactacctgcactggggtgagccaaagacctggtacggcgttcccgggtacgctgctgagcagctggagaccgtgatgaagaagctggccccagagctcttcatctcccagcccgaccttctccaccagcttgtcaccatcatgaaccccaacaccctgatgactcacgaagtgcctgtttaccgaaccaatcagtgtgctggggagtttgtgatcacgtttccaagagcctaccacagcggtttcaatcaaggtttcaattttgctgaggccgttaacttctgcactgttgattggctgcccttaggccggcagtgcgtggagcattaccgtctgctccaccgctactgcgtgttctcccacgacgagatgatctgcaagatggcttccaaggcggatgtcctagacgttgtagtggcctcaactgttcagaaagacatggccatcatgattgaggacgagaaggcactcagggaaactgttcgcaaattgggagtaattgattcggaaagaatggattttgaactgttgccggatgatgagcgtcagtgtgtgaaatgcaaaaccacgtgcttcatgtctgccctctcctgctgctgcaaacctggcctgcttgtttgcctgcatcacgtacaggagttatgttcctgtcctccttataaatacaaactgaggtacagatacactctggatgacctctaccccatgatgaatgcactgaagcttcgagcagagtcttataacgagtgggccttgaatgtgaatgaagctttggaggctaagattaataagaagaaaagccttgtcagctttaaggctttgattgaagaatctgagatgaagaaatttccagacaacgatcttctacgtcacctccgcttagtcacacaggacgcggagaaatgcgcctctgttgcccagcagctacttaatggaaagaggcagacgagataccggtctggtggagggaaatcccacaatcagctgaccgtgaatgagctccggcagtttgtgacccagctgcacgccctcccctgtgtcctcagtcagacaccgctgctgaaggatctcttgaatcgtgtagaagatttccaacagcatagccagaaactgctctctgaggagatgcccagtgcttctgagctgcaggacttgctggatgtcagctttgaatttgatgttgagctcccccagcttgctgagatgcgtgtccggctggagcaggcccattggctggaggaggtgcagcaggcttgcctggacccgagctccctcaccttagatgatatgagacgtctcatagacctgggagtgggcctggccccatattcagccgtggagaaggctatggcgcggcttcaggaactgctcacagtatcagagcactgggatgacaaagccaagagtctcctcaaggccaggcctcggcattcactgaacagcctcgccgccgtggtgaaggagattgaggagatccccgcacacctgcccaacggcgcggcgctgaaagactctgtgcagagagccagagactggcttcaggaagtggaggccctgcaggctggaggacgtgtgccagtgttggacacgctggtggagcttgtcactcgaggccggtgtatcccggtgcatctaccctcgctgccgagactggagtccctagtagctgaagttcacgtttggaaagaatgtgctgctaatacattcctgactaagaactcgtcctattctctcctagaggtgctgtgtcctcgatgtgatattggccttttgggattaaagaggaagcagagaaagttaaaggatcccttgccaagtggcaagaagaagagtccccgtgtagagagtctgagcgacctggagagaacgctgatggaaagcagagagactgcttcagctatggcagcactcggggaagctcgcgcacgagaaatggaagccttgcagtctctccgacaagccaatgaggagaaactgctgtcgcctgtgcacgatgtggagatgagagtctgcgtgtgtcagaaggctcccgcggcccccatggtccagtgtgaactctgcagggatgctttccacaccagctgtgtggcagctcccagcattccacagggcccccgagtctggctgtgtcccaactgtcggaggtcggagaagcctccactcgagaagattctgcccctgctggcctccctgcagcgcatccgcgtccgcctgcccgagggagacgcactgcggtatatgatcgagaggaccgtgagctggcagcacagagcccggcagctgctctcgtcagggcacctcaagtccttgcaggacccagtgggctcaggactgttgtgtggcaggtggcaggccacagcaggacaggtgccagagaccagcaagatatctcagcctcctggccccacgtccttctccttgcccgacgactgggacaacagaacctcctatttacactctcctttctccactggtcggagctgtatccccctccatggtatcagcccagaagtgaatgagctgctgatggaggcccagctgctccaggtgtcccttcctgaaattcaggaactctaccagacgttacttgcaaagccgagccctgcgcagcaggctgaccacagctctccagtgagacccggcagtgagaagagtgacggctgccgagggaagcgagatggagccagcagcctcgagagcaagctgaagagacgcctggaaagagaaggcctctccagtgagcggtgggatcgaatcaccaaaatgcgggcccccaaaaagaagagaatcaaactgagccaccccaaggacacggacagtctcaagttagagagagagtgtagctatgagttggccccctctgccgacacgcagtccctgccctcagactcgtcctcctcggagcaggaggactctgaggacgaggacgccatctgcccggccgtgagctgcctgcagccagaaggagatgaggtggactgggtccagtgtgatggcagctgcagtcggtggttccatcaggtctgtgttggtgtctctcccgagacggccgaggaggaggactacgtgtgcgtgcgctgtgcggagacggacgtgccgggccggagataaaacccgcagagccctcctcagtcagggctcggcccggggactcccagtttctcagcaaagctgcaggactggcgttcaaaccagcctgtgaacggtgctatttctattccttacgggatcatttttccagaactctttgaaaaaaagaaaaaaacgacaaaaattttttgacactttttgtatttttttccttaagagctgtttgtggttgttgaggtttgaaacgctgactgtttttgcaggggttcccaccagtttggaaggattggcgcttgcaccttttcatgtacagcattaaaatcttacctcttcctggggtttaccggctgagtccaactcggtttcagtgcccagaagggcccagtaaatcctcatttgaggaatttctctctcgtttactctgttaccatattggggagcttaagtttttcacttttgaggtttttgtttgttttctttatccacttttgttttccctggtagactaggtttatttatctaagcaataacttccatgttggtttcagtggctggaattaaaaccaaaccaacttccaaatggtgtgtggtgctttagcatctggcggatgtcagaacacggacgtctggcttctggggtcactggtgaactggtgacagaagaggctgctccctaagcacctgtcacagctgtgctggctccccaccccccaaccccccgcctgctcatttggaagcttgtatcaagtgttgcggtttataatgtggaataaatccttggtcctaacataa

SEQ ID NO: 64 - DNA, 8384 bp, Bos taurus
  source 1..8384: mol_type = other DNA
ggcggcggcggcggcagctgcaagttgggctgcaggggcagcgcatacactacaatggctgctggaaagaggcgtaaggaaacaatttccaggcccgccgcgtccagcccgaaatatgagaaaaaaattattagaaattccgcgggcggtgtaaaggcggcggacgggccggagggaggatgttaaagccccgcggttggccaaaatgggaaggactggattccgctctcttccacgaagagtcaatgggactggctaagatcaaggtctgaggcttcgtccatccatcatcaggcacatggctacgcactctcccgagaaaacccacaagtgtaactattgcgagaaaatgtttcaccggaaggaccacctgaagaaccacctgcacacacacgaccccaacaaagagacgttcaagtgcgaagagtgcggcaagagctacaacaccaagctgggcttcaagcggcacctggccctgcacgcggccacgagcggcgacctcacctgcaaagtgtgcctgcagacgttcgagagcaccggcgtgctgctagagcacctgaagtcgcacgcgggcaagtcggcaggcggcgtgaaggagaagaagcacccatgtgagcactgcgaccgccgcttctacacgcgcaaggacgtccgccgacacatggtggtgcacacgggaaggaaggacttcctctgccagtactgcgctcagaggtttgggcgcaaggaccacctgacgcggcacatgaagaagagccacaaccaggagctcctgaaggtcaagacggagcccgtggacttcctggatcccttcacctgcaacgtgtcggtgcccatcaaggacgaactcctcccggtgatgtcgctgccctccagcgagctgctgtccaagccgttccccagcacgctgcagctgaacctctacaacactccgttccagtctgtgcagagctcgggctctgcccaccagatgatcaccaccctgcctttgggcatgacgtgccccatagacatggatgctgtccacccctcccaccacctttccttcaagtacccgttcagttctacctcatacgccatctctattcccgaaaaagaacagcccttaaagggggagattgagagctacctgatggagctgcagggcggcgtgccctcctcctcccaggactctccagcatcctcgtccaaactggggttggagccgcagctcgggtccctggaggacagcacgggggacctctccctgtccaaaagctccatctccatcagtgaccccctgaacacaccagcattggatttctctcagctgttcaatttcataccattaaacggccctccctataacccgatctcggtggggagcctggggatgagctactcccaggaggaggcacactcttcagtgtctcagctccccccgcagagccaggacctgccggatcctgccagcaccatagggctgggctctctgcactcgctgtccgcagccttcaccagtagtctgagcacgagcacaaccctgccccgtttccaccaggctttccagtagagtcccacggtgcgcatcgatggccgaaacgtacgtgcagccctgccttagctgaccgttctggttgtagtgcctactttaagacagtatcacagtttctgcttctgtatagtaccaattttcattaagccagtataaaatagaaactagctttttaaatgagctacagaaccatttgtgttcagctatgtttacctgggtattttgtcctgattcactgccaactgtcatattttaagacgatttctcacataggaaagccattattagcagtcaagtcttacaaatcccatgttctgacttactgttagatcttaaactttgcatttttgtccgagatcaatggctctaccttttgacatttggctccttaaaaatttttgctacagaatgtaagttttctaaaacgtacatgaacgacctgagagtttaaaaaaattttactagcctctaaatggattaataaggaaatgcttcaaatgaatttagaatccagtctggggacatagcatatgtttcttagatataaccataatttcaggtcagtatattttgaagacttgctattgtctggctcaaatgtttgctgtcttttttatgcacatataagaaaaacaagtctagacacaaagcgcaccagtatttgtagcaaacacaccccctaggaaggtgacacggcagtccatgttacttaagagttggccttaaattagtacacaggatgtttgccacgttttatccttgttgctgtctttcttgcattcatgctaatagtttatggcgtcaagagtattcagcgtcagtctttaggtgcatctctttcatcttctccatctttcatggccagagggtaggtcgaggacagccagccacttcccttggcttctcactgcacaattggtccagagtccacagctgttggggaacacaggtgtgcttatcccaggggtggggctgtcccatcttgtagggtgagagaccccaagtagagagaccagtgcgagacctgccactctgctagggacttgtctggggaccacgtcccctgttgccagggtggaaatgaggggtcaagtcacagcatatcacttacatcactcttgctccctttgattcagttttctctccctgagcaccaaaagcagaaaacagaacaacccggctcaccaaccaccaccaccccccgcactgtggtatctttctcattcagtgtttttttttttttttccattcagtgttaattcatttttccctgctgtttttggacagagactttaaagttaaatgcattcaaaaactatttgaggaaagatcacaaaatttaagtgactaaaagtatatacagtaaaccccacttttacaagtccctgggaaagtgggagggacatcacaaagaagctgaatttgcagacggagggagaagagcatagtaaaacaagcactgtcttaggtgatgtgaaaaataacaagagcgtggtgttcagtgaatacagtagttattacaggctttcctcactttagacaaccagcacattttctcaaaggtcaaactattattctatttttctaataatcggttggggcgtcaacctaagaaacatatcttattaagcacttgttaacaccttatttggggaccctgtctgtcggggtgggtttgaggagggagggtttatagaagagtatatatctctttaaaaaaaaaaaaaatatttctgagcactcatgagccctgtgtggaaggttctcttccctttgacagggccagctatcactgcggatttccatgtttaccaagaagttctaaagatgagtgcagattactgaatataatacattatttaaactattggggagtagtataatttgtggagagatgtaaattgtaataatgtaaatggggtttcactatatattatatatatgtacacacacgtgtatcgcacttcatagaatcagagttgctctcgtacggagctttggctccagtatttttatcatgctcctatttgtttttgtttttttaaatcccaggagcagtagtttttatacgtatgtatttaaattttattattaaaagttacattttattaaaaaagtgtgttccaaaggcattaaaattatatatgttaataaggaaatacatttaaaaatttttcaaactgctcctgagcgtttgcctaggagactgtttgctctcaaagtaggcttttagctctgctgtgttgctgcccgctttagtttctatgaaacagattgcttacttaaatcattagttgaatgattagtgttcaatattgctttaatcaccgtcgagaggaaaagaaaaaagaaaaaaaattttggtgacagagcacaaataaatagaaaacctgtttttaaatagaaatcacaaatcacaagtgtggaagcactactttatttttgtctaaaatttgcttaaagagaagtcagatgaacgaactgagatgttggccttttcgtgggctgtatttcactgctaataaaaaagtaaaaagggagtaccaggatttattaaataaagcattttggaaatggggaatggcgccatgtaggtgtatatatatatttttagcctaattctcgcccagcataccttccagggcaccagggctgaaattgtggactctattttcacgtgtacacacccctttgcctgttgctttcccacagacttgaaatcacacttccaagtaagagggaattctgctcattcattttttaaactgactatagtggtcactaaaccccctttgagagaatctctattaaacgtgaggtagactcattttattttaattgcacaatggtctgacaaggctgtaacaccgaactggctctttttttttttttttccttaaaaagaaaattgacacttttgatgtcaactggatgtgattaaaaataatgattgccaattctgttttcattctccagtggccagaatcatcatcctcgaaatttctagtagtgcctttgcttggggttttttttttttttttttcataaagggattgttactaagttaaaaatatttcagtttagaatttggacctcagacaagatactgaacctcattcggtttgaccttccatgtgtttgtacaaattaaggtcaccagataccgaagcacgagcacggaaggatactgacactataagcaatattctccgcggatggacccaaacatcaccttgatagctactggtcactgctacaacgtgcatcttaaacccctgttaccgagttccgttgttcatgtcctgactgatacgcgctactccttgtgcagcttgttaatttcatgtaggtgtttcttccaaactatgtatttttgccgatgtgttgcttgtgtttcattcttcttagtcacttgtgcaacatagtgatatattgtgttgatttggacagtagcggtttttaaaaaccatatactgactgaaaacatgagccagagctgactgctttattaagctaataatgaatgttaaagagtacatattttcaggattattcatctagtaagcaatacacatatataggccaatatttttttaaaaaatagagcttggtcaacctctatactacacatattacaagatatagcactttcaaaacgaatctaaacctttacagaaaaactttcttataggttatgccttttattttaagacttattataatttgtttcaagtgccattagaggatagatatataggcctttgatgtataatgctttgtgttcaagaatggtagacggtattttaaacaggtacagttttacagtgttttcttatcaatttgctatattgcacagaatcagtgtgtcttttcataaggttttacaatggtttattttttttacaaggtttacgtgtctcaaaacacactgtcttcccagtatgtaagttaaaatacaaaaaaaaatattgacccaagttgcttccagcctactgagatccaggcgacattggaggagatcttttagacgtcatgtattcttcattagcaatggcagactgttacctctgagaagtgagtgcctacgttgagcccttgtcttatttttctcacgctgtgtcagcagccacatcaatcacacagatggtctgtggtgatcccatagatccacgttaggctgtgttgtgtagctctgtgctgagaacccatgcagagctcccttctgatccatccaaatggatcctttcttacacatccaaatgatacgacccacaacgggcaaggcacgttcctatcaccccccggcccttctgcaatattgtcaacctctgaggttggaaggcagtgtcttttgataagttacctagcttagaaaggacagaactattgctaatgtgtaaaacacttcattgtataagcttcagtggtacagatgacccagaatgaatgtgtatcttctcagaaacactccttcaatattctgtcggatcatgctgctaatgtaacttgggatacaactcttcatggtgctacagacttctctgtctcgttcagtcgtattttttttttatccataggaaaaaaaaggactacattaggtgtaaaggtgtacaatatatttttatactgtgacttcatttgtctttaacaagacttttacaccacccaccacaatgtattcatgtgcacttgcaaaaggaaaccttggacgtgcaaatgttataccagaacaaacccagctttgtccacaaaggtgaccgtgactcggaaagaaacatgggctttataatatggtgtggagtgaagaacatgctgtacgttactaacagccctttgaatttaacaaaaactgggcatccattcggaaatggattgcatcatgcctgaacataagctggactgctgaaattgtatttttagctgacgaaaaagtgtttggactagtactctaaaaatgttctgatgataaagttttgagtcaaaatagaaaagagaaactgcattccaggctgcattttgtatatttttattgcatttaaaattcgctcttctgtgataccgggaaatcaagtggcttatcctgtatatcatgtacttaaaatgtattcacaaactactgttgtgtttgtataaaatatagacaaagatcatattttctgtgtgtgtataagctctgtaaaatagcaatcacattatgaagctgcagcaagactacattttaaacatccacatccaaagaagcaggctatttattgtccagttacccagttataaaatactcatttgctgctaattaaacaatagtactgcagcttctcatggcctacagtgttatgtttgctgtaagaaaaagatatgtgaactccacaaaaatatatgaataaaaattatagaatggctttagataatgtagatcttcctgaaattataacagatgataaatttctatttggtataacatttttttccctttgaatttgaaatattttctcatttgataaatataagattctaatagctgcctctaggacaatacttggttgttgaaatgtaaagttctagggtttcttttgttttgtcttaacttttgtaccttgaaaggaaggatagaaattgtaaataaaaaagtcaaaatgttgtgcatcaaaactttaatccaggtgaaaattcaaacaaagaatctcccaaaggctgttagaatgtatagagcaatcatgtttttattcatatgagataaaatgggcattaagtatttctttggcacttactgtccatcagtcctgtaaattatctttttagacgttctctttgggctatttgtctaaaacttggctgaaaatcctcccttgaccatttacctggaccagagtaatgagctcacagatgtcattttgtctcaaattcatcagttaaacagtgatgcatctttgaaagaaagagattaattctgaaaatccagttaataatctggccaggaaccagtggcaggaaaaaaattttttttggatttctcatagcacctctttcattatgataggaaaaattgcttcttaacctttgactttcctgtcctaaggttctctaccttcagaaaggtaaagaagatgcacagtgactccccatttggcagttcctcttttcacatacctaattccagcaaatctaaaagcatttctttgttgattcttagcattaacactacatacatttttatttaaattcacgcatatgaaatattttaaggactatttatagaaaggaaaatgctattgctttttatagtcagaatactctgaaaaaggatgtttggggttaattcacttaaggtgtattctcccttatggaaaagcaactagaagtgcagtttaagcaaagcattaagcataagcattgcaccatagatccagtggggctaaaagacttcttcatgcaggtttttgttagatgaaaagttagtactgatgatttatcattaaggggaggaaaacttcagtctgactaaagacagcctgagtttttgctgccacttttccttaaatgagcatttcttaagcagaaggcaccttttcatgcgaagtcctgggctgttcccaaggagtcagctttacagaaagaggctggtttcctgatgaaccgttgcaatgaagatgacaccatcattacaacagtgggaaggtgtaaatgctgtattgagttgtacccagttacccttttttgtagatcaaaaatgatcaaatattggcggtttcagactaaaactaaaaacaaccaagcccagaatgtctcaacttcacctgctttctattcaacacagaggtcaataataaatctgtctactgaagatgtttga

SEQ ID NO: 65 - DNA, 5129 bp, Bos taurus
  source 1..5129: mol_type = other DNA
atgcgatcggagaaatccctgacgctggcggcgccgggggaggtccgcgggccggagggcgagcaacaggatgcgggagacttcccggaggccggcggcggtggcggcggctgctgtagtagcgagcggctggtcatcaacatctcggggctgcgcttcgagacgcagctgcgcaccctgtcgctgttcccggacacgctcctgggggaccccggccgccgcgtccgcttcttcgacccgctgaggaacgagtacttcttcgaccgcaaccggcccagcttcgacgccatcctctactactaccagtccgggggccggctgcgccggccggtcaacgtgccgctggacatcttcctggaggagatccgcttctaccagctgggggacgaggccctggcggccttccgcgaggatgagggctgcctgcccgagggtggcgtggacgagaagccgctgccctcccagcccttccagcgccaggtgtggctgctcttcgagtacccggagagctcggggcccgcccggggcatcgccatcgtttccgtcctggtcatcctcatctccatcgtcatcttctgcctggagacgctgccccagttccgtgcagacggtcgaggtggaagcaacggtggcggcgtctccccagggtccaggggcagtcaggaggaagaggaggatgaaggtgactcctataccttccatcctggcatcgcccccgggggaatggtggcagggggctcatcctcactaagtactctcgggggctccttctttaccgaccccttctttctggtggagacgctgtgcatcgtctggttcaccttcgagctgctggtgcgcttctccgcctgccctagcaagccagccttcttccggaacatcatgaacatcatcgacctggtggccatcttcccgtacttcatcaccctgggcaccgagctggtgcagcagcaggagcagcagtcggccactggtgggggcggccagaacgggcagcaggccatgtccctggccatcctcagagtgatccgcctggtccgcgtgttccgcatcttcaagctgtcacgccactccaaggggctgcagatcctgggcaagacgctgcaggcctccatgcgggagctgggcctgctcatcttcttcctcttcatcggggtcatcctcttctccagcgccgtctatttcgcggaggctgacgacgacgattcgctcttccccagtatcccggatgccttctggtgggcggtggtcaccatgaccaccgtaggttatggggacatgtaccccatgacggtggggggcaagatcgtgggctcgctatgtgccatcgctggggtcctcaccatcgccctgcccgtgcccgtcatcgtctctaacttcaactacttctaccaccgagagacagagcaggaggagcaagggcagtatacccacgtcacttgtgggcagcctgccccggacctgaaggcagctgacagcggacttggcaagcctgaattctcagaggccacccgggagcggagacccagctaccttcccacaccacatcgagggtatgcagagaaaaggatgctcactgaggtttgatggatgcgggcagtgcctgcatggaaggaagacaccaaacacacccttctgtagggacccttcttatcctccactactcgcaccccagctccaggtgacctcctgactccctcaccccacagtggttggtgccaggaccatacacctggactgtcagccttgttacttgacccctgcagcatcccaggtttctccatctaagtgacatttttgaaattctaatggtgccacccattcgtgcccatcttatgtcacgtggatgagatttccatctgaccttccctcgagactctgatttttcacgtttgggacttgtgatgtctctaggaagagtgacgtcgacaggctggaaaccaggcatacctgggtcttgtttctccttagcatccttggctttggggcatgcaccctcccatcgtagcttctggccacatgctaactagccgaagccggtggacagaaacagtactcatcctgttgttttcctccagtgccctgtaaggtcagctggtcattctgcagagaagccttaggagagccttcagttgcacagcctgaacatatgttatctgtctctaatcttggatgcaattagagaaactacgatgacacttggccatttggaggacgtgagaggtcagtcctggaccaaaagggccagtggattcttctagcgtgacctggcacgacagacgttagtctctggtctccacttagctttctcgactctgattcccctgactctctggcttccaggaaggctcctccttctcagagccaaatactctgtctgttcaggtgccttcctgtgcagaggaactgacaaaggggaaccacggagctgcaagaaatggccgaacggaaccaagtacctggcctgaccacttcttagagaagcagtgtcccttagacagtgccatctcagagcggtggaggagtctttttgctgacagcaaattcctttccctcacttcccccagccctctctagccttccatccttcttcctgggaatctgacttaggtttgcagctgaaggcttgctaaagcagactccagcttaaatctgccgccaggtaaaagcacacattggatgctagcgtgggtttctgtttgcattttgcgggcaatgagacaggcggtttagagtaaggaagaagcattgttcttttgctgacagcctcaccggtagagggttgttttttttttttttgctgagtcaggtaaacattgtcctgctccagccctggagatgcagctgttggctgtcaccggggagatgcccggtggtgcccccacctggtctgagccatcttcttccatggtgaactgaactgctcttcctgtaccacctgcccacgtcccctttttctcctctttgcactgccattcatttgctgccttgatcaactgtgatgtactgttctgagctctcctgggtgcagttcagaaaccgaaaggactgttaacatgtttttagttttataatctaggctttaagactccttgatgagatttttttttttctaaagcttttctcgaagagccaacttacgaggtacagcctcttgagggttttcttgagagacactgtgtctggatgttgccgcactttacagggtaacttgggggccacagttccactttgtgcctcggtgtccccacccatttgatgggaacattgatgtctgtccccccacccccacttcttggggaatggcccaggaactcagcttggatcctgggggtgatgctatgcaaatgcgtgaacttctgtaatgatttctagctcgttaccctgcttccccctgtcctataggccaagcctcctcccagggttcccgctttgggtccaaagcccacatccaggccattctgggtccttagctttttaaatccctccctcgcctcttgaactgggccttattctttgcttcttccccttcagcaaggaagatgctctttggagcgattcagtggcatccttttagggccagcaccatcttgattgggagtttggcttctccaaatctgaaatgcagtagaaaatgctcatgattcacacacctgctctgaggctatgaagtgccagttttccacatctgttctggaatattactcctgacagttcagggggaggggggaagagaaggcccacccagaggcaaaagggatgctcatgttgagagtgaatgttgaaacatggatgtgtcattctcagaatgggagtgtgtgtgttttcatcgtcctcaccatttcaagtcagagcatgcccgagtggaaagggtttatgaagagctgggctctccggggattctggttgccgtagcaaccttccagactgttctatcttctgatttgggcagccagggagaacgagagacgatgtagtcccagcttctgaaatggggacgcctctcacataacgttgtttaggggctggcagggctgggacagggatgtgtggcctttggaaaatgagggtaagcagatccccaaagcttcctatctggacctttcttcctggtttccccagcccatcactggagctatctcatctcactcgagtcttcagaaacttgaatgctaggcaactgccagttagaccatattctctctttcgaaatggccttggaggggactagcttctcaagaggcagctgctactagttttagtccataatagcaccatggagcactcacgtcggcccctcccagctctaagacagagagatgatgcttcttcccaaggaaatgtctagctcctgggagggacctcatagatggttcttcttaaagacatccaagacaaactgggaggctgtgacctgacctacttcctctgatggatttcgccaagtgaccccctttgactctgagcctcttgatgtggattttgttccctgagcaccgagggaatctgtgccccatgtcgccgtgacagctggggtggcgttttcacagctgtcagcaaaggggttcaggtctttgttacacctgacttgggccggcaggagctcccagaggtcttcctggatctgtcaggctcagctgagacgtggggggattccttctgcctgagaagccccaggagagaaggggacagctggccgtctttgcacccgagggaactaaggaagaggctggcccagagttccaaaccaggagaactacacctgtatttctgatagcggctggacaaagaggaccgggaccctcaacttctccagaagacgagacaccccccgcccctgctcctagggtggacaggcctgacgctcctccagtggcctcccatgagccatgaccaggctggaaaaaactgggatgggacccatgggcatttaaagatgggacttgggatactgggctgaggctggggcagggaggtgagacagaagaactgagggtgcagacagaagaaaccaggtgtggaggggtcaggggtcgagtgacgccggcaagcgcatggctgcgctgcaggaccgtccaggaaaagggtggccatggaggactcacaataaatacagccggagtctcgga

SEQ ID NO: 66 - DNA, 1270 bp, Bos taurus
  source 1..1270: mol_type = other DNA
gattctgggagaatatggcggccgccgttcacccccgagttgtccgggtcctgccaatgtcacgttcttctgttcctgccctagccgcctctgtgtttcacagcccaccccagcgccagcttcatcatgcagtcataccacatgggaaaggtgggcgttcctccgtcagtggaattgtggccactgtgtttggagcaacgggattcctgggccgctatgtcgtcaaccaccttggtcgcatggggtcacaggtgattgtcccccaccgctgcgagccatatgacaccatgcaccttcgtcccatgggtgacctgggccagatcatctttatggactggaacgggagagacaaggactcaatccgaagagctgtggagcacagcagcgtggtcatcaatctggtcgggcgagagtgggagacccaaaactttgactttgaggatgtttttgtcaagattccccaagccattgctcaagtgtccaaagaagctggagttgaaaaatttattcacatttcacatctgaatgctgatattaagagctcttctaagtatctgagaagcaaggctgttggagagaaggaagtgagagagacgtttccagaagccaccatcataaagccggctgaaatctttggcagagaggacagattcctcaattattttgcaaatatccgctggtttggtggcgtccctcttatttccctgggcaagaagactgtgaaacaaccagtatatattgtggatgtcaccaaaggaattattaatgcaattaaagacccggatgccagagggaaaacttttgcctttgttgggcccagtcggtacctgctgttcgacctcgtgcagtacgtctttgctgtggctcacaggcccttcctgccgtaccctctgccacactttgcctatcgatggatcggtcggctctttgaaattagtccatttgaaccctggaccacgagggataaagtggagcggattcacaccacagacaagatcctgcctcacctgccaggcttggaagacctgggcgttgaggccacgcccctggaactcaaggccatcgaggtgctgcggcgtcaccgcacctaccgctggctgtcttctgagatcgaggacgtgcagccggccaagacgattcccactagtggcccctgagctgcccgcggcttttggtggtgtttgggttgctaccaaccaggccacctttggcggagaatcctgtatatagtgaatgagagcctctattaaaacctgagatgacaaaaaaa

SEQ ID NO: 67 - DNA, 3089 bp, Bos taurus
  source 1..3089: mol_type = other DNA
gcgagtcggagggtggggtggacggtggctcagaagagctcataggaaactgcggggaggggatgagggatgtgagggaggccgtgaggaaagcgggagaaagagaggagtcttgagatcggagttccatgtcaggagctgaccgtaaagatgacgtcggagtatcgaggaacctgaagttcaggattccttatatttcatggaaggagtgggtaaacttaggaatagtgaagacaacaagaaattaatccagagctttcctcatatctcagaacctgtcctcagtaagaatgtcggatagggttgactggttacaaagccaaaatggagtatgcaaagttgacgtctattcccccggagacagccaaccccaagactggaaaatgtcggatgaatccttgtctatttttaaggaagcctcacacgatcccatcagagtgctcagctggctccgcagagacctggaaaaaagcacagcaggattccaagacattaggttcaagcccggagaatcatcgctcggcagggaaatggtcagctcaggagacccacgcaaaggtttctgcgtggactattacaacaccacctccaggggaagtccagggaggctgcattttgagatgagtcacagagagaacccacaccagggccccacctgcaatgggagctccgtagatgaagtctccttctacgccaaccgcctcacaaatcttgtcatcgccatggcccgcaaggagatcaacgagaagattgacggctctgaaaacaggtgtgtccaccaatcagtgtacatgggggatgagcctccacccaacaagagcctgagcaaggtggcgtcggagctggtgaatgagaccgtcactgcctgctccaaaaacaccccatcagataaggctccgggctctggtgacagagcctcggggacattacagagccccccaaatttaaaatacaaaagcactttgaagatcaaggaaagcaacaagggaggcaggggtccagacgacaggcctagctctaagaagtctttcttctacaaggaagtattcgaatctcgtaacgcgggtgatgccaaagagggaggaaggaccttaccggcggagagaaagatgttcagagggtatgaaaggcccgatgacttcacagcttccatcagtcaagggatcatgacctatgccaacagtgtggtgtccgacatgatggtctccatcatgaagaccctgaagatccaggtgaaggacacgaccattgccaccatcgtgctgaagaaggtcctgatcaagcacgccaaagaggtggtctccgacctcatcgactccttcatgaagaacctccacaacgtcacggggaccctcatgacggacacggactttgtctcggccgtgaaaagaagcttcttctctcatggaagccagaaggccacggacatcatggacgccatgctggggaaactctattcggtgatatttgccaagaaaccccctgagacagtcaggaaaaccaaggataagtctgaaagttactccctcgtctctatgaaagggatgggtgaccctaaacaccgaaacgtcaacttcgcctccatgaaatcagaaggtaaagtgagggagagagtatgcacccccacgcccaaacccgagaagtcctgtgtggaaactctgggggaacacattatcaaagaggggctgaccctgtggcacaacagccagcagaaggaaggcatatctgcatgcctccagggctcacccttcgtcactcccaaaagacagtataaacctgtgccggactttcccctggggttcccttttgacccctgcaacttcagcctccctatgcagtgcccagaaaaacctgagaattttatgtgcgactcagactcctgggccaaagacctgcttgtgtctgctttacttttgatccagtaccacctggcccagggaggaaacatggatgcacagagctttctcgaagctgctggcacctccaacctgtatcccactaagtctccagccgtttcccatgagtccagcctccggtctccccaagtgggagctgacccagaagaagtggagaagaaggacctaatgagcgttttcttcaactttatccggaacttgctcagtgagaccattttcaagagcgaccatagctgtgatcccaaggcaaccaaggaagacaacagcccccagtgtgaaagacccgtcaccccttctcccgccaaattaaatgaatgtgatgagactgggggtgcttttgcggggctcaccaagatggtggctaacaaacttgatggccacatgaacgggcagatggtagaccatctgatggactcagtgatgaagttgtgtctcatcattgccaagtcctgcgactctccattggcagaactgggagacgacaagtctggggatgccagcaggccaacatcagccttcccagagagtttatatgagtgtttgtccaccaagggcacagggactgcagagacattcctacagaatgcctatcaggctatccacaacgaactgaggagtctatcagcacagcccccagaagggtgcacagcacccaaagtgatcgtcagcaaccacaacctgactgacaccgtgcagaacaagcagctccaagccgttcttcagtgggtggccgcctctgagctcaacgtccccattctgtactttgcgggtgacgacgaaggaatccaggagaagctacttcagctctcggcagctgccgtggaaaaggggcgcagcgtaggtgaggttctacagtcggtgctgcggtacgagaaggagcgacagctggacgaggcggtgggcaacgtcacacggctgcagctgctggactggctgatggtgaacctgtgatgggggcccgccgccgcccctctttcccgcagtgggccccgccctcgcccctcccttcgtcctcactgccgcggcccccttctcccgcctcctcacagagccccaacattatcttcataccactcacatctaagacacgtcatcgtgcgctagtccctggattttgcagatattcttgtccatgcgagcaaggacgtaaaataaaaattacagataaatggc

SEQ ID NO: 68 - DNA, 2263 bp, Bos taurus
  source 1..2263: mol_type = other DNA
tcgccgcttcactttcgtcatgagagtttactctgcgctgtctccaaaatgcacaataatagtataaatacatcttccgacagtttttaaatgattaaaaataggtaggatggaacaagggaacatttagaccgaggaatttttcacgaggtgccaggaaagaggaaaataattggaagaatcagccgtagcggatcttccggctgtaaaaacaaggcagcagtcagctgatgggcccagcgccaaggccgatggtcaggaccggtactgccgacagctgggaactcggaacaagaggagaatcctgctaaagatgaaaaaaaacagagaaagattctgcaaccgagagagggaatttgtgtataaattcaaagttggaagtcagtgctttgaactgagggtacccctcaaatttcctgttcaagagaatgccagtcatttacatggacgtctaatgctgctgcacaacttaccgtgctttatagaaaaagacttaaaagaagctctgagccagtttattgaagaagaatccctcagagattatgacagagaggccgaagctgccctggaagctgtgaagtcaggtgaagttgacctccatcagctggcaagcacatgggccaaagcctatgcagagaccacactagagcatgcgaggcccgaggagcccagctgggatgaagacttcgcggatgtgtaccatgacctcatccattctccagcctctgaaactctcctaaacttggaacacaactacttcgtcagtatctctgaactcattggcgaaagagacgtggagctgaaaaagttacgggagagacaaggtattgaaatggaaaaagtgatgcaggagttgggaaagtcactgacggaccaagatgtgaacaccctagctgctcagcatttcgaatcccagcaggacttagaaaataagtggtccaatgaattaaaacaatcgactgccattcaaaaacaagagtatcaggaatgggtaattaaacttcatcaagacctaaaaaatcccaacaacagctccctcagcgaagaaattaaagttcagccgaatcagttcagagaatccgctgaagcaaatggaagggtttatgaggaacagagaaagttagaagaaagttttaccatccacttaggggcccagctgaagaccatgcataacctgaggttgctgagagcagacatgctggacttctgcaagcacaagaggaaccaccgaagcggtgtgaagctccaccgactacagacggcgttgtcgctttactccacgtccctttgtggcctggtgttgctggtcgataatcgaattaattcctacagtggtattaaaagagatttcgccacggtttgccaagaatgcactgactttcatttcccccggattgaagagcagctggaggttgtccagcaggttgtactttatgcaagaacccagcgcaggagcaagttgaaagaatctcatgattctggaagccgaaatggaggaagtgacgataagactaaaactgcagatagaaactatttaaatattttacctggagaattttacatcacacggcactctaacctctcagaaatacatgtcgctttccacctttgtgtggatgacaacgtgaaatcgggaaacatcactgctcgggaccccgccatcatggggctccgaaacatcctcaaggtttgctgtacccacgacatcacgaccgtcagtattcccctcctgctggtgcacgatatgtcagaggaaatgactataccatggtgcttaaggagagcggaacttgtgtttaagtgtgtcaaaggtttcatgatggaaatggcctcgtgggacggagggatttctaggacagtgcagtttctggtgccacagagtatttctgaagagatgttttaccaacttagtaacatgcttccccagatcttccgggtgtcatcgacactgaccttgacctccaagcactgagcccccacagaccaacacactgaagaagatgctgggatcctgtcaagcaaaagatgcccagaaagagaacttgccacccaaggcacaaatccgatcatgataaatgtctgctaaacattccaaaaacccatatgttgtacagactggtgatcagccccttacttatatattcattcttaagctctgtttatgggggaaaaaaaagagtcttggtttttttttccctactgtttccctactgtcctatctgtgaatattagtgagttaaaactttgtctgtttcacacatgg

SEQ ID NO: 69 - DNA, 2176 bp, Bos taurus
  source 1..2176: mol_type = other DNA
gggcgggaattgaaattgccgcggcggctgcctgctagagcctgagagctgcaggtttccagcctcggaggggaccatggtgcggcctgcgaggcataagaagccagtcaattactcccagtttgaggactctgatagtgatgatgattttgttcctgcaaccgcaccgttaaataagaaatccagaacaacacaaaaggagttgaaactagaaaaaccagaacctaagctgaagaatctccaggaaaaagacatctcactacaagagaaaacccctaaaaaaaggatggctttggacgataaactctacaagagagacctagaggtcgcactggctttgtcagtgaaggaggttccagctgtcaccattgatgtggagcagtctcaaggctttgacaaacatggcaagaaggaaacaggagcaacgagtaagtctcctcatgtctccaactgcagtgtagccggtgattatttagatctggataagatcactgaggaggatgagtctcaggacactccacggagaaggaaggctgcatcccaagccgtggtccagcagaggaagattctcttggaaaacggttatgacgacggagccaacgactctgagccagactttgcaactggtgaagactctgaggatgactctgattttagtgggagtgaagatgatgaaagcttcactgtgagaagaagtaaagttaaagaaaccaaacggaaagaaatgaaggcgaaacctccagctgaaaagaaagagaagcctaaacccaagcctggtgctagggtgacttcagtagactgcagtccagctgccgtcaagtcagaatctcggtcctcaccaagaaaggaggccagtaggacgccttcacagatatgcagtcctgcagcagaaagcaagagacctaagtgggtcccaccagccatgtctggaagcagcagccgcagccccctggcaggagtggccgtgaagtctccgagtcagagcctccgcctcggcctgtcccgcttagcacgggttaaacctctgcatccaaatgccaccagtagctgcgtgtagccacaggaagatgtttgattaagaagctctcactcgtataagcgggtttagatttgaggagggaaatgtaatataaaggaatcctttaacgtcatataaattacctctacctgttttgtgactgttctctgcaaagctgcccaagacttcagcaagagttctatgtggattctcttggaatgtccttccttctgaagagcatattctcctccctcagtgtatgtaagcagtcactgtgatcaccttgctgtcagcatgacacggaagaggtccacgttacacgctgctcgttgccagtaacaggatgctgcttcctgcctttgtcaagttgatggaccctgaacttcgtgtgcagagtttatctcgcaagaggaagattcccgcttctcccagggacgtttctgcactgacaacctcactgtcagttttattgccatgatgtttcatattttagccaaatgtttttataatgagaattcagaattattttccacattgaaacagttgatgtactatagtgtatgtctcctacctagaagtattaatagatggttatagcaaataaaatttcatgacatttcatgaagccttcattgtcaaaacctttgatatgcactttcccttgataccattgcatccgtctgtctctaactgtctgcatttcatgataaaataagtgctctgcagaaaaaaaaaagttatccgttttgttgtaaatagaaagaggaagagctgtttcttaaagtcatcattgtacgtgaagcccatgtttaacaggtggctgtcaagttgtcatccctgttgtattagcatagctcttcaggaagcttagatacgagttcgctgtgaataaaagtagtaatagatgttttctatttacagtgaaaatttcaaccagctcctgatcactttttaataaaaattctctgaattttaaccattaaaaatgtatataatacttaattcttggtttatgaaatatgttaatgtgaaagtaagtaattcagacttatgattaaagtgagcatttgcccaaaaaaaaaaaaaaaaaaaaaaaaaaaaaaaaaaaaaaaaaaaaaaaaaaaaaaaaaaaaaaaaaaaagaaaaaaaaaaaaaaaa

SEQ ID NO: 70 - DNA, 627 bp, Bos taurus
  source 1..627: mol_type = other DNA
atggcccggggacagacgccgctcatcactatgtcccggggagcaggacgtccgcagggcacgctgcgggctctcgtcttcctcggcgtcctagtgggcatggtggtgccctctcctgcgggcacgagagccaacggcacactgctggcttcgaggggctggggcaccctgctatccaggtctcgagccgggctggccggagagatcgctggcgtgaactgggagagtggctacttggtggggatcaagcggcagcggaggctgtactgcaacgtgggcatcggcttccaccttcaggtgccccccgacggccggatcagcgggacccacgaggagaacccctacagcctgctggagatttccacggtggagcgaggcgtggtgagcctctttggggtgaagagcgccctcttcgtcgccatgaacagtaaaggaaaattatacgccacgcccagcttccaagaagagtgcaagttcagagagaccctcctgcccaacaactacaatgcctacgagtcggacctgtaccgaggcgcctacatcgcgctgagcaaatacggaagggtgaagcggggcagcaaggtgtcccccaccatgactgtcacgcacttcctccctaggatatga

SEQ ID NO: 71 - DNA, 1179 bp, Bos taurus
  source 1..1179: mol_type = other DNA
taactgcccagccagtgtgcgtcactgcctcggcgagcagagagccgagcgagctggggagagcagacaagtttgaaggggagggcagagagagtcagccgccccaggggcgctcctctcccaccgcccatcctatccctttccactcttcttccccggcttctctctctctgcccttaactcccccccccaaaaaaaacccttatttagccaaaggaaggaggtacggggaatcctctcccctcccccctcccaaaaaaaccccccaacttttccagtccggagatcgcaggggcacgagcgagtagccagctggccatggagctgctgtgcggcgaggtagagccggtccgcagggccgtgccggacgccaacctgctccacgacgaccgcgtgttgcagaacttgctgaccatcgaggagcgctacctgccccagtgctcctacttcaagtgcgtgcagaaggacattcagccctacatgcgcaggatggtggccacctggatgctggaggtctgtgaggagcagaagtgcgaagaggaggtctttcctctcgccatcaattacctggaccgcttcttggccggagtcccaactccgaagacccatctgcagctgctgggggccgtgtgcatgttcctggcgtccaagctcaaagagaccatcccgctgaccgctgagaagttatgcatttacaccgacaactccatcaagcctcaggagctgctggagtgggagctggtggtgctgggcaagttgaaatggaacctggcggctgtcaccccccacgacttcatcgaacacatccttcgcaagctgcctcagcccagcgagaagctgtcgctgattcgcaagcacgcccagaccttcatcgctctgtgcgccactgacttcaagtttgccatgtacccgccgtcgatgattgcaactggaagcgtgggggccgccatctgtgggcttcagcaggatgaggatgtgagctcgctcactggagacgccctggtggatctcctggcaaagatcaccaacaccgatgtgcctgcttctcctccaacatggagccttctcgcctgctcacacacgctgaggctggcaggcagggccggagcggggggcccccctgcaccacagggcgtgggagaggagggccccccgctcctgtgaggaggtgtccgcctc

SEQ ID NO: 72 - DNA, 1227 bp, Bos taurus
  source 1..1227: mol_type = other DNA
acttcgagctgtgaccgcgcaccaacaacagcaacaactccactgcgccgggctgaggagcaggaattaggagctcgcgaataatatgaaagggatccgcaaaggggaaagccgagcgaaggaatccaaaccccgggagcctggcacgagaaaatgcgctaaatgtggccgcctagacttcatcctgatgaagaaaatggggattaaaagtggatttacgttctggaacctcgtctttttattgacggtgtcttgtgtgaaaggtaggcttgctttcggggtcccctctgctttggatggaggtgagctgacttgtcgccagagacgggtagcttactttgcaatttgcacggataaaataatcatgattatactgaaggagtcgattcacagctctttttctccctcagataggaaatgcattttgcttgccagatttggcgttcaatgcagtgattgagcttgtgctcaataaatattcttgcagtatgctcaaacgaatacgtgagttggttcctatagcctctaactactggataccaggcagaattcctctgcaagacttttcttggtgatgagggttaccttgatttctagtcaaatcagtagggtttgatgctttggttggaatatttatattaaaggcgctcctggaaatgctttttaaccctttcaggtaggaaagttatgttaggaatgtgctgcttttaaatgcattagggcacaggggtctttcaagaatccccccactccccacccccaaccaacatgaatgtgtgcatgcatttgtaaattatttctacgtgggctcttttgcactgataaataatagaggctgtttcctaggtcctgtggtagaggtttggcttaaaatggggcttttgaacggtctgagacgccaggaagtgtaaccaggtacctttggggatgaatgaaggtaggaggaaagtcatcagctgaagaccaaaaagaaaatccccagcaagtctcggcgttcagacaggtagagtgagtgtcacatgcacagacaaggggaggaggtgggtgggatgtaaaattcaatcctaagcaggaaggcaagggacagtcaggatcactggaaggagagctgtcctctgggagaaaatagattcccagctctgttctacaacttctcctgcgccctgctgcccattaattgctgcaaaaaaagaaaagaaagaaagaaaaccaaaaaaaaaaaaaaaa

SEQ ID NO: 73 - DNA, 1777 bp, Bos taurus
  source 1..1777: mol_type = other DNA
cgcctgcccgcccgaagcgccgtcgcctgccaccgccatgggtcgacagaaggagctggtgaaccgctgcggggagatgctgcacatccgctaccggctgctccgccaggcgctggctgagtgcctggggaccctcatcctcgtgatgtttggctgtggctctgtggcccaggtcgtgctcagccggggcacccacggtggtttcctcaccatcaacctggcctttggcttcgccgtgaccctaggcatccttattgctggccaggtctctggggcccacctgaaccctgccgtgacatttgctatgtgcttcctggcgcgcgagccctggatcaagctgcctgtgtacaccttggctcagactctgggagccttcctgggcgctggaattatcttcgggttgtattacgatgcgatctgggccttcgccaacaaccagcttattgtttcgggccccaatggcacagctggcatctttgccacctacccctctggacacttggacatggtcaatggcttcttcgaccagttcattggcacagcctccctcatcgtgtgtgtgctggccattgtggacccctacaacaaccccgtcccccgaggcctagaggccttcaccgtgggcctggtggtcttggtcatcggtacctcaatgggcttcaactctggctacgccgtcaaccccgcccgggacttcggcccccgccttttcaccgccatcgccggctggggctcggaagtcttcacgacgggccgccactggtggtgggtgcccattgtctctccgctcctgggttccatcgccggtgtcttcgtgtaccagctcatgatcggctgccacctggagccgcccccaccctccaccgacgaggagaatgtgaagttgtcccacgtgaagcacaaggagcagatgtgaatgggcgggggcaccccccacgccctcccatgagcgtccattgactgtgcaggggctgctccccagaagccctcttcatgactcccctcctggactaggaagcttcttgtccaccacctcccactagacagccccctccaggcttttccatcctgaaggaaataggacccagcaggaccttcagcctgtaagcggtggtggcacagggagcctgaccgatctagcccctcatctcccagagggagacagtaggcagcaggtgtgtgagtgtgtgtgcaggtacgtgtgcgtggttttccagatattcagggcaaaagaccgagtggaaaagattctgtcggtggagttgggtgggggtccagaggaagagaggggaagtgtggtgtctatctgtgaatgagaaaagaacgtttcaggggctccatgtggaggagggtccaggtacaggcatttctcagtatgtgtgtgtctgccctttcttctattcagggggctcttataggcttcagtagggtgggaatataagtagggctaggaggagggggccgtagcccagttgtggagctctgtctattcataaataaggagcagcagagtgatgtgttcatgtcacagtggaggcatgaggggagtgggggtgaagtccaggtcacaatttcatgtttgctcttaaaaatatatgttaatatatgtgtattaatacatatatgtagtagatgttacagtttcagggatggggattgggagactccatggtctgccctcctttaatcctccactcaacacatgtatttttgccttttataaaaaaacaataaaatgtatgccaattgtgtaaaaaaaaaaaaaaaaaaaaaaaaa

SEQ ID NO: 74 - DNA, 3088 bp, Bos taurus
  source 1..3088: mol_type = other DNA
tgtctgcacttggctcgagtgacaagggccaaacccctgctccactgacctcttccctggaggagcagagcagagcagcactcgggtcgccctgaggagggctgagaggcgggctctggctggggacatagggaccacagagccctggcaggggctgcagccagagcctgagacggtcatggaggaagctgtccaggggctgctgtgactgagaggccccaaggcaggcaactgggagcataaggcctttcagcagagtccacccgcgtgtccaaggtggtctcgacacctacagcaacaaagatgcaagtcatatggcagaacgagaatgttcgagagttcctggcggagttcatcagcacgtgttcggtctcggcgccgtggcccacatggttctaggaggtaaaatggggagctacctcgctgtcaatttgggttttggcttcggagtcaccataggtgtgcacatggcggggaacatctctggggcccatatgaatgcggccttgaccttcaccagctgtgcgctaggccgcatgtcctggaagaagtttcccgtgtacgttctgggtcagttcctgggctccttcgtggcggcggccaccatctacggcctcttctacaacgctattatcgacttctcggggggaaagctgatgacgaccggccccacagccactgctaacatttttgccacctaccttcctgaccacatgacactgtggaggggcttcctggatgaggtggtggtgactgggatgcttcagctgtgtctcttagccatcgcagacaaggggaacaacccaacgctgttggggacgcaggccattgtgactggcatccttgttgtcatcatcggaatatccctgggcatgaactcaggatatgccatcaacccatcccgggacctgcctccccgcttcttcaccttcctcgctggctggggctcacaagtcttcagaaccaaggactggtggtgggtgccggtggtggcaccacccctgggtgcctactgtggtgccatcatctacttgttcttcattggctccaacaaacgacaggagccccaggtcctggagaatcccttggcgtttgaagaccacaaaatatctgtgttgcccaagaccacgtctcaccagcatggggtcgcttccctcacccctgtttccgtgtaccccgaagatagaccctcagccttgtaccacccttaagtgactccatctgcctagagcccctctaagactatttgtgaccctacatccaccccaataaagaaagccttgtcccacagtggcaaccccgcttcttgggggcctcctgtggtggggcttacctcctgggttctctcagaagctcccaggttatgctgtagcccaaggctgggcaggagctgtggggcctccaactcctgccggccctgggagctgggtggcccccaggggagcaagtggaagggccgatcagtggcctcattaagacaggactagattagaagagcccggacatggaagggtgggctgccaggcacagggcagggaggggatggcatgggggagggattccagctggcaggtgggggctcagccctggaggaaccttgagctgccttgggagaagactctggactctccttcaatcctggccagtccttggaccaggcaaggcaaggccccagtgtgtggatcagagtttctcaacatttttgttttccttagtctcccctcctcaggagccttttaggcacttttcccccttaattgcatgatattttaataccacagatataccgtatatctgtttgtgtactgtacatatatctttgctttatacaaaaaaagtaagattttaaaaaatctctccctttcaaggatccaattttttcacccatgggagcataatcaccccatccttgaaaatgcctgaagtagaatgagaggaggtaagaggcaggctctgccttcttcgtgggggcagagctctcccaccgtggggaattccagagttggaaaggaagaagcagagaggaaaatcctgtcaaaaggcagggggcaggaaatggtctcatgaggttttggggaacaggtgcagagccggggcagccagggtccaccgatcccaggctgggaggatcatcctgcttctgggactcagaaggcagcatcacccatggcttctgtccctgcttctgcttttagcctttcagcagccattgagcacctccggtgtacaggctctgcgggtaaggttgggtctctgtcttcaaggagcacagtccctactcaggacaggtgatcacaaggaaagagctgaggagtgaagaaggagctgtgtgtggggctcagagggggctcacagctcagtcttgagctgaggccacagatggcttcctagaggatgagacagagcaaagggtggtggggtggtcctttctggggctggatgccgaacttcatggtttgaggagacagggcagggtggggggtgggtagaggacgctggttttccttctggaggtggggacctggtgagatgaggtttagaaagcccacatgagctgcagtttgcaggatgctggaaaagggggagactggggcctgtaaggaagtgtggggcaaggctgttccaggtggggcagcaggacagctcacaggtcagtgctggagggtggacagaagagaaaggattcgcctgacaccaaggaggtaaaagcctcaggctaggttcagactggagggaggagtgggtgaggatgggccaggggccaaggctgccttccagttgctggcttgggcctggggtattggagatgtccccactgaacatgaagaatgggaaaggcactggtttgaagaggaagatgataatttatactgaagacacatcgactttgagttgcctatgggacttgggcaataggtgtgtagagtcctgggcaagttactctgacttgctgggcctcggttttctcatttgtaaagtatcaaaatacctaccttttgtgttgatgtgattagatgaaacaaaacatgtacaatgctgaggatggtgcttggcacagagtcagtgctcaataaatgattatgccttt

SEQ ID NO: 75 - DNA, 1906 bp, Bos taurus
  source 1..1906: mol_type = other DNA
cagattctctggggctggttcatcacctccgaatactcctgtgacaggaggcgtttacaaaacccgcctccagccccaagaagcaataaatagaaggcgcccagcccacgagaagcaaggagaagtttctaggcctgcgtccccgggtttattaagctctcggcttcactcagtggctctggctgggagcctggaccgcttgggaggacagtggcggctggctgatctccggcgaggctgtgtggagaggggtccttgagaattctgctggaccaaccggaggtcgccttcgtggctggagtgtctttgggggctcggaccccagccaagtgtgtgcctctgtccctggcagtggatggttctgaatctgaagaataagcttggaaggccagcgcacccacctcaccttgctgtgtgaccttgggcaggtggctccctctctctgagcctctgtttccccttcagctcagcagccatcatggctgacggtcagatgcccttcccctgccactacacgagccgccggcgccgggaccccttcagggactcgcccctgtcctcccgcctgctggacgatggctttggcatggaccccttccccgacgacttgactgcctcttggcctgactgggccctgcctcgactctcctccgcctggccggggaccctgaggtcgggcatggtgccccgggggcccactgccatgaccagatttggggtgcctgctgagggcaggagccccccaccattccccggggagccctggaaagtgtgtgtcaacgtgcacagctttaagccggaggagctgatggtaaagaccaaggacggatacgtggaggtgtctggtaaacacgaagaaaagcagcaagaggggggcatcgtctccaagaacttcacaaagaaaatccagcttcctgcagaggtggatcctgtgacagtgtttgcctccctttccccagaaggcctgctgatcatcgaagctccccaggttcccccttactcaccgtttggagagagcagtttcaacaatgagcttcctcaggacggccaggaagtcacttgtacctgagaccccagtcttggcccttccttgttcccgccccaacccctgggcttctctgattccaggatacattactatagctgaactcatagatttagtgtggctaaaatgttgggggtcggggtggattgaccagattccctggatagtgttagtagtaggtttttctgcaagatggcgcaattgacaggtcatgctccattgcattaggccaaaatgctgggagttttcccttctttaccttttctatcatgatgaaaatgttgcacattttacagttgcaaaacaaataaaaggggacataacatttcactttgtatcttatcttgcagctaatgcaagagttgcttttctctggggaccgggattcccattctgggctcccaatgtccaacctgccccttggtttggttgatggagccctcgtagctcaccccacagtgtttctgcagccctggtttacaaagggttatagacaggtcttatatccccatatgggatttatcctgcaaccacatgaaaacagacagtgccgtgtgccctccacccaggcatttatagcatgttcccaagctggcactccccaaggactttagaagtcctttagtttattctctggttaaagtcccccctttcttgtgtctcctatgtccaagtcgggatttttacagagggggctgtctccagacagctccaccaggaaccaagcaaaggccaaatagtctggcaggcaggctagtggtgttgtgtatatgggtgggacgtgcgtgtcattgttatttgaattgtgctgttgtttagggaaaataacagtaaacaataataaaaggaactgatgttatt

SEQ ID NO: 76 - DNA, 1225 bp, Sus scrofa
  source 1..1225: mol_type = other DNA
cggggcgggtggccggccggacactggacccggaaggggggtaggcggctgggatgagtggcgagctgtccatgggagcacccagcggccccattggcaccagtacaggcaggggcacctgcagcagctgaggggccgaagagtcacccctgaacttgaggacgagcagccggattccagcccccagccccagggccccacatctcctcgggctcagccgcgcgccccagctgccccccagcctgagctgcagcaggccagggctgcccgagaccccagcccccaggtgagctgctgcagcctgtggcccaggagatctccgccggctcagaactgaggcgggcagcccacccagcccacagcgattccaatggggatcccgatgaggaagccgctgctcgtgctgctcgtcttcttggccttggcctcgtgctgctatgctgcttaccgccccagtgagactctgtgcggcggggagctggtggacaccctccagtttgtctgcggggaccgcggcttctacttcagcaggccggcaagccgcgtgaaccgccgcagccgtggcatcgtggaagagtgctgcttccgtagctgcgacctggccctgctggagacctactgcgccacccccgccaagtccgagagggacgtgtcgacccctccgaccgtgcttccggacaacttccccagataccccgtgggcaagttcttccgctatgacacctggaagcagtccgcccaacgcctgcgcaggggcctgccggccctcctgcgcgcccgccggggtcgcacgctcgccaaggagctggaggcggtcagagaggccaagcgtcaccgacccctgaccgcccgtcccacccgagaccccgccgcccacgggggcgcctctcccgaggcgtccggccatcggaagtgagccaaattgtcgtaattctgcggtgccaccatccacctcgtgacctcctctcgaccgggaccgcttccatcaggtcccccttctgagatctctgtacccttctgtctgcgggcatctccgccccgggccccgtgccccaacctccccatgtcaggctagtctctcctcggccccttccatcgggccgagggcatccaaaccacaaacccaattggcttggtctgtatctccccccaaattatgcccccaattatccccaagttacataccaaaaattgaacccctcaaccacacccacataccatcacg

SEQ ID NO: 77 - DNA, 3622 bp, Bos taurus
  source 1..3622: mol_type = other DNA
gcggggggctgggctgatcagtgatctgtccgtgggctcacacgggcggccctgtgcgtgcaggtcggggcaggggtgccccgcaggagctgagacatcaatggggatcacagcaggaaagtcggtgctggtgcttcttgccttcttggccttcgcctcgtgctgctatgctgcttaccgccccagcgagactctgtgcggcggggagctggtggacaccctccagtttgtctgtggggaccgcggcttctacttcagccgaccatccagccgcataaaccgacgcagccgtggcatcgtggaagagtgttgcttccgaagctgcgacctggccctgctggagacttactgtgccacccccgccaagtccgagagggatgtgtctgcctctacgaccgtgcttccggacgacgtcaccgcataccccgtgggcaagttcttccaatatgacatctggaagcagtccacccagcgcctgcgcaggggcctgcccgccttcctgcgagcacgccggggtcgcacgctcgccaaggagctggaggcgctcagagaggccaagagtcaccgtccgctgatcgccctgcccacccaggaccctgccacccacgggggcgcctcttccaaggcatccagcgattagaagtgagccaaagtgtcgtaattctgccaagtggcaccatctacctcgcgccgacctcctgaccgggaccgccccactaggtctctctctgaaatccctgtaccgtcctgtctgcgggctcccctgccccggcctctgtgccccaacctccccacgtcaggcgaatccccctcggccccctccatctggctgaggggatcagaacaacatctctaaaaatgtacaaaaccaattggctttaaatatccccccaaattatcaccccccaaattacccccaaattacacaaccaaaattgcaatcatgaacccctcaatcagcccccttgaaacgaattggctttttagcaacaccagaaaagcaaactagctttccaaaaacttcttaaaaaaaaaaaaatcaattggctgaaaaaaatactaaaaataaattggcttaaaaacaattggcaaaataaaagaattcggcccccccccccttccttctcgttccttttggatctggacttaaattggctttgatctaatcatctaagagaaaggaaggggccaaatttgcaggtaggcttgtccttgctggccagccatctccccccgaccgtcgccaggccctcctcgggcactggcagtgtgacaccaaagacccaaacctgctcctcctccaagtttccatcactgggaccacagagcagtgaggggacgctgtgagaccagaggaggggagcacggagcagaaaccccggatcgcgcagataaagttgaattggcaaaacacccccacactccaaatctgacatcacaatcctaccttggaaagcacatgcaaccctgcacacacacacacacacacacacacacatccatgcacgcacacacacacacacgcatccatgcacgcacacacccacatgcatgcatgcacacacacacacacagcctcacatgcatccatgcacacacacacacacacacacacatccatgcacgcatgcacgcacacacacacacacacacacacacacacacacacacacactctctagggacacccgtgcccagccttgtgcagctgagaccgaattccatcccaaattattctctgagctgccccgcgatgcggttttcctgttccgtgctcccctctgatcccctgggatgcaccattgtgggaaggtgtgtctcgggctcaggcaggtggttccatctttccgagaagagggtgatccccacctcttctcggcacctgttctgaccccacagcccacagaatgtaagtcctgggtgcccctagaccccagaggcatccacgcaccgaccatcgtgcagagtgaggaacggtttggccaggcgggagatggctgcagccaagcgagacgagcccaaccacgtagcctgaccccctgggtgtgcttctgcaagatctggggagccctctcccccgagcacacctagggatcatctttgccagcctcctggggaagcctctcagaaatgagggcccccccggaggctggcaaaggtgacggggagtgtgggaagtttggaggatggcggggtgggtggggggcagtgggggctgggtggggggacattccgaggcaggaagtggtccagcaagatttcggagagaaacgttcggggggaaggaacagcgagacacttgcagaattacagacggaaatcagagcccaaattgacgccaattgatctatctcccctctttaattactcctggggcatctttccttttttttttttatccctccttagctttttacgcgctcttttatcaatttgctccctaccccacgtaacaggggggcagtgaccaaggacgaggaacacacagagtcaccaccgtcacccgcggctccgccgccatcctgccctccgccatttatcgccctgtttttaattttttttttatctgtccctgtcgcttgggttgagtggagagtggagcctctgtgggggtgtcggccactgtgccccccagagaagtcaggggaatggagacggccgctgcctggcctggcccccgggggacagtggctggtccccggggggtcctgaggggcggaggagggggcggggaggcgtctctccaggtgtcaagaaggtgctcggtggccaccaaggtggggcccctggccggcctggccggccaggggggaaggggctacagatgtgtgagcgaagggctccaacgggcgggagcaggtggccgccttctgcgcacttggggaggctccccccgtacccctcgggaacagcttgcccgcctcctcagcgccctgtcttgcccccaggaagctcagagctccgcagggcttcctggcgtccaggcggggtgaggtcgggtggtaggagaggccgggagaaccgggccctgcagagcagaagagctgcgtagttttctgaccgctgggtcgcttgggccccgaggccctcgggtctggtgacgcgacgcagccaccactctcggcacctcgggctgtggcagggacccgggcggctgggggcttacctcacccccgcctctgcccccagcgcagtccccctgcacggcagcccgactagtgagctagaggcccgacacctcgggtccccggtgacggggctgacacgacccagggggcggttgtaccagtccaccccagacacagcgggcagctctccgtgggtcagctccatccgacccggggccccttccgacctccgcgcccagctcgtggccctccatcttgtctcttccccgagtccccccagctgtcttcagcgggaccccctctcgggtctctgagtcatcacgcgaggacccccacgcgcagaacaccaagtcatcgtgtccgcgccccgtccaccgcgcggctgtgttctcctcctcttgcccgtaacgtgctgtttctggcagaatccacagcctgggttttgtctttaacctttatcgctcgcaatcccaataaagcatttaaaattagtt

SEQ ID NO: 78 - DNA, 2679 bp, Sus scrofa
  source 1..2679: mol_type = other DNA
ggcaagcaccagcgctccatccgcggcaagcaccagtgctccggccgccgcagcaagcaccactcctgcctctgcagcaaatgcttcttctgctatttcgagtacacctattgcctccacacccacctcaggttccaggaacccttgccatcaagattcctgtaagggtggttcttcatgtgttagtctgaataatatgtttttttgcctgtgtttagaagggtattactataactcttccacctgcaataaaggaaagattttccctggtaaaattacggtcaaaatatcagaaacaactgatctagaacatgaaaattctgtggcctatcaaaatttataccttcaagttacaaatttttttaatgacacatttagcaattctgagtacggacagaccgtaattcttaaagtaagcacaactccttcagcaagatatgaaatgcgtgccagcaaccagcttgttgaggtagaagtagtaaacatttttagagaaactacaacagaaaatgagagttccatatttaatgccattgaccaagcaattaaaagcaacccaagtgactttacagaatatatccagcaagatcggtgtgattattatggatgtaagaggcaaaatgagcaagatgagtgccccagtggtttactgtgtgagtgcaaagaggggctggagagaccaaacccacagatccctctgtgtgttgctttgggtccagcatgctctgatacctgcaatgcagagaacaataggcaatgcctcgtgaggagcgctgggaacgccgaatgcttgcgcctgcctggctacaaggaggataaaaacaagatctgccaagcgtgtccatttggctacagtggagttggctgtgaaaacccatatcagctgatcctcaccatcgtgggcaccatcgctggcatcctcattctcagcctggtgattgcattcgtcctctcattaagatcaaacaacaaaaacaagaatgtggaagaacagaacttgattgagaacgactttcaaaacctgaaactgcaggagactggctttagcaacctaggagcaggaaacctctttcctaagatcaaggtggacatttccagagatgatcagccacagaatccctatataacccagggtagtataccccgccctgactattagaatggtaaaaatttggaaccatccatggccgtcaacccagcacatgagatttttgtgtgagggtttgatagactgatggagaaaagagatctggcctttagtatttttcctccatccaacatctggtctctctgaatggaagctgtaactgtttgcattgaattcagcgtgtttgaatctaagtacctatgaagttaaatgcacacataaatgctattagcacttgtaagatagttgatttgctccagtacagtggttaggtgtttgtttgttttttcctggtgggacaacaataaccatttccaaactagagggaagctccccaggattgttcattcctggaagagtctaagtgttgctttgagttgatagcatgcagataagtgacctaattctcggaaattcttacacatcagctgtggaattccagtagacgggaggagacaccatccttaaaggtactcagtggaccctgaagattcattctctagcaagttaaaataaggatgctacttctggtcctcaaatgtgggaatttcattacccttaaactgcttgcgacctggcttcacaccaagaggacagaaaacaagacatgtctcccagctttcaggaaggagttcctgggaatccaggacctttgagatgttaattaaaagatgtggacttgggagttcccgtcatggcgcagtggttaacgaatccgactaggaaccatgaggttgcgggttcggtccctgcccttgctcagtgggttaatgatccggcgttgccgtgagctgtggtgtaggttgcagacgcggctcggatcctgcgttgctgtggacctggcgtaggccggaggctacagctccgattcgacccctagcatgggaacctccatatgccgcgggagtggcccaagaaatagcaaaaaaaaaaaaaaaaaaaaaaaaaaaaaagatgtggacttgagatgtcttccatgaggatacattctagagtctttgaaatgctggtgggtgagaccaagagtaggagcaacagaggcagaaggaagctgagctgggaggaagaaggtacccaagacccaccttcatcctcagattcccctccccaataaacaggtgggcactttctctaatgcatgccctacctatgactcaagacctatgaaaggatgtgaagataaaaaccctggagcaagagtttggagtcactttgaaattgtagacattctgtctggctgatggtggagaagtgatgcagatgccagaggtacttcaccaaagtctcagccttccctggggacagaggcttagcagtggccaccaacacacacacgcacatacacagccaagaatggggcagccacctgccgcacaagctttctttaatgttaatagtatacttgtttatgtctgatgtctggtctccttgcaagttatttgtatttgttattattatttgtttttgactgataactgtaaaaggtaataaattatctttagtaaaaaaaaaaaaaaaaaaaaaaaaaaaaa

SEQ ID NO: 79 - DNA, 3494 bp, Sus scrofa
  source 1..3494: mol_type = other DNA
taactccagataaatccaggaacacggtgtttacgaggtgcccttaaaggcgggcagagaacaggagagggtgacaggagagggtgacatcagggccctgagcagaggcactagcacgggggtgggcggtggggcaggtcccaggccttggtggccgcaaacctggacggcaggcgtcccagcctgcatcccgcgctccccccacagctcggctgcataaattccaagggacgccgcagggccctcggtaaatatttgtccaaggcgcacaggagtaaaggcaggagcgggcagggtgtgaccgtccatccgagctcttcccggcagccgcacctcgggggccggtcccgctcggataaaactctccgccgccagcgagggtcgccgcgtcgcagccatgcccaggtacacggtgcacgtgcgcggggagtggctggccgtgccctgcggggacgcgcagctcacggtgggctggctgggccgcgaggccgtgcggcgctacatcaggaacaagcccgacaacggtggcttcgcctcggtggacgatgtgcgcttcctcgtgcgccgctgcaagggcctgggcctgctggaccacgaggacctgctggaggtggccctggaggacaatgagttcgtggaagtggttatagaaggggatgccatgtctccagacttcattccatcgcaaccagaaggcgtttacctttacagcaaataccgggaacccgaaaagtacatcgccttagatggggacagcctgaccacagaggacctggtcagcctgggaaagggacactacaaaatcaagctcacccccactgctgaaaagaaggtgcaaaaatccagggaagtcatagacagcatcgtgaaagaaaaaaccgttgtttatggcattactacaggttttggaaaatttgccagaaccgtaattcctgtcagtaaactagaggagctccaggtcaacctggtgcgctcacattcttcaggcgttgggaaaccgctaatccctgagagatgccggatgctcttggctttaaggatcaatgtcttagccaaaggatacagtggcatttccctggagactctcaaacaagttatcgaagtatttaatgcctcctgcctgccctatgttccagagaaaggaactgttggtgccagcggagaccttgccccgctctctcacctggcactcggtctcatcggggaagggaagatgtggtctccgaagagtggctgggccgacgcgaaatatgtcctggcagctcatggattgaaaccaattgtcttgaaaccaaaagagggcctggcgctcatcaacgggacccagatgatcacctctctgggctgcgaagctgtggaaagagccagtgctattgctcggcaggctgacatcgtggcggccttgaccctggaggtgttgaagggcaccaccaaagccttcgataccgacatccacgcggtccgccctcaccgtgggcaagtcgaagtcgcctttcgctttcgctccctcttggactcggatcaccacccgtctgagatagcagaaagtcacaggttctgcgatcgggttcaggacgcctacaccctgcgctgctgtcctcaggtccacggtgtggcgaatgacacgatcgcatttgtaaagcacatcattaccacagaaattaacagtgccacggataaccctatggtctttgccagcaggggagagactatttctggaggaaacttccatggtgaatatccagccaaagccctggactatctggccattggcatccacgagcttgctgccatcagtgaacgacgaattgaaaggctctgtaacccctccctcagcgagctgcctgccttcctggtggctgaagggggtctcaactccggattcatgatcgcccactgcacagccgcagcccttgtttctgagaacaaggctctgtgccacccctcgtccgtggactctctctccaccagcgctgctacggaggatcacgtctccatgggaggatgggcagccaggaaggcgctcagggtcgtcgagcacgtggagcaagtgctggccatcgagcttcttgccgcctgccagggcatagagtttctgcgccccttgaaaaccaccactcccctggagaaggtctatgacctggtgcgctctgtcgtcaggccctggattaaagaccgcttcatggccccagacatcgaggcggcacataggctgctcctagagcagaaggtttgggaagtggctgcaccatatattgagaaatacagaatggagcatattccagaatcaagacctgtctctccaacagcattttcactggaatttctgcacaagaagtcccccaaaatcccagagtccgaggatctttaatgcgttccgccgtggattagcagatgtgatggtacttagtttcacagcaagcaagcagtatcttgctgaagaagagacctgaggactttcttgtggatcaatctattctatagtccaggtctctaatacctgctctagttaggctggtgacagtattacatttactaaatctagtccgcttcttgttgagctggttagagacacccctggtgttttcagatgataggcgttttcttgtttcttcaccatatcaaaaggccactcactcttaagagtgagaagagtaaccaccactgcatggttctttaattctttttttttttttctgtctttttgcctttttgccttttgagggccgctcccgcagcacatggaggttcccaggccaggggtccaatcggagctatagctactggcctacgcctgagccacagcaatgccagatccttaacccactgagtaaggccagggatcgaaccgcaacctcatggttcctagtcagattcgtcagccaccgagccacgacaggaactccaggttctttaattcttaacatcagatgaactcaaatctagacggcattattttccaaaaacattgtactttcgtcgatctttttggggtttttttttgtttgttttttttgcttgacaagctctccactagctaactttgactgactgacccatgccaagatgtgacaaatgttccttactgtgtcttcataaaatatccttttcaggtcataatttggaagcctttctttctttattaattgaaccctagtgaagaataatcaagggagttccggttgtggctcagcaggaacaagcccgactagtatccatgaggatgtgggtttgatccctggccttgcttagtgggttaaggatcctgtgttgccatgaacggtggtgtaggtggcagacgtggctgtgttgctgtggctgtggtgtaggccagtagttgcagctgatttgactcctagcctgggaatttccatatgctgtgggttcagccctaaaatgagaaaaaaaaaaaaaaattggtcaattggtcaataggagatgtttgcataaaggagtgaaggaatgaatataaagattgctattggagtggctct

SEQ ID NO: 80 - DNA, 2625 bp, Sus scrofa
  source 1..2625: mol_type = other DNA
ggggctcgcgcggcagaaaagcggaggagcagccagaccccggcgctggtcaccgcaccggggacggtaagtgtgggagaacagccctgcatttctgctgacgggtcaacgtcacagcgtcaaagaaaaggaaggtacatttcagctgaactgatcaaggaagaagatggtttattccaactgtgaaagtaaagaacctgattcagtttctgcatccaatcacctgttactaaatgggaatgatgaattggtggagaaaagtcacaaaacagggcctgagaacaacctgtacagccagtacgaggagaaagtgcggccctgcatcgacctcatcgactcactgcgggccctgggcgtggagcaggacctggccctgcccgccatcgccgtcatcggggaccagagttcgggcaagagctccgtgctggaggccctgtcaggggtcgctctccccagaggcagcggaattgtgacaagatgccctcttgtgctgaaattgaaaaaactcgtgaacgaagaagacgaatggaagggcaaagtcagttaccgggacagcgagattgagctttcagatgcttcgcaggtggaaaaggaagtcagcgcagcccagattgccatcgctggggaaggcgtgggaatcagtcatgagctaatcagtctggaggtcagctcccctcacgtcccagatctgaccctcatagaccttcctggcatcaccagggtagctgtaggcaatcagccatacgacatcgaataccagatcaagtctctgatcaagaagtacatctgtaagcaggagaccatcaacttggtggtggtcccctgcaacgtggacattgccaccacggaggcgctgcgcatggcccaggaggtggaccccgaaggagacaggaccatcgggatcttgacgaagccggatctggtggacaaaggcactgaggacaagatagtggacgtggcgagaaacctggtcttccacctgaagaagggctacatgattgtcaagtgcaggggccagcaggacatccaggaccagctgagcctggccaaggccctgcagaaggagcaggccttctttgaaaaccacgaacatttcagggatcttctggaggaagggcgggccacgatcccctgcctggcagaaagactgacctctgaactcatcatgcacatctgtaaaactctgcccctgttagaaaaccaaataaaagagagtcaccagaaaataacagaggagttacagaagtatggctccgatattccagaggatgaaagcgggaagatgttttttctgatagataaaatcgatgcatttaatagtgatatcactgctttgatacaaggagaggaactggtggtggagtacgagtgtcggctgtttaccaagatgcgaaatgagttctgcaaatggagtgctgtggttgaaaagaatttcaaaaatggttatgacgccatatgtaaacaaatccagctcttcgaaaatcagtaccgggggagagagttgccagggtttgtgaattataagacatttgaaaccatcattaagaagcaggtcagtgtcctggaagagccagccgtggacatgctgcacacagtgactgatttagtccggctcgccttcacagatgtttcagaaacaaattttaatgaatttttcaacctccacagaactgccaagtccaaaattgaagacattaaattagaacaagaaaaagaagctgagacgtcgatccggctccacttccaaatggagcagatcgtgtactgccaggaccaggtctatcggggcgcgctgcagaaggtcagagagaaggaggcggaagaagaaaagaacagaaaatcaaaccagtactttctgtcgtcgccggccccctcctcagacccctccatagccgagatctttcagcacctgattgcctaccatcaggaggtcggcaagcgcatctccagccacatccctctgatcatccagttcttcatcctccggacctttgggcagcagctgcagaagagcatgctgcagctgctgcagaacaaggaccaatacgactggctcctgagggagcgcagtgacaccagcgacaagaggaagttcctgaaggagcggctgatgcggctgacccaggctcggcgccggctcgccaagttcccaggctgaaccgggctctccaggcggcccggggtctccagggcacgtctccaggcaacgaggaccaacctccttccctaacagactagcatcatgagctcctgtttcgcacatcctcctgtggttagtagactctaaagccaccgtccctgctgttagtggctgaggacttagcaagaagctgtgataagcacgctggctgcaagcatcaggccatttacttgaatgagccccgccaacgcttccgcctcccgcgcctctctccatccctctctccatccttctctccatccctgtataggatactggtccccgcatagcatcatagaagggtcattctggtttctgtacaagcctttcacgcccaatgtcttaggggcattacagccacctgtgtggatggatgcacatagaagcctatttcttttatttgtaataaacttggttctaccagccgttaaaaaaaaaaaaaaaaa

SEQ ID NO: 81 - DNA, 6609 bp, Sus scrofa
  source 1..6609: mol_type = other DNA
gtgcctgcaggacccaacgtactcaatgagctttccagcgcaatgtccgatcgctcggggccgactgccaaggggaaggatggaaagaagtattcctcgctcaacctgtttgatacgtataagggcaagtccttagagatccagaaacccgctgttgcccctcgccatggcctgcagagtctcgggaaagttgctattgctcggcgtatgccacccccagccaaccttccaagcctgaaagccgagaacaaaggcaatgaccccaacgtctcactagtgccgaaagacggaacaggatgggcaagcaaacaggagcagtccgaccccaagagttccgatgcctcaaccgctcagccgccggaatcgcagccactgccggcttcacagacgcctgcctccaaccaaccgaaacgacccccagcagccccagagaacactccttcggttccaagcggggtaaagtcctgggcacaagccagcgtcacccatggagcacatggagatggtggaagggcatcaagcctactgtcacgattctctcgagaggaatttccgaccctgcaggcggctggcgaccaggacaaggctgccaaggaaagggagtctgccgaacagtcgtctgggcccggaccaagcctccgcccccaaaattccacaacttggagggacggaggtgggcgtggccctgatgagctggagggcccggactccaaacttcatcacggccatgatccccggggtgggctgcagccttcaggcccaccccagttccctccctaccgcggaatgatgccgccctttatgtatcccccatatctcccattccctccgccctatggaccccaggggccttaccgataccccgctcctgatgggcctagccgtttcccccgtgtggcaggcccccgaggctcagggccgccaatgcgcctcgtagagcctgtgggtcggccttccattcttaaagaagataatctcaaagagtttgaccagttggaccaggagaatgatgatggttgggcaggggcccatgaggaggtcgactacactgagaagctcaagttcagcgatgaggaagacgggcgagactctgatgaggagggagctgagggccacaaggagtcccaggcagctgccggtgaggaacggccccctgaagcagatggcaaaaagggcaactcccccagcagcgaaccgccccctcccaagacggcctgggcagagacctctcggcctccagagacagagccggggcctcctgccccaaagcctcccccacccccacctcaccggggccccgccgggaactggggcccccctggggactacccagatcgagggggccctccctgcaagcccccagcacctgaagacgaggatgaggcatggcgacagcggcggaagcagtcttcatctgagatctccctggctgtggagcgggcccggcggcggcgagaggaggaggagcgacgcatgcaagaggagcgcagggctgcctgtgccgagaagctcaagcgacttgatgagaagtttggggcacccgacaagcggctcaaggcagagcctgcggcaccacccgctgtccctcctgcccccgctccaccacctgcagtccccaaagaactccccacacctccagcaccttcaccggcaccagtcccaacacctgaaaaagaaccagaagagccggcacaggcccctcctgcccagtccacccccactccaggtgtggctccagctcccaccctggtgagtggtggtggcagtaccagtagcaccagcagtggcagcttcgaagccagcccagtggaaccccagctgccctcaaaagagggtcctgaatcaccagaagaggttcctgcacctgccacaccgccagcaccaaaggtggaacccaagggtgatgggattggtcctacccggcagccccccagccaaggcttaggctaccctaaatatcagaagtcattgccgcctcgtttccagcggcaacagcaggagcagctcttaaagcagcaacaacagcagcagtggcagcagcatcaacagggctctgccccacctgccccagtacccccgtcaccaccacagcctgtgactatgggggctgtgccggctccacaggctgctccaccacccccaaaggccctgtaccctggtgctctgggccggcccccgcccatgcccccaatgaactttgatccccgatggatgatgattcctccttatgtggacccccggctcctccagggcaggccccctctggacttctacccccccggtgtgcatccctctggcctagttccccgggagcgctcagacagtggaggctcaagttcagagccatttgagcgccatgcagcccccctcttacgagatcggggcaccccaccagtggatccaaagttggcctgggtaggagatgtcttcaccaccacagccactgactcccgcccccttacctcaccactgcgccaggctaccgacgaggaggacaaggggatgaggagcgagacccctccagtacccccaccgccctatctggccagttatccaggctttcccgaaaatggagcccccggacccccgattcctcgcttccctctggaggagccagctcccccagggccccgtcccctcccctggcccacaggcactgatgaggcagccaagatacaagctccaccacccaagaaggaaactcccgaggaggagacaccacagctgacaggcccagaagctggtcgaaagcctacccgtggagtcggaggccaaggccccccaccgcctcgaagagagagccgcactgagacgcgctggggtcctcgtccgggcagcagtcgtcggggaatccctccagaggagccaggggcccctccccgccgggctgggcctatcaagaagcccccactagctacaaaagttgaagagctgccccccaagcccctggaacagggggatgaaacccccaaggttccaaagccagacacactgaagacggccaaggggaagatagggggccccaaggagaccccacccagtgggaatctttcccctgccccgaggcttcggagggactattcctacgaaagagtgggtcctacctcttgccggggtcgtggccgaggggagtactttgccagagggaggggttttcggggaacctatgggggacgagggcggggagcccgaagccgagagttccgtagttaccgagagttccgaggagatgatggtcgtggaggagggacagggggaccaaaccatccgcctgctccccgaggccgcactgccagtgagacacgaagtgagggctcagagtatgaagagatccccaagcggcgtcggcagcggggctcagaaacaggcagtgagacccatgagagtgatctggccccctcagacaaagaggcccccacacccaaggaaggggtactcacccaggtccctcttgctcccccaccatcaggagcgcccccttcgccagccccagcccgtttttccactgcccgaggtgggcgagtcttcactcccagaggggtgccgtcacgccggggccggggcggcgggcggcctcctccccccatctgcccaggctggagccctccaaccaagtccctggctccgaagaagcctccaacgggtcctttgccaccaagtaaggagcctttgaaagagaagctgctcacagggcctctgtcccctgtggcccgcggaggcagcagtggaggcagtaacatgggcctgggtgtggaagatggggagcggcccagaaggaggcgacacgggagggctcagcagcaggacaaaccacctcgcttccggaggctgaagcaggaacgggagaatgctgcgaggggggccgatggcaagccctcttccctatccctgtcaacttcggctccgggaccggaggaggccctccccacggtagcagtgcccccaccgcctcgccgggcagctgccaagtctcccgacctgtcaaaccagaaatcagaccaggccaatgaggaatgggagactgcgtcagagagcagcgactttgccagtgaacgccgaggggacaaagaggctccaccgccagcactactaacttccaaagccgtggggactcccgggggcggtggaggtggaggcggaccaggcctttcaaacatgtcccgtggagatttgagccagagagccaaggatctgagcaagcggagcttctcgagtcagcggcctggcatggaaaggcagaaccggcgcccgggtccaagcagcaaggccagcagtggtggcggcaacagtggaggaggaggtggggggcctggaggaaggacaggcccagggcgaggggacaagagaagctggccatctcccaagaaccgaagccgtcctccggaagagcgggccccagggctgccgctgcctcccccgccccccagcagttctgctgtcttccgcctggaccaagttatccacagcaaccctgctggcatccaacaggctctggcccagctcagcagccgccaaggaaatggggctgcacctggggggcacccaaggcccaaaactgggccttcccaaacccctcaaggaccgccgccgcctcggcccccagcccggtatgaccctcagagggccaacagcaacagtgtcacttccgacccccactttgaggagccggggccgatggtgaggggggtgggcggaacaccccaggactctgccggggttaatcccttcccccccaaacgtcgagaacggcctcccagaaaaccggagctgctgcaggaggaatctttgccatcttctcacacctctggattcttgggccctaaacccgagggcccgggccctcagggagagtccagagatgcaggaacagaggccctaagccctcacatctggaatcgtttacatgccgccgctggccgaaagcgttaccagcctggctccatggagccgtggatggaacccctgagtccgtttgaagatgtggctggcacagagatgagtcagtctgacagtggggtggacctgagtggggattctcaggtgtcatcaggtccctgcagccagcgaagttcccctgatggaggactcaagggggcagcagacgggctccccaagcggcctggaggcccctcacccctgaatgctgttcctggtgagggtccacctggctctgaaccctctgaacctcctaggagacggccacctgccccccatgatggggacagaaaggagctgccacgggagcagcctctgccccctgggcccattggcacagagcgattgcagcgtacagaccgaggcccagagcctagctccctccggtcggcccatcgacctggtcccccagtccaatttggcaccagtgacaaggactcggatttgtgcctagtggttggagacagtttaaaagcagagaaggagctaacggcatcagtcgcggaggccattcccatcgctcgagactgggagctgctccccagtgcttctgcttcagctgagcctcagtccaagagcctgggttctgcacactgtggcccagagcccagctcctcaggccaacgcttgtaccctgaggtcttctatggcagccctgggcctcccagttctcaggtctcggggggagccatagactctcagttgcatcccaacaatggaggcttccgccctgggacaccctcactgcacccttacagatcacagcccctatatctgcccccgggcccagccccaccctcagcactgctctctggggtagccgtcaagggccagtttctggatttctcagcactgcaagcagcagaactggggaagttgccagccggaggagttctcttccctccaccttccttcctctactctccagctttctgccctagccctttgccagatccacccttgctgcaggtgcgccaggatctgccatccccctcagatttttattctactcctctgcaacctggtggccagagtggtttcctcccctcgggggctcctgcccagcagatgcttctacccatggtggactcacagctgcctgtggtaaactttggctccctgccaccagcaccacctcctgctccaccgcccctttctctgttacctgtgggtcctgctctgcagccccccagcctggctgtgcggcctccgcctgctcctgctgctcgggtgctgccttcaccggccaggccctttggccctagcttggggcgagcagagctgcaccctgtagaactaaagccgttccaggattaccgaaaactgaacagcaaccttgggggacctgggtcgtcacgtactcccccagctggaaggtctttctctggcctcaattcccgtctcaaggccccaccttccacctacagtggagttttccgcacccagcgcatcgacctctaccagcaggcctccccaccagatgccctgcgctggatgccgaagccttgggagcggacagggccaccatctcgagaagggccatcccgaagggcagaggagcccgggtcccgaggggacaaggagcctgggttgcccccaccccgctgagggagttccctttgccccccaccccccggggcttgttatatagattataaatatataagggggaaa

SEQ ID NO: 82 - DNA, 3869 bp, Sus scrofa
  source 1..3869: mol_type = other DNA
agcggcgatggcggcggcggcgggagctgcagcggcggcggccgccgagggggaggcccccgctgagatgggggcgctggtgctggagaaggagcccagaggagctaccgagagagttcatggctctttgggggacacccctcgtagtgaggaggccctccccaaggccaaccctgactccctggaacctgctggcccctcatccccagcctctgtcacagtcaccgtcggcgatgagggagctgacactcctataggggccacacctctcattggggatgaacccgagaatctcgagggagatggggacctccatgggggacgcatcctgctgggccatgccacaaagtcattcccgtcttcccctagcaaggggggtgcctgtcccagccgagccaaaatgtcaatgactggggctggaaaatcacccccatcagtccagagtttggctatgaggctgctgagtatgccgggggcccagggggcagccacagcagcaatacctgaaccccctccagccacagccagcccagaggggcagcccaaggtccatcgagccaggaagaccatgtctaaacccggaaacggacagcccccagtccccgagaagcggccccctgaagtgcagcatttccgcatgagcgatgacgtgcactcgctggggaaggtgacctcagacgtggccaaaaggaggaagctgaactcaggaggtggcctgtcggaggagttgggttctgcacggggttcaggagatgtgtccctggagaagggcgaccccgggtccctggaggagtgggagacggtggtgggggatgacttcagcctctactacgattcctactctgtggatgagcgagtggactcggacagcaagtctgaggttgaagctctggctgaacagctgagtgaggaggaggaagaagaggaggaggaagaggaggaagaggaggaggaggaggaagaagaggaggaggaggaggaagatgaagagtcagggaatcagtcggacaggagcggttccagcggccggcgcaaagccaagaagaagtggcggaaggacagtccgtgggtgaagccgtcacggaaacggcggaagcgggagcctccgagggccaaagagccacgaggcgtgtccaacgacacatcctccctggagacagagcgagggtttgaggagttgcccctctgcagctgccgcatggaggcacccaagatagaccgcatcagtgagagagcggggcacaagtgcatggccacggagagtgtggatggagagctatcgggctgcaacgccgcgatcctcaagcgggagaccatgagaccgtcgagccgcgtggccctgatggtgctctgtgagacccaccgcgcccgcatggtcaaacaccactgctgcccaggctgtggctacttctgtacggcgggcaccttcctggagtgccaccccgacttccgagtggcccaccgcttccacaaggcctgcgtgtcccagctcaacgggatggtcttctgtccccactgtggggaggacgcatctgaggcccaagaggtgaccatcccccggggggatggggtgaccccaccagcgggcactgcagcccccgcccccccacccctggcccaggatgcccccgggagagcggacacttcccagcccagcgcccggatgcgaggtcacggggagccccggcggcccccctgcgaccccctggctgacaccatcgacagctcggggccctccctcaccctgcccagtggaggctgcctctcagctgtggggctgccacctgggccaggccgggaggccctggagaaggcactggtcatccaggagtcggagaggcggaagaaactccgtttccacccccgacagctgtacctgtcagtgaagcagggcgagctgcagaaggtgatcctgatgctgttggacaacctggaccccaacttccagagcgaccagcagagcaagcgcacgcccctgcacgcggccgcccagaagggctcagtggagatctgccacgtgctgctgcaggctggagccaacatcaacgcagtggacaagcagcagcggacgccgctgatggaggctgtggtgaacaaccacctggaggtggctcgttacatggtgcagcgcggcggctgcgtctacagcaaggaggaagacggctccacctgcctccaccacgcagccaaaattgggaatctggagatggtgagcctgctgctcagcacgggtcaggtggacgtcaacgcccaggacagcgggggctggacgcccatcatctgggctgcggagcacaagcacatcgaggtgatccgcatgctgctgacacggggcgccgacgtcaccctcacagacaacgaggagaacatctgcctgcactgggcctccttcaccggcagcgccgccatcgccgaggtcctcctgaacgcccgctgtgacctccatgctgtcaactaccacggggacacgccgctgcacatcgcagcccgggagagctaccacgactgcgtgctgttgttcctgtctcgaggggccaaccctgagctgcggaacaaggagggggacacggcgtgggacctgacccctgagcgctccgacgtgtggtttgcactccagctcaaccgcaagcttcggctcggggtgggaaaccgggccattcgcactgagaagatcatctgccgggacgtggcccggggctatgagaacgtgcccattccctgtgtcaacggggtggacagcgagccgtgccccgaggattacaagtacatctcggagaactgcgagacgtccaccatgaacatcgaccgcaacatcacgcacctgcagcactgcacgtgtgtggacgactgctccagctccaactgcctgtgcggccagctcagcatccgctgctggtatgacaaggatgggcggctgctgcaggaatttaacaagatcgagcccccgctgatttttgagtgtaaccaggcgtgctcctgctggagaaactgcaagaatcgcgtagtgcagagcggcatcaaggtgcgactgcagctctaccgaacagccaagatgggctggggggtccgcgccctgcagaccattccccaggggacttttatatgcgagtatgtgggagagctgatctctgatgccgaggctgacgtgagagaggatgattcttatctcttcgacttggacaacaaggatggtgaagtctactgcatcgatgcccgttactacggcaacatcagccgcttcatcaaccacttgtgtgaccccaacatcatccccgtccgggtcttcatgctacaccaagacctgcgatttccgcgcattgccttcttcagttcacgagacatccggaccggggaggagctggggtttgactatggtgaccgcttctgggacatcaaaagcaaatatttcacctgccagtgtggctctgagaagtgcaagcactcagcggaggccatcgccctggagcagagccgcttggcccgcctggacccccaccccgagctgctgcccgagctcagctccttgccccccgtcaacccctgagagtggaccacacacacccctccctaccccaggacggccaccaactcagcagccacctctgccgcttgctgcccacagcccacacctgggggtgccacagtctcctgtccctgcacccctccacacattcctcagccagaaatccccgccaggccctggaggtctgacagcccctctctcccagagctggttcctccctgggagagtgacttcagggctggccacccccctgttccccatccttagctgaagtttgatgaattgaaattggggccctatgctaactggtttcctttgttctcaataaatgttgggtttgtaataa

SEQ ID NO: 83 - DNA, 3980 bp, Bos taurus
  source 1..3980: mol_type = other DNA
atggcggcggcggcgggagctgcagcggcggcggccgccgagggggaggcccccgctgagatgggggcgctggtgctggagaaggagcccagaggagccaccgagagagttcatggctctttgggggacacctctcgtagtgaggagaccctgcccaaggccaaccccgattccctggagactgctggcccctcatccccagcctctgtcacagtcactgtcggcgatgagggggctgacacccctgtaggggccacaccactcattggggatgaacccgagaatctcgagggagatggggacctccatgggggtcgcatcctgctgggccatgccacaaagtcgttcccgtcttcccccagcaaggggggtgcctgtcccagccgagccaagatgtcaatgacaggggccggaaaatcacccccatcagttcagagtttggctatgaggctgctaagtatgccgggcgcccaaggggcgccagcagcagggcctgaaccccctccggccacagccagcccagaggggcagcccaaggttcatcgagccaggaaaaccatgtccaaaccaggaaatggacagcccccagtccctgagaagcggccccctgaagtgcagcatttccgcatgagtgatgacgtgcactcgctggggaaggtgacctcagatgtggccaaaaggaggaagctgaactcaggaggtggcttgtcagaagagttggcttctacgcggagttcgggagaagtgaccctggagaagggggaccccgggtccctggaggagtgggaaacggtggtaggggatgacttcagcctctactatgattcctactctgtggatgagcgggtggactctgacagcaagtctgaggttgaagctctggctgaacaactgagtgaggaagaggaagaggaggaggaggaagaggaggaagaagaagaggaggaggaggaggaggaagaagaagaggaagatgaagagtctggcaatcagtctgacaggagtggttccagcggccgccgcaaagccaagaagaaatggcggaaggacagcccatgggtgaagccatcacggaaacggcggaaacgagagcctccgagggccaaggagccacgaggagtgaatggtgtgggctcctcaggccccagtgagtacatggaggtccctctggggtctctggagctgcccagcgaggggaccctctctcccaaccacgctggggtgtccaatgacacatcttcgctggagacagagcgcgggtttgaggagctgcccctctgcagctgccgcatggaggcccccaagatagaccgcatcagcgagagagcagggcacaagtgcatggccacagagagcgtggacggagagctgtccggctgcaatgctgccatcctcaagcgggagaccatgagaccgtccagccgtgtggccctgatggtgctctgtgagacccaccgtgcgcgcatggtcaaacatcactgctgccccggctgtggctacttctgtacggcgggcaccttcctggagtgtcaccccgacttccgtgtggcccaccgcttccacaaggcctgtgtgtcccagctgaacgggatggtcttctgtccccactgtggggaggacgcatctgaggcccaggaggtgaccatcccccggggggatggggtgaccccaccggctggcactgcagcacccgctcccccacccctggcccaggatgcccccgggagagcggacacttcccagcccagtgcccggatgcgggggcagggggagcctcggcgtccaccctgcgaccctctggccgacaccatcgacagctcagggccctccctaaccctgcccaatgggggctgcctctcagctgtagggctgccaccagggccaggccgggaggccctggagaaggccctggtcatccaggagtcagagaggcggaaaaagctccggttccaccctcggcagttgtacctgtccgtaaagcaaggggagctgcagaaggtgatcctgatgctgttggacaacctggaccccaacttccagagtgaccagcagagcaagcgcacacccctgcacgcggctgcccagaagggctctgtggagatctgccatgtgctgctgcaggctggagccaacatcaatgccgtggacaagcagcagcggacgccgctgatggaggccgtggtgaacaaccatctggaggtggctcgctacatggtgcagcgtgggggctgtgtctacagcaaggaggaagacggctccacctgcctccaccacgccgccaaaattgggaacctggagatggtcagcctgctgctgagcacgggacaggtggacgtcaatgcccaggacagcggcgggtggacccccatcatctgggctgcagagcacaagcacatagaagtgatccgcatgcttctgacacggggcgcggacgtcactctcaccgacaacgaggagaacatctgcctgcactgggcctccttcaccggcagcgccgccatcgccgaggtcctcctgaacgcccgctgcgacctccacgctgtcaattaccacggggacacacccctgcacatcgcggcccgggagagctaccacgactgcgtgctgttgttcctgtcacgcggggcgaaccctgagctgcggaacaaggagggggacacggcgtgggacctgacccccgagcggtccgacgtgtggtttgccctccagctcaaccgcaagctccggctcggggtggggaatcgggccatccgcacggagaagatcatctgccgggacgtggctcggggctatgagaacgtccccatcccctgtgtcaatggtgtggacggggagccctgccccgaggattacaagtacatctcagagaactgcgagacgtccaccatgaacattgaccgcaacattacccacctgcagcactgcacgtgtgtggatgactgctccagctccaactgcctgtgtggccagctcagcatccgctgttggtatgacaaggatgggcggctgctccaggaatttaacaagatcgagcccccgctgatttttgagtgtaaccaggcatgttcctgctggagaaactgcaagaacagagtagtgcagagcggcatcaaggtgcgactgcagctctaccgaacagccaagatgggctggggggtccgtgccctgcagaccattccccagggtaccttcatttgcgagtatgtcggagaactgatctctgatgccgaggctgatgtgagagaggacgattcttatctcttcgacttggacaacaaggatggagaggtgtactgcatcgatgcccgttactacggcaacatcagccgcttcatcaaccacttgtgtgaccccaacatcatccccgtccgagtcttcatgctgcaccaagacctgagatttccacgcatcgccttcttcagttcccgagacatccgggccggggaggagctggggtttgactatggtgaccgcttctgggacatcaaaagcaagtacttcacctgccagtgtggctctgagaagtgcaagcactcggctgaggccattgccctggagcagagccgcctggcccgcctggacccacaccctgagctgctgcctgagctcagctccctgccccccgtcaacccctgagatcggaccacagccactgttccacccctggacagccaccagctcagtagccacttctgccgcttgcccacagcctacgcctgggggcttttaccgtttcctctctcccttacttaccccttcacacattcctcagccagctcccagccaggccctggaggtctgacagcccctgtctcccagagctggttctttcctgggagggtgacttcagggctggccacccctccccccaagcgcctcctcccacccctgttccccaccctcaactgaagtttgatgaaattgagcctctgtgctaattggtttcctttgttctcaataaatgttgggtttataataaa

SEQ ID NO: 84 - DNA, 4924 bp, Bos taurus
  source 1..4924: mol_type = other DNA
atgttggatatttgcttggaaaaacgtgtgggtacgaccttggctgcccccaagtgtagctccagtgctgtgaaggttccagggttggcagaggggactgaagggaccatgaaaatggacatggaggacgcggatatgactctgtggacagaggctgagtttgaggagaagtgtacatacattgtgaatgaccacccctgggattctggtgtggaaggaggcacttcggttcaggcggaggcttccttaccgaggaatctgcttttcaaatatgccacaaacagcaaagagattactggagtggtgagtaaagaatacataccaaagggaacacgttttggacctctaataggtgaaatctacaccagtgatgcagttcccaagaacgccaacaggaaatatttttggcggatctattccagaggggagcttcaccacttcattgatggctttaatgaggagaagagcaactggctacgctacgtgaacccagcgcacactgcccgggagcagaacctggccgcctgtcagaacggcatgaacatctacttctacaccattaagcccatccctgccaaccaggagcttcttgtgtggtactgccgggactttgcagaaaggcttcactacccttattccggagagctgacaatgatgaatctcacacaaacccagagccgtccaaagcagcagagcactgagaaacatgaactgtgcccaaagagtgtcccgaagagagagtatagcgtcaaagagatcctaaaattggactcccacccttccaaagggaaggacttgtaccgctccaacatttcacccctcacttcagagaaggacgcggaagacttcaggaaaaacgggagcccggagatgcccttctacccgcgcgtggtgtacccgattcgcgccccgctcccggaagactttctgaaagcacccctgggatgctacgggatggagcggccgccctacatcactcactcccccatatcgtcctccacgacgcccagcccctcggcgcgcagcagcccggaccggagcctgcagagctgcagcccgcacagcagccccgggaacacggtgtcgccgcgggcccccggcccccaggaggcccgcgactcctacgcctacttgaacgccccctacggggccgccgccgaggggctgggctccctagccggctacgggccgcccacccacctgccgcccgccttcatcccctcctacaacgcgcactacccgaagttcctgctgcccccttacggcatgaactgcaacggcctgggcgccgccgtgagcaacatcaacggccttaacagcttcggcctcttcccgaggctgtacccggtgtacagcagcctcttgggcgggggcggcctgcctcccccggtgctggcgtccgcctcgctccccagctcgctgccctcggagggggcgcgccgcctgctgcagcccgagcacccccgggaggtgctggtgcccgcgccgcacagtgccttctccctccccggggcggccgccagcctgaaggacaaggcctgcacccccaccagcggctccccgacagccgggaccgccgccacggccgagcacgtggtgcagcccaaagctacctcagcggccgctgcggcccccggaggcgaggaggcggtgaatctcatcaagaacaaaaggaacatgactggctacaagacactcccctacccgctgaagaagcagaacggcaagatcaagtatgagtgcaacgtctgcgccaagactttcgggcagctctcaaacctgaaggtccacctcagagtgcacagtggagaacgacctttcaaatgccagacgtgcaacaagggcttcactcagctcgcccatctgcagaaacactacctggtacacacgggagagaagccacatgaatgccaggtttgccacaagcgatttagcagcaccagcaatctcaagacccacctgcgactccactccggagagaaaccttaccagtgcaaggtgtgccccgccaagttcacgcagtttgtgcacctgaaactccacaagcgcctacacacgcgggagcggccccacaagtgcgcccactgccacaagagctacatccatttctgcagcctcaaggtccacctgaaggggcactgtcctgtggccccggccactgggctgcctctggaggacctggccagaatcaacgaagagattgagaagtttgacatcagcgacaatgccgaccggctggaggacatggaggacaacatcgacatgacctctgtggtggaaaacgagattctggccgtggtcagaaaagagaaggaagaaactggcctgaaagcgtctttgcaaagaaacctgggaaatggactgctctcagggtataacctctatgagtcaacagagccgtccctcctgaagttgcctcacagcaacccactacctctgggacctgtaaaggtcaaagaagaaacagtcgaacctatggatccttaagattttcagaaaacaaaaggtgttttgtcttgtttcttaacttacgacttggcgagtcagggtgcctgtcgcaagcgcttgtacataattccaattctgcaaagctctccctgacggcagatggtttccctcacctctctggaattaaagaaggaaccccaaagttactgaaatctcagggcataaatgaggcaaagacaatatatatacctattatatatacttatttacaccccatctatatatttgaacctgttgtattttgaatatttgtgtgaatatgtttgcatagcctccccattgctaagactattgcctagccataattattttttcaatgataatccttcataatttattatacagtttatcattcaaaaagcaataattaaaaaaagtttacaatgactggaagaattccttgtaatttgagttaagtgttatatttttgtctggtggccattctttgtagataatttctgcacatctgttttaagtatcttaagatttaataaacagatacattacttcaagtcaacctccctctataataatggtttgaaagtgaggtttgagtattgccaaagttaggcagttgatgtgttcattcataggattatattatttgaacagccttgtatcatttgaacataatttgggggtatgtttgcagaattaaccagtaataggtggaagaaataaaaaccttgtatgtttctgtagagagttcagcattcctgcccccaatcacagatttaccagaaaggtgataaggcgttgccagtctgtctccctcttcaaaaaaacagggttgtcccatccccaaagtcatgtgaccatttgcagtggccatgagactttagcatcccaatgtattatctgaaaatgtgaagaagacaaatgccatgtttaaaaccactgcaaaatgtttccaaagcacaggtggttttgtatgggtgaggtctgggggcatgagtccaggtgtttttgttgttggtcttttgttattacgtcagaattgcacaagcatggtgctccaccaggaaggatttgagttagataggctcaggccacactttaaaaaccaaaagccagaaaacgggtattctcgttgtcttagggtcaaagcgggtaatgaacattcctgtccccaacacatcagttgtatttgttttttctgtgcaactcaagacttttcctcagtatttgtgtttttacattttatggttaatttaattgaagaagaaagggcattgcaaagttgttcaacaacaattacctcattgagtgtgtccagtagtgcaggaagtgatggcatctttaatgatttgctactcaagaggagagagcacgtgtgcaccagagagatggaatgcatcccattctgcctcgtcctctgctaagcccaaagataacacgttggcattcaaggtgtagcggacaatgatgtatatttataaatctatttataccactataccatgtatatagaatatatttataaccacttaaaattgtgagccaaaacatgtaaaagatccttcttttcctaatggcaaaaaaaaaaaaaaaaaaatcaaaccctctctgactctttgcttaattagcactcgatgacctcacaattacattggtgagcttgggcacccccttggtattataagagatcctaaaattttggtgcaaaggtggggaccgcataacaaccagggagaaagagacataccttttttaactattaaggacaatctaagatggcttttttttttttttttttgtcacattctgatgtggtcacacccaagtcccagaaatagaaatcccatcaaccactgtaacttttctctcttcctctccttccttcttaaataactgatggatcactaatgaatctggtttatatattcagcattttggtctgtggcaactagtatcagcctacagaagagaacaacaaatgtaggataatccagggaaacctaagagccttactgatctgttcctttaaataatgaaactgacagttttttttaatattttttttaacagtgtttgacaaacagtccttgattataagtaaaccaaagcatcatactgacactgacatcaaatattttctattccccatcactcatttgttctcttttttccctctcatgctttctcattagtggaaatttttactgagacctgcacagtggcttgagaccccgtaattctctaccatctgactttccctctgaggcctcttacatgtgaatattgagcccacaatcaagagtggttttgtttttttctttcttctactcaaaactgaccaaagttactggctttttactttgctagaacaaactatcttatgtttacgtactggtttacattgttatttatgtgcaaattgtcaaaatgtaaattaaatataaatgttgatgcttta

SEQ ID NO: 85 - DNA, 4944 bp, Sus scrofa
  source 1..4944: mol_type = other DNA
gggtttctgagaggcgagctgtttggatcattcatgatttgctgccattgtgaacactcttaaggagagaactctgcctgagaaatgctgattaataaaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 86 - DNA, 1716 bp, Bos taurus
  source 1..1716: mol_type = other DNA
atggccctgcggccgccgggggaggccggcccccaggacaagggatgctaccctcccgactggctccagggcagcccccaacacctggccgcctactacacgcctttcccgccctacggccactacgggaacaccctgaccgcggcagaggagggcttccagcctttccggcccctggaagccctggcagcgtccccggctctgccccccttcggcttccggaccgcgcctcccttgctaagccagggcctggccctcccgagggagccgctatgcgacctgccctggtacagcaagctgccgacctggtaccctgttccccacctccccagggaggtgccgcacttccagaacagcagtcgtgagtttaccggcgccgccagcgaggacttggcacacaccgccggccggagcgacggtggccagtgttggggacctgagattttcgttccgccgccccccgtggatgcttcgttgttacctgagaggccgaagccctcgcagctgttatcggggtcccccagcgagccctctgaggatggccccaaactcgtgaaccgaaaagggaaggcttctcgccgctaccacttcacccaagaagacctgcacttggttctgtacggggtcatccccagcccgcagcactcagcccgcctgcagcacgcgatctcgggcctcctgatccccaccgatagcgccggggctgattctcttcctcagactctggataacgactcccttcagctaccagaaggtctctgcctccagcagacgaagtttggtgaagtcgcacattttggggtgttctgcagtagtcttgtggccaaaggagtcaggtttgggccctttcagggtaaagtggtcaacaccagcgaagtcaagacacacggagacaattccctgatgtgggagatctttgaagatggtcagttgagccactttattgatggaaaaggaggtgcagggaactggatgtcctatgtcaactgtgcccgtttcccgtcggagcagaacctggtggcagtacaatgtcaagggcagatcttctacgagagctgcaaggagatctaccagaaccaggagctcctcgtgtggtacggagattgctatgagaagtttctggacattcccgtgagcctgcaggttctcaagcaggggaagcaggcatctgggtcctctgaagagtctgcggaagggtacaggtgtgaaagatgtggcaaagtatttacctacaaatattacagagacaagcatctaaagtacacgccttgtgtggacaagggtgacaggaaatttccctgttctttatgcaaacgatcctttgagaagagagaccggctccggatccacatccttcacgttcatgagaagcatcggcctcacaagtgttctacatgcgggaaatgtttctctcagtcttccagtctaaacaaacacatgagagtccactctggagacagaccctaccactgtgtgtattgtactaagaaattcactgcctccagtatactccgcacgcatatcaggcagcactcgggggagaagcccttcaaatgcaagtactgtggtaaatcttttgcatcgcacgctgctcacgacagccatgtccggcgctcgcacagggaggatgacggctctccctgtaacacctgtgggaagaccttctcagatcaagaagccttccactcccacatgaagtctcatgaagactgctag

SEQ ID NO: 87 - DNA, 1915 bp, Sus scrofa
  source 1..1915: mol_type = other DNA
tcgcggtggcccaggatggctctgcggctgccaggtgagaccggtccccaggacaaggagtgctactcgcccgacaggcttcagagcagcccccaacacctggccgcctactacacccctttcccgcactatggccattacgggaacaccctcaccacggcggaggagggtttccaacctttccggcagctggaggccctgtcggcgtccccggctctgccccccttcgccttccggacagcgcctcccttgctgagccccgggctggcgctgccgagggagccgctctatgatgtgccctggtacagcaagctgccgacgtggtacccgatcccccacctgcccagggaggtgccacacttcctgaacagcccgcgcgagttcacgggcgcccccgccgaaaacttgggccaggtcggcggccggagcgatggtggccagtgttggggacctgaagttttccttccgccgcccccagtggatgcttctctgctacccgagaggccgaagccctcccagttgttacctggctcccccggcgagcgctctgaggatggccccaaactcgtgaaccaagagagaaaggcatcgccccgttaccacttcacccaggaagacctgcacttagttctgtacggggtcattcccagccctcagcacccagcccgcttgcaccacgcgatgtcgggcctcctggtccccaccaacagctctgggtctgattctcttcctcagactctggataacagctcccttcagctcccggaaggtctctgcctgcggcagacgatgtttggtgaggtcgcacattttggtgtgttctgcaatagcctcgtggccaaaggagtcaggtttggaccctttcagggtaaagtggtcaatgccagtgaagtcaagacccacggagacaattctctgatgtgggagatttttgaagatggtcatttgagccactttattgatggaaaaggaggtgctgggaactggatgtcctatgtcaactgtgcccgtttccccacggagcagaacctagttgcggtgcaatgtcaagggcagatattctatgaaagctgcaaggagatccaccagaaccaagagctcctggtgtggtatggagattgctacgaaaagtttttggacattcctgtgagcctgcaggtcataaagcaggggaagcaggcatctgggccctcagaagagtctgcagaagggtacagatgtgaaagatgtggaaaagtatttacctacaaatattacagagacaagcatctcaagtacaccccctgtgtggacaagggtgacaggaaatttccctgtaatttgtgcagacgatcctttgagaagagagaccggctccggatccatatccttcacgtccatgagaagcaccgaccccacaagtgttctacgtgtgggaaatgtttctctcaatcttccagcctaaacaagcacatgagagtccactctggagacagaccctaccagtgtgtgtattgtacaaagaagttcactgcctctagtatactccgcacccacatcaggcagcactccggggagaagcccttcaaatgcaagtactgtggtaaatcttttgcgtcccacgccgcgcacgacagccatgtccggcgttcacacaaggaggatgacagctgttcctgtagcatctgtggcaagaccctccccaatcaagacgctttctatgcccacatgaagtttcatgaagactactaactagcctttagagattttgaggatatttcttgtttatgggtgtgtcttgtggggaaaagtgatgcttatattgagatgagacacagagtagtggaactgaccaattagcagaatttgccagagttcttggttgtggtattaacatcctttgcagaaatgttttttgttttttgtttttgtcttt

SEQ ID NO: 88 - DNA, 1788 bp, Sus scrofa
  source 1..1788: mol_type = other DNA
atgaccatgaccctacacaccaaagcgtccggcatggccctgctgcaccagatccaagccaacgagctggagcccctgaaccgcccgcagctcaagatccccctggagcggcccctgggcgaggtgtacgtggacagcagcaagcccgccgtgtataactaccccgagggcgccgcgtacgacttcaacgccgcggccgccgcctccgcgcccgtctacggccagtcgggcctcgcctacggcccggggtcggaggcggcggcgttcggcgccaacggcttggggggcttccagccgctcaacagcgtgtctccgagcccgctggtgctgctgcacccgccgccgcagctctcgcccttcctgcacccccacggccaacaggtgccctattacctggagaatgagccgagcggctatgcggtgcgcgaggccggccctcccgccttctacaggccaaattcagataatcggcgccagggtggcagagagagattggccagcaccagtgacaagggaagcatggccatggaatctgccaaggagactcgctactgtgcagtgtgcaatgactatgcctcaggctaccattatggagtttggtcttgcgagggctgtaaggccttcttcaagagaagtattcaaggacataatgactacatgtgtccagctaccaaccagtgcacaattgataagaacaggaggaagagctgtcaggcctgccggctacgcaagtgctacgaagtgggcatgatgaaaggggggatacggaaagaccggagaggagggagaatgttgaagcacaagcgccagagagatgatggagagggcaggaatgaagcggtgccccctggagacatgagatctgccaacctttggccaagccctctcttgattaaacacactaagaagaacagcccggtcttgtccctgacagccgaccagatgatcagtgccttgttggaggctgagccccccataatctattccgagtatgatcctaccagacccctcagtgaggcttcaatgatgggcttgctgaccaacctcgcagacagggagctggtacacatgatcaactgggcaaagagggtgccaggatttttggatttaagcctccatgatcaagtgcatcttctggaatgtgcctggctagagatcctcatgattggtcttgtctggcgctccatggagcacccagggaagctcctgtttgctcctaacttgctcctggacaggaaccagggcaagtgtgtcgagggaatggtggagatctttgacatgttgctggctacatcatctcgcttccgtatgatgaatctccagggagaggagtttgtgtgcctcaaatccatcattttgcttaattctggagtgtacacgtttctgtccagcaccctgaagtctctggaagagaaggaccatatccaccgtgtcctggacaagatcacagacaccttgatccacctgatggccaaagcgggcctgactctgcagcagcagcaccggcgtctcgcgcagctcctcctcatcctgtctcacttcaggcacatgagtaacaaaggcatggagcatctgtacaacatgaagtgcaagaacgtggtgcccctctatgacctgctgctggagatgctggacgcccaccgcctgcacgccccaaccaaccttgggggcccacccccggaggacatgagccagagccagctggccacctcgggctcaactccatcgcattccttgcaaatgtattacatcacaggggaggcggagaacttccccaccacaatctga

SEQ ID NO: 89 - DNA, 4092 bp, Bos taurus
  source 1..4092: mol_type = other DNA
atggatgcaaaagctcgaaattgtttgcttcaacatagagaagccttggaaagggacatcaaaacatcctacattatggatcacatgattagtaatggagttttaacagtgtcagaggaggaaaaagtgaaaaatgagcccacccaacggcaacgagcagctatgctaattaaaatgatacttaaaaaagataatcattcctatatatcgttctacaatgctctacttcatgaaggttacaaagatcttgctgcccttctgcatggtggcattcctgttatctcttcttccgatgtaaggacagtcctgtgtgaaggtggagtaccacagaggcccgttgtttttgttactaggaagaagctagtgaatgcaattcagcagaagctctccaaactgaatggtgaaccaggatgggtcaccatatatgggatggcaggttgtgggaagtctgtattagctgcagaagctgttcgagatcattctttcctagaagattgtttcccaggaggcgtgcattgggtgtcagttgggaaacaggacaaatccgggcttctgatgaaactacagaacctttgtgcacgattggatcaggatgagagcttctcccagaggcttccacttaatattgaagaggctaaagaccgtctccgcattctgatgctgcgcaaacatccaaggtctttattgatcttggatgatatttgggatccttgggtgttaaaagcttttgacaatcagtgtcaaattcttctcacaactagagataagagtgttacagattcagtgatgggtcctaaatatgtagtgcctgtggagagtggtttaggaaaagaaaaaggacttgaaattttatccctttttgttaatatgaagaaagcagatttgccagaacaagctcacagtattatacaagaatgtaaaggttcccctcttgtagtatctttaattggtgcacttttacgagattttcccaaccgctgggagtactacctcaggcaacttcagaataagcaatttaagaggataagaaaatcttcatcttatgactatgaggctctagatgaagccatgtctataagtgttgaaatgctcagagaagacatcaaagattattacacagatctttccatcttacagaaagatgttaaggtgcctacaaaggtgttatgtatcctctgggacttggaaacggaagaagttgaagacatattacaggaatttgttaataagtctctcttgttctgtgaccggaatggaaaatcatttctttattatttacatgatctgcaagtagatttccttgctgaaaagaatcgcagccagcttcaggatctccacaagaagatggtcactcagttccagagacatcaccagctatattctctttcaccagatcaggaggattgcatgtattggtacagttttctggcctatcacatggccagtgctaatatgcacaaagaactgtgtgctttaatgttttccctggattggattaaagcaaaaacggaacttgtaggccctgctcatctgattcatgaatttgtggaatacaggcatatattggatgaaaaggatggtatagactgtgagaatttccaggagtttttatctttaaatggacatcttcttggacgacagccatttcctaatattgtgcaactgggcctctgtgaactggaaacttcagaggtttatcggcaagctaagctgcaggccaagcaggaggtcgataacggaatgctttacctggagtgggtaaacaaaaaaaacatgaagaacctctcccgcttggttgtccgcccccatacagatgctgtttaccatgcttgcttttctgaagatggtcaaagaatagcgtcttgtggagctgataaaactttacaggtattcaaagctgaaacaggagagaaacttctagaaatcaaggctcatgaggatgaagtgctttgctgtgcattctctgcagatgacaaatttatagcaacctgctcagtggataaaaaagtgaagatttggaattcaatgactggggaactagtgcgtatctatgatgagcactcagagcaagtcaattgctgccacttcaccaacaatagtaatcaccttctcttagccactggttcaaatgactacttccttaaactctgggatctgaatcaagaagaatgtcgaaataccatgtttggccatacaaattcggtcagtcactgcagattttcaccagatgatcaggttttggccagttgttcagctgatggaacattaaagctttgggatgtgaaatctgcaaatgagaggaaaagcattaatgtgaagcaattcttcctgaattcagaggagcctcaggaggatctggaagtgatagtgaaatgctgttcgtggtctgctgatggtgctaagataatggtggctgcgaaaaataaagtctttctttttgacattcactccagtggcctgttggcagaaatgcacatgggccatcatagcgctgtccagtactgtgacttctccccccacaaccatttggcagtggtggctttgtcccagtactgtgtggagttgtggagtatggactcaggtttaaaagtagctgattgcagaggacatttaagttgggtccattgtgtgatgttttctcctgatggatcatcgtttttgacatcttctgatgaccagacaatcaggctctgggagacaaagaaagtatgtaagaactctgctacagtgttaaagcaagaaatagatgttgtgtttcaagaaaatgaagtgacggttcttgcagttgacaatataagacgtctgcaactcattaatggaaacacaggtcagattgattacctaactgaagctcaagtgagttgctgttgtttaagtccacatcttcagtaccttgcattcggaggtgaagatggagccattcagattttagaactcctgaacaacagaatcttccaatccagaaatgggcacaagaccactgttcggcacatccagttcacagctgatggaaagactcttatctcaagctctgatgattctgcagttcagatatggaactggcaatcagaggaccctttctttctccaagcccaccaggaaacagtgaaagacttcagactcttgaaaaattcaagacttctttcctggtcatttgatggaacagtaaaggtatggaatattattaccagaagaatagaaaaagatttcgtgtgtcaccaggatacagttctttcttgtgatatttcctctgatgctactaagttttcttctacctctgctgacaagactgcaaagatttggagttttgaactcctttctcctcttcatgaattgagaggccacaaaggctgtgtgcgctgctcttccttctctgtgaacaataccttgttggcgactggagacgacaatggagaaatcaggatatggaatgtctcaaacggtgagcttcttcatttgtgtgctccgatttcagtagaagaaggagctgctactcatggaggctgggtgaccgacctttgcttttctccagacaccaaaatgcttgtttctgctggaggatatctcaagtggtggaatgttgtcactggggaatcctcacagaccttctacacaaacggaaccaatcttaagaaaatacacgtgtcccctgacttcaaaacatatgtgactgttgataatcttggtattttatatattctaaagattttagagtaaaatagttaagtggtgatgtaagttgacctcttttaattttgaattggaaaaaaatcctaatgaaactctgtactttaacttttataaagctgtgaattgttttgtagtattgcattcattacaaaagtgtttgtggttggatgaataatattaatgtaatttttcccaaatggacatgcctttaatcttgtttttcataatcaccatcattttttaacagttcgtccttaagatgcaaatgaaaatgtaaatatgtacctagttataccattggtaaaattctcccttgctacattcaaattggttgacataataatgaaaataattggaaggaaatatatatattttaatactatcattggtgcaggccatgcc

SEQ ID NO: 90 - DNA, 5998 bp, Sus scrofa
  source 1..5998: mol_type = other DNA
ctttcagacttggcaccgcccagaagccagccccctccctcgcccggtcatccccgcggttccactgcgactcctatccacctgcctttctagttcctccagctcatggttgacagctcagagaaagaaaaatttgaggaaggatggatgcaaaagctcgaaattgtttgcttcaacatagagaagccctggaaagggacatcaaaacatcctacatcatggatcacatgattagtaatggcgttttaacactttcagaggaggaaaaagtgaaaaatgagcctactcaatgccaacgagcagctctgctaattaaaatgatacttaaaaaagataattatgcctacatatcattctacaatgctctgctacatgaagggtataaagatcttgctgcccttctccatggtggccttcctgtcgtctcttcttccaacagcaaagattcagttgctggaataacttcctatgtaaggacagtcctgtgtgaagggggagtgccacagaggcccgtcgtctttgttatgaggaagaagctcgtgaatgcagttcagcagaagctcttcaaactgaatggtgaaccgggatgggtcaccatatatggaatggcgggttgtgggaagtctgtgttagctgcagaggctgttcgagatcattctctcttagaagtttgtttcccagggggtgtgcattgggtttcaatagggaaacaagacaaatctgggcttctgatgaaactgcagaatctttgtgcacggttggatcaggatgagagtttctcccagaggcttccacttaatattgaagaggctaaagaccgtctccgaattctgatgctgcgcaaacacccaaggtctctgttgatcttggatgatatttgggatccttgggtattaaaagcttttgacaatcagtgtcagattcttcttacaactagagacaagagtgttacagattcagtaatgggtcctaaatatgtaatacctgtggagagtggcttaggaaaagaaaaaggacttgaaattttatccctttttgttaatatgaagaaagcagatttgccagaacaagctcacagtattataaaagaatgtaaaggttctcctcttgtagtgtctttaattggtgcacttttacgtgattttcccaaccgctgggattactacctcaggcaacttcagaataagcaatttaagaggataaagaaatcttcatcttatgattatgaagctctggatgaagccatgtcgataagtgttgaaatgctccgagaagacatcaaagattattacacagatctttccatccttcagaaagatgttaaggtgcctacaaaggtgttatgtattctctgggacatggaaaccgaggaagtcgaagacatactgcaggagtttgttaataagtctctcttattctgtgatcggaatggaaagtcatttctttattatttacatgaccttcaagtagatttccttacagagaagaatcgcagccagcttcaggatctgcacaagaagatgatcactcagttccagagacatcaccggctacacactctttcaccagatcaggaggactgcatgtattggtacaattttctggcttatcacatggccagtgctaatatgcacaaagaactgtgtgctttaatgttctccctggattggattaaagcaaaaacggaacttgtaggccctgctcatctgattcatgagtttgtggaatataggcatattttggatgaaaaggatggcacagtctgtgagaatttccaggagtttttatctttaaatggacatcttcttggacgacagccgtttcctaatattgtgcaactgggtctctgtgaactggaaacttcagaagtgtatcggcaagctaagctgcaggccaagcaggaggtcgataacggaatgctttacctggagtggataaacaaaaaaaacatcaagaacctctcccgcttagttgtccgcccccatacagatgctgtttaccatgcttgcttttctgaggatggtcaaagaatagcttcttgtggagctgataaaaccttacaggtgttcaaagctgaaacaggagaaaaacttctagaaatcaaggctcacgaggatgaagtgctttgctgtgcattctctgcagatgacagatttatagcaacttgctcagtggataaaaaagtgaagatttggaattctgtgaccggggaactggtacaaagctatgatgagcactcagagcaagtcaactgctgccacttcaccaacaacagtaatcaccttctcttagccactggctcaagtgactacttcctcaaactctgggatctgaatcaaaaagaatgtcgaaataccatgtttggtcatacaaattcagtcagtcactgccgattttcaccagatgataagcttttggctagttgttcagctgatggaacattaaagctttgggatgtgaagtcagcaaatgagaggaaaagcattaatgtgaagcagttcttcctaaattcagaggagtctcaagaggatatggaagtgatggtgaaatgctgttcgtggtcagctgacggtgccagcttagtggtggcagcaaaaaataaagtcctgctttttaacattcatacaggtggcctgttggcagaaatccacacaggccatcacagcaccatccagtactgtgacttctcccccagtaaccatttggcagtagtggctttgtcccagtactgtgtggagttgtggaatatggattcatgtttaaaagtagctgactgcagaggacatttaagttgggttcattgtgcaatgttttctcctgatggatcatcgtttttgacatcttctgatgacgagacaatcaggctctgggagacaaagaaagtgtgtaagaactctgcgatagtgttaaaacaagaaatagatgttgtgtttcaagaaaatgaagtgatggttcttgcagttgacaacataagacgtctgcaagtcattaatggaaaaacaggtcagattgattatctgacggaagcccaagtgagttgctgctgcttaagtccacatcttcagtaccttgcgtttggaagcgatgatggagccattcaggttttagaactcctaaacaacagactcttccagtccaggactaggcacaagaacactgtacggcatatccagttcactactgatggaaagaatgttatctcaagctctgatgattctgcaattcaggtgtggaattggcagtcagaggagactgtctctctgcaagcccatcaggaaacagtgaaagactttagactcttgaaaaattcgagactgctttcctggtcatttgatggaacagtaaaggtatggaatattataactggaagaatagaaaaggactttgtctgtcaccaggatacagtactttcttgtgatatttctcctgatgctaccaaattttcttctacctctgctgacaaaactgcaaagatttggagttttgacctcctttcccctcttcatgaattgagaggccacaaaggctgcgtgcgctgctctgccttctccgtggacagtaccctgttggcgactggagatgacaacggagaaatcaggatatggaatgtctcaaacggtgagcttcttcattcgtgtgctccagtcttagtagaaggaggagctgctacccacggaggctgggtgaccgaccttcgcttttctccagatggcaaaatgcttgtttctgctggagggtatcttaagtggtggaatgttgactctgggaaatccatacagacgttctacacaaatggaaccaatcttaagaaaatacatgtgtcccctgacttcaaaacatatgtgactgtggataatcttggtattctgtatgttttaaagattttagagtaaaatagttgagcagtgatgcaagttgaactcttaattttgaattggaaaaaatcgtaatgaaactctgtatttcaactttttttataaagctgtgaattgttttgcagtattgcattcattacaaaagtatttgtggttggatgaataatattaatgtagcttttcccaaatgaacacacttttaatcttgtttttcataatcatcatcattatttttaacagtttgtcctgaagatgcaaatggaaatgtaaatatgtacctcgttatactgttggtaaaattctcccttgatgcactcaaattggttgacataattaataagaataatttgaaggaaatgtgtattttaatattatcattatgcaggctgtgctacagggtagtagtggcttggctttatgaaccacacttatcccaagagggtatagttcccccaaatatcatctcagaccttatttgtacccctgtaatttgcttgaaaaatattgtgtctgtgtacacaatgtaaaaaatgtcctttaatgtacttgataagtgagtcttagattccaaaagatctttctgtgagtcaaaatttatgcacgttggagagagtagattttgttggtctaccctttctcatccactgatggcagcccgggctggacctggactcaagctgtcagtctttaagttggtccgtgtcctgctctctgcttttttatataccagacttggtatacttgactgaccttgacctttcttaatcctcacttcagtacttgctccacagctaactgttagtaaatcagagtaatcagggaagagccaattttaatcataagttgaaacaacagaatttttctaagatagggaaaatagtaaaaaataatttttgtctaattccatttacttgtagatgaatagcattgctaatgctgttagtgaatttcaagagaattctatttggttttctgagcaatgctggaagagtcatgtgactctggaagatgtcacctttaaccttccagcctgtcgctttctatccccttctctccctccgcatttccttcctttccctccctccttcattctaagcttcatcaggaaaaataatttttgataaggaagaggcgtatcaatatcagtaactagctttttttacctcctggtcaggtacttacattgcagcttaactctaaacatttcccctgaagtatactgttaagggaaatgtgataaatataaaattaataaaattatttatgtaaaagacaaccaaaattcagaagtttttgctgctgcaagagcgaagtcagattagtgttactagtaggtaagcattgagctagtttctgttgttgatacatgtgaggtgggtacggggaatagaccactccagatgtgctttcagatgttcctctttagtatttgagggaaccttaagagtgaataaagtctttcacattgttaaggaggcttgttttttgttttgtcgcatagtgggttgaggagctcatttataggttcactgtgacttaagcaaatgtgatgtaaaaaatcttacttttataaaagaatttctataaatagtatgttctacttagagtactcaaatcataatttgattggtttacaatgtttaggaactcatagctcagagtataaagtattaacttttagaattaaatgtcacttgactgtaaaagtatgacaacaaacatcagcttttaaaaagccttgattggcccttttatcttaccaataaattaaaaagaaaaaatgagaagagagactgggtaacatgaggaaagaagagaagcctttcatggcagtgtaagtggccagtcaagggggacggttttgttttgttctttggggtgagtgtttttcaaggctttccccctgctttattttttaattttttttttgtattgaacatacactgcattttgtgtgtataaaggtttttttgttttttgtattggatatgtgttatgatgttttggaaacagttttgtatttatgatacagcttacatttttttttttttgccttggtatacacttaaatacatgaattaaaaaattttataaaaataaaattcctaaaatatttttgaaaa

SEQ ID NO: 91 - DNA, 5454 bp, Sus scrofa
  source 1..5454: mol_type = other DNA
gccagtgtccactgccagcgaggggcggagccagaacgcaacccacagtcctcagttcgttcagcacctccccagccccagcaaggagcagcctccatttttgacgaatctgagccaccccagggtgatgcttctaaagcagaaagaggacaccgaactgcattgtggcgtgtttggtttctagcagcgatttacctttccaagtcagtagtgaaaatgcacattaagtcaatcattcttgagggattcaagtcctatgcacagaggactgaagtcaatggttttgaccccctcttcaatgctatcactggtttaaacggtagtgggaaatccaacattttggactccatctgctttttgctgggcatctccaatttgtctcaggttcgggcttctaatttacaagatttagtttacaaaaatggacaggctggtattaccaaagcctcagtgtcaatcacctttgataattctgacaaaaagcaaagtcctttaggatttgaggttcatgatgaaattacagtaacaaggcaggtggttattggtggcagaaataaatatttaatcaatggagtaaatgcaaataataccagagtacaggatctcttctgttctgttggccttaacgttaacaaccctcactttctcatcatgcagggccgaattacaaaggtgttgaatatgaaacctccagagattttgtccatgatagaagaagcagctggaactaggatgtatgaatacaaaaaaatagctgcccagaagacaatagagaaaaaggaggctaagctaaaagaaattaagacgattcttgaagaagaaattactccaaccattcaaaaattaaaagaggaaagatcttcctatttggagtaccaaaaagtaatgagagaaatagaacatttgagtcatttatacattgcttatcagtttttgctggctgaagataccaaagaacgctcagctgaggagttaaaagaaatgcaagataaaattctaaagcttcaagaagaactgtctgagaatgataaaaaaataaaagcacttagtcatgaaatagaagagttggaaaaaagaaaagataaggaaactggaggtatacttcgatctttagaagacgctcttgcagaagctcagcgaattaatactaaatctcaaagtgcctttgatctcaagaagaagaatctggcaagtgaagagaacaaacgcaaagagctggaaaaaaatatggttgaagattctaaaacgttagcagccaaggaaaaagaggttaaaaagataacagatggactgaatgctcttcaagaagcaagcaataaagatgctgaagctttggctgccgcacaacagcacttcaatgctgtttctgctggcctgtccagtaatgaagatggagcagaagcaactcttgctggtcaaatgatggcctgtaaaaatgatataagtaaagctcagacagaagccaaacaggctcaaatgaagttgaaacatgcccagcaagaattaaagactaaacaaaccgaagttaagaagatggatagtggctacaggaaagatcaagaagcattagaagctgtgaagaaacttaaagaaaaacttgaagctgaaatgaaaaagctaaattatgaagaaaacaaagaggaaagccttctggaaaagcgcaggcagctgtctcgtgatatcagtagattgaaagaaacatatgaagctctcttggccagatttcccaatcttcgatttgcctatagggatccagagaagaactggaatagaaattgtgtgaaaggacttgtagcttcactgattagtgtgaaagatatttctgcaaccacagctttagaattggtggctggggacagactctataatgttgtagtcgacacggaggttactggaaaaaagctactagaaaagggagagttgaagcgtcgatatactataattccactcaataaaatttcagccagatgtattgctccagaaactctgaaaattgctcagactcttgttggccctaataatgttcatgtggctctttctctggttgagtataaaccagaacttcagaaagcaatggaatttgtatttggaacaacatttgtttgtgacaatatggataatgcaaaaaaaatagcttttgataaaagaataatgactagaactgtaactctgggaggtgatgtatttgatcctcatggaacattgagtggaggtgctcgatcccaggcagcttctattttaacgaagtttcaagaactcaaagatgttcaagatgagctgagaatcaaggagaatgagctacaggctctagaagaggaattggcacatcttaaaaacactgctgaaaagtatcgccaactaaaacagcagtgggagatgaaaactgaggaggcagatttattacaaaccaagcttcagcaaagctcctatcataagcaacaagaagaattagatgctcttaaaaaaatcattgaggaaagtgaggagaccttaaaaaacaccaaagaaattcaaaaaaaagcagaagaaaaatatgaagtattggaaaataaaatgaaaaacgcagaagctgaaagagagagagagctaaaggatgctcagagaaaactagatagtgccaaaactaaagcagatgcttctagcaagaaaatgaaagagaaacagcaggaagttgaagctattaccctggagctggaagagctcaagagagagcatgcatcctgtgaacaacagcttgaagctgtaaatgaagctatcaaatcctatgaaggccagattgcagtagtggcagcagaggtggctaaaaataaggagtcagtaaataaagcccaagaagaggtgaccaagcaaaaagaagtaataacagcccaagacaacatagttaaagctaaatatgcagaagtagcaaaacataaggagcaaaacaatgaatatcagcttaaaattaaggaactggatcacaacatcaacaaacataaacgggaagctgaagatgctgctgcaaaggtgtccaaaatgttgaaagattatgactggattaatacagacaaacacctctttgggcaacccaatactgcctatgatttcaaaactaccaacccaaaagaagctagtcagaggcttcagaagttggaagaaatgaaggaaaaactaggaagaaatgttaacatgagagctatgaatgtactgacagaagctgaagagcggtataatgatttgatgaagaagaagagaattgtagaaaatgacaaatctaaaatcctggcaactatagaagatcttgaccagaagaaaaaccaagccctaaatattgcttggcaaaaggtgaacaaggactttggatctattttttctactcttttgcctggtgctaatgctatgcttgcaccaccagaaggacaaactgtattggatggtctggagtttaaggttgccttaggaaatacttggaaagaaaacttgactgaacttagtggtggtcagaggtctttagtggctttgtcattaatactgtccatgcttctcttcaaacctgctccaatttatatcctggatgaggtagatgcagccttagatctttcccatacccagaatattggacagatgctacgtactcatttcacacattctcagttcattgtggtgtccctaaaggaaggtatgttcaacaatgccaacgtccttttcaaaaccaagtttgtggatggtgtttctacagtagccagatttactcagtgtcaaaatggaaaagttccaaaggaaacaaaatctaagacaaaaggacccaaatgatcacatatggaagttaaaagtacagatttattccattccttcatcttgacctgttttttaaacataaacttttttaagaatttggggttcattattttgttacttaaccacttatttaatcacttataaatgagcctaaattcctcgtctgaattagtgttaactatggtccaattactgtgatagtgattttctgtatgttatcaactgttttttcttacacaagtcttttatatatcagggagcctgagacatatagttggtggtaaattttgtatgtaaaagcaaagtataaaaagatgaaattaaattataggataaatatagctgatagtcagcttttaataattagttaattcctaaggtaatattactgtgttttctattactaataatatcattcgggtaaggaagaattataaagcaagcaagtaggtggaccaacaaagggaacacaaagaaaactattagacatgcactaagctttttgtgatttagttcttcgaggacataatgccagatccctgatttatactcacatgagaaacagatcttgaaaggtttctcttgttttaatgatgtttgaatatcatgtttctatatatttagtaattggtctgtgcaatcttaaattttttaaaaagtctatgaatatttaataaatgcttgggagaaggtatgttgcatgttataagtttatccaaagtctcctaatatttttttttaaagatttttgtcagccttttcataatccagtcaataacaatctattggtgagtgaaaatgtaggagccagcagaccaaaccaaattatctttttcctttagttctgatatatatgtcatatgtatatatatatatatatatatatagatccatttgttctatcaaaagggctcagaattcattgattattatatatgttagtgtccctgctttccattgcaaaaacatgaatttctgttaagaactcaaaagctaggcctaacaaacaccataagaatactatatttggccccttagtaccttagtatctcttagtgtgtcactagaaccatgtctccaaactctaaatcttgaaagttctgacatctaatctgaccttggatgtgttattttggctccacgttgtttcttagttcctttgtggtatccttttcttaacatatgctcatattccctggccctaggtttattttttctcatccttcatcctcatgcatatgtgaggatgtcaggtaactgatactctggggcacattgtaaaggattgaatgtggaattagttcagtctaccgtaagaggaggagacaaagctgtaaatgctgttatataggctgtccattatcacatgcaatagatgtaagtttatttcttttcaagtatattgcagattgtttgtcaaaggaattaatcagttcacactgtcaccaatatagtatgttgaccacaacctacacagcactggctgttatttgtaatgcttctgatttactagttataaaacagtatttaattgctatttttaattatcattttataagtaacttgtgggattgaatattttgccatattattaattttctttcctcagccaacaattctgtttgctttcctaatttttgttacggaaatcctcaaacattaaatttttgtgagtaa

SEQ ID NO: 92 - DNA, 4944 bp, Bos taurus
  source 1..4944: mol_type = other DNA
gaagttcgttcggcgccgccccagccgcaacaaggtgcagcttccatttagaggaccctggcccagcccagagggattctgaggcagaacgaggagaccgacacgaagccatcctgcttccggtacttggagaccacagaggcctgccccactgaatcccgccgggcctgagaggaggcggggcttcccgggcgcgatgacggccagacccggcctgcggcagggcggggccggcgagcgcgaggcccccaggctgaggtgcggggctgcccgaggtgtgccggataccgagctcaacaattgccgccagcttctttagtcctcagaactgctttgtggcttgatttctagcagacctgtccaagtcagtagtgaaaatgcacattaagtcagtcattcttgagggattcaagtcctatgctcagaggactgaagtcaatggctttgaccctctgttcaatgctatcactggtttaaatggtagtggaaaatccaacatattggactccatctgctttttgctgggcatctccaacctgtctcaggttcgggcttctaacttacaagatttagtttacaaaaacgggcaagctggtattacaaaagccttggtgtcaatcacctttgataattctgacaaaaagcagagtcctttaggatttgaggttcatgatgaaattacagtaacaaggcaggtagttattggtggcagaaataaatatttaatcaatggagtaaatgcaaacaataccagagtatacgatctcttctgttctgttggccttaatgttaacaaccctcactttctcatcatgcagggccgaattaccaaagtgatgaatatgaaacctccagagattttgtccatgatagaagaagcagctggaaccaggatgtatgaatacaaaaaacaaaatgctcagaggactatagaaaaaaaggaggctaagctgagagaaattaagacgatacttgaagaagaaattactccaaccattcaaaaattaaaagaggaaagatcttcctacttggagtaccaaaagctcttgagagaaatagaacatctgagtcgtttatatatagcttatcagtttttgctggctgaagatactaaagaacgctcagctgaggcattaaaagaaatgcaggataaaattaagaagcttcaagaagaagtgtatgagaatgataaaaaaataaaagcacttagtcatgaaatagaagagttggaaaaaagaaaagataaggaagttggaggtatacttcgatctttagaagatgctcttacagaagctcagcgagttaatactaaatctcaaagtgcctttgatttcaagaagaaaaatctggcgagtgaagagaacaaacgcaaagaactggagaaaaatatggtagaggattctaaaactttagcagcaaaggaaaaagaggttaaaaaaataacagatggactaagtgcccttcaagaagcaagtaataaagatgctgaagctttggccgttgcacagcagcactttaatgctgtttccgctggtctgtccagtaatgaagatggagcagaagcaactcttgctggtcagatgatggcctgtaaaaatgaaataagtaaaactcagacagaagccaaacaggctcagatgaagttgaaacatgcccaacaagaattaaagactaaacaaacagaaattaagaagatggatagtggctataggaaggatcaagaagcattagaagctgtgaaaaaacttaaagaaaaacttgaaactgaaatgaaaaggctaaattatgaagaaaacaaggaggaaagccttctggaaaggcgaaggcagctatctcgtgatatcagtagactgaaagaaacagaagaagctctcttggccagatttcccaatcttcgatttgcctatagggatccagagaagaactggaatagaaattgtgtgaaaggacttgtagcttcactgatcagtgtaaaagatagttctgcaaccactgctttagaattagtggccggagaccgactctacaatgttgtagtagacacagaggttactggaaaaaagctactagaaaagggggaattgaagcgtcgatacactataattccactcaataaaatttcatccagatgtattgctccagaaactctgaggattgctcagaatcttgttggccctaataatgttcatgtggctctttccctggttgaatacaaaccagaacttcagaaagcaatggaatttgtctttggaatgacatttgtttgtgacaatatggataatgcaaaaaaagtagcttttgataaaaggataatgactagaactgtaactctaggtggcgatgtgtttgatcctcatgggacgttgagtggaggtgctcgatcccaggcagcttccattttaatgaagtttcaagaactcaaagatgttcaagatgagctcagaaccaaggagaatgagctacgggctctagaagaggaattagccaatcttaaaaacactgctgaaaagtatcgccacctaaaacagcagtgggagatgaaagctgaggaggtagatttattacaaaccaagcttcagcaaagctcgtatcacaagcaacaggaagaattagatgctcttaaaaaaatcattgaggaaagtgaggagaccttaaaaaacaccaaagaaattcagaaaaaagccgaagaaaaatatgaggtgttggagaataaaatgaaaaatgctgaagctgaaagagagagagaactgaaggatgcacagaaaaaactagattgtgccaaaactaaagcagatgcttctagcaaaaaaatgaaagaaaaacaacaggaagttgaagctatcaccctggagctggaggagctcaggagagagcatgcatcctatgaacaacagcttgaagctgtaaaggaagctatcatatcctacgagggtcagattgcagtgatggcctctgaggtggctaaaaatgaggagttagtaaataaagctcaagaagaggtaagcaagcaaaaagaagtgataacagcccaagacaacataattaaagctaaatatgcagaaatagcacaacacaaggagcaaaacaatgaatctcagcttaaaattaaggaactggaccataacatcagcaaacataagcgggaagctgaagatgctgctgcaaaggtatccaaaatgttgaaagactatgactggattactgtagaaaaacacctctttgggcaacccaatagcacctatgatttcaaagctaacaatccaaaagaagctggtgagagacttcagaagttgcaagaaatgaaggagaaactaggaagaaatgtcaacatgagagccatgaatgtactgactgaagctgaagagcgttataatgacttgatgaagaagaagagaattgtggaaaacgacaaatccaaaatcctggcaactatagaagaccttgaccagaagaaaaaccaagccataaatattgcttggcaaaaggtgaacaaggactttggatctattttttctactcttttgcctggtgctaatgctatgcttgcaccaccagaagggcagactgtgttggatggtctggagtttaaggttgccttaggaaatacttggaaagaaaacctaactgaacttagtggtggtcagaggtctttagtggctctgtcattaatcctgtccatgctccttttcaaacctgccccaatctacatcctggatgaggtcgatgcagccctggatctttctcatactcagaatattggacatatgctacgtactcatttcacacattctcagttcattgtggtatccctaaaagaaggtatgttcaacaacgcaaacgtccttttcaaaaccaagtttgtggatggtgtttctacagtagccagatttactcagtgtcaaaacggaaaagttccaaaggaaaacgaatccaagtcaaaaagacccaaatgatcacacatgaagttaaaagtaaagattcactccttcaccttgacctgttttttaaatatacactttataagaacttgggattcatgttgtttgtataaaaatgtaatattgttacctaacccattttctcttccttatataatcacttataactgagcataaattcctcttttctgaatcagtgttaactatggtccaattactgtaattgtgatttcatgtatgtcaccaagtgttttttcttacacaagtcttttctatattagggagcctgagatacatagttggtggtaaattttgtatgtaagagcaaaattaccaagaagcagatgaaattaaattacagaataaatatagctgataatcagcttttaatagctaattcctaaggtaatattagtgttttctgttgctaataatgtcgttcaggtaaggaagaatcaagtagcaagtagctggaccaacagagggaacacaaagaaagctgttaggcatgccctgagctatttgtgtttagttccttgaggacagtgccaggtctctgatttagactcaaatagaaagcagatcttaagaagattcctgttattttaatgatgtttgaataccatattatgcttagtaattggtctgtgcagtcttcttttttttttttgatctttatgagtattttaataagagcttaggacaaaggtatcctctgcgtgctgctctctgctgcaggtttatctgaagtctcctaatatggtcagagattttgttagccttttcataattcaatcaataacagtctgttggtgagtgagaatataggagccagcagactaaaccaaattaatctttttcttttagtcatatacatatgtaaatagattcatctgttttaccaaaaggacttaaaatttcatgactatttcttagtgtcctgttttccaattcaaaaccatgaatttccttttaagatttcaaaagatggcgctaacaatcagca

SEQ ID NO: 93 - DNA, 3788 bp, Bos taurus
  source 1..3788: mol_type = other DNA
cgctcggcgaaggtctacgcgccatccccaaggtcccccctggatcgccggctttcatcttcctgctgaattcctgctttgcagatagcgcaatggcagcccgagtacttgtcattggcaatggaggacgagaacacacgctggcctggaaacttgcacagtctactcatgtcaaacaagtgttggttaccccaggaaatgcaggcactgcctgctctgagaagatttcaaatactgacatctcaatcagtgatcatactgcccttgcgcagttctgcaaagatgagaaaattgaattcgtagttgttggaccagaggcacctctagctgctggaattgttgggaacctgaactctgtgggagttcggtgctttggccccacagcacaagcagctcagttagagtccagcaagagatttgccaaagagtttatggaccgacatggaatctcaactgcaagatggagagctttcactaaacctaaagaagcctgtgacttcattatgagtgcagacttccctgctttggttgtgaaggccagtggtcttgcagctgggaaaggggtaattgttgccaagagcaaagaggaggcctgtgaagctgtcagagagattatgcagggtaaagctttcggagaagcaggagaaacagttgtcattgaagaacttcttgaaggagaagaggtgtcttgtctgtgcttcactgatggacggactgtggcccccatgcccccagcacaggaccataagcgattgctggagggcgatgagggccccaacacgggaggaatgggagcctattgtccagcccctcaggtttctaaggatttgttgctaaaaattaaaaataatattcttcagaggacagtggatggcatgcaggaggagggtatgccatacacaggtgtcctctatgctggtataatgctgaccaagaacggccccaaagttctggaatttaattgccgtttcggtgatccagagtgccaagtgatcctcccacttcttaaaagtgatctttatgaagtgattcagtctatcttagatggcttgctctgcacttctctgcctgtttggctggacaactgtgctgccgtaactgtggtcatggcaagtaaaggttatccaggagactacaccaagggtgtagagataacagggtttcctgaggctcaggctttaggactggaggtgttccaagcaggcactgccctaaaagatggcaaggtggtgactaacgggggtagggtccttactgtcacggccatcagggaaaatctcatctcagctcttgaagaagccaggaaaggacttgccgccataaagtttgagggagctgtttataggaaagacatcggctttcgagccatagctttcctccagcagcccaggggcctgacttacaaggaatctggggtagacattgcagctggtaatatgctggtccagaaaattaaacctttagcgaaggccacctccagaccaggctgtgatgttgatcttggaggttttgctggtctttttgatttgaaagcagctggtttcactgatcctcttctggcttgtggaacagatggtgttgggacgaaactgaagattgcccagcagtgcagtaaacatgacaccattggtcaggatttggttgcgatgtgtgtaaatgacattctggcacaaggagcagagcccctctttttcctcgattacttttcctgtggaaaacttgacctccgtactactgaagctgttatcactgggatcgccaaagcttgtaaaaaggctggatgtgctctcctcggaggtgaaacggcagagatgcctgacatgtaccctcctggagagtatgatctagctggttttgccgttggcgccatggagcgggatcagaaacttcctcaactggaaagaatcacggaaggagacgctgttattggaatcgcttcatctggtcttcacagcaacggattcagccttgtgaggaaaattgtagcaaaatcttctctcgagtactcttctccagcacctggtggctgtggtgaccagaccttaggtgacttacttctaaccccaaccaaaatctacagccgctcactgttacctgtcctgcgttcaggacgtgttaaggccgttgcacatattactggtggaggattactggaaaacatccccagagtcctccctcagaaattgggggtgaatttagatgcccagacctggagggtccccaggatcttctcatggttacagcaggaaggccacctctctgaagaggagatggccagaacatttaactgtgggattggggctgccctcgtggtatcagaggacctggtgaagcagactctgcaggatattgagcagcaccaggaagaagcctgcgtgatcggcagagtggttgcatgtcccaagggttctcctcgtgtgaaagtcgagcatctgattgaaaccatgcaaataaatggatcagtattggagaatggaaccctgagaaatcatttctctgttcaaccaaaaaaggcaagagtagctgtcttaatttctgggacaggatcaaacctccaagctctcatagacagcactagggagccaagtagcttggcacacattgtcattgtgatctccaacaaagctgcagtggctgggttagataaagcagaaaaagccgggattcccacgagagtaattaatcataaattatataaaaatcgtgcagcatttgacactgcaattgacgaagtccttgaagagttctccacagacatagtctgtcttgcaggattcatgagaattttgtctggccccttcgtcagaaaatggaatgggaaaatgctcaatatccacccatccttgctcccttcgtttaagggttcaaatgcccatgaacaagtcctggatgctggggtcacagtcaccgggtgcaccgtgcactttgtagctgaagatgtagatgccggtcaaattatcttacaggaagccgttccagtgaagaggggtgacactgttgaaactctatctgagagagtaaaattagcagaacataaaatatttccctcagccctccagctggtggccagtggagctgtacggcttggagaaaatggcagaatctgttgggtcacagaggactgaagcgtcatcccccgggaatggagacagctgagagcattgtaggtgctgcagggggacttttatcctggactccacctaaaagatgtgtgagacatctgtgtgttgcctccagtggtctttcaggttttacctgtctggggccttcttgttcataatcaaactaggccttcagaattccttttactttcagccaagtattataccaaaacttagtcttttctttttcaaagagctaacaagtctaaaaagttagtatcttgtcctcatattgtatatctgtactaatttgaattgagacttctcgatttattctgatcataagttgttatcataaaatttctgggtagttcccattgagggatagtgttctctgtgctttgaagaatcattacactttcctgtaataattgacgatttctataatacccagatttcagcttcacagtttgggaggatacaaggcaatctactcagaccagtaaagcgtttattttttttttttatgttatttaattattgcaatagtaattttctgtttttcataacatgtacagattccacagtgttatggattaaacagtcttctgtctaaaaaaaaaaaaaaaaaaaaaaaaaaaaaaaaaaaaaaaaaaaaaaaaaaaaaaaaaaaaaaaaaaaaaaaaa

SEQ ID NO: 94 - DNA, 4609 bp, Sus scrofa
  source 1..4609: mol_type = other DNA
acagacagcaatggcagcccaagtacttgtaattggcaatggaggaagggaacatacactggcctggaaacttgcacagtctaatcatgtcaaacaagtgttggttaccccgggaaatgccggcactgcctcctccgaaaagatttcaaatactgatgtctccgtcagtgatcatgctgcccttgctcagttctgcaaagaggagaaaattgagtttgtagttgttggaccagaggcacctctggctgctggaattgttgggaacctgacatctgcaggagtacggtgctttggccccacagcagaagcagctcagttagagtccagcaaaagatttgccaaagagtttatggatcgacatggaatcccaacggcacaatggagagctttcaccaaacctgaagaagcctgtagcttcattatgagtgcagatttccctgctttggttgtgaaggccagtggtcttgcagcagggaaaggggtgattattgcggagagcaaagaagaggcctgcaaagctgtacaggagatcatgcaggatagggctttcggagaagctggagaaacaattgtcattgaagaacttcttgaaggagaagaggtgtcttgcctgtgcttcactgatgggaggactgtggcccccatgcccccagcacaggaccataagcgattgctggatggagatcatggccccaacacggggggaatgggagcctattgtccagcacctcaggtttctaaggatttgttgctaaaaattaaagatacgattcttcagaagacagtggctggcatgcagcaggagggtgtcccatacacaggtattctctatgctggtataatgctgaccaagaatggtccaaaagttttggagtttaactgccgtttcggtgatccagagtgccaagtgatcctcccgcttctgaaaagtgatctttatgaagtgatccaggctaccttagatggtagactgtgcacatctctgcctgtttggcatgacaaccgcgctgccgtaactgtggtcatggcaagtaaaggttatccaggagactacaccaagggtgtagagataacagggtttcatgaggctcaagctttaggactggaggtgtttcaagcaggcactgccctaaaagatggcaaagtggtgactaatgggggtagggtccttacggtaacggccatccaggaaaatctcatctcagccctggaagaagccaagaaaggactcgctgctataaagtttgagggagccatttataggaaggacattggctgtcgtgccatagctttcctccagcagcccaggggcctgacgtacaaggaatctggggtagacattgcagctggaaatatgctggtcaaaaaaattaagccattagcaaaagccacttccagaccaggttgtgatgttgatcttggaggttttgctggtctttttgatttgaaagcagctggtttcagtgatcctcttctagcctgtgggacagacggcgttggaaccaaactgaagattgcccagcagtgccataaacatgataccattggtcaagatttggttgcaatgtgtgtaaatgatattttggcacaaggagccgagcccctctttttcctcgattacttttcctgtggaaaacttgacctcaatacgacggaagctgttgtggctggaatcgctgaagcttgtaaaaaagccggatgtgctctcctcggtatgatcatccattttaatcacacggggtgaaacgcagaaatgcccgacatgtaccctcctggagagtatgacttagccggttttgccgttggtgccatggaacgagatcagaaactccctcatctggaaagaatcacggaaggggacgctgttattggagtggcttcatctggtcttcacagcaatggatttagccttgtgaggaaaatcatagcaaaatcttccctccagtactcctctccagcacctgatggctgtggtgaccagaccttgggggacttacttctgactccgaccagaatctacagccattcgctgttacctgtactacgctcgggacatgttaaggcctttgcccatattactggtggaggattgctggaaaacatccccagagtcctccctcagaaatttggggtggatttagacgcccggacctggaggatccccaggatcttctcctggttacagcagctaggacatctctcagaagaagagatggccagaacatttaactgtgggattggggctgccctcgtggtgtcaaaggatctgacagagcagattctgcaagatattgagcggcataaggaagaagcctgggtgattggcaaggtggtagcgtgtcctgaaggttctcctcgtgtgaaagtcaagcatctgattgaaagcatgcaaataaatggatcagtgttggagaatggcaccctgaaaaatcatttctctgttcaaccgaaaaaggcaagagtagctgtcttaatatctggcacaggatcgaacctccaagccctcatcgagagcactcaggcaccaagtagctctgcacacatcattgttgtgatctccaacaaagcagcagtggccggcttagataaagcagcaagagccggcatccccaccagggtaattaatcacaaattatataaagatcgtgtagcattcgacatggcagttgaccaagtccttgaagagttctccacagacatagtctgtcttgcaggattcatgagaattttatctggcccctttgttagaaaatggaatgggaaaatgctcaacatccacccatccttgctcccttcatttaagggttcaaatgcccatgagcaagtcctggatgccggggtcacggtcactgggtgcactgtgcatttcgtagctgaggatgtcgatgctggacaaattgttttacaagaagctgttccagtgaagaggggagacaccattgaaactctctctgaaagagtaaaattagcagagcataaaatatttccctcagccctccagctggtggccagtggcactgtacagcttggagaaaatggcaagatccgttgggtcagagaggaatgaaatgtccttgtccagggatggagccagctttgaacaaattctgaccactgcagagtggctttcctcatggactcagccctaaaagatccaaagcataccggtatagtctccagtggtcttgcaggttttacctgcgtggggccttcttggttacagtcaaactggtcattcagaattccttttgctctcagccaagcaggataccaaaacttaatcttatgttctttctcatagagttagttaacaagtctaggaagttaatagctcttcccccatgttgagtatatctatgccgatttgaattgagatttcgctaattattctcatcataagttggtgtcataaaacttctggggagctcccacttctgagggtagtatgctttgaagtatcattaaactttcctctaataattgacaatttctaccatacccatattttagcttcatagtttgagaggatggaaggcaatctacccagactagtaaagagtatcatgagactgcgtgcatttcccactctagccagtatttccaatttggatattcagttcgggaaccaggaggtctgggttgacatactgggcacatgcacttgaccctacacagaggaccaaccacatttctttctttaatagctgagcgaacaggcaatggccacctggcccacagccaggcaaccagcccgcctgagactgccgagcctccttccgggtggcctaagagccctgcctgcccaacaggacgaccttcccgcaagggaacagcacagatgtacactagttacaaacagaagataatcaactcccaccttaagacaggagggagcacagagggtcccggggaagcagagtgccagaaaaaagaaacgagacctttgccaagagaccctcctcactgcctctggtccaagtaggtttccccaggcagctggcagcattaccctacctcttgttgaccgcagtcagcaactgccatcagatgggaagtccaggtacatcatttcacataggtgcctttgctgcaaaaacaactgattcactttttttctagagttcactcaacataagtaagcatctatacgccaaacactgtcctatgccatttttaggacgttagaagatacaaaggaacatgttactggttgataaaatcgcatattgtgataaaagtagaagctggtactaggatgtaaattagaaaaatcaatgtgaatttatgcttttagaaatatagttgtctagctcttaactttgacactagtctccacaaaaaggaacccaaactccacagaaggtagctaattcagtatcttgggataggaagaatcaaagtgtgtagaaactaggcgtttgcttaaagggtatcctactgtctagtttgtgataaactgaatgttatttgtacatgttgagtgtgaacagccattggcttataatactcaaagacaaaaggtcattgaagttatgaagagtcataatgcataaaagggtgaaaacaagatgttcatcgtttaataaatatagatgtttgtcttttatta

SEQ ID NO: 95 - DNA, 2317 bp, Bos taurus
  source 1..2317: mol_type = other DNA
gaggagccttggaggaacgtccggcgtgggatctgtacagccagttcagggttcatggcttcctccaggacgctcggcactttccgtctcccgccgttacccacgattcgagaaatcattaagttgtttagactgcaagccgtgaagcagctatcccagaattttcttctggacttaaggctgacagataaaattgtaaggaaagctggcaatctgacaaatgcttatgtatatgaagtgggccctgggcctgggggaatcacaagatccattctcaatgccggtgttgctgaacttctggtggttgaaaaggatagtcgatttattcctggattacagatgctttctgatgcagcacctggaaaactgagaattgttcatggagatgtcttgacgtttaagatagaaagggcttttccagaaagtctcaaaagacagtgggaggatgatcctccaaatgtacatattattggaaatctgccttttagtgtatcaactccactgattatcaaatggcttgaaaatgtttctcaaagaaatggaccatttgcatatggtagaacccggatgatgttgacttttcaaaaggaagtggcagagagacttacagctactactggaagcaaacagcgaagtcgcctctccatcatggcccagtacctctgcgacgttcagcacatcttgacaattccaggtcaggcttttgtgcccaaaccagagaatgttattccctgaagctcggcgattagaaagcacgggaaaactgttagagttggcagatgtggacccgactctccgacctacccaactcactgtctcacattttaagagcctctgcgatgtgtaccgaaaaatgtgtgatgaagacccacacctctttgcttacaatttcagagaagaactcaggcaaaagtcaaaaaaagaggatgacaagcagagctgcagactgtagctgctactcaaggggctgccagcctgccaggtgctgaacttctcatgtgtgcgcacgactgtgacttactccccttttacagcttggtaaagagaataaatatcaagtaagatagagtacaagctttaatatacatatatatactgcatagatgtgacatgtttagtataaacagcatctattacaaccttacaaaaatgaagtatctgcacattgtacccctaacccacattaaaataatttaaatatgctttaaataaagtgaaacctaatagctgaaagtattggagaaaaaatgaccttttaaaagctattaatcacaacttccccacctttttctacataaattagaatgcattcatcttaaggattctaaatcattttgatacctgctcaaaagggatataaactaagtaatttacaaattctgaccagtttaagctagttttctgatccacaataaattaaaataacttaagtctttacaactatttgtgggaaaatgtatcctgcacaaaacctaataatgaatagtgtttctgcaattttcattttgccaccagtttaaaagtgaaaccagcaatgtaagccgtatctgggttggactgtgctaggactttccatggctctctgtttcataaaagcactcttcgttgacaaccgatgttaagctctggacgctgaattctcgctcaaggacagagctgagatcaaggttggcgtttccaggatgactgtccagggactggtgacgcgtctgcgccttcagcagggtgcccctgtcccgcttggtgctgttggccttgcgtttgatggcgcagaagtgcccccgcaccagcttgggctgcccatccttgctgggcggctcttcagccgggtgagcatcggggtcctcagagatcctcagtctctggaactggatctcgatgtctttggtggggctgccctgcctccgagcctgggggtagtcgtcctcgtcgtcgctcagaatgtcgctctctttgatgagactgtcggagaagtcggccaggcccgcgtgggggtgctgctgggtgtcctgcccccttccggcaggggggcagccgctgctctgggtgctgctgctcaggctgccctcgtccgagtccagggagctgcccttgcccggctcccccggccactcgctgctggaggaattcttaaggacttttaaggacctggatgaagctgcaacgagaaaagcacacgttcccttatgacagaggaactgtttccacaatgtgtaacgttggccttggaggttatgagagttaacatcttaattaaaattttttcatctcactgaaattactaactgtcctgggacttgagcaaaaaaaaaaaaaa

SEQ ID NO: 96 - DNA, 1023 bp, Sus scrofa
  source 1..1023: mol_type = other DNA
atggctgtctccgggccgctcggcactttccgtctcccgccgttgcccacaattcgagaaatcattaggttgtttaaactgcaagcagtgaagcagctatcccagaattttctcttggacttgaggctaacagataaaattgtaaggaaagctggcagtctgacaaatgcttatgtttatgaagtgggccctgggcctggtggaatcacaagatccattctcaatgctggtattgctgaacttctggtggttgaaaaggatagtcggtttattcctggattacagatgctttctgatgcagcacctggaaaactgagaattgtccatggagatgtgttgacatttaagatagaaagggcttttccagaaagtctcaaaagacagtgggaggatgatcctccaaatgtacatattattggaaatctgccttttagtgtatcaactccactgattattaagtggcttgaaaacgtttctcatagaaatggaccttttgcttatggcagaacccagatgacactgacttttcagaaggaagtggcagagagacttatagccagtaaaggaagcaagcagcgtagtcgtctttccatcatggcccagtacctctgcggcgttcagcacgtcttgacaattccaggtcgggcttttgtccccaaaccagaggtggatgtgggggtggtgcactttacccccctgactcagcccaagatagagcagccgttcaagctggtggagaaagtggttcagaatgcatttcagttccgaaggaaatactgctatcgagggcttggaatgttattccctgaagctcagcgcatggaacgcactgggaaactgttagagtcggcagacgtggaccctactcttcgacccacccagctctcggtctctcatttccagagcctctgtgacgtgtaccggaaaatgtgtgatgaagacccacacctctttgcttacaacttcagagaagaactcaggcacacgtcagaaaagagagagagagatgatggggagagttag

SEQ ID NO: 97 - DNA, 3992 bp, Sus scrofa
  source 1..3992: mol_type = other DNA
agaggcagttgaaagatggcggacgaggcggcgctcgcccttcagcccggcggctccccctcggcggtggcggctgagagggaggccccgtcgccccccgccggggagccgctccgcaagaaaccgcggagagacggcccgggcgtcgggcggagcccgggcgagcctggtggggcggccttggagcgtgagctgccggcggcggcgggcggctgcccggcggcggcggtgctgtggcgggagaccgctgcgggcggggagcgggaggcccaggcggctgcggcggcaggagaaggaaacaatgggccgggcttacagggcctatcccgggaggcgcccccggccgacgacttctacgacgacgacgacgacgacgagggcgaggaggaggaagaggcggcggcagcggcggctattgggtaccgagataaccttctgtttggtgatgaaatcgtcaccaatggtttccattcttgtgaaagtgatgaggatgatagagcctcacatgccagttctagtgactggactccaagaccacggataggtccatatacttttgttcagcaacatctcatgattggcacagatcctcgaacaattcttaaagatttgctaccagaaacaattcctccacctgaattggatgatatgacactgtggcagattgttattaatatcctttcagaaccaccaaaaaggaaaaaaagaaaagatattaatacaattgaagatgctgtgaaattactacaagagtgcaaaaaaataatagttctaactggagctggggtttctgtttcttgtggaatacctgacttcaggtcaagagatggcatttatgcacgccttgcagtagactttccagaccttcctgatcctcaagcaatgtttgatattgaatatttcagaaaagatccaagaccattcttcaagtttgcaaaggaaatatatcctggacagttccagccatctctctgtcacaaattcatagccttgtcagataaggaaggaaaactgcttcgcaactatactcagaacatagatacactggaacaggttgcaggaatccagaggataatccagtgtcatggttcctttgcaacagcatcttgcctgatttgtaaatataaagttgactgtgaagctgtacgaggagatatttttaatcaggtggttcctcgatgtcctaggtgcccagctgatgaaccacttgctatcatgaaaccagagattgtcttttttggtgaaaatttaccagagcagtttcatagagccatgaagtatgacaaagatgaagttgatcttctcattgttattgggtcttccctgaaagtaagaccagtagcactaattccaagttccataccccatgaagtgcctcagatattaattaatagggaacctttgcctcacctgcattttgatgtagagcttcttggagactgtgatgtcattattaatgaattgtgtcataggttaggaggtgaatatgccaagctttgctgcaaccctgtaaagctttcagaaattactgaaaagcctccacgaacacaaaaagagttggctcacttgtcagagttaccacccacacctcttaatatttcagaaggctctagttcaccagaaagaacttcaccaccagcttcttcagtgactctccctcttttagaccaagcaacaaagagtaatgttgatgatctagatgtgtctgagtcaaaagactgtgttgaagagaaatcacaggaagtacagacttctaggagcattgaaagtgttaaagaaccgatggagagtccaggtttgaagaatgttgcctgcagtaatggagagaaaaatgaaagaacttcagttgccgaaacagtaagaaaatgctggccagctagacttgcaaaggagcagattagtaaacggcttgatggtaatcagtatctgtttttaccaccaaaccgttatattttccatggcgctgaggtatattcagactctgaagatgatgtcttatcctctagttcttgtggcagtaacagtgagagtggaacttgccagagtccaagtctagaagaacccatggaggatgaaagtgagattgaagaattttacaatggtttggaagatgatgctgatgttaatatgcgagctggaggaactggatttggagctgatggaagtgaccaagaggcagttaatgaagctatatccatgaaacaggaagcaacaggcgttaactatccatcaaacaaatcataatgtaataattgtccaggtacaggaattgttccaccagcattagggactttagcatgtcaaattgaacatttacttgtgaacctagtagcgcaaggaaaccagaaaggtgtaatatttataaactggtaaaagattttcttcccatgggtaatttttaacttcttgtttctgtgcttatatactcaaacactaactttttttttttttttttaaagaaaggtactaagtatctttaatcagctgttggacaagactttattttaaagattcatttgtatgatatatccccatgtatatataatttttttttttttgcctagtgagtttcaacattttaaagttttcaaaaagccattggaatgttaaattaatgtaaagggaacagcttatctagaccaaagaatggtactttcacacttttctttgtaacattgaatggtttaaaatcctcaaatctgtctgctgaacttttgattctttattagcacaattacctatttttaaacactggcatttttccaaaacttgtggcagctaactttttaaaaatctcatgacatgcagtgtgagtagaaagaagtcaacggtatgtgggagagcacttagttgtctttgctttttaaagtgagacttggtgctaatcgtttcaggaatattgtattgttcaaatgaagatggcttttgtacttcctgtggacatgtggtaatgtctatattgcctcataaaactaacctaaaaaacaaataaatgctttggaaatgtttcagttgctttagaaacagtagtgcctgccttggtccccttagttataagaatatttgctattgtttaaatacctatcactgtggtagagcttgcattgatccttttccacaagtattaaactgccaagatgtgaatttgcaaagtctttatgaaccaataataacggtacttcaacttggggagcgtgtaataatttggactgttgctcccccttacccctgcaccccccattaagtgagagcaacaggccctgatttaggttccaaagccagtaagtacatgtttcctgttgggaagatttggtgtaataaacaccaaactattagacggagacgaaggcgatgtgacttgtaataccagaatagctgatgaaactagttcctataaaaagcttagcctgccttaaaactagacatcaattttctcagctgcagaagcttctagcctttcaaaaaagttcatactttataaagttgcagagaagcatttattttccagtcctttccccaatgttacttccaaataaagtattcctatgttgctttagtatttattacaatttaacaaaaaagggtttgaaatgtagctgttctttaagtataaaatacccagcttggaccattactgccggagaagagttttattgaatggctatttccctacctaaaagaggtctcaaactgaatttattgggctacgctaaagaatgcagtatatttagttttccatttgcatgatgtgtgtttgtgctatagatgatattttaaattgaagaatttgttttaaattatttttacagtgaagactgttttcagctctttttatattgtacatagtcttttatgtaatctactggcatatgttttgtagactgtttaatgactggatatcttcctccaacttttgaaatacaaaaccagtgttttatacttgtacactgttttaaagtctattaaaattgacatttgacaaaaaaaaaaaa

SEQ ID NO: 98 - DNA, 3956 bp, Bos taurus
  source 1..3956: mol_type = other DNA
gcgagggaggagggccagagaggcggttgaaagatggcggacgaggcggcgctcgcccttcagcccggcggctctccctcggtagtggcggctgagagggaggccccgtcgccgcctgctggggagccgctccgcaagaggccgcgaagagacggccccggcgtcgggcggagctcgggcgagcccggtgggacggcccccgagcgggaactgccggcggcggccggcagttgcccggcggcggcggcggcgctgtggcgggaggcccaggcggcggcggcggcggcggccgaagaagaagacaatgggccgggcttacagggcctatccagggaggcgccaccggccgacgacttctacgacgacgacgacgagggcgaggaggaggaagaggcggcggccgctattgggtaccgagataaccttctgtttggtgatgaaatcatcaccaacggtttccattcgtgtgaaagtgacgaagatgatagagcctcgcatgcgagttctagtgactggactccaagacctcggataggtccatatacttttgttcagcaacatctcatgattggcacagatcctcgaacaattcttaaagatttactaccagaaacaattcctccacctgaattggatgatatgacactgtggcagattgttattaatatcctttcagaaccaccaaaaaggaaaaaaagaaaagatattaatacaattgaagatgctgtgaaattactgcaagagtgcaaaaaaataatagttctaactggagctggggtttctgtttcttgtggaatacctgacttcaggtcaagagacggtatttatgctcgccttgcaatagactttccagaccttccagatcctcaagcaatgtttgatattgaatatttcagaaaagacccaagaccattcttcaagtttgcaaaggaaatatatcctggacaattccaaccatctctttgtcacaaattcatagccttgtctgataaggaaggaaaactacttcgcaactatactcagaacatagatacactggagcaggttgcaggaatccaaaagataattcagtgtcatggttcctttgcaacagcatcttgcctgatttgtaaatataaagttgactgtgaagctgtacgaggagatatttttaatcaggtggttcctcgatgtcctagatgcccagctgatgaaccgcttgctatcatgaaaccagagattgtcttttttggtgaaaatttaccagaacagtttcatagagccatgaagtatgacaaagatgaagttgatcttctcattgttattggatcttccctgaaagtaagaccagtagcactaattccaagttccataccccatgaagtgcctcagatattaattaatagggaacctttgcctcacctgcattttgatgtagagcttcttggagactgtgacgtaattattaatgaactgtgtcataggttaggtggtgaatatgccaaactttgctgtaaccctgtgaagctttcagaaattactgaaaaacctcctcgaatacaaaaagagttggcacatttgtcagaattgccacccacacccctcaatatttcagaaggctcaagttcaccagaaagaacttcaccaccagattcttcagtgattgtcactcttttagaccaagaaacaaagagtaacgttgatgatccagatgtgtctgaatcaaaagaccatgtcacagaaaaatcacaggaagtacagacttctactaggagcattgaaagtgttaatgaacagttggagagtccagatttgaagaatgctgtctccaattctggtgagaaaaatgaaagaacttcagtagctgaaacagtgagaaaatgctggccagctagacttgcaaaggagcagattagtaaacgccttgatgataatcagtatctgtttttaccaccaaaccgttacattttccatggcgctgaggtatattcagactctgaagatgatgtcttatcctctagttcttgcggcagtaacagtgatagtggaacgtgccagagtccaagtttagaagaacccatggaggatgaaagtgagaatgaagaattttacaatggtttggaagatgatgctgatgttaatgagagagctggaggaactgtatttgaagctgatggaggtgatcaagaggcaattaatgaagctatatctgtgaaacaggaagcaacatgcattaactatccatcaaacaaatcataatatagtaattgtccaggtacagaaattgttccaccagcattagaatctttagcatgtcaaattgaatgtttacttgtgaactcaatagagcaaggaaaccagaaaggtataatatttatagactggtatcccctcaatgggtaatttttaacttccttatttctgtgcttatacactcaaacactaactttttttttttaaacaaaggtacgtaagtatctttagtcagctattggactagactttctgttaaaggttcatttgtatgatacatcgacatgtatatataattgtttttgcctagtgagtttcaacatttcagagtcttcaaaaagccattggaatgttaaattaatgtaaagggaacagcttatctagaccaaagaatggtattttcacacttttcgttttaacattgaatggtttgaaatcctcaaatctgttctgctaaacgtttgattctttattagcacaattacctatttttaaacactggcattttccaaaacttgtggcagctaactttttaaaaatctcatgacatgcaatgtgagaaggaagtcaacgtgggagagcactcagttgtttttgcttttttaaaatgaaacttggtgctaacagtttcagaaatgtatcgttcaaatgaagatcgcttttgtacttcctgtggacatgtagtaatgtctatattggctcataaaactaacttaaaaaataaatgctttggaaatgtttcagttgctttagaaacaatagtacctgcctgggtccccttagttttaagaatatttgccattgttgtttaaacacctatcactgtggtagagcttgcattgatcctttccacaagtattaaactgccaatgtcaatctgcaaagcctttatgatcaataataatggtactttaactggggagagtgtaatattttggactgttgccttttttgcccattcatgagagcagcagacccagattacggttccaaagccagaaagtacatgtttcctgttgggaggacttggtgtaagaaacaccaaacatactagcctagtattatggagatgaacgtgatgtaacttgtaatagcagaatagttaatgaaactagttcctataatgtctttatttaaaagcttagcctgccttaaaactagagaacaactttttcagctgcagaagcttctagcctttcaacaaaagtttgtactttataaaattgcacagtaacgatttatttctcagacctttccccaacattgttttcaaaaagtatttttatgttgctttagtatttattacagtttaacaagaagggtttgaaaaacagctgttcttaagcttaaaatatccagctaggaccattactgccagaggaaaaaattttattgaatggccatttccctacctaaaagatgtttcaatctgaatttatttgactacactaaagaatacagtatatttagttttccatttgcatgatctgtgtttgtgctatagatgatattttaaattgaaaaatttgttttaaattatttttacagtgaagactgttttcagctctttttatattgtacatagtcttttatgtaatctactggcatatgttttgtagactgtttaatgactggatatcttcctcaaacttttgaaatacaaaaccagtgttttatacttgtacactgttttaaaagtctattaaaattgtcatttgactttt

SEQ ID NO: 99 - DNA, 1826 bp, Sus scrofa
  source 1..1826: mol_type = other DNA
gctgctgggagttgtagttctctgccttgtctcggccccctcctgtttccggtgccgtcacgggacagagcagtcggtgacagtcccgagggcccccgccccgcgcccatggccgagccggatccctcagaccctgtggagacccagacagggaaggtgcaagaggctcaggactccgattccgattcggacactgaggaaggagcagctggtggagaagccgagatggacttcctgcggaatttcttctcccagaccctgggcctgggcacccagaaggagcgtctgctggacgagctcaccctggaaggggtgtcccgctacatgcagagtgagcgctgtcgcagggtcatctgtttggtgggagctggaatctccacccgcggcatccctgacttccgctccccgtccacgggcctctatgccaacctggagaagtaccgtctgccctacccggaggccatctttgagattggctacttcaagaaacatccagagcccttcttcgccctcgccaaggaactctatcctgggcagttcaagccgaccatctgtcactacttcatccgcctgctgaaggagaaagggctgctcctgcgctgctacacgcagaacatagacaccctggagcgagtggcgggactagagcccgaggacctggtggaggcccatggcaccttctacacgtcacactgcatcagccccctctgccggcaggagtacacgctaggctggatgaaagagaagatcttctccgaggtgacccccaagtgtgagaaatgtcagagcgtggtgaagcctgatatcgtgttcttcggtgagaacctcccagcgcgtttcttctcctgcatgcagtcagacttcctgaaggtggaccttctcatcgtcatgggcacctccctgcaggtgcagcccttcgcatccctcatcagcaaggcgcccctctccaccccacgcctgctcatcaacaaggagaagaccggccagactgacccgttcctcgggatgatgatgggcctcggaggaggcatggacttcgattcaaaaaaggcgtacagggacgtggcctggctgggtgactgcgaccagggctgcttggccctcgctgatctcctcggatggaagaaggagctggaggaccttgtccggaaagagcatgccagcatagatgcccagtcgggatcgggcacccccaatcccaccacttcagcttcccccaggaagtctccgcctcctgccaaggcagaggccaggacctcggagggagagaaaccccagtgacagctgcgtctcccaggcaggacgcccagctttgggacagctgagtcccaaccagccctggcctcctctaacttacagctcttttctggggagctcagaacgtttccgcaatcttttaactactccctcctaaacctggggtcccggcagccccgaccccagcaaatctctaacattctttggggccgaggcttgggcagcccgcctctaacagcccacagcctctaaccacccccgggactgaggatcagcctccctaatctctaactgctcagggggccgggtgtacctccaaacctctaactgtcccaggccaagggtccccagggcccccacgccctctactgtccccaggagcctgtggccaagtagctgaaacctaacctacccgtgggtggggggatctgagcctcaaggtctcccaagtctcttcctccagctccccaagtgggtgctggaccccctcactcgggacccacttcccatgccaaggagggcagatggtgtcacttattagagatgaattaaaaacaaagaacaactaa

SEQ ID NO: 100 - DNA, 1849 bp, Bos taurus
  source 1..1849: mol_type = other DNA
gacctatctcggccctctcctgtttccggtgccgtcacgggatagagcagtcggtgacagtcccgagggcccccgccccgcgcccatggccgatccggatccctctgaccctgaggagacccaggcagggaaggtgcaggaggctcaggactcagattcagacactgaggcaggagccactggcggagaagcagagatggacttcctgcggaatttcttctcccagactctgggcctgggcacccagaaggagcgactcctggacgaactaaccctggaaggagtgagccgctacatgcagagcgagcgctgtcgcagggtcatctgtttggtgggagctggaatctccacttccgcgggtatccctgactttcggtccccaaacacgggcctctatgccaacctggagaaataccgtcttccctacccggaggccatctttgaaatcagctacttcaagaaacatccagagcccttctttgctctcgccaaggaactctatcctgggcagttcaagccgaccatctgccactacttcatccgcctgctgaaggaaaaaggactgctcctgcgctgctacacacagaacatagacaccctggagcgagtggctgggctggagcccgaagacctggtggaggcccacggcaccttctacacgtcccactgcatcagctcaggctgccggcaggagtactcactaagctggatgaaagagaagatcttctccgaggtgactcccaagtgtgagaaatgtcagagcgtggtgaagcctgatatcgtgttcttcggcgagaacctcccagcgcgtttcttctcctgcatgcagtcagacttcctaaaggtggacctccttatcatcatgggcacctccctgcaggtgcagcccttcgcgtccctcatcggcaaggcgcccctgtcgaccccgcgcctgctcatcaacaaggagaagactggccagactgaccctttcctcgggatgatgatggccctcggaggaggcatggactttgactccaagaaggcctacagggacgtggcctggctgggcgactgtgaccagggctgcctggccctcgccgacctccttggatggaagaaggagctggaggaccttgttcggaaggagcatgccagcatagatgcccagtcggggtcgggggcctccaaccccgctacttcagcttcccccaggaattctccaccacctcccaccaaggaggagcccaggaccactgagggagagaaaccccagtgaccactgcgtctcccaggcaggacacccagccttgggacagctgagccccaaccagccctggccctcctctaacttacagctcttttctggggagctcagaacatttcttcaatgtattaactattccctcccaaaactggtgtcccagcacccctgacctcaccaaatctctaacattcttgggggcagaggcttgggcagcctgtctctaacagcccacagcctctaaccacccctacggccaaggatcagcccctctaatctctaactgctcagggggccacaggtacctccaaactgctaactgtcccaggacaagagccccagggcccacacgctctctactgctcccaagggtctgtggccaagtagataaaacctaacctaccctgggtggggagttgtgccctccacgcacccagtggggagatccgagcctcaaggcctcccaggcctctgcctccaactctgaaagtgggtgctaggcccctctcttggatcccgcttcccaggcaggggtgggcagatggtgttgcttattagagatgaattaaaaacaaaaaacaactaacgattaaaaaaaaaaaaaaa

SEQ ID NO: 101 - DNA, 3646 bp, Bos taurus
  source 1..3646: mol_type = other DNA
ctcctcctccgaggaaacctgccgcttctagctccccaccctcccctttaaagggtgacttgctctgtgccagaccgctgctccagcctgtgccgccttgggttcagcgggtctactgttctgtccgcgctcgcgcctggtgccctgcatctcctacagagggacatcccccgagatggagagcaaggccctacttctgctggctctgagcgtgtgcctgcagagtctgaccgtctcccgcggagggctggtcgccgcagacaggattacaggaggaaaagattttagagacattgaaagtaaatttgctctcaggactcccgaagacacagctgaggacacttgccacctcattcctggagtgaccgaatctgtggctaactgtcacttcaaccacagcagcaaaacctttgtggtgatccatggctggacggtgacaggaatgtatgagagttgggtgccaaaactcgtggctgccttgtacaagagggaaccggactccaacgtcatcgtggtggactggctgtcacgggcccagcagcattatccagtgtctgcagggtacaccaagctggtgggacaggatgtggccaagtttatgaactggatggcggatgaatttaactatcccctgggcaatgtgcatctcttgggatacagccttggggcccatgctgctggtattgcaggaagtctgaccaataagaaggtcaacaggataaccggcttagatccagctggacctaacttcgagtatgcagaagctccaagtcgcctttctcctgatgatgcggattttgtagacgttttacacacattcaccagggggtcaccaggtcgaagtatcggaatccagaaaccagtagggcatgtggacatttaccctaacggaggcactttccaaccaggatgtaacattggggaagctctccgtgtgattgcagagagaggccttggagatgtggatcagctggtgaagtgctcccacgaacgttccgttcatctcttcattgactctctgttgaatgaagaaaatccaagtaaggcctaccggtgcaattccaaagaagcctttgagaaaggtctctgcctgagctgcagaaagaaccgttgcaacaacatgggctacgagatcaacaaggtcagagccaaaagaagcagcaagatgtacctgaagactcgttctcagatgccttacaaagtcttccattaccaagtaaagatacatttttctggaactgagagtaatacatacaccaaccaggccttcgagatctctctgtatggcactgtggctgagagcgagaacatcccttttaccctgcctgaagtttccacaaataagacatactcctttctactttacacggaggtggatattggagaattgctaatgttgaaactcaaatggattagtgattcctacttcagctggtccaactggtggagcagccccggctttgatattgggaagatcagagtaaaggcaggagagactcaaaaaaaggtgatcttctgttcccgggagaaaatgtcttatctgcagaaaggaaagtcacctgtgatatttgtgaaatgccatgacaagtccctgaatagaaagtctggctgaaacttggcaaagctacggaagaaagaacagcatatgaattctatgaagaatgaagtaacttttacaaaagatgcccagtgctttagatggtgaaatgtggattttccggagtattaaccccagctctagccttattagttattttaggagacagtctcaaatactaaaactaattcaatttgaggtgtatagtggccaaatagcacatcttccaacattaaaaaaataacagatatgaaaagcactgcattctgtcttttgaaaaaatatgagttatttaaaatgataaaataatcagatctcttcatgtagtaagtttagccatagcctaaaattgattaaaatctcatttttaattgggattctgcgttttctagactgataaccttctcagagttttctccgagtctaaatataggaagtaagtttttttgcagcgtgagtaggacccgtttacctatcaatcaaaatccctactttcttggaattactctcctcttgccattggaatgtagtccaagaatgaaccaggaactagtgacttggagatagaaatgaagaatagagttgataaagcactgaacctttaaaccccctctacggttggttgcatcataactaagttaccaattaaaggagatatataaagttgagatcaattaaatcttaataaacttaacagtttatggtttagtatttccccttccttttcctgctttgtctcaagattatattttaatagtttttccctagataggcttttcaactgagcctgttatcatcagctgatacagaattttaactacagaggaaaaaaaatgatactgtaattttattatcagactttcaaaattacattcattgaagtcctttaatgtatgatagttactgttcaattttaggcttatttcagccatgcttcagtcagactttgagcatgtctctttcttccctaactttgataagaaaacacgtattcaagatgaaactttgcatcatgcaaagtcctacaaattttagacaaagattgtttttactaagtaaagaaacagaaaagttacagtcagtcatatagtcactggcagtgcttaaaaccgttgtggacaatgaaaaggatcaattgctatatccacgtgaattgtcatgaaagttgtttttaacaactagtcaagagtgagtgaacatttatttataaactagatctcatattttcagaatgcttttctatatattattatcaaatgatagagaagtcaaatatttcagaggctattactgaaaatccaaactgtgaatgtgtgtgggtgtctgaacacatattcacgtatgaagccccacatacacgattattgctcagcatggctcggaaggtaaatgattaagaggtacattagaacacactgagtagaaaatgttccagtggtgccggaacactccttcatctgagagatacggttgtgcctgtacaaagtagaatgaatgctgtgattgacatcagccagtttggaatgaattctctctaaaaataaaatgttgtgtgacttgttgttggcatcccccttcattgttataattcattaattctcgggatttgggttttgagcaagggtacatcaacttaagaaagtataaattcagtcagtgtatggccctgggaactctgacttctacagacttcactatatatatatcagcaatgctttgactttcaatgttatttattagctgtaaatatatgtgtgtatgtgtaaagggagattgtacatactggaaaggtcattgtggctatctgcatttatagatgtttggtgctaattttgtacgtgtttttatcagtgacagtttcactgagccaacttactctgatgaaacaatacaataaaatgggctttaaaaaaacacaaaacttacttgtgtgaagaaatggaatctacttttaataaaattgacaacattttattacaaaaaaaaaaaaaaaaaaaaaaaaaaaaaaaaaaaaaaaaaaaaaaaaaaaaaaaaaaaaaaaaaaaaaaa

SEQ ID NO: 102 - DNA, 2946 bp, Sus scrofa
  source 1..2946: mol_type = other DNA
ggaaagctgccacttctggctgccccgcttcttctttaaagggtgacttgccctgcaacagatcgctgctccagcctgctcccgcctcgggctgtcggcgctcccgcccgtgccctgtaacttctaccccaggaacgcgtccccagatggagagcaaagccctgctcctagtggctctgagcgtgtggcttcagagtctgatcgtctcccgcgaagggttggccaccgccgacagaatttcaggaggaagagattttacagacatcgaaagtaaatttgctctcaggactcctgaagacacagttgaggacacttgccatctcattcctggagtgacagaatctgtggctaactgtcatttcaaccatagcagcaaaacctttgtggtgatccatggctggacggtgacaggaatgtatgaaagttgggtccccaaacttgtggctgccctatacaagagggaaccggattccaacgtcattgtggtggactggctgtctcgggcccagcagcattatccaatatctgcgggatacaccaagctggtgggacaggatgtggccacgtttatcgactggatggcggttgaatttagctatcctcccaacaatgtccacctcctgggatacagccttggagcccacgctgctggcattgcaggaagtctgaccaagaagaaggtcaatagaattactggcctagatccagctggacctaacttcgagtatgcagaagctccaagccgcctttctcccgacgacgcagattttgtagacgtcttacacacattcaccagagggtcacctggccgaagtattggcatccagaaaccagtagggcatgttgacatttatcccaatggaggcactttccaaccagggtgtaacattggagaagccatccgtgtgattgcagagagaggccttggagatgtggaccagctcgtgaagtgctcccatgagcgctccatccatctcttcatcgactctctgttgaatgaagaaaatcctagtaaggcctaccggtgcaattcaaaggaagcctttgagaaagggctctgcctgagttgcagaaagaaccgttgcaacaacttgggctatgagatcaataaggtcagagccaaaagaagcagcaaaatgtacctgaagactcgtgctcagatgccctacaaagtcttccattaccaagtaaagatgcgtttttctgggactgagagtgatacccacaccaaccaggcctttgagatctctctatatggcaccgtggccgagagtgaaaacatccctttcaccctgcctgaagtctccacaaataagacatactcctttctaatttacacagaggtggatattggagagttgctaatgttgaaactcaaatgggtcagtgattcatacttcagctggtccaactggtggagcagccctggctttgctattgagaagatcagagtgaaggcaggagagactcaaaaaaaggtgatcttctgttcccgggagaagaagtctcacctgcagaaaggaaagtcctctgtggtgtttgtgaaatgccacgacaagtctctgaatagaaagtctggctgaaactgagcaaatgtacagaagaaggaacagcacgtggagtctatgaagaacaaaacaaattttgacaaagatgcccagtgctttgggagattaaaagtgaattttcccaagtattaatcccagctctagccttattagttattttaggagacagtcttaaacattaaaaatgtggctacttcagtttgaggagagagtggccaaatgacacatcttccaacattagaaaacagcagatatgaaaagcactgcattctgtcctttgggaaagaaagaagaattgtttgggcttgtggtaaaataatcagcggtctttgtgtagtaagtttagccgtagcctaaaattgatgagaacctcatactttaattggaattctgcatttttcagactgataccttctcaaaattgtcttcaagtctaaatatagaaggtgatttttttatagcctgagttggacccatgtacttaattgtcaaaatactttcttggaatgaatcttctcttgccattgggatgtagcctaggagttaaccaggaactagtaacttggagagaggaagggaaaacagagttgataaagcactgaagctttaaacactcttgcagttggttgcatcataactaagttacaaattaaaggagatataaaagttagatcaattaagtcttaagaggctttgcagtttattggtcggtcttttcccttcggtttttttttttgctttgtctcaaattgtattttaacgatgttctttgggtaggcatttaaaatgagcctgttatcatcatctgacacgtaattttaagctatagagaaagaaattggtagccataattttattcatcagattgtcaaaattacattcgttgatttgaaatcctttaatatatgatggttactgttcaattttaggcttacttcagccatacttcagtcagtttttgagcatgtttttcttctctaattttgataagataacgtgttcaaggtcaaattttgcatcatgcaaactcctacaaattttgggcaaggattgtttttacaataggaaagttgggagctccaccatggcccaggggttaacgaacccgactagcatccatgaggatgtgggtttgatccctggccctgctcagtgggttacggatccagcgttgccatgagctgtggtctaggtcccacatgcagctcagatcccgagttgctgtggctgtggtgtaggccagtggctacagctctgattggacccctagcctgggaacttccatatgccgtgggtgcagccctaaaaagacaaaagaaagaaaagatactgggatacattcataagcagtgcttaaaaccattgtgggcaattaaaa

SEQ ID NO: 103 - DNA, 23 bp, Sus scrofa
  source 1..23: mol_type = other DNA
ctggcttactcctatcatgaagg

SEQ ID NO: 104 - DNA, 23 bp, Sus scrofa
  source 1..23: mol_type = other DNA
tcccatgccatgaagagggtagg

SEQ ID NO: 105 - DNA, 20 bp, Bos taurus
  source 1..20: mol_type = other DNA
tgtcgagtctcgattatggg

SEQ ID NO: 106 - DNA, 20 bp, Bos taurus
  source 1..20: mol_type = other DNA
ataatcgagactcgacatgg

SEQ ID NO: 107 - DNA, 20 bp, Bos taurus
  source 1..20: mol_type = other DNA
aggcgatgacgagatcgcgg

SEQ ID NO: 108 - DNA, 20 bp, Bos taurus
  source 1..20: mol_type = other DNA
ccccgtgggactcaccggac

SEQ ID NO: 109 - DNA, 20 bp, Bos taurus
  source 1..20: mol_type = other DNA
cttccaagggcaacaatgag

SEQ ID NO: 110 - DNA, 20 bp, Bos taurus
  source 1..20: mol_type = other DNA
gtaagggagactgacctgtg

SEQ ID NO: 111 - DNA, 19 bp, Bos taurus
  source 1..19: mol_type = other DNA
tgcctgaaggacaagacta

SEQ ID NO: 112 - DNA, 20 bp, Bos taurus
  source 1..20: mol_type = other DNA
acctctctctcaatctcctg

SEQ ID NO: 113 - DNA, 4061 bp, Sus scrofa
  source 1..4061: mol_type = other DNA
ctaggtgtgccacctgctctgaggccaaagggcctgtgaatgactccaggtgttgttccagcccctgagatgggtgtcaggttacaagacgcataatcattccagtttggcaacttcacctgtggggctgttttaatcgacctgccccacagtccccaaaccactcctggaagacagatagcagtgggacacggccagctccccagggggagagagatgctggacgcaagggaccgaggcccaccctccacttaagcctcctccgaccagtacaagctgggcatcgctgacctgcacgccagccagggccccattctggggccctgctcctgacttgggctgctgacctcagctccaggctatagaaaattcaggaagatcacagagctggcatgatgtcgccccccagcgcaaaccccgcacccccctgcagaccttcaaaaaggtggggatccccatcatcgcggcactgctgggcctggcagccatcgtcgtcgtggctattctcatcaaggtgattctggacaaatactacttcctctgctggcagcccctccacttcatcccgagagagcagctgtgtgacggccagcaggactgtgcctcaggggaggatgagcagctctgtgtcaagaaattccccgatgggcccccagtggcagtccgcctctccagggaccgatctaccctgcaggtgctgcacccagccacacagacctgggcctctgcctgtttcgacaacttcacggaagctctggccaagacagcctgtgggcagatgggctatgacagcaaacccaccttcagagctgtgaggattggcccagaccaagatctggatgttgtgaaaatcacagagaatgaccaggagcttcgggtgcaaaactcaagcggcccctgtctctcaggctccctggtttcgctgcactgccttgcctgcggggagagcctgaaggcccctcgggtggtgggcggggagaaggcctctgtggattcctggccctggcaggtcagcatccagtacaacaaacagcacatctgtggagggagcatcctggacccccactggatcctcacggcagcccactgcttctggaagcatctcgatgtgcccaactggaaggtgagggccggctcagacaaactgggcaacttcccatccctgcctgtggccaagatcttcatcactgagcccaacagcacgtcccccaaagagaaagacattgcccttgtgaagctgcagctcccgctcacgttctccggcacagtcaggcccatctgcctgcccttctctgacgaggaactcactccaggcaccccagtctgggtcatcggctggggctttacggaacctaatggagggaagatgtctgatcatctgctgcaggcgtccgtccaggtcattaaccgcactcggtgcaacgcggaggatgcgtaccagggggaagtcacggagaagatgctgtgtgcaggcctcctggagggcggtgtggacacctgccagggtgacagtggtgggcctctgatgtatcactctggccggtggcaagcagtaggcatcgtgagctggggctatggctgtggggggccgagtacccctggagtatacaccaaggtcacagcctacctcgactggatctacaatgttcggaagtctgatccatgatgctgctgcccacctgcagtgctggaaactgcttccctcctgccctgcccacctggggatccaccagaagtcaggcacagagcaagagcccccttgggcccatctctctgcaatctcggcattccttggcacagcaaaggacctccactcctgtaagagaccccacagcccacaggcacccaggagaaagtcagcagccccagctcagataccctgacgcttccagcatcccaaggaaggagagacacagcccacaagccagcctccaggccaaggtcttggaagacattgctaggccaaaaaggaaatttctactgaagaaaagaatggacatagatgctcttctcagagcccagatcaccacgagctagagtggagggaggaaagggtgtgtgccagccctctccttccctacccatccccaagcccacccaaacgaggaagcatttagaatgtaaaatgcactgtcctgctgtttgcactatcatttcttactattgttattactgctacctctatggccactgtttattatactggtgggtgagctccttgaggctgcatggtgagagctgagctcaaactactgacgcatgatctgtactttggcctgggtaggaaaaggtcttggggagatgattgacactcaaagcagaagaagaggaaaaatggagaccagatctgatgggtagaagcaacagtgaccagccaactggtaaagccagcatcagtcagtaagtctcctttgcacacacgtctcatcccactgtccttctctccttcctccctgccgtgatataccctcaacttgggaggaagagaatttggaccttcaaggctgacattacatcctggctccccaagtggctagaaggaggtattgcacctctaagtcacaacctggaaagccctggctgtggtttttatctttcgctttttaaaaaagtcatttatggggttccttagtagcccagtggttaagtattcagcattgtcactgctgtggcttgggttcaatccctggcctggaaacttccacatgcagccaaaattaaataaataaaaaccaaaaataaaaacaaataaacaggagttcccgtcgtggcgcagtggttaacgaatccgactaggaaccatgaggttgcaggttcgatccctgccgttgctcagtgggttaacgatccggtgttgcggtgagctgtggtgtaggtcacagacacggctcgaatcccgagttgctgtggttctggcataggctggcagctacggctccgattagacccctaggctgggaacctccatgtgccgcgggagtggcccaagaaatggcaaaaaaaaataaaaataaaaataaaataaacaaaatatttatcatctgccaggtatgagatagtctccatgctgggttctgaggatataaacatgaataaattcctacactcaaaatggccagagaagttagcccagttggtagcttgatgaaggcccaggtattctgaagaccctcttgctacagctgggtcaaatcctggataaaatagacttgttagggagttcccattgtggtacagcagaaactaatctgactagtatccatgaggatgcgagtttgatccctggcctcgctcagtggtcaaggatccggcgttgctgtgagttgtggttgtaggtggcagacacagcttggcttctgagttgctgtggctgtggtataggccagcagctgcagttccaattgggcccctagcctgggaacctccatatgccgcacatgcagccctaaaaagccaaaaaaaaaaaaaaaaaaaaaagacttgttaatggtgtattaggttcttagtaaataatataaatcctcaaaggcaaaaaaaaaatcaaaatacaataaaactgtcagaatcatgagctgaaactaaggtggagatctgggagcagctaactataatcttggggtcccctagggacaaagacttacatgaaggctgagatcagaactaaacccagactcactgcaggtagggaagctgaacctgggactctcacatgcaccagagacaatcaaaggaggcatatgtggtcccagatggcagatcaaatgcaggaggaaaacatcccaaatttagagccttgggataccccaagatcctccaaatagaagctcacaatcaaagatcagtcaagattttaaacaagaaaaaccaccgtatgccgcatatgaaaatcatcaaaaccagaaaatagaacctcaaggatttcagatgttagaattatcaggtaattacaataataactaataaagtatttctatgtcctgggcactattctaagtatttcatcatcccccagcacaggagctttcttctgacacaggcacagccccctccaaaaaaaaaaa

SEQ ID NO: 114 - DNA, 4030 bp, Sus scrofa
  source 1..4030: mol_type = other DNA
tctaggtgtgccacctgctctgaggccaaagggcctgtgaatgactccaggtgttgttccagcccctgagatgggtgtcaggttacaagacgcataatcattccagtttggcaacttcacctgtggggctgttttaatcgacctgccccacagtccccaaaccactcctggaagacagatagcagtgggacacggccagctccccagggggagagagatgctggacgcaagggaccgaggcccaccctccacttaagcctcctccgaccagtacaagctgggcatcgctgacctgcacgccagccagggccccattctggggccctgctcctgacttgggctgctgacctcagctccaggctatagaaaattcaggaagatcacagagctggcatgatgtcgccccccagcgcaaaccccgcacccccctgcagaccttcaaaaaggtggggatccccatcatcgcggcactgctgggcctggcagccatcgtcgtcgtggctattctcatcaaggtgattctggacaaatactacttcctctgctggcagcccctccacttcatcccgagagagcagctgtgtgacggccagcaggactgtgcctcaggggaggatgagcagctctgtgtcaagaaattccccgatgggcccccagtggcaggtgctgcacccagccacacagacctgggcctctgcctgtttcgacaacttcacggaagctctggccaagacagcctgtgggcagatgggctatgacagcaaacccaccttcagagctgtgaggattggcccagaccaagatctggatgttgtgaaaatcacagagaatgaccaggagcttcgggtgcaaaactcaagcggcccctgtctctcaggctccctggtttcgctgcactgccttgcctgcggggagagcctgaaggcccctcgggtggtgggcggggagaaggcctctgtggattcctggccctggcaggtcagcatccagtacaacaaacagcacatctgtggagggagcatcctggacccccactggatcctcacggcagcccactgcttctggaagcatctcgatgtgcccaactggaaggtgagggccggctcagacaaactgggcaacttcccatccctgcctgtggccaagatcttcatcactgagcccaacagcacgtcccccaaagagaaagacattgcccttgtgaagctgcagctcccgctcacgttctccggcacagtcaggcccatctgcctgcccttctctgacgaggaactcactccaggcaccccagtctgggtcatcggctggggctttacggaacctaatggagggaagatgtctgatcatctgctgcaggcgtccgtccaggtcattaaccgcactcggtgcaacgcggaggatgcgtaccagggggaagtcacggagaagatgctgtgtgcaggcctcctggagggcggtgtggacacctgccagggtgacagtggtgggcctctgatgtatcactctggccggtggcaagcagtaggcatcgtgagctggggctatggctgtggggggccgagtacccctggagtatacaccaaggtcacagcctacctcgactggatctacaatgttcggaagtctgatccatgatgctgctgcccacctgcagtgctggaaactgcttccctcctgccctgcccacctggggatccaccagaagtcaggcacagagcaagagcccccttgggcccatctctctgcaatctcggcattccttggcacagcaaaggacctccactcctgtaagagaccccacagcccacaggcacccaggagaaagtcagcagccccagctcagataccctgacgcttccagcatcccaaggaaggagagacacagcccacaagccagcctccaggccaaggtcttggaagacattgctaggccaaaaaggaaatttctactgaagaaaagaatggacatagatgctcttctcagagcccagatcaccacgagctagagtggagggaggaaagggtgtgtgccagccctctccttccctacccatccccaagcccacccaaacgaggaagcatttagaatgtaaaatgcactgtcctgctgtttgcactatcatttcttactattgttattactgctacctctatggccactgtttattatactggtgggtgagctccttgaggctgcatggtgagagctgagctcaaactactgacgcatgatctgtactttggcctgggtaggaaaaggtcttggggagatgattgacactcaaagcagaagaagaggaaaaatggagaccagatctgatgggtagaagcaacagtgaccagccaactggtaaagccagcatcagtcagtaagtctcctttgcacacacgtctcatcccactgtccttctctccttcctccctgccgtgatataccctcaacttgggaggaagagaatttggaccttcaaggctgacattacatcctggctccccaagtggctagaaggaggtattgcacctctaagtcacaacctggaaagccctggctgtggtttttatctttcgctttttaaaaaagtcatttatggggttccttagtagcccagtggttaagtattcagcattgtcactgctgtggcttgggttcaatccctggcctggaaacttccacatgcagccaaaattaaataaataaaaaccaaaaataaaaacaaataaacaggagttcccgtcgtggcgcagtggttaacgaatccgactaggaaccatgaggttgcaggttcgatccctgccgttgctcagtgggttaacgatccggtgttgcggtgagctgtggtgtaggtcacagacacggctcgaatcccgagttgctgtggttctggcataggctggcagctacggctccgattagacccctaggctgggaacctccatgtgccgcgggagtggcccaagaaatggcaaaaaaaaataaaaataaaaataaaataaacaaaatatttatcatctgccaggtatgagatagtctccatgctgggttctgaggatataaacatgaataaattcctacactcaaaatggccagagaagttagcccagttggtagcttgatgaaggcccaggtattctgaagaccctcttgctacagctgggtcaaatcctggataaaatagacttgttagggagttcccattgtggtacagcagaaactaatctgactagtatccatgaggatgcgagtttgatccctggcctcgctcagtggtcaaggatccggcgttgctgtgagttgtggttgtaggtggcagacacagcttggcttctgagttgctgtggctgtggtataggccagcagctgcagttccaattgggcccctagcctgggaacctccatatgccgcacatgcagccctaaaaagccaaaaaaaaaaaaaaaaaaaaaagacttgttaatggtgtattaggttcttagtaaataatataaatcctcaaaggcaaaaaaaaaatcaaaatacaataaaactgtcagaatcatgagctgaaactaaggtggagatctgggagcagctaactataatcttggggtcccctagggacaaagacttacatgaaggctgagatcagaactaaacccagactcactgcaggtagggaagctgaacctgggactctcacatgcaccagagacaatcaaaggaggcatatgtggtcccagatggcagatcaaatgcaggaggaaaacatcccaaatttagagccttgggataccccaagatcctccaaatagaagctcacaatcaaagatcagtcaagattttaaacaagaaaaaccaccgtatgccgcatatgaaaatcatcaaaaccagaaaatagaacctcaaggatttcagatgttagaattatcaggtaattacaataataactaataaagtatttctatgtcctgggcactattctaagtatttcatcatcccccagcacaggagctttcttctgacacaggcacagccccctccaaaaaaaaaaa

SEQ ID NO: 115 - DNA, 4096 bp, Sus scrofa
  source 1..4096: mol_type = other DNA
tctaggtgtgccacctgctctgaggccaaagggcctgtgaatgactccaggtgttgttccagcccctgagatgggtgtcaggttacaagacgcataatcattccagtttggcaacttcacctgtggggctgttttaatcgacctgccccacagtccccaaaccactcctggaagacagatagcagtgggacacggccagctccccagggggagagagatgctggacgcaagggaccgaggcccaccctccacttaagcctcctccgaccagtacaagctgggcatcgctgacctgcacgccagccagggccccattctggggccctgctcctgacttgggctgctgacctcagctccaggctatagaaaattcaggaagatcacagagctggcatggatcccgccagtgatcaacccctgaacagccttgatgtcgccccccagcgcaaaccccgcacccccctgcagaccttcaaaaaggtggggatccccatcatcgcggcactgctgggcctggcagccatcgtcgtcgtggctattctcatcaaggtgattctggacaaatactacttcctctgctggcagcccctccacttcatcccgagagagcagctgtgtgacggccagcaggactgtgcctcaggggaggatgagcagctctgtgtcaagaaattccccgatgggcccccagtggcagtccgcctctccagggaccgatctaccctgcaggtgctgcacccagccacacagacctgggcctctgcctgtttcgacaacttcacggaagctctggccaagacagcctgtgggcagatgggctatgacagcaaacccaccttcagagctgtgaggattggcccagaccaagatctggatgttgtgaaaatcacagagaatgaccaggagcttcgggtgcaaaactcaagcggcccctgtctctcaggctccctggtttcgctgcactgccttgcctgcggggagagcctgaaggcccctcgggtggtgggcggggagaaggcctctgtggattcctggccctggcaggtcagcatccagtacaacaaacagcacatctgtggagggagcatcctggacccccactggatcctcacggcagcccactgcttctggaagcatctcgatgtgcccaactggaaggtgagggccggctcagacaaactgggcaacttcccatccctgcctgtggccaagatcttcatcactgagcccaacagcacgtcccccaaagagaaagacattgcccttgtgaagctgcagctcccgctcacgttctccggcacagtcaggcccatctgcctgcccttctctgacgaggaactcactccaggcaccccagtctgggtcatcggctggggctttacggaacctaatggagggaagatgtctgatcatctgctgcaggcgtccgtccaggtcattaaccgcactcggtgcaacgcggaggatgcgtaccagggggaagtcacggagaagatgctgtgtgcaggcctcctggagggcggtgtggacacctgccagggtgacagtggtgggcctctgatgtatcactctggccggtggcaagcagtaggcatcgtgagctggggctatggctgtggggggccgagtacccctggagtatacaccaaggtcacagcctacctcgactggatctacaatgttcggaagtctgatccatgatgctgctgcccacctgcagtgctggaaactgcttccctcctgccctgcccacctggggatccaccagaagtcaggcacagagcaagagcccccttgggcccatctctctgcaatctcggcattccttggcacagcaaaggacctccactcctgtaagagaccccacagcccacaggcacccaggagaaagtcagcagccccagctcagataccctgacgcttccagcatcccaaggaaggagagacacagcccacaagccagcctccaggccaaggtcttggaagacattgctaggccaaaaaggaaatttctactgaagaaaagaatggacatagatgctcttctcagagcccagatcaccacgagctagagtggagggaggaaagggtgtgtgccagccctctccttccctacccatccccaagcccacccaaacgaggaagcatttagaatgtaaaatgcactgtcctgctgtttgcactatcatttcttactattgttattactgctacctctatggccactgtttattatactggtgggtgagctccttgaggctgcatggtgagagctgagctcaaactactgacgcatgatctgtactttggcctgggtaggaaaaggtcttggggagatgattgacactcaaagcagaagaagaggaaaaatggagaccagatctgatgggtagaagcaacagtgaccagccaactggtaaagccagcatcagtcagtaagtctcctttgcacacacgtctcatcccactgtccttctctccttcctccctgccgtgatataccctcaacttgggaggaagagaatttggaccttcaaggctgacattacatcctggctccccaagtggctagaaggaggtattgcacctctaagtcacaacctggaaagccctggctgtggtttttatctttcgctttttaaaaaagtcatttatggggttccttagtagcccagtggttaagtattcagcattgtcactgctgtggcttgggttcaatccctggcctggaaacttccacatgcagccaaaattaaataaataaaaaccaaaaataaaaacaaataaacaggagttcccgtcgtggcgcagtggttaacgaatccgactaggaaccatgaggttgcaggttcgatccctgccgttgctcagtgggttaacgatccggtgttgcggtgagctgtggtgtaggtcacagacacggctcgaatcccgagttgctgtggttctggcataggctggcagctacggctccgattagacccctaggctgggaacctccatgtgccgcgggagtggcccaagaaatggcaaaaaaaaataaaaataaaaataaaataaacaaaatatttatcatctgccaggtatgagatagtctccatgctgggttctgaggatataaacatgaataaattcctacactcaaaatggccagagaagttagcccagttggtagcttgatgaaggcccaggtattctgaagaccctcttgctacagctgggtcaaatcctggataaaatagacttgttagggagttcccattgtggtacagcagaaactaatctgactagtatccatgaggatgcgagtttgatccctggcctcgctcagtggtcaaggatccggcgttgctgtgagttgtggttgtaggtggcagacacagcttggcttctgagttgctgtggctgtggtataggccagcagctgcagttccaattgggcccctagcctgggaacctccatatgccgcacatgcagccctaaaaagccaaaaaaaaaaaaaaaaaaaaaagacttgttaatggtgtattaggttcttagtaaataatataaatcctcaaaggcaaaaaaaaaatcaaaatacaataaaactgtcagaatcatgagctgaaactaaggtggagatctgggagcagctaactataatcttggggtcccctagggacaaagacttacatgaaggctgagatcagaactaaacccagactcactgcaggtagggaagctgaacctgggactctcacatgcaccagagacaatcaaaggaggcatatgtggtcccagatggcagatcaaatgcaggaggaaaacatcccaaatttagagccttgggataccccaagatcctccaaatagaagctcacaatcaaagatcagtcaagattttaaacaagaaaaaccaccgtatgccgcatatgaaaatcatcaaaaccagaaaatagaacctcaaggatttcagatgttagaattatcaggtaattacaataataactaataaagtatttctatgtcctgggcactattctaagtatttcatcatcccccagcacaggagctttcttctgacacaggcacagccccctccaaaaaaaaaaa

SEQ ID NO: 116 - DNA, 1847 bp, Sus scrofa
  source 1..1847: mol_type = other DNA
tccaaactcctccacctcccacaaccgcaaacaaaaagcccacacagtgagtaagaagtcacttaactggaagcgagattgaaagggagagatggggggaggtgccttctgacatcacccacagccaacacccggttgggtggaagtcattcaggcgggaaggattttgcgacatctaggtgtgccacctgctctgaggccaaagggcctgtgaatgactccaggtgttgttccagcccctgagatgggtgtcaggttacaagacgcataatcattccagtttggcaacttcacctgtggggctgttttaatcgacctgccccacagtccccaaaccactcctggaagacagatagcagtgggacacggccagctccccagggggagagagatgctggacgcaagggaccgaggcccaccctccacttaagcctcctccgaccagtacaagctgggcatcgctgacctgcacgccagccagggccccattctggggccctgctcctgacttgggctgctgacctcagctccaggctatagaaaattcaggaagatcacagagctggcatggatcccgccagtgatcaacccctgaacagccttgatgtcgccccccagcgcaaaccccgcacccccctgcagaccttcaaaaaggtggggatccccatcatcgcggcactgctgggcctggcagccatcgtcgtcgtggctattctcatcaaggtgattctggacaaatactacttcctctgctggcagcccctccacttcatcccgagagagcagctgtgtgacggccagcaggactgtgcctcaggggaggatgagcagctctgtgtcaagaaattccccgatgggcccccagtggcagtccgcctctccagggaccgatctaccctgcaggtgctgcacccagccacacagacctgggcctctgcctgtttcgacaacttcacggaagctctggccaagacagcctgtgggcagatgggctatgacagcaaacccaccttcagagctgtgaggattggcccagaccaagatctggatgttgtgaaaatcacagagaatgaccaggagcttcgggtgcaaaactcaagcggcccctgtctctcaggctccctggtttcgctgcactgccttgcctgcggggagagcctgaaggcccctcgggtggtgggcggggagaaggcctctgtggattcctggccctggcaggtcagcatccagtacaacaaacagcacatctgtggagggagcatcctggacccccactggatcctcacggcagcccactgcttctggaagcatctcgatgtgcccaactggaaggtgagggccggctcagacaaactgggcaacttcccatccctgcctgtggccaagatcttcatcactgagcccaacagcacgtcccccaaagagaaagacattgcccttgtgaagctgcagctcccgctcacgttctccggcacagtcaggcccatctgcctgcccttctctgacgaggaactcactccaggcaccccagtctgggtcatcggctggggctttacggaacctaatggagggaagatgtctgatcatctgctgcaggcgtccgtccaggtcattaaccgcactcggtgcaacgcggaggatgcgtaccagggggaagtcacggagaagatgctgtgtgcaggcctcctggagggcggtgtggacacctgccagcagcccctgctctggcctcccacagggtgacagtggtgggcctctgatgtatcactctggccggtggcaagcagtaggcatcgtgagctggggctatggctgtggggggccgagtacccctggagtatacacca

SEQ ID NO: 117 - DNA, 2103 bp, Bos taurus
  source 1..2103: mol_type = other DNA
actcctggaagacggcagtgggaggcacgcgccagctacgccaggggatagagatgctggacgccgggaaccaccaaggcctgccctgcacttagggctcctccagccaatgttgcctggggatcgctgacctgctggccagccagggcccggtcccgagagaccctcatcctgacttgggctggcgacctcagctccaggccatggaaaattcagaagatcgacgagccagggtggatcctgagatcaatcgacccctgaacagcctcgatgtcacccccctgcgcaaatcccgcaccccgttggagatcttcaaaaaagtagggatcccgatcatcgcagcagtgctgatcctggcaaccatcattgtggtggccgtcctcatcaaggtgattctggacagttacttcttcttctgtgggcagcccctcctcttcatcccaagggagcaggtgtgtgatggccaccaggactgtgcctcgggggaggatgagaagtactgcgtcaagaaactccccgacggacctccagtggcagtccgcctctccagggaccgatccaccctgcaggtgctgaaccccgccacgaagaactgggcctccgcctgtttcgacagcttcacagaagctctggccaagaccgcctgtcggcagatgggctatgacagcaaacccaccttcagagctgtggacgctggcccagcccaagatctggacatcgtcaaagtcacagagaacctgcaggagcttcaggtgcaaaactcaagtggcccctgtctctcaggttccctggtttccctgcagtgccttgcctgcggacagagtgtgaaggcccctcgggtggtgggtgggaaggaggcctctgtggattcttggccttggcaggtcagcatccagtatgacaaacaacacatctgtggagggagcatcctagacccacactggatcctcacggctgcccactgcttctggaagcatctcgatgtgcccaactggaaggtgagggctggctcagacagattgggcagcttcccatccctgcctgtggccaagatcttcatccttgagcctaacgccacatatcccagagagcacgacattgccctcgtgaagctgcagctcccgctcacgttctccggcacaatcaggcccatctgcctgcccttctctgatgaggagctcactccaggcaccccactctgggtcatcggatggggctttacagaagagaacggagggaagatgtctgacatactgcagcagggatccgtccagctcatcaacagaactcggtgcaatgcagaggatgcgtaccagggggaggtcaccaagacgatgatgtgtgcaggcctcccggagggcggcgtggacacctgccagggtgacagtggtggccctctgatgtatcactctgactggtggcaagtggtgggcatcgtgagctggggccatggctgtggggggcctactaccccaggagtatacactaaggtcacagcctatctcaactggatctacaatgtccggaagtctgatccatgatgctcctgcccttgaagtgctggaaactgcttccccctgccctgcccacatgggaatcccccagaagtcagacacagaccaagagccccctggggcacacctctctgcccacactctcagcatttctaggtgcagcaaaggacctgcaatcctgtaagtcaccccacagctcacaggcacgcaggaaatcagcagctccagttcagctgcccatgatgctctcggcatcccaaggagggagagacacagcccaccgaacaagccagcctccaagactgaggtctcagaaggcattgctaagccaagaactttctactctattgaatggaaacagacgctcttctaagagcctagatcaccaagggctggagtggagggaggagagggtctgagccagccctctgcctccccacccatccccaagcccacctgaacaagaaaccgtttataatgggaaatgtgctgtcctgctcttggctcaagtactgtttcttactgtttcgtattgtggccactgtttattaaactggtgcatgaagtctttgaaaaaaaaaaaaaaaaa

SEQ ID NO: 118 - DNA, 2237 bp, Sus scrofa
  source 1..2237: mol_type = other DNA
cagtttcattttgtgtctatttctttgcagctgttgcctgctgggtttgatttcccatcatcaggtttctgaaaaaaatctgcatgcttccaggactccaatggaataaagtaaagcctaaactgccagggagagagaaacgagaaaccagggcagctcagctcagaagaaaaggaagaggttaaacactataactttcacattgaagtagctgtctttttttttttctcaccctgaaggatctctgctccagatcgggtgacaccctggagatggctacagagatcctcatggcaggcccggagtgcctcattgagaacatcaatgggcgtctcatggttaatctggaaactgcaaagatcctgtctgccatcacgcagcctgtggtggttgtggttatcgtgggcccctaccgcacaggcaaatcctacctgatgtacaagctggctgggaagaacaagggcttctctctgggctccacggtgcagtctcacacgaagggcatctggatgtggtgtgtgcctcaccccaggaatccagaccacaccctggttctccttgacaccgagggcctgggggatgtagagaagggagacaaccagaatgactcctggctctttgccctggcagttctcctgagcagcacccttatatacaacagcgtggggaccatcaaccagcaggccatggaccaactgcactatgtgacagagctgacagattgcatcagggcaaaatcctcgcctggcagacatgaggtcaaagactcagctgactttgtaagcttctttccagactttgtgtggaccctgagagatttctccctggacctacaagcaaatggggaacccatcactcctgacgagtacctggagaattcactgaagctgaagcaaggtaatgataaaaaaactaaaagctttaatgcacctcggttatgtattcgaaagttcttcccagaaaagaagtgcttcatctttgaccgccctgctcagaggaagtaccttgtccacctggagcagctaccggaagaagatgtggatcctgaattcagagaacaggttgaagaattctgctcttacatcctcagccattccaaggtcaagactctctcaggaggcatcacagtcaatgggcctcgtctggagaccctggtgctgacctttgtcaacaccatcagcagtggggaccggccctgcatggagaatgcagtcctggccctggctgagatcgagaactcggccgcagtgcagaaggccattgcccactatgagcagcagatggctgagaagctgcagctgcccacggagaccctgcaggagctgctggacctgcaccgggccagtgagaaggaggccattgacgtcttcatgaccaagtctttcaaggatgtggaccaagtgttccaaaacagattaatggaacagttagaagcaaaactcgatgacttttgtgaccagaacatggaagcctcctcagatcactgtatgactttacttcaagatatttttgggcctctgtatgaagatgtgaagcagggaacattctctaaaccaggaggttttcgtctctctattcggaagaggcaggagctaaagaataagtactatcaggcccccagaaaggggatacaggcagaggagaccctgaagaaatatttggattccagggagggaatggatgctgcacttctccagactgatcagtcactaacagaaaaggaaaaagagattgaagtgcaatggatgaaatccgaatctttagaagctgcaaacaaaatttcagaggaaatgcaaaagaagcatgagcagttgatgcaggagaaagagaagagttttcatgaacatatgaaacagctggctgagaaaatggaaaaggagagggctcagttactggctgagcaagagagggttcttacgcttaaatttcaggaaaaagaacgtcttctcgaacagggattcaagaaggagagcctgaaacttcttaaagagatgaaagctatccagttaagaaacagtgaatcattcccgatgtgtagcattctctaaaaatccaaggaacaaaatcccctaaccagcttccatctgctaagaacaaggtgaacaagcaacttcaagatgccaaaccattgccactcaactaagcttaagacctgtaaagtctgcaaggatgaaataaatagtgaaggcaacatgtttcaaaaaaaaaaaaaaaaaaagaaaaaaaaaaaaaaaaaa

SEQ ID NO: 119 - DNA, 1808 bp, Sus scrofa
  source 1..1808: mol_type = other DNA
ccagttggggaggaggtagctaggtgtgcagggattggctgagccactcaagccccacctctgccagttcagagataaggacaggaaagaagactccctaggaagaggacagaagtttcgggggattaagtgctccagacagaaggatgctgcagctgagcctatcctggctgggactggggccagtggaagcctcaccatggctactcctgctcctggttggggcttcctggctcctggcccgagtcctggtctggacctgcacctcccttgacaatgttcgccgcctccgaggtttcccgcagcctccaaaaccgaactggctcttgggtcacatgggccaggtcccccccacggaggagggcatgacaaaactcactcagatgatgaacacataccccaatggatttatgatctggatgggccccatcactcccatcattgttttgtgccatcctgacttgatccggactatggccaatgcctcagctgccgtcgcacccaaggatgtggtcttctacgactttctgaagccctggctgggggatgggctcctgctgagtgctggtgacaagtggagcagccaccgtcgcatgctgacacctgccttccacttcaacatcctgaagccctacatgaagattttcaatgacagcgtgaatgtcatgcatgccaagtggcagcgtctggtcacagagggccacaaccgtttggacatgtttgaacacatcagcctcatgaccctggacagtctgcagaaatgtgtcttcagctttgacagcaattgccaagagaagcctagtgaatatattgctgccatcttggagctcagtgctcttgtggcaaaacggcaccagcagatcttcctgcacctggacttcctgtactacctcacccctgatgggtggcgcttccataaggcctgccgcctagttcacgacttcacagatgccgtcatccaggagcggcgcaacacccttcccacagagggcattgatgacttcctcaaggccaaggctaaggctaaaactttggacatcattgatgtgctcctgctgaccaaggatgaagatgggaagggattgtcagatgaagatatccgagctgaagctgacaccttcatgtttgagggccatgacaccacagccagtggcctctcctgggtcctgtacaacctggcaaagcacccagaataccaggagcgctgccggcaggaggtgcatgagctcctgagggaccgtgagcctaaagagattgaatgggatgacctggcacagttgcccttcctgaccatgtgcatcaaggagagtctgcggttgcatcctcccgtcactgtcatctcccatcgctgtacccaggacattgtgctcccagatggccggatcatccccaaaggtgttatctgcctcatcagtatttttgggacccaccacaacccattggtgtggcaagatcctgaggtctatgatccattccgcttcgacccagaaaacatcaaggagaggtcacctctggctttcatccctttttcagcggggcccaggaactgcattgggcagacattcgctatgacagagatgaaagtggtcctggctctcacactactgcgcttccgtgttctgccggtggaggaagagccgcgcaggaagccagagctgattctgcgcgcggagggcggactttggctgcgggtggagccgctgagcgcaagccctcagtgaggcatttcctccaggcctgggactcagtccaataccatccccatacttgcgcagattcccagcaataaaactgtgttgtcactttta

(19) United States
(12) Patent Application Publication
(10) Pub. No.: US 2025/0129385 A1
(43) Pub. Date: Apr. 24, 2025

(54) GENE-EDITING METHODS FOR EMBRYONIC STEM CELLS

(71) Applicant: ABS Global, Inc., DeForest, WI (US)

(72) Inventors: Benjamin Beaton, DeForest, WI (US); Rebecca Krisher, Cottage Grove, WI (US); Andrew Mark Cigan, Madison, WI (US); Michael McLachlan, Verona, WI (US)

(73) Assignee: ABS Global, Inc., DeForest, WI (US)

(21) Appl. No.: 18/835,162

(22) PCT Filed: Jan. 31, 2023
(86) PCT No.: PCT/US2023/061656

§ 371 Date: Aug. 1, 2024

Related U.S. Application Data
(60) Provisional application No. 63/305,369, filed on Feb. 1, 2022.

(51) Int. Cl. C12N 15/873; C12N 5/0735; C12N 9/22; C12N 15/11; C12N 15/90

(52) U.S. Cl. CPC C12N 15/873; C12N 5/0606; C12N 9/22; C12N 15/11; C12N 15/902; C12N 15/907; C12N 2310/20; C12N 2510/00

(57) ABSTRACT

The present disclosure relates generally to methods for improving traits or fitness in cattle, pigs, or aquatic organisms via gene editing. In particular, the methods comprise modifying genes in embryonic stem cells of livestock (e.g., cattle, pigs, or fish) for the purpose of improving a trait (e.g., promoting pathogen resistance, fertility, lactation, and native traits that support more rapid growth or feed efficiency). The methods particularly include CRISPR-CAS9 editing of porcine and bovine embryonic stem cells. Also disclosed herein are methods of producing and cultivating porcine embryonic stem cells under feeder free conditions.

### CROSS REFERENCE TO RELATED APPLICATION

This application claims the benefit of and priority to U.S. Provisional Application, 63/305,369 filed on Feb. 1, 2022. Application 63/305,369 is hereby incorporated by reference in its entirety.

### SEQUENCE LISTING

The instant application contains a Sequence Listing which has been submitted in ST_26 xml format and is hereby incorporated by reference in its entirety. Said xml copy, created on Jan. 19, 2023, is named TD-28-2022-WO1-SEQ.xml.

### TECHNICAL FIELD

The present technology relates generally to methods for improving traits or fitness in cattle, pigs, sheep or aquatic organisms via gene editing. In particular, the methods comprise modifying genes in embryonic stem cells of cattle, pigs, sheep or aquatic organisms for the purpose of improving a trait (e.g., promoting pathogen resistance, fertility, lactation, and native traits that support more rapid growth or feed efficiency).

### BACKGROUND

The following description of the background of the present technology is provided simply as an aid in understanding the present technology and is not admitted to describe or constitute prior art to the present technology.

In domestic pigs and other livestock species, the conventional means for generating genetically engineered animals is somatic cell nuclear transfer (SCNT) or cloning, where somatic cells (typically fetal fibroblasts) are modified to introduce the intended genetic modification and then used as nuclear donors for SCNT. However, the efficiency of somatic cell gene targeting is typically low and requires a laborious and time-consuming screening process. Correct recombination events are rare (1 in 10⁶-10⁷ cells), often monoallelic, and require a second round of targeting for generating homozygous/biallelic modifications.

Microinjection of CRISPR reagents directly into the embryos is a straightforward and preferred option for breeding genetically modified live offspring. The prevailing consensus is that the genome in the early zygote is in an open conformation and is thus ideal for targeting most loci with relative ease. That said, the use of microinjected zygotes also has limitations, chief among them are the large number of donor animals required to retrieve in vivo derived embryos for embryo transfers, the high degree of mosaicism, and the lack of predictable efficiencies in generating a cohort of edited animals that are required of a study. More efficacious methods of CRISPR editing are needed.

### SUMMARY OF THE PRESENT TECHNOLOGY

In one aspect, the present disclosure provides a method for improving a trait in a non-human animal subject such as cattle, pigs, sheep or aquatic organisms comprising: (a) contacting embryonic stem cells obtained from cattle, pigs or aquatic organisms with an effective amount of a gene editing agent to obtain genetically modified embryonic stem cells comprising a modification in a target gene of interest, wherein the gene editing agent is configured to modify the target gene of interest; (b) transplanting or injecting a nucleus from the genetically modified embryonic stem cells into an enucleated oocyte to obtain an oocyte comprising the modification in the target gene of interest; and (c) establishing an embryo from the oocyte comprising the modification in the target gene of interest.

In some embodiments, the embryonic stem cells are obtained from cattle and the method further comprises implanting the embryo into a cow or heifer recipient and allowing the embryo to develop into a fetus having the improved trait. In certain embodiments, the embryonic stem cells are obtained from swine and the method further comprises implanting the embryo into a gilt or sow recipient and allowing the embryo to develop into a fetus having the improved trait. In any of the preceding embodiments of the methods disclosed herein, the embryonic stem cells are isolated from inner cell mass (ICM) of blastocyst stage embryos. The blastocyst stage embryos may be obtained via in vitro fertilization. In some embodiments, the blastocyst stage embryos are cultured in vitro in cell culture medium before the embryonic stem cells are isolated. Additionally or alternatively, in certain embodiments, the embryonic stem cells express one or more genes including, but not limited to, DUSP6, ZIC3, DNMT3A/B ZIC2, OTX2, TET1, NCAM1, TET3, MYC, CD47, HOXA1, FOXA2, GATA6, TBX3, OCT4, CDX2, and SOX2. In any of the foregoing embodiments of the methods disclosed herein, the cell culture media can comprise: (i) inactivated feeder cells; (ii) an effective amount of Fibroblast Growth Factor 2 (FGF2); and (iii) an effective amount of one or more of an inhibitor of Wnt signaling. Additionally or alternatively, in some embodiments of the methods disclosed herein, the embryonic stem cells are cultured in vitro in cell culture medium prior to step (a) or step (b).

In other embodiments, the embryonic stem cells are fish embryonic stem cells and the method further comprises cultivating the embryo until hatching under conditions appropriate for culturing fish. In some embodiments, the fish embryonic stem cells are obtained from blastula-stage embryos by adopting feeder layer or feeder-free culture conditions. The blastula stage embryos may be obtained via external or in vitro fertilization. Examples of feeder cells include zebrafish embryonic fibroblasts, buffalo rat liver cells and the rainbow trout spleen cell line RTS34st. In feeder-free culture systems, components of ES cell-conducive medium may include fish embryo extract from medaka, basic fibroblast growth factor and fish serum, which are capable of supporting self-renewal of disassociated midblastula embryo (MBE) cells on a gelatin-coated culture dish. Additionally or alternatively, in certain embodiments, the fish embryonic stem cells express one or more of oct4, nanog, klf4, sox2, myc, ronin, sall4, and tcf3 (tcf7ll). Additionally or alternatively, in some embodiments of the methods disclosed herein, the fish embryonic stem cells are cultured in vitro in cell culture medium prior to step (a) or step (b). Additionally or alternatively, in some embodiments of the methods disclosed herein, the embryonic stem cells can be from aquatic organisms other than fish, including shrimp and other crustaceans, and mollusks (e.g., gastropods, cephalopods, and bivalves).

Exemplary traits include, but are not limited to, high altitude adaptation and response to hypoxia, cold acclimation, body size and stature, resistance to disease and bacterial infection, reproduction, and growth and feed efficiency. In particular, exemplary cattle traits include, but are not limited to, high altitude adaptation and response to hypoxia, cold acclimation, body size and stature, resistance to disease and bacterial infection, reproduction, milk yield and components, growth and feed efficiency, or polled phenotype. In some embodiments of the methods, the modification in the target gene of interest is a knock-in, a knock-out, a deletion or a point mutation. Additionally or alternatively, in some embodiments, the modification in the target gene of interest comprises a modification of at most 200 nucleotides.

Additionally or alternatively, in some embodiments, the target gene of interest is ANP32, ANPEP, TMPRSS1, TMPRSS2, TMPRSS4, NANOS1, NANOS2, CD163, Melanocortin-4 receptor (MC4R), HMGA, IGF2, HAL, RN, Mx1, BAT2, EHMT2, PRDM1, PRDM14, or ESR. Additionally or alternatively, in certain embodiments, the target gene of interest is PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR, or ZBTB.

In any and all embodiments of the methods disclosed herein, the gene editing agent comprises a DNA editing system such as a meganuclease, a zinc finger nucleases (ZFN), a transposon, a transcription-activator like effector nuclease (TALEN) or CRISPR. In some embodiments, the gene editing agent comprises an endonuclease, such as Cas9. In other embodiments, the gene editing agent does not comprise an endonuclease. Additionally or alternatively, in some embodiments, the gene editing agent comprises at least one gRNA operatively linked to a constitutive or inducible promoter. The gene editing agent may be provided to the embryonic stem cells as DNA, RNA or Ribonucleoprotein (RNP).

In various embodiments, a method for improving a trait in terrestrial or aquatic livestock can comprise: (a) contacting embryonic stem cells obtained from terrestrial or aquatic livestock with an effective amount of a gene editing reagent to obtain gene edited embryonic stem cells comprising a desired gene edit; (b) producing an embryo from the gene edited embryonic stem cells wherein at least some cells of the embryo comprise the desired gene edit. In various configurations, wherein the embryonic stem cells can be obtained from a non-human mammal; and can further comprise: (c) implanting the embryo into a surrogate mother. In various configurations, the embryonic stem cells can be obtained from fish; and can further comprise: (c) culturing the embryo in conditions suitable for hatching. In various configurations, the producing an embryo can comprise contacting one or more gene edited embryonic stem cells obtained in (a) with an oocyte.

In various embodiments, a method for establishing a gene edited cell line with an improved trait in terrestrial or aquatic livestock can comprise: (a) contacting embryonic stem cells obtained from terrestrial or aquatic livestock with an effective amount of a gene editing reagent to obtain gene edited embryonic stem cells comprising a desired gene edit; (b) producing a gene edited embryonic stem cell line from the gene edited embryonic stem cells. In various configurations, the method can further comprise (c) producing an embryo from one or more gene edited embryonic stem cells obtained from the gene edited embryonic stem cell line produced in (b).

In various configurations, the embryonic stem cells can be obtained from cattle, pigs, or fish. In various configurations, the embryonic stem cells can be obtained from cattle. In various configurations, the embryonic stem cells can be obtained from pigs. In various configurations, the desired gene edit can be in an ANP32, ANPEP, TMPRSS2, TMPRSS4, NANOS2, CD163, Melanocortin-4 receptor (MC4R), HMGA, IGF2, HAL, Mx1, BAT2, EHMT2, PRDM1, PRDM14, or ESR gene. In various configurations, the desired gene edit can be in a PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, or TIGAR gene.

In various configurations, the desired gene edit can affect a trait comprising high altitude adaptation and response to hypoxia, cold acclimation, body size and stature, resistance to disease and bacterial infection, reproduction, or growth and feed efficiency. In various configurations, the desired gene edit can affect a trait comprising high altitude adaptation and response to hypoxia, cold acclimation, body size and stature, resistance to disease and bacterial infection, reproduction, milk yield and components, growth and feed efficiency, or polled phenotype.

In various configurations, the gene editing reagent can comprise a meganuclease, a zinc finger nuclease (ZFN), a transcription-activator like effector nuclease (TALEN) or at least one guide RNA (gRNA). In various configurations, the gene editing reagent can comprise at least one gRNA. In various configurations, the gene editing reagent can further comprise a CAS protein. In various configurations, the gene editing reagent can further comprise CAS9. In various configurations, the desired gene edit can comprise a deletion, a nucleotide substitution that encodes a substituted amino acid, or a premature stop codon. In various configurations, the contacting the embryonic stem cells with the gene editing reagent can comprise electroporation or nucleofection.

The present teachings can encompass an embryo produced by the methods of any configurations of the present teachings. The present teachings can encompass a line of embryonic stem cells produced by the methods of any configuration of the present teachings. In some configurations, the cells can be grown under feeder free conditions.

Also disclosed herein are genetically modified pigs, cattle, sheep or aquatic organisms produced by any and all embodiments of the methods disclosed herein.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Definitions

Throughout this application, various embodiments of the present technology may be presented in a range format. It should be understood that the description in range format is merely for convenience and brevity and should not be construed as an inflexible limitation on the scope of the present technology. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “biophysically effective amount” refers to an amount of nucleic acid in a system under physiological conditions (such as temperature, pH, exposure to percent oxygen, etc.) sufficient to produce a biological effect. In the case of embodiments drawn to the use of CRISPR, the biophysically effective amount refers to an amount of nucleic acid in a system under physiological conditions (such as temperature, pH, exposure to percent oxygen, etc.) sufficient to associate to or bind a Cas protein or functional fragment thereof in the presence of a Cas protein or functional fragment thereof. In some embodiments, the nucleic acid is a sgRNA, or a crRNA/tracr RNA duplex in a biophysically effective amount. In some embodiments, the Cas protein or functional fragment thereof is chosen from any of cas1, cas2, cas3, cas4, cas4, cas5, cas6, cas6e, cas6f, cas7, cas8a1, cas8a2, cas8b, cas8c, cas9, cas10, cas10d, csy1, csy2, csy3, cse1, cse2, csc1, csc2, csa5, csn2, csm2, csm3, csm4, csm5, csm6, cmr1, cmr3, cmr4, cmr5, cmr6, csb1, csb2, csb3, csx17, csx14, csx10, csx16, csaX, csx3, csx1, csx1, csx2, or csx15 or functional fragments thereof.

“Cas binding domain” refers to a nucleic acid element or domain within a nucleic acid sequence or polynucleotide sequence that, in a biophysically effective amount, will bind or have an affinity for one or a plurality of proteins (or functional fragments thereof) encoded by one or a plurality of CRISPR-associated genes. In some embodiments, in the presence of one or a plurality of proteins (or functional fragments thereof) and a target sequence, the one or plurality of proteins and the nucleic acid element forms a biologically active CRISPR complex and/or can be enzymatically active on a target sequence. The terms “CRISPR-associated genes” refer to any nucleic acid that encodes a regulatory or expressible gene that regulates a component or encodes a component of the CRISPR system. In some embodiments, the terms “CRISPR-associated genes” refer to any nucleic acid sequence that encodes any of the CRISPR enzymes selected from among cas1, cas2, cas3, cas4, cas4, cas5, cas6, cas6e, cas6f, cas7, cas8a1, cas8a2, cas8b, cas8c, cas9, cas10, cas10d, csy1, csy2, csy3, cse1, cse2, csc1, csc2, csa5, csn2, csm2, csm3, csm4, csm5, csm6, cmr1, cmr3, cmr4, cmr5, cmr6, csb1, csb2, csb3, csx17, csx14, csx10, csx16, csaX, csx3, csx1, csx1, csx2, or csx15 (or functional fragments or variants thereof that are at least about 70, 75, 80, 85, 90, 95, 96, 97, 98, or 99% homologous to the CRISPR enzymes described herein). In some embodiments, the terms “Cas-binding domain” or “Cas protein-binding domain” refers to a nucleic acid element or domain within a nucleic acid sequence or polynucleotide sequence that, in a biophysically effective amount, will bind to or have an affinity for one or a plurality of CRISPR enzymes selected from among cas1, cas2, cas3, cas4, cas4, cas5, cas6, cas6e, cas6f, cas7, cas8a1, cas8a2, cas8b, cas8c, cas9, cas10, cas10d, csy1, csy2, csy3, cse1, cse2, csc1, csc2, csa5, csn2, csm2, csm3, csm4, csm5, csm6, cmr1, cmr3, cmr4, cmr5, cmr6, csb1, csb2, csb3, csx17, csx14, csx10, csx16, csaX, csx3, csx1, csx1, csx2, or csx15 (or functional fragments or variants thereof that are at least about 70, 75, 80, 85, 90, 95, 96, 97, 98, or 99% homologous to the CRISPR enzymes described herein). In some embodiments, the Cas binding domain consists of no more than about 10, 11, 12, 13, 14, 15, 16, 17 18, 19, 20, 25, 30, 35, 40, 41, 42, 43, 44, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active CRISPR system at a concentration and within microenvironment suitable for CRISPR system formation. In some embodiments, the composition or pharmaceutical compositions comprises one or a combination of sgRNA, crRNA, and/or tracrRNA that consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active CRISPR enzyme or functional fragment thereof at a concentration and within microenvironment suitable for CRISPR system formation and CRISPR enzymatic activity on a target sequence. In some embodiments, the Cas protein derived from the Cas9 family of Cas proteins or a functional fragment thereof.

As used herein the terms “culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic culture media such as, for example, minimum essential media a (MEM-α) (HyClone Thermo Scientific, Waltham, Mass., USA), DMEM/F12, GlutaMAX (Life Technologies, Carlsbad, Calif, USA), Neurobasal Medium (Life Technologies, Carlsbad, Calif., USA), KO-DMEM (Life Technologies, Carlsbad, Calif, USA), DMEM/F12 (Life Technologies, Carlsbad, Calif, USA), supplemented with the necessary additives as is further described herein. In some embodiments, the cell culture media can be a mixture of culture media. In some embodiments, all ingredients included in the culture media of the present disclosure are substantially pure and tissue culture grade. “Conditioned medium” and “conditioned culture medium” are used interchangeably and refer to culture medium that cells have been cultured in for a period of time and wherein the cells release/secrete components (e.g., proteins, cytokines, chemicals, etc.) into the medium.

The term “DNA-binding domain” refers to an element or refers to a nucleic acid element or domain within a nucleic acid sequence or sgRNA that is complementary to a target sequence. In some embodiments, in a biophysically effective amount upstream from a Cas-binding domain, the DNA-binding domain will bind or have an affinity for one or a plurality of target nucleic acid sequences such that, in the presence of a biologically active CRISPR complex, one or plurality of Cas proteins can be enzymatically active on the target sequence. In some embodiments, the DNA binding domain consists of no more than about 20, 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming Watson Crick base pairs with a target sequence as part of a biologically active CRISPR system at a concentration and microenvironment suitable for CRISPR system formation.

“Embryo” is a multicellular diploid eukaryote in early stage of development.

The term “eukaryotic cell” as used herein refers to any cell of a eukaryotic organism. Eukaryotic organisms include single- and multi-cellular organisms. In some embodiments, the eukaryotic cell is an animal cell or a cell of a vertebrate. According to a specific embodiment, the eukaryotic cell is a cell of a fish. Exemplary fish include, but are not limited to, salmon, tuna, medaka, pollock, catfish, cod, haddock, sea bass, tilapia, Arctic char, trout, and carp. According to a one embodiment, the eukaryotic cell is a mammalian cell of pigs, sheep, or cattle. The eukaryotic cell may be a primary cell, a cell line, a somatic cell, a germ cell, a stem cell, an embryonic stem cell, an adult stem cell, a hematopoietic stem cell, a mesenchymal stem cell, an induced pluripotent stem cell (iPS), a gamete cell, a zygote cell, a blastocyst cell, an embryo, a fetus and/or a donor cell.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, such as embryonic cells within the first couple of cell divisions after fertilization are the only cells that can differentiate into embryonic and extra-embryonic cells and are able to develop into a viable animal. The phrase “pluripotent stem cells” refers to cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPS). The multipotent stem cells include adult stem cells and hematopoietic stem cells.

As used herein, “embryonic stem cells” refer to embryonic cells that are capable of differentiating into cells of all three embryonic germ layers (i.e., endoderm, ectoderm and mesoderm), or remaining in an undifferentiated state. In some embodiments, the embryonic stem cells are derived from blastula stage embryos or inner cell mass (ICM) of the blastocyst. In certain embodiments, embryonic stem cells may comprise cells which are obtained from the blastula stage embryos, blastocysts before implantation of the embryo (e.g., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and/or cells originating from an unfertilized ova which are stimulated by parthenogenesis (parthenotes).

The embryonic stem cells of some embodiments of the present technology can be obtained using well-known cell-culture methods. For example, embryonic stem cells can be isolated from blastula stage embryos or blastocysts. Blastocysts are typically obtained from in vivo preimplantation embryos or from in vitro fertilized (IVF) embryos. Alternatively, a single cell embryo can be expanded to the blastocyst stage. It will be appreciated that commercially available stem cells can also be used according to some embodiments of the present technology. Embryonic stem cells have been previously obtained from various species, including zebrafish (Collodi, P., et al., 1992, Cell Biol Toxicol. 8:43-61), medaka (Wakamatsu, Y., et al., 1994, Mol Mar Biol Biotechnol. 3:185-191; Hong, Y., et al., 1996 Mech Dev. 60:33-44), mouse (Mills, A. A. and Bradley, A., 2001, Trends Genet. 17: 331-339), golden hamster (Doetschman, T., et al., 1988, Dev Biol. 127: 224-227, rat (Iannaccone, P. M., et al., 1994, Dev Biol. 163: 288-292), rabbit (Giles, J. R., et al., 1993, Mol. Reprod. Dev., 36: 130-138; Graves, K. H. & Moreadith, R. W., 1993, Mol. Reprod. Dev., 36: 424-433), several domestic animal species (Notarianni, E., et al., 1991, J. Reprod. Fertil. Suppl. 43: 255-260; Wheeler, M. B., 1994, Reprod. Fertil. Dev. 6: 563-568; Mitalipova, M., et al., 2001, Cloning 3: 59-67) and non-human primate species (Rhesus monkey and marmoset; Thomson, J. A., et al., 1995, Proc. Natl. Acad. Sci. USA. 92: 7844-8; Thomson et al., 1996, Biol Reprod. 55: 254-9], shrimp, prawn, crab, crayfish, lobster [WO2020149791], and mollusks [Yoshino et al., 2013, Can J Zool. 91(6): doi: 10.1139/cjz-2012-0258.].

As used herein, the term “stable embryonic stem cell (ESC)” refers to ESCs (e.g., bovine embryonic stem cells) that are pluripotent, easy to propagate using single cell dissociation by trypsin, and that maintained long-term stable morphology, karyotype, pluripotency markers expression, and epigenetic features typical of pluripotent cells. In one aspect, the ESCs have been maintained in culture for over 50 weeks, or for over 60 weeks, or for over 70 weeks, or for over 80 weeks, or for over 90 weeds, or for over 100 weeks.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “fertility” includes one or more of the following: a level or degree of ability of an animal to conceive and bear young; a level or degree of ability of an animal to become pregnant; a level or degree of ability of an animal to reproduce; a level or degree of ability of a spermatozoa to fertilize an oocyte; a level or degree of ability of an oocyte to be fertilized by a spermatozoa; and a level or degree of ability of an oocyte fertilized by a spermatozoa to develop into a zygote capable of progressing through embryonic and fetal development. Various methods of evaluating fertility, such as in gametes, are described in U.S. Pat. Pub. 2020/0347347 by Roti-Roti, which is incorporated herein by reference.

The term “fluorescent protein” refers to a polypeptide that emits fluorescence and is typically detectable by flow cytometry, microscopy or any fluorescent imaging system, therefore can be used as a basis for selection of cells expressing such a protein.

As used herein, the term “gene” means a segment of DNA that contains all the information for the regulated biosynthesis of an RNA product, including promoters, exons, introns, and other untranslated regions that control expression.

“Gene of interest” refers to a nucleotide, nucleotide sequence, DNA, RNA, polypeptide, sequence on a chromosome or within the genome of an organism which is to be genetically modified or altered in some way. The gene of interest can be mutated or its nucleotide sequence may be altered.

“Genetically modified” or “genetic modification” means a gene or other DNA sequence that is altered from its native state (e.g. by insertion mutation, deletion mutation, nucleic acid sequence mutation, or other mutation), or that a gene product is altered from its natural state (e.g. by delivery of a transgene that works in trans on a gene's encoded mRNA or protein, such as delivery of inhibitory RNA or delivery of a dominant negative transgene). The present technology relates to methods for producing a livestock animal, e.g. a sheep, goat, cow, pig, or fish comprising a targeted germline genetic modification.

In one embodiment, the genetic modification results in reduced expression of one or more genes and/or proteins in the animal and/or progeny thereof. Thus, in this embodiment, a gene knockout animal can be produced. As used herein, “reduced expression” of one or more genes and/or proteins is meant that the translation of a polypeptide and/or transcription of a gene in the cells of an animal produced using the methods of the technology, or progeny thereof, is reduced at least 10%, or at least 20%, or at least 30%, or at least 40%, or at least 50%, or at least 60%, or at least 70%, or at least 80%, or at least 90%, or at least 95%, or at least 98%, or at least 99%, or 100% relative to an isogenic animal lacking the genetic modification.

In an alternate embodiment, the genetic modification is the insertion of a transgene. The ability to target the transgene to a site of interest can be beneficial in that the transgene is interested at a site known or suspected to not cause any deleterious effects on the animal. The transgene may encode any functional protein or polynucleotide (such as an antisense polynucleotide or a dsRNA for RNAi). In some embodiments, the transgene encodes a protein which is expressed in the animal.

In some embodiments, the transgene comprises one or more regulatory (promoter) elements operably linked to an open reading frame of interest (such as encoding a protein). “Operably linked” as used herein refers to a functional relationship between two or more nucleic acid (e.g., DNA) segments. Typically, it refers to the functional relationship of a transcriptional regulatory element to a transcribed sequence. For example, a promoter is operably linked to a coding sequence, such as an open reading frame encoding, if it stimulates the transcription of the coding sequence in an appropriate cell. Generally, promoter transcriptional regulatory elements that are operably linked to a transcribed sequence are physically contiguous to the transcribed sequence, i.e., they are cis-acting. The transgene may also comprise a 3′ non-translated sequence, for example from about 50 to 1,000 nucleotide base pairs, which may include a transcription termination sequence. A 3′ non-translated sequence may contain a transcription termination signal which may or may not include a polyadenylation signal and any other regulatory signals capable of effecting mRNA processing.

“Genetic modification associated with the gene of interest” means a mutation or other genetic modification which corresponds to a gene that is being studied or selected for. The genetic modification may involve either endogenous or exogenous genes.

As used herein, the term “targeted germline genetic modification” refers to any genetic modification, such as but not limited to deletion, substitution or insertion, made by way of human intervention at a predetermined location in the genome. In some embodiments, the targeted germline genetic modification is in a sex chromosome. In an alternate embodiment, the targeted germline genetic modification is a somatic chromosome. In some embodiments, all cells of the animal comprise the targeted germline genetic modification. Animals produced using the methods of the technology can be screened for the presence of the targeted germline genetic modification. This can step can be performed using any suitable procedure known in the art. For instance, a nucleic acid sample, such as a genomic DNA sample, can be analyzed using standard DNA amplification and sequencing procedures to determine if the genetic modification is present at the targeted site (locus) in the genome. In some embodiments, the screening also determines whether the animal is homozygous or heterozygous for the genetic modification.

As used herein, a “genetically modified animal” refers to an animal in which one or more, cells of the animal contains the targeted germline genetic modification.

As used herein, the term “gene edit”, “gene edited”, or “genetically edited” refers to an organism where human intervention, such as but without limitation by using a gene editing effector, has created a genetic difference in its genome when compared to a wild type genome of the same organism. These differences can include but are not limited to nucleotide substitutions, excision of a start codon, or small deletions that do not introduce frame shift mutations into the genome but may excise an exon or form a premature stop codon when the ends of the deletion are ligated together. A gene edit does not introduce DNA from another species into an organism.

A “gene edited animal” refers to an animal with one or more cells comprising a gene edit.

The term “heterologous” refers to a combination of elements not naturally occurring. For example, heterologous DNA refers to DNA not naturally located in the cell, or in a chromosomal site of the cell. In some embodiments, the heterologous DNA includes a gene foreign to the cell. A heterologous expression regulatory element is such an element operatively associated with a different gene than the one it is operatively associated with in nature. A heterologous nucleus is a nucleus that has been transferred from another cell.

“Addition of heterologous sequence” is meant to be any introduction of deoxyribonucleotide, nucleotide or DNA sequence within a gene, chromosome or genome of an organism. Also known as a “knock-in” which is meant an alteration in the nucleic acid sequence that replaces the endogenous, normal or wild-type allele with an exogenous allele. The exogenous allele includes but is not limited to a full length gene of the same or a different species, a section of a gene of the same or different species, a replacement cassette and reporter or selection genes and markers. Knock-in mutations can be produced by homologous recombination, site-specific deletion, repair mechanism provocation via targeting proteins, as well as site specific targeted DNA transposons. A “coding sequence” or a sequence “encoding” an expression product, such as a RNA, polypeptide, protein, or enzyme, is a nucleotide sequence that, when expressed or translated, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “individual”, “patient”, or “subject” can be an individual organism, a vertebrate, or a mammal. In some embodiments, the individual, patient or subject is cattle, pig, or sheep.

The term “mutation,” as used herein, refers to a substitution of a residue within a sequence, e.g., a nucleic acid or amino acid sequence, with another residue, or a deletion or insertion of one or more residues within a sequence, a frameshift mutation, or a nonsense mutation. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4.sup.th ed., Cold Spring Harbor Laboratory Press, Cold Spring Harbor, N.Y. (2012)).

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after synthesis or polymerization, such as by conjugation with a labeling component.

In the context of this disclosure, the term “oligonucleotide” also refers to a plurality of nucleotides joined together in a specific sequence from naturally and non-naturally occurring nucleobases. Nucleobases of the disclosure are joined through a sugar moiety via phosphorus linkages, and include any one or combination of adenine, guanine, cytosine, uracil, thymine, xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, other aza and deaza thymidines, other aza and deaza cytosines, other aza and deaza adenines, other aza and deaza guanines, 5-trifluoromethyl uracil, and 5-trifluoro cytosine. The sugar moiety may be deoxyribose or ribose. The sugar moiety may be a modified deoxyribose or ribose with one or more modifications on the C1, C2, C3, C4, and/or C5 carbons. The oligonucleotides of the disclosure may also comprise modified nucleobases or nucleobases having other modifications consistent with the spirit of this disclosure, and in particular modifications that increase their nuclease resistance in order to facilitate their use as therapeutic, diagnostic or research reagents.

The oligonucleotides of the disclosure also include those nucleic acid sequences disclosed herein that comprise nucleosides connected by charged linkages, and/or whose sequences are divided into at least two sub-sequences. In some embodiments, a first, second, and third sub-sequence or domains include a nucleotide binding domain (or DNA-binding domain), a Cas-binding domain, and a transcription terminator domain. In some embodiments, a first, second, and third sub-sequence or domains include a nucleotide binding domain, a Cas-binding domain, and a transcription terminator sequence, but, if any two domains are present then they must be oriented such that the nucleotide binding domain precedes the Cas-binding domain which, in turn precedes the transcription terminator domain in a 5′ to 3′ orientation. Any of the nucleosides within any of the domains may be 2′-substituted-nucleosides linked by a first type of linkage. The second sub-sequence includes nucleosides linked by a second type of linkage. In some embodiments, there exists a third sub-sequence whose nucleosides are selected from those selectable for the first sub-sequence, and the second sub-sequence is positioned between the first and the third sub-sequences. Such oligonucleotides of the disclosure are known as “chimeras,” or “chimeric” or “gapped” oligonucleotides.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “transcription terminator domain” refers to a nucleic acid element or domain within a nucleic acid sequence (or polynucleotide sequence) that, in a biophysically effective amount, prevents bacterial transcription when the CRISPR complex is in a bacterial species and/or creates a secondary structure that stabilizes the association of the nucleic acid sequence to one or a plurality of Cas proteins (or functional fragments thereof) encoded by one or a plurality of CRISPR-associated genes such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of no more than about 25, 30, 35, 40, 45, 50, 60, 70, 80, 90, 100, 110, 120, 130, 1s 140, 150, 160, 170, 180, 190, 200, 210, 220, 230, 240, 250 or more nucleotides in length and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

As used herein, the term “probe” refers to any molecule that may bind or associate, indirectly or directly, covalently or non-covalently, to any of the substrates and/or reaction products and/or nucleic acid sequences disclosed herein that are heterologous as compared to the genome that they modify and whose association or binding is detectable using the methods disclosed herein.

In some embodiments, the probe is a fluorogenic, fluorescent, or chemiluminescent probe, an antibody, or an absorbance-based probe. In some embodiments, an absorbance-based probe, for example the chromophore pNA (para-nitroanaline), may be used as a probe for detection and/or quantification of a nucleic acid sequence comprising a modification from an in vitro gene editing technique disclosed herein relative to a wild-type cell. In some embodiments, the probe may comprise any molecule that may bind or associate, indirectly or directly, covalently or non-covalently, to a protein or peptide or chemical substance that is known to be present in unmodified cells and absent in modified cells, such that detecting an absence of a signal corresponds with the particular genetic modification being absent. Such is the case, in some embodiments, when trying to identify a knock-out type of genetic modification. A probe may be immobilized, adsorbed, or otherwise non-covalently bound to a solid surface, such that upon exposure to an embryonic stem cell for a time period sufficient to perform an enzymatic reaction, it can be enzymatically cleaved. In some embodiments, cleavage of the substrate causes a biological change in the nature or chemical availability of one or more probes such that cleavage enables detection of the reaction product. For instance, if the step of detecting comprises use of FRET, cleavage of the substrate disclosed herein causes one of the chromophores to emit a fluorescent light under exposure to a wavelength sufficient to activate such a fluorescent molecule. The intensity, length, or amplitude of a wavelength emitted from fluorescent marker can be measured and is, in some embodiments, proportional to the presence, absence or quantity of enzyme present in the reaction vessel, thereby the quantity of enzyme can be determined from detection of the intensity of or fluorescence at a known wavelength of light.

An “activity-based probe,” as used herein, refers to a certain embodiment of probe comprising a small molecule that binds to or has affinity for a molecule such as a substrate that binds an enzyme in the presence of such an enzyme, such that its bound or unbound state confers an activity readout to the enzyme. In some embodiments, the activity-based probe covalently or non-covalently binds to an enzyme or functional fragment herein. In some embodiments, the binding of the activity-based probe modifies the physical or biological activity of the enzyme. In some embodiments, the activity-based probe can be fluorescent or chemiluminescent. In some embodiments, the activity-based probe has a measurable activity of one value if the enzyme is inactive and another measurable activity if in an activated state. In some embodiments, any of the methods disclosed herein comprise a step of detecting the presence or absence of a genetic modification by exposing an activity-based probe to the surface of an embryonic stem cell.

As used herein, the terms “fluorogenic” and “fluorescent” probe refer to any molecule (dye, quantum dot, peptide, or fluorescent marker) that emits a known and/or detectable wavelength of light upon exposure to a known wavelength of light. In some embodiments, the substrates or peptides with known cleavage sites recognizable by any of the enzymes expressed by one or a plurality of mucinous cysts are covalently or non-covalently attached to a fluorogenic probe. In some embodiments, the attachment of the fluorogenic probe to the substrate creates a chimeric molecule capable of a fluorescent emission or emissions upon exposure of the substrate to the enzyme and the known wavelength of light, such that exposure to the enzyme creates a reaction product which is quantifiable in the presence of a fluorimeter. In some embodiments, light from the fluorogenic probe is fully quenched upon exposure to the known wavelength of light before enzymatic cleavage of the substrate and the fluorogenic probe emits a known wavelength of light, the intensity of which is quantifiable by absorbance readings or intensity levels in the presence of a fluorimeter and after enzymatic cleavage of the substrate. In some embodiments, the fluorogenic probe is a coumarin-based dye or rhodamine-based dye with fluorescent emission spectra measurable or quantifiable in the presence of or exposure to a predetermined wavelength of light. In some embodiments, the fluorogenic probe comprises rhodamine. In some embodiments, the fluorogenic probe comprises rhodamine-100. Coumarin-based fluorogenic probes are known in the art, for example in U.S. Pat. Nos. 7,625,758 and 7,863,048, which are herein incorporated by reference in their entireties. In some embodiments, the fluorogenic probes are a component to, covalently bound to, non-covalently bound to, intercalated with one or a plurality of markers from a genetic modification in the one or plurality of cells. In some embodiments, the fluorogenic probes are chosen from ACC or AMC. In some embodiments, the fluorogenic probe is a fluorescein molecule. In some embodiments, the fluorogenic probe is capable of emitting a resonance wave detectable and/or quantifiable by a fluorimeter after exposure to one or a plurality of enzymes disclosed herein. “Fluorescence microscopy,” which uses the fluorescence to generate an image, may be used to detect the presence, absence, or quantity of a fluorescent probe. In some embodiments, fluorescence microscopy comprises measuring fluorescence resonance energy transfer (FRET) within a FRET-based assay.

A “chemiluminescent probe” refers to any molecule (dye, peptide, or “chemiluminescent” marker) that emits a known and/or detectable wavelength of light as the result of a chemical reaction. Chemiluminescence differs from fluorescence or phosphorescence in that the electronic excited state is the product of a chemical reaction rather than of the absorption of a photon. Non-limiting examples of chemiluminescent probes are luciferin and aequorin molecules. In some embodiments, a chemiluminescent molecule is covalently or non-covalently attached to a substrate disclosed herein or an enzyme, such that the excited electronic state can be quantified to determine directly that an enzyme, such as an aspartyl protease, is in a reaction vessel, or, indirectly, by quantifying the amount of reaction product was produced after activation of the probe on the substrate or a portion of the substrate.

### Gene Editing Agents

The present disclosure provides a method for improving a trait or improving fitness in cattle, pigs, sheep or aquatic organisms including fish, crustaceans—including but not limited to shrimp, and mollusks (e.g., gastropods, cephalopods, and bivalves)—the method comprising contacting an embryonic stem cell of the cattle, pigs, sheep or aquatic organisms with an effective amount of a gene editing agent, wherein the gene editing agent is configured to modify a target gene of interest, thereby improving a trait in the cattle, pigs, sheep or aquatic organisms. The methods disclosed herein are useful for generating gene edited cattle, pigs, sheep, or aquatic organisms, which show an improvement in fitness or in one or more traits without negatively affecting survival of the animals. Examples of gene editing agents include, but are not limited to transposons, Xanthomonas TAL nucleases, Zinc Finger Nucleases, or CRISPR/Cas systems. The gene editing agent may be provided to a cell as DNA, RNA or RNP.

### Transposons

In some embodiments, the genetic modification agent is a DNA transposon. Genetic modification of stem cells using DNA transposons is described, for example, in WO/2010/065550, which is incorporated by reference herein in its entirety. DNA transposons can be viewed as natural gene delivery vehicles that integrate into the host genome via a “cut-and-paste” mechanism. These mobile DNA elements encode a transposase flanked by inverted terminal repeats (ITRs) that contain the transposase binding sites necessary for transposition. Any gene of interest flanked by such ITRs can undergo transposition in the presence of the transposase supplied in trans. As noted, a “transposon” is a segment of DNA that can move (transpose) within the genome. A transposon may or may not encode the enzyme transposase, necessary to catalyze its relocation and/or duplication in the genome. Where a transposon does not code for its transposase enzyme, expression of said enzyme in trans may be required when carrying out the method of the present technology in cells not expressing the relevant transposase itself. Furthermore, a transposon must contain sequences that are required for its mobilization, namely the terminal inverted repeats containing the binding sites for the transposase. The transposon may be derived from a bacterial or a eukaryotic transposon. Further, the transposon may be derived from a class I or class II transposon. Class II or DNA-mediated transposable elements are preferred for gene transfer applications, because transposition of these elements does not involve a reverse transcription step, which pertains in transposition of Class I or retro-elements and which can introduce undesired mutations into transgenes. For example, see Miller, A. D., RETROVIRUSES 843 (Cold Spring Harbor Laboratory Press, 1997), and Verma, L. M., et al., 1997, Nature 389: 239-242. Transposons also can be harnessed as vehicles for introducing “tagged” genetic mutations into genomes, which makes such genomic sites of transposon integration/mutation easy to clone and defined at the DNA sequence level. This fact makes transposon-based technology especially attractive in cultures of germline stem cells derived from a variety of species. For example, the first mutagenesis screens in mammals have established that the Sleeping Beauty transposon system can generate a high number of random mutations in both mouse and rat germinal cells in vivo. Alternatively, where mutagenic events can first be selected and then used to produce experimental animal models, random mutagenesis would be more feasible in tissue culture.

Similarly, transposons can be harnessed as vehicles for introducing mutations into genomes. Specifically, genes may be inactivated by transposon insertion. For example, such genes are then “tagged” by the transposable element, which can be used for subsequent cloning of the mutated allele. In addition to gene inactivation, a transposon may also introduce a transgene of interest into the genome if contained between its ITRs. Moreover, to insert or knock in a DNA construct or gene of interest into an existing site of transposition, stem cell lines or animals produced with transposons are designed to contain recognition sequences (e.g., pLox sties) within the transposon that act as substrates for DNA recombinase enzymes (e.g., Cre-recombinase). This would allow a gene of interest flanked by compatible recombinase recognition sequences to be delivered into the cells or animals in trans with a recombinase to catalyze integration of the gene of interest into the genomic locus of the transposon. The transposon may carry as well the regulatory elements necessary for the expression of the transgene, allowing for successful expression of the gene. Examples of transposon systems that can transpose in vertebrates have recently became available, such as Sleeping Beauty, piggyBac, Tol2 or Frog Prince. Each transposon system can be combined with any gene trap mechanism (for example: enhancer, promoter, polyA, or slice acceptor gene traps) to generate the mutated gene, as discussed below. Sleeping Beauty (SB) and Frog Prince (FP) are TcI transposons, whereas piggyBac (PB) was the founder of the PB transposon family and Tol2 is a hAT transposon family member. Both the Sleeping Beauty and the Frog Prince transposon are found in vertebrates as inactive copies, from which active transposon systems have been engineered. The Tol2 transposon also has been found in vertebrates as an active transposon. The piggyBac transposon was originally found as an active transposon in insects but was subsequently shown to have high levels of activity in vertebrates, too, as shown in Ding S et al, Cell 122:473 (2005). Each of these elements has their own advantages; for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore mutagenize overlapping, but distinct sets of genes. Therefore, to achieve the best possible coverage of genes, the use of more than one element is particularly preferred. In addition to naturally occurring transposons, modified transposon systems such as those disclosed in European patent documents EP1594973, EP 1594971, and EP1594972 also may be employed. In some embodiments, the transposons used possess highly elevated transpositional activity. In some embodiments, the transposon is a eukaryotic transposon, such as the Sleeping Beauty transposon, the Frog Prince transposon, the piggyBac transposon, or the Tol2 transposon, as discussed above.

The use of gene-trap constructs for insertional mutagenesis in tissue culture, where trapped events can easily be selected for, is advantageous over the random mutagenesis in animals. Gene trap vectors report both the insertion of the transposon into an expressed gene, and have a mutagenic effect by truncating the transcript through imposed splicing. Cells selected for a particular gene trap event can be used for the generation of animal models lacking this specific genetic function.

When transposons are used in insertional mutagenesis screens, transposon vectors typically constitute four major classes of constructs, suitable for identifying mutated genes rapidly. These contain a reporter gene, which should be expressed depending on the genetic context of the integration. Specific gene traps include, but are not limited to: (1) enhancer traps, (2) promoter traps, (3) polyA traps, and (4) splice acceptor traps. In enhancer traps, the expression of the reporter requires the presence of a genomic cis-regulator to act on an attenuated promoter within the integrated construct. Promoter traps contain no promoter at all. These vectors are only expressed if they land in-frame in an exon or close downstream to a promoter of an expressed gene. In polyA traps, the marker gene lacks a polyA signal, but contains a splice donor (SD) site. Thus, when integrating into an intron, a fusion transcript can be synthesized comprising the marker and the downstream exons of the trapped gene. Slice acceptor gene traps (or exon traps) also lack promoters, but are equipped with a splice acceptor (SA) preceding the marker gene. Reporter activation occurs if the vector is integrated into an expressed gene, and splicing between the reporter and an upstream exon takes place. The splice acceptor gene trap and polyA gene trap cassettes can be combined. In that case, the marker of the polyA trap part is amended with a promoter so that the vector also can trap downstream exons, and both upstream and downstream fusion transcripts of the trapped gene can be obtained. The foregoing constructs also offer the possibility to visualize spatial and temporal expression patterns of the mutated genes by using, e.g., LacZ or fluorescent proteins as a marker gene.

In some embodiments, the present technology relates to a method based on the combination of transposon-mediated insertional mutagenesis with a tissue culture system, e.g. culture of embryonic stem cells, which allows for the ready generation of in vitro embryonic stem cell libraries carrying a large number of different insertion events. Compared to classical nuclear transfer technologies and in vivo mutagenesis, moreover, this method is less costly and less labor-intensive, and it allows for the selection of the appropriate insertion(s) before establishing the corresponding animal models. Additionally, using these cells or libraries allows for establishment of a broader variety of animal models.

Libraries of embryonic stem cell lines can be generated by isolating and then pooling individual clonal lines with mutated genes. First, embryonic stem cell lines are genetically modified with a DNA construct that harbors a selectable marker, such as a gene encoding resistance to G418. Then, due to stable integration of the DNA construct into different locations within the genome, a mixed population of genetically distinct embryonic stem cell lines is selected using the selectable marker. By pooling these selected individual clonal lines with mutated genes, a library of mutant embryonic stem cell lines is generated.

The phrase “selectable marker” is employed here to denote a protein that enables the separation of cells expressing the marker from those that lack or do not express it. The selectable marker may be a fluorescent marker, for instance. Expression of the marker by cells having successfully integrated the transposon allows the isolation of these cells using methods such as, for example, FACS (fluorescent activated cell sorting). Alternatively, expression of a selectable marker may confer an advantageous property to the cell that allows survival of only those cells carrying the gene. For example, the marker protein may allow for the selection of the cell by conferring an antibiotic resistance to the cell. Consequently, when cells are cultured in medium containing said antibiotic, only cell clones expressing the marker protein that mediates antibiotic resistance are capable of propagating. By way of illustration, a suitable marker protein may confer resistance to antibiotics such as ampicillin, kanamycin, chloramphenicol, tetracycline, hygromycin, neomycin or methotrexate. Further examples of antibiotics are penicillins: ampicillin HCl, ampicillin Na, amoxycillin Na, carbenicillin disodium, penicillin G, cephalosporins, cefotaxim Na, cefalexin HCl, vancomycin, cycloserine. Other examples include bacteriostatic inhibitors such as: chloramphenicol, erythromycin, lincomycin, spectinomycin sulfate, clindamycin HCl, chlortetracycline HCl. Additional examples are marker proteins that allow selection with bactericidal inhibitors such as those affecting protein synthesis irreversibly causing cell death, for example aminoglycosides such as gentamycin, hygromycin B, kanamycin, neomycin, streptomycin, G418, tobramycin. Aminoglycosides can be inactivated by enzymes such as NPT II which phosphorylates 3′-OH present on kanamycin, thus inactivating this antibiotic. Some aminoglycoside modifying enzymes acetylate the compounds and block their entry in to the cell. Marker proteins that allow selection with nucleic acid metabolism inhibitors like rifampicin, mitomycin C, nalidixic acid, doxorubicin HCl, 5-flurouracil, 6-mercaptopurine, antimetabolites, miconazole, trimethoprim, methotrexate, metronidazole, sulfametoxazole are also examples for selectable markers.

In some embodiments, the present disclosure relates to methods of integrating an exogenous nucleic acid into the genome of at least one embryonic stem cell of a non-human animal such as cattle, pigs, sheep or aquatic organisms comprising administering directly to the embryonic stem cell: a) a transposon comprising the exogenous nucleic acid, wherein the exogenous nucleic acid is flanked by one or more inverted repeat sequences that are recognized by a transposase; and b) a transposase to excise the exogenous nucleic acid from a plasmid, episome, or transgene and integrate the exogenous nucleic acid into the genome.

Methods of genetically modifying cells of an animal using transposon are described, for example, in WO/2012/074758, which is incorporated by reference herein in its entirety. In some embodiments, the transposase is administered as a nucleic acid encoding the transposase. In some embodiments, the transposon and nucleic acid encoding the transposase are present on separate vectors. In some embodiments, the transposon and nucleic acid encoding the transposase are present on the same vector. When present on the same vector, the portion of the vector encoding the hyperactive transposase is located outside the portion carrying the inserted nucleic acid. In other words, the transposase encoding region is located external to the region flanked by the inverted repeats. Put another way, the tranposase encoding region is positioned to the left of the left terminal inverted repeat or to the right of the right terminal inverted repeat. In the aforementioned methods, the hyperactive transposase protein recognizes the inverted repeats that flank an inserted nucleic acid, such as a nucleic acid that is to be inserted into a target cell genome.

The elements of the PiggyBac transposase system are administered to the cell in a manner such that they are introduced into a target cell under conditions sufficient for excision of the inverted repeat flanked nucleic acid from the vector carrying the transposon and subsequent integration of the excised nucleic acid into the genome of the target cell. As the transposon is introduced into the cell “under conditions sufficient for excision and integration to occur,” the method can further include a step of ensuring that the requisite PiggyBac transposase activity is present in the target cell along with the introduced transposon. Depending on the structure of the transposon vector itself, such as whether or not the vector includes a region encoding a product having PiggyBac transposase activity, the method can further include introducing a second vector into the target cell that encodes the requisite transposase activity, where this step also includes an in vivo administration step.

The amount of vector nucleic acid comprising the transposon element, and in many embodiments the amount of vector nucleic acid encoding the transposase, which is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed.

The particular dosage of each component of the system that is administered to the cell varies depending on the nature of the transposon nucleic acid, e.g. the nature of the expression module and gene, the nature of the vector on which the component elements are present, the nature of the delivery vehicle and the like. Dosages can readily be determined empirically by those of skill in the art.

Once the vector DNA has entered the target cell in combination with the requisite transposase, the nucleic acid region of the vector that is flanked by inverted repeats, i.e. the vector nucleic acid positioned between the PiggyBac transposase-recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell.

The methods may be used to integrate nucleic acids of various sizes into the target cell genome. Generally, the size of DNA that is inserted into a target cell genome using the subject methods ranges from about 0.5 kb to 100.0 kb, usually from about 1.0 kb to about 60.0 kb, or from about 1.0 kb to about 10.0 kb.

The methods may result in stable integration of the nucleic acid into the target cell genome. By stable integration is meant that the nucleic acid remains present in the target cell genome for more than a transient period of time, and is passed on a part of the chromosomal genetic material to the progeny of the target cell. The subject methods of stable integration of nucleic acids into the genome of a target cell find use in a variety of applications in which the stable integration of a nucleic acid into a target cell genome is desired. The hyperactive transposase can be delivered as DNA, RNA, or protein.

In some embodiments, the present disclosure relates to a colony of transgenic animals each such transgenic animal comprising one or more exogenous nucleic acid sequences and one or two internal tandem repeat sequences of a transposon. The present disclosure also relates to one or more progeny from an animal comprising the one or more exogenous nucleic acid sequences and one or more internal tandem repeat sequences of the transposons. The present disclosure also relates to a colony of transgenic animals each such transgenic animal comprising one or more exogenous nucleic acid sequences and one or two internal tandem repeat sequences of a transposon described herein. The present disclosure also relates to one or more progeny from an animal comprising the one or more exogenous nucleic acid sequences and one or more internal tandem repeat sequences of the transposons described herein.

The hyperactive PiggyBac transposase system described herein can be used for germline mutagenesis in a vertebrate species. One method would entail the production of transgenic animals by, for example, pronuclear injection of newly fertilized oocytes. Typically, two types of transgenes can be produced; one transgene provides expression of the transposase (a “driver” transgene) and the other transgene (the “donor” transgene) comprises a transposon containing gene-disruptive sequences, such as a gene trap. The transposase may be directed to the germline via a ubiquitously active promoter, such as the ROSA26 (Gt(ROSA)26Sor), pPol2 (Polr2a), or CMV/beta-actin (CAG) promoters.

### Zinc Finger Nucleases

In some embodiments, the gene editing agent is a zinc-finger nuclease (ZFN). Methods of genetically modifying stem cells with ZFNs are described, for example, in WO2015200805, which is incorporated by reference herein in its entirety. In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a Fok1 endonuclease. In one embodiment, the nuclease agent comprises a first ZFN and a second ZFN, wherein each of the first ZFN and the second ZFN is operably linked to a Fok1 nuclease subunit, wherein the first and the second ZFN recognize two contiguous target DNA sequences in each strand of the target DNA sequence separated by about 5-7 bp spacer, and wherein the Fok1 nuclease subunits dimerize to create an active nuclease to make a double strand break. See, for example, US20060246567; US20080182332; US20020081614; US20030021776; WO/2002/057308A2; US20130123484; US20100291048; WO/2011/017293A2; and Gaj et al. (2013) Trends in Biotechnology, 31(7):397-405 each of which is herein incorporated by reference.

### TAL Nucleases

In some embodiments, the gene editing agent is a TAL nuclease. Xanthomonas TAL nucleases from the bacterium Xanthomonas, bind DNA sequences in a site-specific manner as a mechanism to regulate their genes. Methods of using TAL nuclease for genetic modification of stem cells are described, for example, in WO/2012/158986, which is incorporated by reference herein in its entirety. TAL nucleases can be modified in order to specifically bind to sites within the genome of many organisms. TAL nucleases may be used to introduce targeted double-stranded or single-stranded breaks in the DNA, which can lead to small deletions at the site of the break during the Non-Homologous End Joining (NHEJ) process, thereby producing gene knockouts in cells and organisms. TAL nucleases can also generate breaks in the DNA which can increase the frequency of exogenous sequence introduction by homologous recombination, thereby enabling specific gene editing (e.g. correction or mutation) or producing gene knock-ins in cells and organisms.

A central repeat domain containing multiple repeat units consisting of 33-35 amino acids determines nucleotide binding sites. Two essential adjacent amino acids known as repeat variable di-residue or RVDs are present in each repeat domain and separately specify a targeted base. The repeat domains and RVDs can be modified in order to target a gene or locus with high specificity (Mahfouz et a. (2011) PNAS 108, 6, 2623-2628). By fusing nuclease cleavage domains such as Fok1 to the TAL nucleases, a nuclease is produced which is able to generate mutations in the genome of organisms in a site-specific manner. In one embodiment, TAL nucleases are used to generate site specific mutations in embryonic stem cells. TAL nuclease DNA binding specificity depends on the number and order of repeats in the DNA binding domain. Repeats are generally composed of 34-35 amino acids. Nucleotide binding specificity is determined by the 12 and 13 amino acids, called the repeat variable diresidue (RVD), within the DNA binding domain repeats. The RVDs bind to one or more nucleotides and the code has been deciphered using arbitrary RVDs as follows: asaparagine/isoleucine (NI)=A; histidine/aspartic acid (HD)=C; asparagine/glycine (NG)=T; asparagines/asparagines (NN)=A, G; asparagines/serine (NS)=A, C, G and T. Since the RVD binding code is deciphered, natural or codon-optimized versions of natural TAL nucleases can be used as a scaffold to generate sequence specific DNA binding TAL nucleases. The repeats and RVDs in the DNA binding domains of TAL nucleases may be modified and synthesized to generate site specific DNA binding TAL nucleases. The DNA cleavage domain of nucleases are fused into the TAL nuclease to produce a hybrid TAL nuclease which binds to a specific site on the DNA and produces mutations.

The methods used in the present technology are comprised of a combination of genetic introduction methods, site-specific genetic modification or mutagenesis mechanisms of stem cells, and generation of site-specific genetically modified organisms from the stem cells. For all genetic modification or mutagenesis mechanisms one or more introduction and delivery method may be employed. The present technology may include but is not limited to the methods described below.

In some embodiments, the site-specific genetic modification is produced in a stem cell, e.g. an embryonic stem cell. These stem cells can proliferate as cultured cells and be genetically modified without affecting their ability to differentiate into other cell types, including germ line cells. Generating site-specific mutations in stem cells, which can then be used to produce a gene edited organism, first involves the design and development of a protein such as a TAL nuclease whose DNA binding domain is engineered for a specific target site within the genome. A protein consisting of both a DNA binding domain and a cleavage or insertional mutagenesis domain is developed.

In one embodiment of the present technology, a site-specific mutagenesis technology is expressed in stem cells generating site-specific mutations. The binding domains of the site-specific mutagenesis technologies are modified to bind a particular location in the genome. The site-specific mutagenesis technology may be introduced into stem cells via transfection using lipofectamine. A transfection mixture may be prepared by mixing transfectamine with the site specific mutagenesis technology TAL nucleases. After harvesting undifferentiated stem cells, one may then add transfection mixture to the cell suspension, incubate, wash and plate the stem cells onto fresh embryonic fibroblast (EF) feeder layers.

Screening for TAL nuclease mediated site specific modification such as knockout mutations via NHEJ or knockin mutations using homologous recombination (HR) is done by selection with co-transfected vectors. Stem cells are co-transfected with a TAL nuclease and a selection marker vector such as a fluorescent marker or antibody resistance within a lipid-based transfection reagent, 1 μg total DNA is transfected with a ratio of 500 ng TAL nuclease to 500 ng selection vector. Clones are isolated and propagated to sufficient numbers to isolate DNA for screening and sequencing.

### CRISPR Cas

In some embodiments, the gene editing agent comprises a CRISPR/Cas system. Methods of genetically modifying stem cells with the CRISPR/Cas system are described, for example, in WO2015200805, which is incorporated by reference herein in its entirety. Such systems can employ a Cas9 nuclease, which in some instances, is codon-optimized for the desired cell type in which it is to be expressed. The system further employs a fused crRNA-tracrRNA construct that functions with the codon-optimized Cas9. This single RNA is often referred to as a guide RNA or gRNA. Within a gRNA, the crRNA portion is identified as the “target sequence” for the given recognition site and the tracrRNA is often referred to as the ‘scaffold’. This system has been shown to function in a variety of eukaryotic and prokaryotic cells. Briefly, a short DNA fragment containing the target sequence is inserted into a guide RNA expression plasmid. The gRNA expression plasmid comprises the target sequence (in some embodiments around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid. The gRNA expression cassette and the Cas9 expression cassette are then introduced into the cell. See, for example, Mali P et al. (2013) Science 2013 Feb. 15; 339 (6121):823-6; Jinek M et al. Science 2012 Aug. 17; 337(6096):816-21; Hwang W Y et al. Nat Biotechnol 2013 March; 31(3):227-9; Jiang W et al. Nat Biotechnol 2013 March; 31(3):233-9; and, Cong Let al. Science 2013 Feb. 15; 339(6121):819-23, each of which is herein incorporated by reference.

The methods and compositions disclosed herein can utilize Clustered Regularly Interspersed Short Palindromic Repeats (CRISPR)/CRISPR-associated (Cas) systems or components of such systems to modify a genome within a cell. CRISPR/Cas systems include transcripts and other elements involved in the expression of, or directing the activity of, Cas genes. A CRISPR/Cas system can be a type I, a type II, or a type III system. The methods and compositions disclosed herein employ CRISPR/Cas systems by utilizing CRISPR complexes (comprising a guide RNA (gRNA) complexed with a Cas protein) for site-directed cleavage of nucleic acids.

### A. Cas RNA-Guided Endonucleases

Cas proteins generally comprise at least one RNA recognition or binding domain. Such domains can interact with guide RNAs (gRNAs, described in more detail below). Cas proteins can also comprise nuclease domains (e.g., DNase or RNase domains), DNA binding domains, helicase domains, protein-protein interaction domains, dimerization domains, and other domains. A nuclease domain possesses catalytic activity for nucleic acid cleavage. Cleavage includes the breakage of the covalent bonds of a nucleic acid molecule. Cleavage can produce blunt ends or staggered ends, and it can be single-stranded or double-stranded. Examples of Cas proteins include Cas1, Cas1B, Cas2, Cas3, Cas4, Cas5, Cas5e (CasD), Cas6, Cas6e, Cas6f, Cas7, Cas8al, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

Cas proteins can be from a type II CRISPR/Cas system. For example, the Cas protein can be a Cas9 protein or be derived from a Cas9 protein. Cas9 proteins typically share four key motifs with a conserved architecture. Motifs 1, 2, and 4 are RuvC-like motifs, and motif 3 is an HNH motif. The Cas9 protein can be from, for example, Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Additional examples of the Cas9 family members are described in WO 2014/131833, herein incorporated by reference in its entirety. Cas9 protein from S. pyogenes or derived therefrom is an exemplary enzyme. Cas9 protein from S. pyogenes is assigned SwissProt accession number Q99ZW2.

Cas proteins can be wild type proteins (i.e., those that occur in nature), modified Cas proteins (i.e., Cas protein variants), or fragments of wild type or modified Cas proteins. Cas proteins can also be active variants or fragments of wild type or modified Cas proteins. Active variants or fragments can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

Cas proteins can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

Some Cas proteins comprise at least two nuclease domains, such as DNase domains. For example, a Cas9 protein can comprise a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337:816-821, hereby incorporated by reference in its entirety.

One or both of the nuclease domains can be deleted or mutated so that they are no longer functional or have reduced nuclease activity. If one of the nuclease domains is deleted or mutated, the resulting Cas protein (e.g., Cas9) can be referred to as a nickase and can generate a single-strand break at a CRISPR RNA recognition sequence within a double-stranded DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839) or H840A (histidine to alanine at amino acid position 840) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Research 39:9275-9282 and WO 2013/141680, each of which is herein incorporated by reference in its entirety. Such mutations can be generated using methods such as site-directed mutagenesis, PCR-mediated mutagenesis, or total gene synthesis. Examples of other mutations creating nickases can be found, for example, in WO/2013/176772A1 and WO/2013/142578A1, each of which is herein incorporated by reference.

Cas proteins can also be fusion proteins. For example, a Cas protein can be fused to a cleavage domain, an epigenetic modification domain, a transcriptional activation domain, or a transcriptional repressor domain. See WO 2014/089290, incorporated herein by reference in its entirety. Cas proteins can also be fused to a heterologous polypeptide providing increased or decreased stability. The fused domain or heterologous polypeptide can be located at the N-terminus, the C-terminus, or internally within the Cas protein.

A Cas protein can be fused to a heterologous polypeptide that provides for subcellular localization. Such heterologous peptides include, for example, a nuclear localization signal (NLS) such as the SV40 NLS for targeting to the nucleus, a mitochondrial localization signal for targeting to the mitochondria, an ER retention signal, and the like. See, e.g., Lange et al. (2007) Biol. Chem. 282:5101-5105. Such subcellular localization signals can be located at the N-terminus, the C-terminus, or anywhere within the Cas protein. An NLS can comprise a stretch of basic amino acids, and can be a monopartite sequence or a bipartite sequence.

Cas proteins can also be linked to a cell-penetrating domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, for example, WO 2014/089290, herein incorporated by reference in its entirety. The cell-penetrating domain can be located at the N-terminus, the C-terminus, or anywhere within the Cas protein. Cas proteins can also comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g. eBFP, eBFP2, Azurite, mKalama1, GFPuv, Sapphire, T-sapphire), cyan fluorescent proteins (e.g. eCFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism.

Nucleic acids encoding Cas proteins can be stably integrated in the genome of the cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell.

B. Guide RNAs (gRNAs)

A “guide RNA” or “gRNA” includes an RNA molecule that binds to a Cas protein and targets the Cas protein to a specific location within a target DNA. Guide RNAs can comprise two segments: a “DNA-targeting segment” and a “protein-binding segment.” “Segment” includes a segment, section, or region of a molecule, such as a contiguous stretch of nucleotides in an RNA. Some gRNAs comprise two separate RNA molecules: an “activator-RNA” and a “targeter-RNA.” Other gRNAs are a single RNA molecule (single RNA polynucleotide), which can also be called a “single-molecule gRNA,” a “single-guide RNA,” or an “sgRNA.” See, e.g., WO/2013/176772A1, WO/2014/065596A1, WO/2014/089290A1, WO/2014/093622A2, WO/2014/099750A2, WO/2013142578A1, and WO 2014/131833A1, each of which is herein incorporated by reference. The terms “guide RNA” and “gRNA” include both double-molecule gRNAs and single-molecule gRNAs. An exemplary two-molecule gRNA comprises a crRNA-like (“CRISPR RNA” or “targeter-RNA”or “crRNA” or “crRNA repeat”) molecule and a corresponding tracrRNA-like (“trans-acting CRISPR RNA” or “activator-RNA” or “tracrRNA” or “scaffold”) molecule. A crRNA comprises both the DNA-targeting segment (single-stranded) of the gRNA and a stretch of nucleotides that forms one half of the dsRNA duplex of the protein-binding segment of the gRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA and the corresponding tracrRNA hybridize to form a gRNA. The crRNA additionally provides the single-stranded DNA-targeting segment that hybridizes to a CRISPR RNA recognition sequence. If used for modification within a cell, the exact sequence of a given crRNA or tracrRNA molecule can be designed to be specific to the species in which the RNA molecules will be used. See, for example, Mali et al. (2013) Science 339:823-826; Jinek et al. (2012) Science 337:816-821; Hwang et al. (2013) Nat. Biotechnol. 31:227-229; Jiang et al. (2013) Nat. Biotechnol. 31:233-239; and Cong et al. (2013) Science 339:819-823, each of which is herein incorporated by reference. The DNA-targeting segment (crRNA) of a given gRNA comprises a nucleotide sequence that is complementary to a sequence in a target DNA. The DNA-targeting segment of a gRNA interacts with a target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

The DNA-targeting segment can have a length of from about 12 nucleotides to about 100 nucleotides. For example, the DNA-targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. Alternatively, the DNA-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

The nucleotide sequence of the DNA-targeting segment that is complementary to a nucleotide sequence (CRISPR RNA recognition sequence) of the target DNA can have a length at least about 12 nt. For example, the DNA-targeting sequence (i.e., the sequence within the DNA-targeting segment that is complementary to a CRISPR RNA recognition sequence within the target DNA) can have a length at least about 12 nt, at least about 15 nt, at least about 18 nt, at least about 19 nt, at least about 20 nt, at least about 25 nt, at least about 30 nt, at least about 35 nt, or at least about 40 nt. Alternatively, the DNA-targeting sequence can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the DNA-targeting sequence can have a length of at about 20 nt.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise or consist of all or a portion of a wild-type tracrRNA sequence (e.g., about or more than about 20, 26, 32, 45, 48, 54, 63, 67, 85, or more nucleotides of a wild-type tracrRNA sequence). Examples of wild-type tracrRNA sequences from S. pyogenes include 171-nucleotide, 89-nucleotide, 75-nucleotide, and 65-nucleotide versions. See, for example, Deltcheva et al. (2011) Nature 471:602-607; WO 2014/093661, each of which is incorporated herein by reference in their entirety. Examples of tracrRNAs within single-guide RNAs (sgRNAs) include the tracrRNA segments found within +48, +54, +67, and +85 versions of sgRNAs, where “+n” indicates that up to the +n nucleotide of wild-type tracrRNA is included in the sgRNA. See U.S. Pat. No. 8,697,359, incorporated herein by reference in its entirety.

The percent complementarity between the DNA-targeting sequence and the CRISPR RNA recognition sequence within the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting sequence and the CRISPR RNA recognition sequence within the target DNA can be at least 60% over about 20 contiguous nucleotides. As an example, the percent complementarity between the DNA-targeting sequence and the CRISPR RNA recognition sequence within the target DNA is 100% over the 14 contiguous nucleotides at the 5′ end of the CRISPR RNA recognition sequence within the complementary strand of the target DNA and as low as 0% over the remainder. In such a case, the DNA-targeting sequence can be considered to be 14 nucleotides in length. As another example, the percent complementarity between the DNA-targeting sequence and the CRISPR RNA recognition sequence within the target DNA is 100% over the seven contiguous nucleotides at the 5′ end of the CRISPR RNA recognition sequence within the complementary strand of the target DNA and as low as 0% over the remainder. In such a case, the DNA-targeting sequence can be considered to be 7 nucleotides in length.

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Guide RNAs can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ poly adenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and the like); and combinations thereof.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the RNA. The DNA encoding the gRNA can encode a single RNA molecule (sgRNA) or separate RNA molecules (e.g., separate crRNA and tracrRNA). In the latter case, the DNA encoding the gRNA can be provided as separate DNA molecules encoding the crRNA and tracrRNA, respectively. DNAs encoding gRNAs can be stably integrated in the genome of the cell and operably linked to a promoter active in the cell. Alternatively, DNAs encoding gRNAs can be operably linked to a promoter in an expression construct.

Alternatively, gRNAs can be prepared by various other methods. For example, gRNAs can be prepared by in vitro transcription using, for example, T7 RNA polymerase (see, for example, WO 2014/089290 and WO 2014/065596). Guide RNAs can also be a synthetically produced molecule prepared by chemical synthesis.

### C. CRISPR RNA Recognition Sequences

The term “CRISPR RNA recognition sequence” includes nucleic acid sequences present in a target DNA to which a DNA-targeting segment of a gRNA will bind, provided sufficient conditions for binding exist. For example, CRISPR RNA recognition sequences include sequences to which a guide RNA is designed to have complementarity, where hybridization between a CRISPR RNA recognition sequence and a DNA targeting sequence promotes the formation of a CRISPR complex. Full complementarity is not necessarily required, provided there is sufficient complementarity to cause hybridization and promote formation of a CRISPR complex. CRISPR RNA recognition sequences also include cleavage sites for Cas proteins, described in more detail below. A CRISPR RNA recognition sequence can comprise any polynucleotide, which can be located, for example, in the nucleus or cytoplasm of a cell or within an organelle of a cell.

The CRISPR RNA recognition sequence within a target DNA can be targeted by (i.e., be bound by, or hybridize with, or be complementary to) a Cas protein or a gRNA. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001)). The strand of the target DNA that is complementary to and hybridizes with the Cas protein or gRNA can be called the “complementary strand,” and the strand of the target DNA that is complementary to the “complementary strand” (and is therefore not complementary to the Cas protein or gRNA) can be called “noncomplementary strand” or “template strand.”

The Cas protein can cleave the nucleic acid at a site within or outside of the nucleic acid sequence present in the target DNA to which the DNA-targeting segment of a gRNA will bind. The “cleavage site” includes the position of a nucleic acid at which a Cas protein produces a single-strand break or a double-strand break. For example, formation of a CRISPR complex (comprising a gRNA hybridized to a CRISPR RNA recognition sequence and complexed with a Cas protein) can result in cleavage of one or both strands in or near (e.g., within 1, 2, 3, 4, 5, 6, 7, 8, 9, 10, 20, 50, or more base pairs from) the nucleic acid sequence present in a target DNA to which a DNA-targeting segment of a gRNA will bind. If the cleavage site is outside of the nucleic acid sequence to which the DNA-targeting segment of the gRNA will bind, the cleavage site is still considered to be within the “CRISPR RNA recognition sequence.” The cleavage site can be on only one strand or on both strands of a nucleic acid. Cleavage sites can be at the same position on both strands of the nucleic acid (producing blunt ends) or can be at different sites on each strand (producing staggered ends). Staggered ends can be produced, for example, by using two Cas proteins, each of which produces a single-strand break at a different cleavage site on each strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the CRISPR RNA recognition sequence of the nickase on the first strand is separated from the CRISPR RNA recognition sequence of the nickase on the second strand by at least 2, 3, 4, 5, 6, 7, 8, 9, 10, 15, 20, 25, 30, 40, 50, 75, 100, 250, 500, or 1,000 base pairs.

Site-specific cleavage of target DNA by Cas9 can occur at locations determined by both (i) base-pairing complementarity between the gRNA and the target DNA and (ii) a short motif, called the protospacer adjacent motif (PAM), in the target DNA. The PAM can flank the CRISPR RNA recognition sequence. Optionally, the CRISPR RNA recognition sequence can be flanked by the PAM. For example, the cleavage site of Cas9 can be about 1 to about 10 or about 2 to about 5 base pairs (e.g., 3 base pairs) upstream or downstream of the PAM sequence. In some cases (e.g., when Cas9 from S. pyogenes or a closely related Cas9 is used), the PAM sequence of the non-complementary strand can be 5′-NiGG-3′, where Ni is any DNA nucleotide and is immediately 3′ of the CRISPR RNA recognition sequence of the non-complementary strand of the target DNA. As such, the PAM sequence of the complementary strand would be 5′-CC N2-3′, where N2 is any DNA nucleotide and is immediately 5′ of the CRISPR RNA recognition sequence of the complementary strand of the target DNA. In some such cases, Ni and N2 can be complementary and the Ni-N2 base pair can be any base pair (e.g., Ni=C and N2=G; Ni=G and N2=C; Ni=A and N2=T, Ni=T, and N2=A).

Examples of CRISPR RNA recognition sequences include a DNA sequence complementary to the DNA-targeting segment of a gRNA, or such a DNA sequence in addition to a PAM sequence. For example, the target motif can be a 20-nucleotide DNA sequence immediately preceding an NGG motif recognized by a Cas protein (see, for example, WO 2014/165825). The guanine at the 5′ end can facilitate transcription by RNA polymerase in cells. Other examples of CRISPR RNA recognition sequences can include two guanine nucleotides at the 5′ end (e.g., GGN2ONGG) to facilitate efficient transcription by T7 polymerase in vitro. See, for example, WO 2014/065596.

The CRISPR RNA recognition sequence can be any nucleic acid sequence endogenous or exogenous to a cell. The CRISPR RNA recognition sequence can be a sequence coding a gene product (e.g., a protein) or a non-coding sequence (e.g., a regulatory sequence) or can include both.

In one embodiment, the target sequence is immediately flanked by a Protospacer Adjacent Motif (PAM) sequence. In one embodiment, the gRNA comprises a third nucleic acid sequence encoding a Clustered Regularly Interspaced Short Palindromic Repeats (CRISPR) RNA (crRNA) and a trans-activating CRISPR RNA (tracrRNA). In another embodiment, the genome of the pluripotent embryonic stem cell comprises a target DNA region complementary to the target sequence. In some such methods, the Cas protein is Cas9. Active variants and fragments of nuclease agents (i.e. an engineered nuclease agent) may also be used. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus, in some embodiments, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site. The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally or constitutively expressed within the cell. Thus, the polynucleotide encoding the nuclease agent can be contained in an expression cassette and be operably linked to a conditional promoter, an inducible promoter, a constitutive promoter, or a tissue-specific promoter. Alternatively, the nuclease agent is introduced into the cell as an mRNA encoding a nuclease agent.

In specific embodiments, the polynucleotide encoding the nuclease agent is stably integrated in the genome of the cell and operably linked to a promoter active in the cell. In other embodiments, the polynucleotide encoding the nuclease agent is in the same targeting vector comprising the insert polynucleotide, while in other instances the polynucleotide encoding the nuclease agent is in a vector or a plasmid that is separate from the targeting vector comprising the insert polynucleotide.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example, the polynucleotide encoding the nuclease agent can be modified to substitute codons having a higher frequency of usage in a given eukaryotic cell of interest, as compared to the naturally occurring polynucleotide sequence.

The present disclosure also provides a method of genetically modifying an embryonic stem cell comprising contacting the embryonic stem cell with one or more components of a CRISPR system, such CRISPR system comprising a nucleic acid sequence comprising an sgRNA comprising at least one or a combination of domains from a 5′ to 3′ orientation: a DNA-binding domain, a Cas-protein binding domain, and a transcription terminator domain, wherein each domain comprises from about 1 to about 150 nucleotides. In some embodiments, the entire sgRNA comprises no more than about 110, 105, 104, 103, 102, 101, 100, 99, 98, 97, 96 or 95 or fewer nucleotides. In some embodiments, the methods comprise contacting a biophysically effective amount of one or a plurality of CRISPR system components to the embryonic stem cell for a period of time sufficient to modify the genome of the embryonic stem cell.

The sgRNA may be generated in situ such that they hybridize with or bind to a target gene of interest to thereby inhibit expression of the target gene. The hybridization can occur via Watson-Crick base pairing to form a stable duplex.

Additionally or alternatively, in some embodiments, expression of the sgRNA may be regulated by operably linking the sgRNA to a gene regulatory sequence (e.g., promoter or enhancers). The gene regulatory sequence may be a heterologous or an endogenous gene regulatory sequence (e.g., promoter or enhancer). Additionally or alternatively, in some embodiments, the gene regulatory sequence (e.g., promoter or enhancer) may be constitutive or inducible. Additionally or alternatively, in some embodiments, the gene regulatory sequence (e.g., promoter or enhancer) may drive ubiquitous expression of the sgRNA molecule, or limit expression of the sgRNA in cell type-specific or a tissue specific manner.

### Methods for Isolating and Culturing Mammalian Embryonic Stem Cells

Mammalian embryonic stem cells (ESCs) may be derived from the inner cell mass of pre-implantation blastocysts. In some embodiments, ESCs can be captured and expanded from the inner cell mass (ICM) of blastocyst stage embryos. The methods as disclosed herein use the disclosed compositions and culture conditions for the establishment of ESCs (e.g., bovine embryonic stem cells) that are pluripotent, easy to propagate using single cell dissociation by trypsin or other proteases, and that maintained long-term stable morphology, karyotype, pluripotency markers expression, and epigenetic features typical of pluripotent cells.

In certain aspects the present disclosure, embryonic stem cells are isolated from a developed zygote, for example a zygote that has been generated or isolated in vitro. The in vitro zygote may be cultured for several days to generate the embryonic stem cell line. For example, in some embodiments, the zygote is cultured in vitro for at least 1, 2, 3, 4, 5, 6, 7, 8, 9, or 10 days to generate the embryonic stem cell line. Any of these values may be used to define a range for the number of days that the zygote is cultured in vitro. For example, the zygote may be cultured for 1 to 10 days, for 4 to 6 days, or from 4 to 10 days. In some embodiments, the embryonic stem cells are isolated from a sheep, a goat, a cow, or a pig.

The isolated embryonic stem cell may be identified by a particular expression profile of marker genes. For example, in some embodiments, the embryonic stem cell line expresses one or more of DUSP6, ZIC3, DNMT3A/B ZIC2, OTX2, TET1, NCAM1, TET3, MYC, CD47, HOXA1, FOXA2, GATA6, TBX3, OCT4, CDX2, and SOX2 genes.

The isolated embryonic stem cell line may comprise a genetic modification. In some embodiments, the genetic modification is performed on the zygote cultured in vitro, and the embryonic stem cell line develop from the genetically modified zygote. In other embodiments, the embryonic stem cell line develop from a zygote cultured in vitro that is not genetically modified. For example, in some embodiments, the genetic modification is performed on the embryonic stem cell line. Methods of genetically modifying cells are known in the art and described herein.

The genetically modified embryonic stem cell may be used as the source of a nucleus for somatic cell nuclear transfer (SCNT). For example, in some embodiments the present disclosure provides an oocyte comprising a heterologous nucleus from a genetically modified embryonic stem cell described herein. For example, the nucleus of the oocyte may be removed and replaced with the nucleus from a genetically modified embryonic stem cell described herein. In some embodiments, the present disclosure relates to an embryo that develops from the oocyte comprising the heterologous nucleus. In some embodiments, the disclosure provides an embryo that comprises a nucleus isolated from a genetically modified embryonic stem cell described herein. The embryonic stem cell may be isolated from the developing embryo and stored for further use.

Provided herein are methods for producing stable embryonic stem cells (ESCs), the methods comprising, or alternatively consisting essentially of, or yet further consisting of culturing a blastocyst cell or a pluripotent cell isolated from an embryo, in a cell culture media, the cell culture media comprising: (i) inactivated feeder cells; (ii) an effective amount of Fibroblast Growth Factor 2 (FGF2) or an equivalent thereof; and (iii) an effective amount of one or more of an inhibitor of Wnt signaling. In some aspects, the culture can be feeder-free, instead relying on vitronectin or other ECM proteins coated onto the culture vessel for attachment. In one aspect, the blastocyst, the pluripotent cell or the ESC is bovine, porcine, caprine, or ovine. In a further aspect, the blastocyst cell, the pluripotent cell or ESC is detectably labeled.

The one or more inhibitors of Wnt signaling are selected from among: IWR1, XAV-939, ICG-001, Wnt-059, LGK-974, LF3, CP21R7, NCB-0846, PNU-74654, Salinomycin, SKL2001, KY02111, IWP-2, IWP-L6, Wnt agonist 1, FH535, WIK14, PRI-724, IQ-1, KYA1797K, 2,4-diamino-quinazoline, Ant1.4Br, Ant 1.4C1, apicularen, bafilomycin, C59, ETC-159, G007-LK, G244-LM, IWR, Niclosamide, NSC668036, PKF115-584, pyrvinium, Quercetin, Shizokaol D, BC2059 and any combination thereof. These are commercially available from vendors, for example Torcris Bioscience (tocris.com, last accessed on Jan. 11, 2018) or Santa Cruz Biotechnology, or available following methods provided in the technical literature.

In a further aspect of the method, the blastocyst cell or pluripotent cell is cultured in the absence of an effective amount of Transforming Growth Factor Beta (TGFβ) or an equivalent of TGFβ. In a further aspect, the TGFβ or an equivalent thereof is completely absent in the culture medium.

The steps of the method are not limited by any specific order. For example, one or more of the (i) inactivated feeder cells; (ii) the effective amount of Fibroblast Growth Factor 2 (FGF2) or an equivalent thereof; and (iii) the effective amount of the one or more inhibitors of Wnt signaling can be combined and then the blastocyst cell is added to the culture. Alternatively, in one aspect, the one or more of (i), (ii), or (iii) is provided in the cell culture media after to the addition of the blastocyst or the pluripotent cell. Alternatively, the one or both of (ii) the effective amount of Fibroblast Growth Factor 2 (FGF2) or an equivalent thereof; and (iii) the effective amount of the one or more inhibitors of Wnt signaling are added to the blastocyst cell or the pluripotent cell maintained on the inactivated feeder cells. In various configurations, the cells may be maintained feeder free.

The amount of FGF2 or an equivalent thereof added to the culture conditions can be empirically determined; however non-limiting amounts range from about 5 ng/mL to about 50 ng/mL, or alternatively from about 10 ng/mL to about 100 ng/mL, or alternatively from about 10 ng/mL to about 50 ng/mL, or alternatively from about 10 ng/mL to about 40 ng/mL, or alternatively from about 10 ng/mL to about 30 ng/mL, each per mL of cell culture media. In one specific aspect, the effective amount of FGF2 or an equivalent thereof is about 20 ng/mL of cell culture media.

With respect to the one or more inhibitors of Wnt signaling, the effective amount can vary depending on the Wnt signaling inhibitor and can be empirically determined by those of skill in the art. Non-limiting amounts range from to a final concentration from about 0.1 mM to about 100 mM, after addition to the cell culture media. In another aspect, the effective amount of the one or more inhibitors of Wnt signaling is about 2.5 mM after addition to the cell culture media.

In a further aspect, the cell culture medium further comprises, or alternatively consists essentially of, or yet further consists of, an effective amount of a Rho-associated coiled-coil kinase (ROCK) inhibitor. Non-limiting examples of ROCK inhibitors include one or more of AS1892802, Fasudil hydrochloride, GSK 269962, GSK 429286, H1152, Glyclyl-H 1152, HA 1100, OXA 06, RKI 1447, SB 772077B, SR 3677, TC-S 7001, or Y-27632, or an equivalent of each thereof, or a combination thereof. In one aspect, the ROCK inhibitor is Y-27632 or an equivalent thereof. These are commercially available, e.g., Tocris Bioscience (tocris.com, last accessed on Jan. 11, 2018). The effective amount of the ROCK inhibitor will depend on the specific inhibitor, and can be empirically determined using methods known in the art. Non-limiting examples of effective concentrations of ROCK inhibitors is about 1 μM to about 1000 μM, or from about 1 μM to about 100 μM, or from about 1 μM to about 50 μM, or from about 0.1 μM to about 100 μM, or about 0.1 μM to about 10 μM, each after addition to the cell culture media. In one aspect, the concentration of ROCK inhibitor is about 10 μM after addition to the cell culture media. In a further aspect, the ROCK inhibitor is Y-27632 or an equivalent thereof and the effective amount is about 10 μM after addition to the cell culture media.

In a further aspect, cell culture medium further comprises an effective amount of a low fatty acid BSA. In a yet further aspect, the cell culture medium can comprise TeSR™1 (Wisconsin Alumni Research Foundation, Madison, WI). In a yet further aspect, the cell culture medium is modified TeSR™1. The composition of TeSR™1 is described in U.S. Pat. No. 7,442,548B2. mTeSR™ and modifications thereon are commercially available from STEMCELL™ Technologies (Vancouver, Canada).

In various aspects, the cells may also be maintained on simple media comprising DMEM/F12 chemically defined media. Such media may be supplemented with various growth factors or other additives such as ascorbic acid 2-phosphate, insulin, transferrin, sodium selenite, bFGF, TGFβ1, 1-glutamine or L-alanyl-L-glutamine (sold under the tradename GLUTAMAX®; Thermo Fisher Scientific, Waltham, MA), and Wnt antagonists such as IWR1. Alternatively or in addition, such simple media may also contain BSA, Albumax II, PVA, sodium bicarbonate, ITS, lipids, and other trace elements. Alternatively, or in addition, such media may be supplemented with whey protein isolate, lecithin, and proteins such as bovine serum albumin, and lipids.

In one aspect, the inactivated feeder cells are murine embryonic fibroblasts, or alternatively the inactivated feeder cells are replaced by an organic extracellular matrix or feeder cell conditioned medium. Non-limiting examples of an organic matrix include matrigel, vitronectin, fibronectin, or laminin. In a yet further aspect, the inactivated feeder cells are replaced by an organic matrix, such as matrigel or vitronectin, and supplementation with an effective amount of Activin-A (commercially available from for example, R & D Systems (rndsystems.com). In some embodiments of the above methods, the blastocyst cell is an isolated inner cell mass cell. In a further aspect, the inner cell mass cell has been isolated by mechanical isolation or immunosurgery. The cells can be cultured for an effective amount of time or passages, non-limiting examples of such include passaging the cells for at least 3 weeks or for at least 4 weeks, or for up to 10 weeks or more as noted herein.

In a further aspect, the ESCs prepared by the methods or can be genetically modified or the blastocyst cell or the pluripotent cell can be genetically modified. Thus the methods can further comprise modifying the ESC, or the pluripotent cell, or the blastocyst prior to culturing as noted above.

In one aspect, the embryo from which the pluripotent cells are isolated has been prepared by a method comprising nuclear transfer cloning or parthenogenetic activation. In another aspect, the embryo has been prepared by a method comprising natural or artificial insemination. In a further aspect, the blastocyst cell is isolated from a preimplantation embryo, optionally a morula or a cleavage stage embryo. In another aspect, the pluripotent cell of the embryo is isolated from the embryonic disk of a post-hatch embryo

The embryonic cell or the pluripotent cell can be genetically modified prior to the culturing method or during the culturing method. Thus, the methods further comprise genetically modifying the blastocyst, or the pluripotent cell or ESC prior to culturing. In any of the above methods, the blastocysts, or the pluripotent cell or ESCs can be detectably labeled prior to, during or subsequent to the culturing methods.

In a yet further aspect, the ESCs are isolated from the cell culture media after being cultured for an effective amount of time. In addition, one can also isolate one or more microvesicles or exosomes from the culture media.

Further provided are methods for performing genomic selection, the method comprising, consisting essentially of, or yet further consisting of: (i) screening ESCs that have been produced according to the method as prepared above for a preferred genotype; and (ii) selecting ESCs comprising the preferred genotype.

One can modify the embryo, by any appropriate method, e.g., comprising establishing an embryo from a nuclear transfer process wherein the ESC nuclei is inserted into an enucleated oocyte.

Further provided is a method comprising, or alternatively consisting essentially of, or yet further consisting of, (a) establishing an embryo from a nuclear transfer process wherein nuclei of the ESC as disclosed herein is inserted into an enucleated oocyte, and (b) implanting the embryo into a non-human animal recipient such as cattle, pigs, or sheep. In a further aspect, the embryo is gestated in the non-human animal recipient such as cattle, pigs, or sheep. The resulting non-human animal (e.g., cattle, pigs, sheep) and its offspring from the implanted embryo is further disclosed herein. In a yet further aspect, the method can further comprise introducing the ESC to a genetically modified embryo or an embryo produced by nuclear transfer cloning.

Yet further provided is a method of creating a chimeric non-human animal (e.g., cattle, pigs, sheep), the method comprising, or alternatively consisting essentially of, or yet further consisting of: (a) generation of ESC; (b) introduction of ESC into a preimplantation embryo; and (c) transfer of embryo to a non-human recipient. Yet further provided is the non-human animal (e.g., cattle, pigs, sheep) prepared by the method.

Yet further provided is a method for creating germ cells (sperm and oocytes) from ESC, the method comprising, or alternatively consisting essentially of, or yet further consisting of: (a) generating ESC; (b) in vitro differentiation of the ESC to germ cells by addition/subtraction of growth factors and/or co-culture with somatic cells; (or) (c) transplantation of the ESC or intermediate differentiated cells into an non-human animal (e.g., cattle, pigs, sheep) for generation of germ cells. In one aspect, the ESC are transplanted to a germline-deficient non-human animal embryo. Yet further provided are the non-human animals prepared by these methods, as well the offspring from the transplanted ESC.

### Methods for Isolating and Culturing Fish Embryonic Stem Cells

Fish ESCs may be derived from blastula stage embryos by adopting feeder layer or feeder-free culture conditions. The fish ESCs may be derived from salmon, tuna, medaka, perch, pollock, catfish, cod, haddock, sea bass, tilapia, Arctic char, trout, and carp (see Collodi P., et al., 1992, Cell Biol. Toxicol., 8, 43-61; Wakamatsu, Y., et al., 1994, Mol. Mar. Biol. Biotechnol., 3, 185-191; and Hong, Y., et al., 1996, Mech. Dev., 60, 33-44). Blastula stage embryos may be obtained via external or in vitro fertilization. Examples of feeder cells include zebrafish embryonic fibroblasts, buffalo rat liver cells and the rainbow trout spleen cell line RTS34st. In feeder-free culture systems, components of ES cell-conducive medium may include fish embryo extract from medaka, basic fibroblast growth factor and fish serum, which are capable of supporting self-renewal of disassociated midblastula embryo (MBE) cells on a gelatin-coated culture dish.

In some embodiments, the fish ESCs are diploid cell lines derived from fertilization blastulae. Examples of diploid fish ESC lines include MES1, MES2 and MES3. In certain embodiments, diploid fish ESC lines display features such as stable growth, a typical ES cell phenotype (a round/polygonal shape, a small size, large nuclei and prominent nucleoli), high alkaline phosphatase activity (a general marker of mouse ES cells), a normal karyotype and the ability for spontaneous differentiation into various cell types including pigment cells, muscle cells, nerve cells and fibroblasts. Additionally or alternatively, in some embodiments, diploid fish ESC lines undergo clonal growth, forming compacted cell colonies of undifferentiated ES cells capable of expansion into ES cell clones.

Additionally or alternatively, in certain embodiments, the fish embryonic stem cells express one or more of oct4, nanog, klf4, sox2, myc, ronin, sall4, and tcf3 (tcf7/1). Additionally or alternatively, in some embodiments, the fish embryonic stem cells do not express tert splicing variants.

A fish embryo may be prepared by transplanting or injecting nuclei from the donor ESCs into enucleated oocytes. The nuclear transplantation can be carried out by any of the methods known in the art, without particular restriction. As for the techniques, for example, cell fusion via a chemical substance, viral or electric technique, intact or damaged cell injection, dissolved cell injection, and nucleus injection can be employed.

In some embodiments, the fish ESCs are haploid cell lines derived from haploid blastula embryos for cell initiation (instead of fertilization diploid blastulae). For example, sperm of i³ strain are treated with an elaborated dose of ultra-violet light irradiation to such an extent to destroy their nuclei but retain their ability to trigger egg activation. These genetically inactivated sperm are mixed with mature oocytes of i¹ strain for artificial insemination, resulting in eggs only with a haploid female nucleus. Such embryos undergo all-female embryogenesis called gynogenesis. Until the midblastula stage, the gynogenetic haploid embryos are dissociated into single cells and seeded for feeder-free culture on gelatin-coated substrata. Details for induced haploid gynogenesis and cell derivation have been described in Yi, M., et al., 2010, Nat. Protoc., 5, 1418-1430. In certain embodiments, haploid ES cell derivation further comprises media conditioned by MES1 and MO1, a medaka ovary-derived cell line. Once established, haploid ES cells are not different from diploid ES cells in conditions for maintenance. Haploid ES cells show all characteristics of MES1, including stable and competitive growth, genetic stability and pluripotency in vitro and in vivo. The recent establishment of haploid ES cells provides an excellent opportunity for direct nuclear transfer into normal eggs without the need for enucleation, known as semi-cloning (SC). In this approach, a mitotic haploid nucleus is transferred to a mature oocyte without removal of its nucleus, leading to the combination of a haploid somatic nucleus from one parent and a haploid gamete nucleus from the other parent to yield a mosaic oocyte, which subsequently develops into viable and fertile offspring.

### Uses of the Gene Editing Agents of the Present Technology

In one aspect, the present disclosure provides a method for improving a trait in a non-human animal subject such as cattle, pigs, sheep or aquatic organisms including fish, shrimp and other crustaceans, and mollusks (e.g., gastropods, cephalopods, and bivalves), the method comprising: contacting embryonic stem cells of the non-human animal subject with an effective amount of a gene editing agent to obtain genetically modified embryonic stem cells comprising a modification in a target gene of interest, thereby improving a trait in the non-human animal subject, wherein the gene editing agent is configured to modify the target gene of interest. In some embodiments, the method further comprises (a) transplanting or injecting a nucleus from the genetically modified embryonic stem cells into an enucleated oocyte to obtain an oocyte comprising the modification in the target gene of interest, and (b) establishing an embryo from the oocyte comprising the modification in the target gene of interest.

In some embodiments, the embryonic stem cells are obtained from cattle and the method further comprises implanting the embryo into a cow or heifer recipient and allowing the embryo to develop into a fetus having the improved trait. In certain embodiments, the embryonic stem cells are obtained from pig and the method further comprises implanting the embryo into a gilt or sow recipient and allowing the embryo to develop into a fetus having the improved trait. In any of the preceding embodiments of the methods disclosed herein, the embryonic stem cells are isolated from inner cell mass (ICM) of blastocyst stage embryos. The blastocyst stage embryos may be obtained via in vitro fertilization. In some embodiments, the blastocyst stage embryos are cultured in vitro in cell culture medium before the embryonic stem cells are isolated. In some embodiments, the embryonic stem cells are isolated from a blastocyst and cultured in vitro in cell culture medium for about 1, 2, 3, 4, 5, or more days. In some embodiments, the blastocyst is in culture from about 3 to about 5 days before the embryonic stem cells are isolated.

In some embodiments of the methods disclosed herein, the embryo is cultured in vitro before implantation into the female non-human animal recipient, and laparoscopic or non-surgical transfer may be practiced in many variations. Variations are described more fully in PCT/US2005/034641, which is herein incorporated by reference in its entirety. In certain embodiments, the embryo is porcine, bovine, or ovine. In some embodiments, the embryo is transferred when it is at least at the 2-cell stage, at least at the 4-cell stage, at least at the 8-cell stage, at least at the 16-cell stage, at least at the morula stage, at least at the blastocyst stage, at least at the expanding blastocyst stage, at least at the hatching blastocyst stage, or at least at the blastula stage. In other embodiments, the embryo is transferred when it has been cultured in vitro for at least about 18 hours, at least about 24 hours, at least about 48 hours, at least about 72 hours, at least about 76 hours, at least about 80 hours, at least about 84 hours, at least about 90 hours, at least about 96 hours, at least about 102 hours, at least about 108 hours, at least about 114 hours, at least about five days, at least about five and one-half days, at least about six days, at least six and one-half days, at least seven days, at least seven and one-half days, at least eight days, at least eight and one-half days, or at least nine days after injection of nuclear material from the genetically modified embryonic stem cells.

In one embodiment, the embryos are cultured in a media such as PZM or NCSU at a temperature range of 36° C. to 40° C. under humid atmosphere containing 3.5% to 6.5% CO₂ with any appropriate range of 02, more preferably 38.5° C. in 5% CO₂:5% O₂. In another embodiment, the embryo may be stored in any atmosphere where the media is under oil to prevent evaporation.

In various embodiments, the embryo transfer can be accomplished by surgical or non-surgical methods or by minimally invasive methods, i.e., laparoscopic methods. In preferred embodiments, the site of transfer is the uterus, most preferably, the tip, middle or base of the uterine horn, or in the uterus body itself.

Fish Embryo Cultivation. In other embodiments, the embryonic stem cells are fish embryonic stem cells and the method further comprises cultivating the embryo until hatching under conditions appropriate for culturing fish. Fish embryos may be cultivated for development until hatching under conditions appropriate for the fish species. The cultivation conditions for development until hatching can be properly selected according to the fish species. In the case of medaka, for instance, the cultivation is carried out at about 18° C. for about 24 hours after nuclear transplantation and, thereafter, the cultivation can be carried out at a temperature of about 26° C. A balanced salt solution (BSS) containing methylene blue or the like solution can be used as the culture medium. Fish offspring can be obtained by causing the fish embryo to hatch in the above manner. The fish offspring obtained can be bred by a method suited for the fish species. It is also possible to carry out further genetic modification by mating or chromosome manipulation. Additionally or alternatively, in some embodiments of the methods disclosed herein, the fish embryonic stem cells are cultured in vitro in cell culture medium prior to step (a) or step (b).

Additionally or alternatively, in any and all embodiments of the methods disclosed herein, the modification in the target gene of interest comprises a modification of about 1-200 nucleotides (nts). In some embodiments, the modification in the target gene of interest comprises a modification of no more than 1 nt, no more than 5 nts, no more than 10 nts, no more than 15 nts, no more than 20 nts, no more than 25 nts, no more than 30 nts, no more than 35 nts, no more than 40 nts, no more than 45 nts, no more than 50 nts, no more than 55 nts, no more than 60 nts, no more than 65 nts, no more than 70 nts, no more than 75 nts, no more than 80 nts, no more than 85 nts, no more than 90 nts, no more than 95 nts, no more than 100 nts, no more than 105 nts, no more than 110 nts, no more than 115 nts, no more than 120 nts, no more than 125 nts, no more than 130 nts, no more than 135 nts, no more than 140 nts, no more than 145 nts, no more than 150 nts, no more than 155 nts, no more than 160 nts, no more than 165 nts, no more than 170 nts, no more than 175 nts, no more than 180 nts, no more than 185 nts, no more than 190 nts, no more than 195 nts, or no more than 200 nts.

Additionally or alternatively, in some embodiments, the method further comprises selecting or screening for genetically modified embryonic stem cells, optionally wherein the selecting or screening comprises contacting embryonic stem cells that have been contacted with the gene editing agent with a probe specific for the modification in the target gene of interest.

Exemplary bovine traits include polled (lack of horns), sterility or fertility, milk production, growth (which increases meat production), fat production, conception rates, stillborn rates, calving ease, or content of produced milk such as the amount of protein or the amount of fat. Further bovine traits can include backfat thickness, intramuscular fat, ultrasound loin muscle area, loin muscle area and intramuscular fat content, chest circumference, withers height, body length, hip height, rump length, and heart girth. Other exemplary native traits include, but are not limited to, high altitude adaptation and response to hypoxia (DCAF8, PPPTRT2A, SLCT6A3, UCP2, UCP3, TIGAR), cold acclimation (AQP3, AQP7, HSPB8), body size and stature (PLAG1, KCNA6, NDUFA9, AKAP3, C5H12orf4, RAD51AP1, FGF6, TIGAR, CCND2, CSMD3), resistance to disease and bacterial infection (CHI3L2, GBP6, PPFIBP1, REP15, CYP4F2, TIGD2, PYURF, SLC10A2, FCHSD2, ARHGEF17, RELT, PRDM2, KDM5B), reproduction (PPP1R12A, ZFP36L2, CSPP1), milk yield and components (NPC1L1, NUDCD3, ACSS1, FCHSD2), growth and feed efficiency (TMEM68, TGS1, LYN, XKR4, FOXA2, GBP2, GBP5, FGD6), and polled phenotype (URB1, EVA1C).

Other exemplary target genes can include PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR, and ZBTB.

Exemplary porcine traits include meat production traits such as growth rate, backfat depth, muscle pH, purge loss, muscle color, firmness, marbling scores, intramuscular fat percentage, tenderness, average daily gain, average daily feed intake, feed efficiency, back fat thickness, loin muscle area, and lean percentage. Exemplary health traits include the absence of undesirable physical abnormalities or defects (like scrotal ruptures), improvement of feet and leg soundness, resistance to specific diseases or disease organisms, or general resistance to pathogens. Further health traits can include melanotic skin tumors, dermatosis vegetans, abnormal mamae, shortened vertebral column, kinky tail, rudimentary tail, hairlessness, woolly hair, hydrocephalus, tassels, legless, three-legged, syndactyly, polydactyly, pulawska factor, heterochromia iridis, congenital tremor a iii, congenital tremor a iv, congenital ataxia, hind leg paralysis, bentleg, thickleg, malignant hyperthermia, hemophilia (von Willebrand's disease), leukemia, hemolytic disease, edema, acute respiratory distress (“barker”), rickets, renal hypoplasia, renal cysts, uterus aplasia, porcine stress syndrome (pss), halothane (hal), dipped shoulder (humpy back, kinky back, kyphosis), hyperostosis, mammary hypoplasia, undeveloped udder, and epitheliogenesis imperfecta. Exemplary target genes can include, but are not limited to, ANP32, ANPEP, TMPRSS1, TMPRSS2, TMPRSS4, NANOS2, CD163, Melanocortin-4 receptor (MC4R), HMGA, IGF2, HAL, RN, Mx1, BAT2, EHMT2, PRDM1, PRDM14, and ESR.

Exemplary nucleic acid sequences associated with traits such as pathogen resistance, fertility, lactation, or native traits that support more rapid growth or feed efficiency in cattle, sheep, pigs, or fish include, but are not limited to: Gene GenBank Ace. No.

| Gene | GenBank Acc. No. |
|---|---|
| DCAF8 | NM_001206419.2 (SEQ ID NO: 1) |
| SLC16A3 | NM_001109980.3 (SEQ ID NO: 2) |
| TIGAR | NM_001076370.1 (SEQ ID NO: 3) |
| Deadend (Dnd) | JN712911.1 (SEQ ID NO: 4) |
| CD163 | NM_213976.1 (SEQ ID NO: 5) |
| | XM_021091121.1 (SEQ ID NO: 6) |
| | GQ184570.1 (SEQ ID NO: 7) |
| ANP32 | XM_003121759 (SEQ ID NO: 8) |
| (ANP 32A and ANP 32B) | XM_021066477.1 (SEQ ID NO: 9) |
| | NM_001195019.1 (SEQ ID NO: 10) |
| | NM_001035074.1 (SEQ ID NO: 11) |
| ANPEP | XM_005653524.3 (SEQ ID NO: 12) |
| | XM_021098304.1 (SEQ ID NO: 13) |
| | NM_001075144.1 (SEQ ID NO: 14) |
| TMPRSS2 | NM_001386131.1 (SEQ ID NO: 15) |
| | NM_001081585.1 (SEQ ID NO: 16) |
| NANOS1 | XM_001928298 (SEQ ID NO: 17) |
| | XM_005225796 (SEQ ID NO: 19) |
| NANOS2 | XM_003127232.2 (SEQ ID NO: 18) |
| Melanocortin-4 receptor | NM_214173.1 (SEQ ID NO: 20) |
| (MC4R) | NM_174110.1 (SEQ ID NO: 21) |
| PRLR | NM_001039726.2 (SEQ ID NO: 22) |
| | NM_174155.3 (SEQ ID NO: 23) |
| | NM_001001868.1 (SEQ ID NO: 24) |
| HMGA | XM_005663957.3 (SEQ ID NO: 25) |
| | XM_021091958.1 (SEQ ID NO: 26) |
| | XM_002687648.5 (SEQ ID NO: 27) |
| CRTC2 | XM_005663405.3 (SEQ ID NO: 28) |
| | NM_001076250.1 (SEQ ID NO: 29) |
| SIX4 | XM_024998078.1 (SEQ ID NO: 30) |
| | NM_001244614.1 (SEQ ID NO: 31) |
| UCP2 | NM_001033611.2 (SEQ ID NO: 32) |
| | NM_214289.1 (SEQ ID NO: 33) |
| UCP3 | NM_174210.1 (SEQ ID NO: 34) |
| | NM_214049.1 (SEQ ID NO: 35) |
| URB1 | NM_001205980.1 (SEQ ID NO: 36) |
| EVA1C | XM_024988259.1 (SEQ ID NO: 37) |
| TMEM68 | NM_001076009.1 (SEQ ID NO: 38) |
| TGS1 | XM_015474433.2 (SEQ ID NO: 39) |
| LYN | XM_025001638.1 (SEQ ID NO: 40) |
| XKR4 | XM_002692650.4 (SEQ ID NO: 41) |
| FOXA2 | XM_025001047.1 (SEQ ID NO: 42) |
| FGD6 | XM_005206128.4 (SEQ ID NO: 43) |
| NPC1L1 | XM_002686890.5 (SEQ ID NO: 44) |
| NUDCD3 | XM_005205595.3 (SEQ ID NO: 45) |
| ACSS1 | XM_025000325.1 (SEQ ID NO: 46) |
| FCHSD2 | XM_024975871.1 (SEQ ID NO: 47) |
| PPP1R12A | XM_024991981.1 (SEQ ID NO: 48) |
| ZFP36L2 | NM_001191191.1 (SEQ ID NO: 49) |
| CSPP1 | XM_025001590.1 (SEQ ID NO: 50) |
| CHI3L2 | XM_024990025.1 (SEQ ID NO: 51) |
| GBP6 | NM_001075995.1 (SEQ ID NO: 52) |
| GBP5 | HF564747.1 (SEQ ID NO: 53) |
| PPFIBP1 | XM_010805392.3 (SEQ ID NO: 54) |
| REP15 | XM_005206922.4 (SEQ ID NO: 55) |
| CYP4F2 | XM_010806565.3 (SEQ ID NO: 56) |
| TIGD2 | XM_005207816.4 (SEQ ID NO: 57) |
| PYURF | NM_001038102.2 (SEQ ID NO: 58) |
| SLC10A2 | XM_025000165.1 (SEQ ID NO: 59) |
| ARHGEF17 | NM_001282586.1 (SEQ ID NO: 60) |
| RELT | XM_002693451.5 (SEQ ID NO: 61) |
| PRDM2 | XM_015475202.2 (SEQ ID NO: 62) |
| KDM5B | XM_024976577.1 (SEQ ID NO: 63) |
| PLAG1 | XM_005215434.3 (SEQ ID NO: 64) |
| KCNA6 | NM_001206413.3 (SEQ ID NO: 65) |
| NDUFA9 | NM_205817.1 (SEQ ID NO: 66) |
| AKAP3 | XM_015471316.2 (SEQ ID NO: 67) |
| C5H12orf4 | XM_005207206.4 (SEQ ID NO: 68) |
| RAD51AP1 | NM_001076403.1 (SEQ ID NO: 69) |
| FGF6 | NM_001192400.1 (SEQ ID NO: 70) |
| CCND2 | XM_024992177.1 (SEQ ID NO: 71) |
| CSMD3 | NM_001105499.2 (SEQ ID NO: 72) |
| AQP3 | NM_001079794.1 (SEQ ID NO: 73) |
| AQP7 | XM_024995941.1 (SEQ ID NO: 74) |
| HSPB8 | NM_001014955.1 (SEQ ID NO: 75) |
| IGF2 | NM_213883.2 (SEQ ID NO: 76) |
| | NM_001367627.1 (SEQ ID NO: 77) |
| MUC13 | NM_001105293.1 (SEQ ID NO: 78) |
| HAL | XM_001925061.4 (SEQ ID NO: 79) |
| Mx1 | NM_214061.2 (SEQ ID NO: 80) |
| BAT2 | NM_001114675.1 (SEQ ID NO: 81) |
| EHMT2 | NM_001101823.1 (SEQ ID NO: 82) |
| | NM_001206263.2 (SEQ ID NO: 83) |
| PRDM1 | NM_001192936.1 (SEQ ID NO: 84) |
| | XM_005659341.3 (SEQ ID NO: 85) |
| PRDM14 | NM_001191264.1 (SEQ ID NO: 86) |
| | XM_021090784.1 (SEQ ID NO: 87) |
| ESR | NM_214220.1 (SEQ ID NO: 88) |
| APAF1 | NM_001191507.1 (SEQ ID NO: 89) |
| | XM_021093024.1 (SEQ ID NO: 90) |
| SMC2 | XM_021066949.1 (SEQ ID NO: 91) |
| | XM_024996295.1 (SEQ ID NO: 92) |
| GART | NM_001040473.2 (SEQ ID NO: 93) |
| | XM_021070945.1 (SEQ ID NO: 94) |
| TFB1M | NM_001076896.2 (SEQ ID NO: 95) |
| | NM_001128475.1 (SEQ ID NO: 96) |
| SIRT1 | NM_001145750.2 (SEQ ID NO: 97) |
| | NM_001192980.3 (SEQ ID NO: 98) |
| SIRT2 | XM_021093185.1 (SEQ ID NO: 99) |
| | NM_001113531.1 (SEQ ID NO: 100) |
| LPL | NM_001075120.1 (SEQ ID NO: 101) |
| | NM_214286.1 (SEQ ID NO: 102) |
| TMPRSS4 | XM_021062910.1 (SEQ ID NO: 113) |
| | XM_013977479.2 (SEQ ID NO: 114) |
| | XM_005653708.3 (SEQ ID NO: 115) |
| | XM_021062909.1 (SEQ ID NO: 116) |
| | NM_001101973.2 (SEQ ID NO: 117) |
| GBP2 | EU259888.1 (SEQ ID NO: 118) |

In any and all embodiments of the methods disclosed herein, the gene editing agent inhibits expression of target gene encoded by any one of SEQ ID NOs: 1-102.

In one aspect, the present disclosure provides a method of producing a germline mutation in a non-human mammalian subject (e.g., cattle, pigs, sheep) comprising: (a) contacting embryonic stem cells of the non-human mammalian subject with an effective amount of a gene editing agent to obtain genetically modified embryonic stem cells comprising a knock-in, a knockout, a deletion or a point mutation in a target gene of interest; and (b) screening the embryonic stem cells that have been contacted with the gene editing agent for the knock-in, knockout, deletion or point mutation in the target gene of interest. In some embodiments, the embryonic stem cells of the non-human mammalian subject are obtained by culturing blastocysts from a zygote on feeder cells in culture medium for a sufficient time period.

The disclosure also relates to a method of detecting the presence or absence of a genetic modification in a cell based upon measurements of the probes specific for the genetic modification of interest. The method may comprise a step of quantifying the amount of RNA or protein expression on a cell or plurality of cells by exposing cells known to comprise or suspected of comprising the genetic modification to one or a plurality of probes known to associate, bind or enzymatically alter the expression products above. The data collected on the number and concentration of expression products, can be used as evidence to correlate the signature or detection of the presence of modified embryonic stem cells in a cell culture device.

### Kits

Compositions of some embodiments of the present technology may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for veterinary administration. Such notice, for example, may be of labeling approved by the U.S. Food and Drug Administration for prescription drugs or of an approved product insert. Compositions comprising a preparation of the present technology formulated in a compatible pharmaceutical carrier may also be prepared, placed in an appropriate container, and labeled for treatment of an indicated condition.

The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

Furthermore, the kit may comprise other materials desirable from a commercial and user standpoint, including other buffers, diluents, filters, needles, syringes, culture medium for one or more of the suitable hosts.

### EXAMPLES

It is to be understood that the present technology is not necessarily limited in its application to the details set forth in the following description or exemplified by the Examples. The present technology is capable of other embodiments or of being practiced or carried out in various ways. Also, it is to be understood that the phraseology and terminology employed herein is for the purpose of description and should not be regarded as limiting.

### Example 1

This example illustrates a method of nucleofection of embryonic stem cells.

The gRNAs will be generated by in vitro transcription and complexed with SpyCas9 in water, using 3.2 μg of Cas9 protein and 2.2 μg of gRNA in a total volume of 2.23 μl. Half volumes (1.115 μl) of the resulting ribonucleoprotein (RNP) complexes will then be combined 1:1 in a total volume of 2.23 μl to generate pairs, and nucleofected into embryonic stem cells (ESCs) using a Lonza electroporator. In preparation for nucleofection, ESCs will be harvested using TRYPLE EXPRESS™ (ThermoFisher Scientific, Waltham, MA, recombinant Trypsin). Specifically, the culture medium will be removed from cells, cells will be washed once with Hank's Balanced Salt Solution (HBSS) or Dulbecco's Phosphate-Buffered Saline (DPBS), and incubated for 3-5 minutes at 38.5° C. in the presence of TrypLE. Cells will then be harvested with complete medium. Cells will be pelleted via centrifugation (300 g×5 minutes at room temperature), supernatant will be discarded, and then the cells will be resuspended in 10 mL PBS to obtain single cell suspensions to allow cell counting using trypan blue staining. After counting, cells will be pelleted via centrifugation, the supernatant will be discarded, and the cells will be resuspended in nucleofection buffer P3 (Lonza) at a final concentration of 7.5×10⁶ cells/ml. 20 μl of the cell suspension will be added to each well of a nucleofection cuvette containing the RNP mixture and then mixed gently to resuspend the cells. The RNP/cell mixture will then be nucleofected with program CM138. Following nucleofection, 80 μl of warm Embryonic Stem Cell Medium (ESCM) [DMEM/F12 medium (11320-033, Gibco), Neurobasal medium (21103-049, Gibco), 0.5% v/v N-2 Supplement (17502-048, Gibco), 1% v/v B-27 Supplement (17504-044, Gibco), 2 mM MEM (Non-Essential Amino Acid Solution (M7145, Sigma), 1% v/v GlutaMAX Supplement (35050-061, Gibco), 0.1 mM 2-mercaptoethanol (M6250, Sigma), 100 U/mL Penicillin, and 100 μg/mL Streptomycin (15140-122, Gibco) supplemented with 20 ng/mL human FGF2, 2.5 μM IWR-1, and 10 μM ROCKi](Soto et al, 2021). The suspensions will be mixed gently by pipetting, and then individual cells will be transferred to a 96 well tissue culture plate containing CF1 (mouse feeder cells) which had previously been cultured in feeder cell medium (DMEM, 10% FBS, 1% v/v GlutaMAX Supplement (35050-061, Gibco), 100 U/mL Penicillin, and 100 μg/mL Streptomycin (15140-122, Gibco)). The plate will then be incubated at 38.5° C., 5% 02, 5% CO₂ for 7 days. Seven days after nucleofection, single ESC colonies are harvested. Two thirds of harvested cells will be transferred to a single well of a 24-well tissue culture plate for expansion during genomic analysis. The remaining cells (˜1,000 cells) will be used for genomic DNA isolation. Specifically, 15 μl of QUICKEXTRACT™ DNA Extraction Solution (Lucigen Corporation, Middleton, WI) will be added to pelleted cells, and the cells will then lysed by incubating for 10 minutes at 37° C., for 8 minutes at 65° C., and for 5 minutes at 95° C. Lysate will be held at 4° C. until used for DNA sequencing to confirm that the desired edit is in the cells. ESC colonies identified to contain the intended edit will be expanded further or cryopreserved for downstream applications (i.e. Nuclear Transfer).

### Example 2

This example illustrates comparison of porcine stem cell derivation using different media conditions.

Porcine blastocysts were generated from in vitro fertilization experiments and were grown until day 7; some had hatched naturally or through laser-assisted hatching, but the remaining were treated with Pronase or Acid Tyrodes to remove the zona pellucida. The antibiotics penicillin (10 Units/mL) and streptomycin (10 microgram/mL), and the antifungal amphotericin B (0.25 microgram/mL) were included during the generation step.

The zone pellucida-free blastocysts were then plated across a 96 well plate coated with a mix of Laminin 521 (1:15 dilution=6.66 μg/mL) and vitronectin (1:75 dilution=6.66 μg/mL). All initial media contained Activin A (20 ng/mL), Lif (10 ng/mL), and Y-27632 (10 μM). The medias tested were “NBFR” (see Soto, D. A., et al., 2021, Scientific Reports, 11, 11045) and mTeSR™ Plus (STEMCELL™ Technologies). NBFR has been used for the culture of bovine stem cells and consists of a 1:1 mix of DMEM/F12 and Neurobasal media (0.5×N2, 0.5×B27, 1% GLUTAMAX™ (Life Technologies Corporation, Carlsbad, CA), 1% MEM non essential amino acids, 100 μM beta-mercaptoethanol, 1% BSA, 20 ng/mL FGF2, and 2.5 μM IWR1). mTeSR™ Plus is a commercially available media used for human stem cell culture. Both medias were tested with and without IWR1. A 1:1 mix of NBFR+mTeSR™ Plus was also tested. Incubator conditions were 38.5 C, 5% CO₂, 5% O₂. The first derivation experiment resulted in two lines—C9 from mTeSR™ Plus+IWR1, and G3 from NBFR+mTeSR™ Plus+IWR1. Both lines expressed the common stem cell markers Nanog, OCT4, and SOX2 by qRT-PCR at levels greatly higher than fibroblast controls.

### Example 3

This example illustrates further optimization of ESC establishment conditions.

A 96 well plate was coated with a mix of Lamin 521 and vitronectin as described in Example 2. Blastocysts were plated as described in media comprising mTeSR™ Plus, IWR1, Activin A, Lif, and Y27632 in the same concentrations as described in Example 2. From 29 blastocysts, two more lines were generated—C2 and D2. These lines expressed the common stem cell markers Nanog, OCT4, and SOX2 by qRT-PCR at levels greatly higher than fibroblast controls.

### Example 4

This example illustrates testing of different media growth parameters.

Blastocysts were plated as described in Example 2 onto a 5 μg/mL laminin 521 (1:20 dilution) coated 12 well plate in mTeSR™ Plus supplemented with 2.5 μM IWR1+10 μM Y27632 (no Lif or Activin A). One line (A8) was generated from 10 starting blastocysts. This line looked similar to C2 and C9, was alkaline phosphatase positive, and expressed the common stem cell markers Nanog, OCT4, and SOX2 by qRT-PCR at levels greatly higher than fibroblast controls.

### Example 5

This example illustrates that the conditions of Example 4 repeatably lead to the production of porcine cell lines.

26 blastocysts were matured and treated as describe in Example 4. Two lines E3 and F4 were successfully generated.

### Example 6

This example illustrates the characterization of lines C2 (male) and C9 (female) from Examples 2 and 3.

The two lines were karyotyped according to standard methods and found to have normal karyotypes.

Cells from each line were tested for alkaline phosphatase expression using STEMAB™ Alkaline Phosphatase Staining Kit II (REPROCELL®, Beltsville, MD). The cells were positive for alkaline phosphatase expression.

Stem cell lines C2 and C9 growth rate was determined by seeding 15000 cells per well of a 12 well plate and counting cell numbers at 24, 48, and 72 hours post seeding in triplicate. The doubling time was calculated for the intervals 24-48 h, and 48-72 h. When averaged this gave the fast doubling time of 13.5 hours for C2 and 10 h for C9.

### Example 7

This example illustrates trilineage differentiation of stem cell lines of the present teachings.

The STEMdiff™ Trilineage Differentiation Kit (STEMCELL™ Technologies) was used according to manufacturer directions to direct the porcine stem cell lines into ectoderm, mesoderm, and endoderm cell types through the addition of specialized media. Cells were harvested at day 5 (mesoderm and endoderm) or day 7 (ectoderm) for RNA isolation and mRNAseq was performed on lines C2 and C9 (from Examples 2 and 3). Marker genes commonly used to identify the different germline lineages were seen to increase expression as expected in the relevant conditions eg TBXT and MSGN1 in the mesoderm samples, Pax6 and NR2F2 in ectoderm, and LEFTY2 and CER1 in endoderm. Expression of these markers in the expected differentiated germlines illustrates that stem cells made according to the present teachings are pluripotent and capable of differentiating into different germ layers.

### Example 8

This example illustrates the differentiation of a stem cell line of the present teachings into macrophages.

Lines C2 and C9 (Examples 2 and 3) were treated according to the method published in Meek, S., et al., 2022, Bmc Biol., 20, 14. Briefly, cells were treated in mesoderm induction media containing FGF, BMP4, VEGF, SCF, and Y-27632 overnight. Cells were fed daily with the same media, but without the Y-27632 and fed daily until day 4. Cells were then transferred to macrophage induction medium which comprised CSF and IL-3 until day 12, at which point any floating cells were harvested every four days until day 24 for maturation. The harvested cells were then matured into macrophage cells in medium containing CSF-1 for at least two days.

Once mature (in this instance on day 23), macrophages were assayed for phagocytosis by adding 20 microliters of 0.5 mg/mL phrodo-Zymosan (Invitrogen) particles to the macrophage in a well of a 96 well plate. These particles fluoresce only after uptake into digestive vesicles. Uptake was observed in a few cells after one hour, with a majority of cells taking up the particles after 30 hours. This example illustrates that ESCs of the present teachings are capable of being induced into macrophages.

### Example 9

This example illustrates modification of embryonic stem cells using a PiggyBAC transposon.

The sequence for mEGFP was synthesized and cloned into a pcDNA™3.1+ expression plasmid (Genscript, Piscataway NJ) to give the plasmid pTD001. The piggyBac transposase sequence was obtained from Genbank accession J04364.2, underwent codon optimization for mammalian expression, and was synthesized and cloned into a pcDNA™3.1+expression plasmid (Genscript, Piscataway NJ) to give the plasmid pTD003. PiggyBac terminal repeat sequences were synthesized by taking the 311 bp at the 5′ and the 235 bp at the 3′ of J04364.2, inserting restriction sites between the two regions, flanked the insert with TTAA, and synthesized to give plasmid pTD005 (Genscript, Piscataway NJ). Restriction enzyme cloning was used to insert the mEGFP expression and neomycin resistance cassette from pTD001 into pTD005, forming the mEGFP piggyBac plasmid pTD008.

C2 line was cultured as described in TI (mTesrPlus+endo-IWR1 media) on laminin 521 coated plates. To create the green fluorescent line, cells were harvested from one well of a 6 well plate via ACCUTASE® treatment (Life Technologies, Grand Island NY), washed with PBS (Life Technologies, Grand Island NY), and resuspended in 200 μL of P3 nucleofection solution (Lonza, Walkersville MD). To 100 μL of cells were added 2 μg of mEGFP piggyBac plasmid pTD008 with or without 2 μg of the transposase plasmid pTD003, the mix was transferred to an electroporation cuvette and pulsed with condition DS137 in a 4D-Nucleofector system (Lonza, Walkersville MD). Cells were plated in TI+10 μM Y27632, with TI media addition the following day, and media change with TI on day two. Beginning day 3, selection was carried out with TI+250 or 500 μg/mL GENETICIN™ (Thermo Fisher Scientific, Waltham, MA). Cells were expanded under selection for two passages before cryopreservation.

The piggyBac construct successfully expressed mEGFP, as green cells were visible by fluorescence microscopy by day two post electroporation. The condition that included the transposase plasmid had increased numbers of green cells. Antibiotic selection led to the death of essentially all cells in the absence of transposase but resulted in GENETICIN™-resistant green cells when both pTD003 and pTD008 had been electroporated. Both 250 μg/mL and 500 μg/mL of geneticin produced resistant green cells. Cells with differing levels of green expression were seen, which was expected as these cells were a pool and not a clonal line. Without being limited by theory, the piggyBac construct integrates at TTAA sites randomly in the genome so individual cells will have both differing copy numbers of integration as well as some level of context dependent expression. These results are consistent with transposase-dependent insertion of the piggyBac cassette into the genome. No obvious morphological difference was noted in the ESC after cells were engineered in this experiment. This example illustrates that ESCs of the present teachings are amenable to genetic modification.

### Example 10

This example illustrates CRISPR-CAS9 editing of embryonic stem cells of the present teachings.

As proof of concept, the present inventors sought to create the CD163 edit disclosed in U.S. Pat. No. 11,535,850. Two guides, ctggcttact cctatcatga agg (SEQ ID NO: 103) and tcccatgcca tgaagagggt agg (SEQ ID NO: 104) were used to excise the seventh exon of CD163. These guides were electroporated into the C9 line by mixing cells in P3 nucleofection solution with ribonucleoprotein (20 pmol SpCas9 complexed with 60 pmol of the guide sgRNA) and using the pulse condition DS137 in a 4D-Nucleofector system (Lonza, Walkersville MD). Between day 7 and 12 post-electroporation, single cells were deposited one per well into a 96 well plate (coated with laminin 521 and containing TI media with Y27632) using a Hana Single Cell Dispenser instrument (Namocell, San Jose CA). Cells were allowed to grow until day 21, and then colonies were screened by PCR genotyping (see 11,535,850). Two clones that were homozygous for the edit were found.

### Example 11

This example illustrates culturing of bovine embryonic stem cells.

Bovine ESC lines were established and cultured essentially as described (Soto, D. A., et al., 2021, Sci Rep-uk, 11, 11045). The line gbESC_H2682 was cultured in NBFR+Activin A media (NBFRA, composition in Table 1) on Vitronectin coated plates. Cells were passaged using either ReLeSR (Stemcell Technologies) or 0.5 mM EDTA in PBS, and the ROCK inhibitor Y27632 (10 micromolar) was included during passaging.

TABLE 1

| | |
|---|---|
| Reagent | 250 mL |
| DMEM/F12 Medium | 100 mL |
| BSA stock 100 mg/mL | 25 mL |
| N-2 Supplement | 1.25 mL |
| Neurobasal Medium | 125 mL |
| B-27 Supplement | 2.5 mL |
| MEM Non-essential Amino Acid Solution | 2.5 mL |
| 100x | |
| GlutaMAX Supplement 100x | 2.5 mL |
| Penicillin-Streptomycin 100x | 2.5 mL |
| 2-Mercaptoethanol 1 M Stock | 25 μL |
| Recombinant Human FGF-basic | 20 ng/mL |
| Endo-IWR1 | 2.5 μM |
| Activin A | 20 ng/mL |

This example illustrates culture conditions of bovine ESCs used in methods of the present teachings.

### Example 12

This example illustrates optimization of bovine embryonic stem cell electroporation.

An optimization experiment was performed to find conditions allowing efficient electroporation of the bovine ESC using the Amaxa 4D-NUCLEOFECTOR® and P3 NUCLEOFECTOR® kit (Lonza, Walkersville, MD). Cells from line gbESC_H2682 were harvested from one well of a 6 well plate via ACCUTASE™ treatment (Innovative Cell Technologies, San Deigo, CA, distributed by Life Technologies, Grand Island, NY), washed with PBS (Life Technologies, Grand Island NY), and resuspended in 400 μl of P3 solution plus 8 μg of pmaxGFP vector. 20 μl of the solution was dispensed into each well of a 16-well electroporation cassette. The P3 optimization program was performed (according to manufacturer instructions) and cells were incubated overnight in a 24 well plate coated with vitronectin in NBFRA media+Y27632. The following day cells were observed by fluorescence microscopy for viability and green fluorescence. Several electroporation programs resulted in prominent green fluorescence with good cell viability compared to the no pulse control, including programs CM138, DN100, and EH100.

### Example 13

This example illustrates gene editing of bovine embryonic stem cells.

Test edits were performed in the ROSA and CD18 loci. CRISPR guides targeting the CD18 (ITGB2) gene at chr1:144,776,517, or the ROSA safe harbor locus region chr22:17,210,883-17,210,982, (genome assembly ARS-UCD1.2/bosTau9) were designed and synthesized as sgRNA molecules (IDT, Coralville IA) as shown in Table 2.

TABLE 2

| SEQ ID NO | Name | Guide Sequence of sgRNA |
|---|---|---|
| 105 | bROSA1 | TGTCGAGTCTCGATTATGGG |
| 106 | bROSA2 | ATAATCGAGACTCGACATGG |
| 107 | bROSA3 | AGGCGATGACGAGATCGCGG |
| 108 | CD18tgt16 | CCCCGTGGGACTCACCGGAC |

Ribonucleoprotein complexes formed from 20 pmol SpCas9 (IDT, Coralville IA) and 60 pmol sgRNA were electroporated into gbESC_H2682 cells using P3 nucleofection solution and the Amaxa 4D program CM138. After four days DNA from the cells was harvested using QUICKEXTRACT™ reagent (Lucigen, Middleton WI), and PCR was performed using the primers in Table 3.

TABLE 3

| SEQ | | |
|---|---|---|
| ID | | |
| NO | Name | Sequence |
| 109 | CD18 Ex2 Flank F | CTT CCA AGG GCA ACA ATG AG |
| 110 | CD18 Ex2 Flank R | GTA AGG GAG ACT GAC CTG TG |
| 111 | bROSA-F2 | TGCCTGAAGGACAAGACTA |
| 112 | bROSA-R2 | ACCTCTCTCTCAATCTCCTG |

Sanger sequencing (Functional Biosciences, Madison WI) showed the presence of overlapping chromatograph traces beginning in the region of the guide RNA cut sites, indicating the presence of gene editing induced indels. Analysis of the overlapping traces by TIDE (tide.nki.nl) (Brinkman, E. K., et al., 2014, Nucleic Acids Res, 42, e168-e168) showed editing frequency at the CD18 locus of around 35% and editing frequency at the ROSA locus between 58-74% (Table 4).

TABLE 4

| Edit | Guide sequence | Efficiency (%) |
|---|---|---|
| CD18 | 754: CCCCGTGGGACTCACCGGAC | 34.9 |
| ROSA | 748: TGTCGAGTCTCGATTATGGG | 74.3 |
| ROSA | 749: ATAATCGAGACTCGACATGG | 66.5 |
| ROSA | 750: AGGCGATGACGAGATCGCGG | 58.2 |

This example illustrates that bovine embryonic stem cell lines of the present teachings can be used for gene editing.

### Example 13

This example illustrates the development of a chemically defined, minimal media suitable for commercial quantities of embryonic stem cells.

A simple defined media was formulated to test porcine ESC growth, the components are shown in Table 5.

TABLE 5
Composition simple defined media # 1

| DMEM/F12 + HEPES, minus | |
|---|---|
| L-glutamine (Hyclone SH30126.01) | |
| Ascorbic acid 2-phosphate (Sigma 49752) | 200 μg/mL |
| Insulin (2% Gibco ITS-G 4140045) | 20 μg/mL |
| Transferrin (2% Gibco ITS-G 4140045) | 11 μg/mL |
| Sodium selenite (2% Gibco ITS-G 4140045) | 13.4 ng/mL |
| Recombinant Human bFGF | 100 ng/mL |
| (Stemcell Technologies 78134) | |
| TGFb1 (Peprotech 100-21) | 1 ng/mL |
| Glutamax (Gibco 35050061) | 1% |
| Endo-IWR1 (Stemcell Technologies 72564) | 2.5 μM |

Two porcine ESC lines C2 (Example 2) and C9 (Example 3) were passaged in TI media (mTeSR™ plus with Endo-IWR and Y27632 during passaging) on Laminin 521 as usual but were changed to simple defined media #1 the following day. Both lines grew more slowly than in TI, but after 12 days and two more passages in the simple media both lines had adapted and were growing reliably. At the next passage the lines expanded 5.8- and 13.2-fold over four days for lines C2 and C9 respectively. After one more passage the cells were stained for the stem cell marker alkaline phosphatase, which showed that most cells were AP positive.

This example illustrates that the simple defined media #1 allows the growth of these porcine stem cells, although with lower performance than in the TI condition.

### Example 14

This example illustrates a second simple defined media and the effects of various additives on its ability to promote stem cell growth.

A revised simple defined media #2 (Table 6, with lower ascorbic acid and FGF2) was used to determine any immediate benefit of certain additives over the timeframe of one passage.

TABLE 6
Composition simple defined media #2

| DMEM/F12 + HEPES, minus | |
|---|---|
| L-glutamine (Hyclone SH30126.01) | |
| Ascorbic acid 2-phosphate (Sigma 49752) | 50 μg/mL |
| Insulin (2% Gibco ITS-G 4140045) | 20 μg/mL |
| Transferrin (2% Gibco ITS-G 4140045) | 11 μg/mL |
| Sodium selenite (2% Gibco ITS-G 4140045) | 13.4 ng/mL |
| Recombinant Human bFGF (Stemcell Technologies 78134) | 50 ng/mL |
| TGFb1 (Peprotech 100-21) | 2 ng/mL |
| Glutamax (Gibco 35050061) | 1% |
| Endo-IWR1 (Stemcell Technologies 72564) | 2.5 μM |

Porcine stem cell line C2 (see Example 2) growing in the standard TI media conditions was passaged at a 1:80 ratio into a laminin 521 coated 24 well plate containing simple defined media #2 alone or with the addition of 1% bovine serum albumin, 1% Albumax, 1 mg/mL polyvinyl acetate, 1:500 chemically defined lipids (Gibco), 1:500 lipid mixture solution (Peprotech), 1:1000 Trace Elements B or C (Corning), sodium bicarbonate, and double the amount of ITS (insulin/transferrin/selenium). Cells were incubated at 38.5° C./5% 02 and media and additives were changed daily. On day five cell growth was measured using the CELLTITER-GLO® 2.0 kit (Promega, Madison WI). The results are shown in Table 7 and indicate that both lipids and protein (BSA or Albumax) are beneficial to cell growth.

TABLE 7
Single passage cell growth in media #2 with additives

| | Cell Growth |
|---|---|
| | (% of simple |
| Condition | media) |
| Simple defined media #2 | 100 |
| + 1% BSA (Gibco A10008-01) | 187 |
| + 1 % Albumax II (Gibco 11021-029) | 164 |
| + PVA (Sigma 363138) | 64 |
| + Sodium bicarbonate (24 mM final, Sigma | 75 |
| S5761) | |
| + Gibco lipid 1:500 (Gibco 11905031) | 116 |
| + Peprotech lipid 1:500 (Peprotech LM200) | 148 |
| + Trace element B 1:1000 (Corning 25-022- | 72 |
| CI) | |
| + Trace element C 1:1000 (Corning 25-023- | 98 |
| CI) | |
| + ITS (2% Gibco ITS-G 4140045) | 83 |

This example illustrates that additives such as lipids and proteins can increase stem cell growth.

### Example 15

This example illustrates the effect of protein or lipid on porcine stem cell growth.

The ESC line C2 was passaged at a 1:80 ratio in TI+Y27632 onto laminin 521 coated plates but media was changed to simple defined media #3 (Table 8) the following day.

### Composition Simple Defined Media #3

| DMEM/F12 + HEPES, minus L-glutamine (Hyclone | |
|---|---|
| SH30126.01) | |
| Ascorbic acid 2-phosphate (Sigma 49752) | 50 μg/mL |
| Insulin (2% Gibco ITS-G 4140045) | 10 μg/mL |
| Transferrin (2% Gibco ITS-G 4140045) | 5.5 μg/mL |
| Sodium selenite (2% Gibco ITS-G 4140045) | 6.7 ng/mL |
| Recombinant Human bFGF (Stemcell Technologies 78134) | 50 ng/mL |
| TGFb1 (Peprotech 100-21) | 1 ng/mL |
| Glutamax (Gibco 35050061) | 1% |
| Endo-IWR1 (Stemcell Technologies 72564) | 2.5 μM |
| Sodium bicarbonate (Sigma S5761) | 24 mM final |

On day 5 the cells were passaged at a 1:40 ratio in simple defined media #3+Y27632 onto laminin 521 coated plates, and passaged again at a 1:30 ratio on day 9 into the simple media with the addition of protein and lipid at different concentrations. Bovine serum albumin (Gibco A10008-01) was tested at levels of 0.25%, 0.5%, and 0.75% in conjunction with chemically defined lipids (Gibco 11905031) at levels of 1:300, 1:200, and 1:100. To determine if other protein sources may improve growth, a sample of whey protein isolate plus soy lecithin (Glanbia 1062115) was also tested at levels of 0.25% and 0.5%. On day 12 (after three full days of exposure to the additives and daily media changes) the comparative cell growth was averaged from duplicate wells by using CELLTITER-GLO® 2.0 reagent (Promega, Madison WI). The results are shown in Table 9 and indicate that the lowest combination of 0.25% BSA plus 1:300 defined lipid gave the best growth, with a 7.7-fold increase compared to the simple media. The whey protein isolate plus soy lecithin was also beneficial and gave a five-fold improvement in growth.

TABLE 9
Growth improvement in media #3 from protein and lipid

| | Cell Growth |
|---|---|
| | (% of simple |
| Condition | media) |
| simple defined media #3 | 100 |
| 0.25% Whey protein isolate + lecithin | 545 |
| 0.5% Whey protein isolate + lecithin | 462 |
| 0.25% BSA/1:300 Lipid | 774 |
| 0.25% BSA/1:200 Lipid | 700 |
| 0.25% BSA/1:100 Lipid | 604 |
| 0.5% BSA/1:300 Lipid | 763 |
| 0.5% BSA/1:200 Lipid | 698 |
| 0.5% BSA/1:100 Lipid | 690 |
| 0.75% BSA/1:300 Lipid | 714 |
| 0.75% BSA/1:200 Lipid | 675 |
| 0.75% BSA/1:100 Lipid | 686 |

This example illustrates that sources of lipid and/or protein greatly increases cell growth in porcine embryonic stem cells.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NOs expressed in a DNA sequence format (e.g., reciting T for thymine), but it can refer to either a DNA sequence that corresponds to an gRNA nucleic acid sequence, or the RNA sequence of a RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

All patents, patent applications, provisional applications, and publications referred to or cited herein are incorporated by reference in their entirety, including all FIGURES and tables, to the extent they are not inconsistent with the explicit teachings of this specification.

1. A method for improving a trait in terrestrial or aquatic livestock comprising:
(a) contacting embryonic stem cells obtained from terrestrial or aquatic livestock with an effective amount of a gene editing reagent to obtain gene edited embryonic stem cells comprising a desired gene edit;
(b) producing an embryo from the gene edited embryonic stem cells wherein at least some cells of the embryo comprise the desired gene edit.

2. The method according to claim 1, wherein the embryonic stem cells are obtained from a non-human mammal; and further comprising:
(c) implanting the embryo into a surrogate mother.

3. The method according to claim 1, wherein the embryonic stem cells are obtained from fish; and further comprising:
(c) culturing the embryo in conditions suitable for hatching.

4. The method according to claim 1, wherein the producing an embryo comprises contacting one or more gene edited embryonic stem cells obtained in (a) with an oocyte.

5. A method for establishing a gene edited cell line with an improved trait in terrestrial or aquatic livestock comprising:
(a) contacting embryonic stem cells obtained from terrestrial or aquatic livestock with an effective amount of a gene editing reagent to obtain gene edited embryonic stem cells comprising a desired gene edit;
(b) producing a gene edited embryonic stem cell line from the gene edited embryonic stem cells.

6. The method according to claim 5, further comprising (c) producing an embryo from one or more gene edited embryonic stem cells obtained from the gene edited embryonic stem cell line produced in (b).

7. The method according to claim 5, wherein the embryonic stem cells are obtained from cattle, pigs, or fish.

8. (canceled)

9. (canceled)

10. The method according to claim 5, wherein the desired gene edit is in an ANP32, ANPEP, TMPRSS2, TMPRSS4, NANOS2, CD163, Melanocortin-4 receptor (MC4R), HMGA, IGF2, HAL, Mx1, BAT2, EHMT2, PRDM1, PRDM14, or ESR gene.

11. The method according to claim 1, wherein the desired gene edit is in a PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, or TIGAR gene.

12. (canceled)

13. The method of claim 7, wherein the desired gene edit affects a trait comprising high altitude adaptation and response to hypoxia, cold acclimation, body size and stature, resistance to disease and bacterial infection, reproduction, milk yield and components, growth and feed efficiency, or polled phenotype.

14. The method according to claim 5, wherein the gene editing reagent comprises a meganuclease, a zinc finger nuclease (ZFN), a transcription-activator like effector nuclease (TALEN) or at least one guide RNA (gRNA).

15. (canceled)

16. The method according to claim 14, wherein the gene editing reagent further comprises a CAS protein.

17. (canceled)

18. The method according to am claim 5, wherein the desired gene edit comprises a deletion, a nucleotide substitution that encodes a substituted amino acid, or a premature stop codon.

19. The method a of claim 5, wherein the contacting embryonic stem cells obtained from terrestrial or aquatic livestock with an effective amount of a gene editing reagent comprises electroporation or nucleofection.

20. An embryo produced by the method of claim 6.

21. A line of embryonic stem cells produced by the method of claim 5.

22. The line of embryonic stem cells according to claim 21, wherein the cells are grown under feeder free conditions.

23. The method according to claim 1, wherein the embryonic stem cells are obtained from cattle, pigs, or fish.

24. The method according to claim 1, wherein the desired gene edit is in an ANP32, ANPEP, TMPRSS2, TMPRSS4, NANOS2, CD163, Melanocortin-4 receptor (MC4R), HMGA, IGF2, HAL, Mx1, BAT2, EHMT2, PRDM1, PRDM14, ESR, PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, or TIGAR gene.

25. The method of claim 23, wherein the desired gene edit affects a trait comprising high altitude adaptation and response to hypoxia, cold acclimation, body size and stature, resistance to disease and bacterial infection, reproduction, milk yield and components, growth and feed efficiency, or polled phenotype.

26. The method according to claim 1, wherein the gene editing reagent comprises a CAS protein, a meganuclease, a zinc finger nuclease (ZFN), a transcription-activator like effector nuclease (TALEN) or at least one guide RNA (gRNA).

27. The method according to claim 1, wherein the desired gene edit comprises a deletion, a nucleotide substitution that encodes a substituted amino acid, or a premature stop codon.

28. The method according to claim 1, wherein the contacting embryonic stem cells obtained from terrestrial or aquatic livestock with an effective amount of a gene editing reagent comprises electroporation or nucleofection.

29. An embryo produced by the method of claim 1.
